SEQUENCE LISTING (192 sequences)

SEQ ID NO: 1 - protein, 26 aa, synthetic construct
  REGION 1..26: note = Av3bM170
KSCCPCYWGGCPWGQNCYPEGCGGPG

SEQ ID NO: 2 - protein, 27 aa, Anemonia viridis
RSCCPCYWGGCPWGQNCYPEGCSGPKV

SEQ ID NO: 3 - protein, 26 aa, synthetic construct
  REGION 1..26: note = Av3b
KSCCPCYWGGCPWGQNCYPEGCSGPK

SEQ ID NO: 4 - protein, 4 aa, synthetic construct
  REGION 1..4: note = IGER
IGER

SEQ ID NO: 5 - protein, 5 aa, synthetic construct
  REGION 1..5: note = EEKKN
EEKKN

SEQ ID NO: 6 - protein, 8 aa, synthetic construct
  REGION 1..8: note = ETMFKHGL
ETMFKHGL

SEQ ID NO: 7 - protein, 6 aa, synthetic construct
  REGION 1..6: note = ALKFLV
ALKFLV

SEQ ID NO: 8 - protein, 6 aa, synthetic construct
  REGION 1..6: note = ALKLFV
ALKLFV

SEQ ID NO: 9 - protein, 6 aa, synthetic construct
  REGION 1..6: note = IFVRLR
IFVRLR

SEQ ID NO: 10 - protein, 6 aa, synthetic construct
  REGION 1..6: note = LFAAPF
LFAAPF

SEQ ID NO: 11 - protein, 8 aa, synthetic construct
  REGION 1..8: note = ALKFLVGS
ALKFLVGS

SEQ ID NO: 12 - protein, 8 aa, synthetic construct
  REGION 1..8: note = ALKLFVGS
ALKLFVGS

SEQ ID NO: 13 - protein, 8 aa, synthetic construct
  REGION 1..8: note = IFVRLRGS
IFVRLRGS

SEQ ID NO: 14 - protein, 8 aa, synthetic construct
  REGION 1..8: note = LFAAPFGS
LFAAPFGS

SEQ ID NO: 15 - protein, 8 aa, synthetic construct
  REGION 1..8: note = LFVRLRGS
LFVRLRGS

SEQ ID NO: 16 - protein, 6 aa, synthetic construct
  REGION 1..6: note = LGERGS
LGERGS

SEQ ID NO: 17 - protein, 681 aa, Bacillus thuringiensis
MGGIDMNPYQNKNEYEIFNAPSNGFSKSNNYSRYPLANKPNQPLKNTNYKDWLNVCQDNQQYGNNAGNFASSETIVGVSAGIIVVGTMLGAFAAPVLAAGIISFGTLLPIFWQGSDPANVWQDLLNIGGRPIQEIDKNIINVLTSIVTPIKNQLDKYQEFFDKWEPARTHANAKAVHDLFTTLEPIIDKDLDMLKNNASYRIPTLPAYAQIATWHLNLLKHAATYYNIWLQNQGINPSTFNSSNYYQGYLKRKIQEYTDYCIQTYNAGLTMIRTNTNATWNMYNTYRLEMTLTVLDLIAIFPNYDPEKYPIGVKSELIREVYTNVNSDTFRTITELENGLTRNPTLFTWINQGRFYTRNSRDILDPYDIFSFTGNQMAFTHTNDDRNIIWRAVHGNIISQDTSKVFPFYRNKPIDKVEIVRHREYSDIIYEMIFFSNSSEVFRYSSNSTIENNYKRTDSYMIPKQTWKNEEYGHTLSYIKTDNYIFSVVRERRRVAFSWTHTSVDFQNTIDLDNITQIHALKTLKVSSNSKIVKGLGHTSENLVILKDSMNFRVRFLKNVSQQYQVRIRYATNAPKTTVFLTGIDTISVELPSTTSRQNPNATDLTYADFGYVTFSRTVSNKTFEGEDTFINDLYYGTPNHSYNIYIDKIEFIPITQSVLDYTEKQNIEKTQKIVNDLFVN

SEQ ID NO: 18 - protein, 109 aa, Bacillus thuringiensis
FAVYTILDIHYANKPNQPLKNTNYKDWLNVCQDNQQYGNNAGNFASSETIVGVSAGIIVVGTMLGAFAAPVLAAGIISFGTLLPIFWQGSDPANVWQDLLNIGGRPIQE

SEQ ID NO: 19 - protein, 69 aa, Bacillus thuringiensis
MEVRTQTFYQNPNNEPIAPRDIINQILTAPAPADLFFKNADINVKFTQWFQSTLYGWNIKLGTQTVLSM

SEQ ID NO: 20 - protein, 1176 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEGQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVQLLLMEE

SEQ ID NO: 21 - protein, 1156 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHERLGNLEFLEGRAPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 22 - protein, 1181 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYKKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVGIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENCVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 23 - protein, 859 aa, Bacillus thuringiensis
MDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 24 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 25 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHRIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNSSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKRETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYGRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 26 - protein, 1181 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGLSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSRYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENSTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVDLLLMEE

SEQ ID NO: 27 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGNMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 28 - protein, 793 aa, Bacillus thuringiensis
MQCVPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFRAWEADPTNPALRVEMRIQFNDMNSALTTAIPLFAVQNYQVPLLSVYVQAANLHLSVLRDVSVFGQRWGFDATTINSRYNDLTRLIGNYTDYAVRWYNTGLERVWGPDSRDWIRYNQFRRELTLTVLDIVSLFPNYDSRTYPIRTVSQLTREIYTNPVLEDFNGSFRGSAQGIEQSIRSPHLMDILNSITIYTDAHRGYYYWSGHQIMASPVGFSGPEFTFPLYGTMGNAAPQQRIVAQLGQGVYRTLSSTFYRSPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTAGFTTPFNFSNGSSVFTLSAHVFNSGNEVYIDRIEFVPAEVTFEAEYDLERAQKAVNALFTSSNQIGLKTDVTDYHIDQVSNLVECLSDEFCLDEKQELSEKVKHAKRLSDERNLLQDPNFRWINRQLDRGWRGSTDITIQGGDDVFKENYVTLPGTFDECYPTYLYQKIDESKLKAYTRYQLRGYIEDSQDLEIYLIRYNAKHETVNVPGTGSLWPLSAQSPIGRCGEPNR

SEQ ID NO: 29 - protein, 373 aa, Bacillus thuringiensis
RDMTVIVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDKIEFIP

SEQ ID NO: 30 - protein, 1177 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDHAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTFYRRSFNIGINNQQLSVLDGTEFAYGTSSNLPSAVYRKSGTVDSLDEIPPQNNNVPPRQGFSHRLSHVSMFRSGSSSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLYVNWGNSSIFSNTVPATATSLDNLQSSDFGYFESANAFTSSLGNIVGVRNFSGTAGVIIDRFEFIPVTATLEAEYNLERAQKAVNALFTSTNQLGLKTNVTDYHIDQVSNLVTCLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYLIRYNAKHETVNVSGTGSLWPLSAQSPIGKCGEPNRFAPHLEWNPDLDCSCRDGEKCAHHSHHFSLDIDVGCTDLNEDLGVWVIFKIKTQDGHARLGNLEFLGEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVPQEVRVCPGRGYILRVTAYKEGYGEGCVTIHEIENNTDELKFSNCVEEEVYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVQLLLMEE

SEQ ID NO: 31 - protein, 609 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDHAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTFYRRPFNIGINNQQLSVLDGTEFAYGTSSNLPSAIYRKSGTVDSLDEIPPQNNNVPPRQGFSHRLSHVSMFRSGSSSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLYVNWGNSSIFSNTVPATATSLDNLQSSDFGYFESANAFTSSLGNIVGVRNFSGTAGVIIDRFEFIPVTA

SEQ ID NO: 32 - protein, 609 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDHAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTFYRRPFNIGINNQQLSVLDGTEFAYGTSSNLPSAVYRKSGTVDSLDEIPPQNNNVPPRQGFSHRLSHVSMFRSGSSSSVSIIRAPMFSWIHRSAEFNNIIASDSITQIPAVKGNVLFNGSVISGPGFTGGDLVRLNSSGNNIQNRGYIEVPIHFPSTSTRYRVRVRYASVTPIHLYVNWGNSSIFSNTVPATATSLDNLQSSDFGYFESANAFTSSLGNIVGVRNFSGTAGVIIDRFEFIPVTA

SEQ ID NO: 33 - protein, 1171 aa, Bacillus thuringiensis
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATFEEEYDLERAQKAVNALFTSTNQLGLKTDVTDYHIDQVSNLVECLSDEFCLDEKRELSEKVKHAKRLSDERNLLQDPNFRGINRQPDRGWRGSTDITIQGGDDVFKENYVTLPGTFDECYPTYLYQKIDESKLKAYTRYELRGYIEDSQDLEIYLIRYNAKHETVNVPGTGSLWPLSAQSPIGKCGEPNRCAPHLEWNPNLDCSCRDGEKCAHHSHHFSLDIDVGCTDLNEDLGVWVIFKIKTQDGYARLGNLEFLEEKPLLGEALARVKRAEKKWRDKCEKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIEDNTDELKFSNCVEEEVYPNNTVTCNNYTATQEEHEGTYTSRNRGYDEAYESNSSVHASVYEEKSYTDRRRENPCESNRGYGDYTPLPAGYVTKELEYFPETDKVWIEIGETEGTFIVDSVELLLMEE

SEQ ID NO: 34 - protein, 1168 aa, Bacillus thuringiensis
MKNNIQNQCVPYNCLSNPEVEILSEERSTGRLPLDISLSLTRFLLSEFVPGVGVAFGLFDLIWGFITPSEWSLFLLQIEQLIEQRIETLERNRAITTLRGLADSYEVYLEALREWEENPNNAQLREDVRIRFANTDDALITAINNFTLTSFEIPLLSVYVQAANLHLSLLRDAVSFGQGWGLDIATVNNHYNRLINLIHRYTEHCLDTYNQGLENLRGTNTRQWSRFNQFRRELTLTVLDIVALFPNYDARAYPIQTSSQLTREIYTSSVIEDSPVSANIPNGFNRAEFGVRPPHLMDFMNSLFVTAETVRSQTVWGGHLVSSRNTAGNPINFPIYGVFNPGGAIWIADEDPRPFYRTLSDPVFVRGGFGDPHYVLGLRGVGFQQTGTNHTRTFRNSGTIDSLDEIPPQDNSGAPWNDYSHVLNHVTFVRWPGEIAGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGDNLESGNFRTAGFSTPFSFSNAQSTFTLGTQAFSNQEVYIDRIEFVPAEVTFEAESDLERAQKAVNALFTSTSQLGLKTNVTGYHIDQVSNLVACLSDEFCLDEKRELSEKVKHAKRLSDKRNLLQDPNFRGINRQPDHGWRGSTDITIQGGDDVFKENYVTLPGTFDECYPTYLYQKIDESKLKAYTRYQLRGYIEDSQDLEIYLIRYNSKHEIVNVPGTGSLWPLSVENQIGPCGEPNRCAPHLEWNPDLHCSCRDGEKCVHHSHHFSLDIDVGCTDLNEDLGVWLIFKIKTQDGHARLGNLEFLEEEPLLGEALGRVKRAEKKWRDKREKLQLETNIVYKEAKESVDALFVNSQYDRLQADTNIAMIHAADKRVHRIREAYLPELSVIPGVNAAIFEELEGRIFTAYSLYDARNVIKNGNFNNGLLCWNVKGHVDVEEQNNHRSVLVVPEWEAEVSQEVRVCPGRGYILRVTAYKEGYGEGCVTIHEVDNNTDELKFSNCEKEQVYPGNTVACNDYNKNHGANACSSRNRGYDESYESNSSIPADYAPVYEEEAYTDGQRGNPCEFNRGHTPLPAGYVTAELEYFPETDTVWVEIGETEGTFIVDSVELLLMEE

SEQ ID NO: 35 - protein, 719 aa, Bacillus thuringiensis
MKLKNPDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQIYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFSTQPDNGVPRVDFHWKFVTHPIASDNFYYPGYAGIGTQLQDSENELPPEATGQPSYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 36 - protein, 722 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYPDHCVKWYSTGLNNLRGTNAESWVRYNQFRRDMTLMVLDLVALLPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNMVDA

SEQ ID NO: 37 - protein, 717 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGEPWPKGKNQWEIFMEHVEEIINQKISTYARNKALTDLKGLGDALAVYHDSLESWVGNRNNTRARSVVKSQYIALELMFVQRLPSFAVSGEEVPLLPIYAQAANLHLLLLRDASIFGKEWRLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRSPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGYIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDSEKAQEKVTALFTSTNPRGLKTDVKDYHIDQVSNLVESLSDEFYLDEKRELFEIVKYAKQLHIER

SEQ ID NO: 38 - protein, 722 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKSQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNHQVERAGDYSDHCVKWYSTGLNNLRGTNAESWVRYNQFRRDMTLMVLDLVALFPSYDIQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNMVDA

SEQ ID NO: 39 - protein, 719 aa, Bacillus thuringiensis
MKLKNPDKHQSLSSNAKVDKIATDSLKNETDIELKNMNNEDYLRMSEHESIDPFVSASTIQTGIGIAGKILGTLGVPFAGQIASLYSFILGELWPKGKSQWEIFMEHVEEIINQKILTYARNKALSDLRGLGDALAVYHESLESWVENRNNTRARSVVKNQYIALELMFVQKLPSFAVSGGEVPLLPIYAQAANLHLLLLRDASIFGKEWGLSASEISTFYNRQVERTRDYSDHCIKWYNTGLNNLRGTNAKSWVRYNQFRKDMTLMVLDLVALFPSYDTLVYPIKTTSQLTREVYTDAIGTVHPNQAFASTTWYNNNAPSFSAIEAAVIRSPHLLDFLEKVTIYSLLSRWSNTQYMNMWGGHRLESRPIGGALNTSTQGSTNTSINPVTLQFTSRDVYRTESWAGLNLFLTQPVNGVPRVDFHWKFPTLPIASDNFYYLGYAGVGTQLQDSENELPPETTGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFSDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQIHIERNM

SEQ ID NO: 40 - protein, 719 aa, Bacillus thuringiensis
MKLKNPDKHQTLSSNAKVDKIATDSLKNETDIELKNMNNEDYLRMSEHESIDPFVSASTIQTGIGIAGKILGTLGVPFPGQIASLYSFILGELWPKGKSQWEIFMEHVEAIINRKISTYARNKALTDLKGLGDALAVYHESLESWVGNRNNTRARSVVKNQYIALELMFVQKLPSFAVSGEEVPLLPIYAQAANLHLLLLRDASIFEKNGGLSASEISTFYNRQVERTRDYSYHCVKWNNTGLNNLRATNGQSWVRYNQFRKDIELMVLDLVRVFPSYDTLVYPIKTTSQLTREVYTDAIGTVDPNQALRSTTWYNNNAPSFSAIEAAVIRSPHLLDFLEKVTIYSLLSRWSNTQYMNMWGGHRLESRPIGGALNTSTQGSTNTSINPVTLQFTSRDFYRTESWAGLNLFLTQPVIGVPRVDFHWKFPTLPIASDNFYYLGYAGVGTQLQDSENELPPETTGQPNYESYSHRLSHIGLISASHVKALVYSWTHRSADRTNTIEPNSITQIPLVKAFNLSSGAAVVRGPGFTGGHILRRTKSGTFGHIRVNINPPFAQRYRVRMSYASTTDLQFHTSINGKAINQGNFSATMNRGEDLDYKTFRTVGFTTPFSFSDVQSTFTIGAWNFSSGNEVYIGRIEFVPVEVTYEAEYDFEKAQEKVTALFTSTNPRGLKTDVKDYHIDQVSNLVESLSDELYLDEKRELFEIVKYAKQIHIERNM

SEQ ID NO: 41 - protein, 381 aa, Bacillus thuringiensis
RDVIVIVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDKIEFIP

SEQ ID NO: 42 - protein, 1167 aa, Bacillus thuringiensis
MEINNQKQCIPYNCLSNPEEVLLDGERILPDIDPLEVSLSLLQFLLNNFVPGGGFISGLVDKIWGALRPSEWDLFLAQIERLIDQRIEATVRAKAITELEGLGRNYQIYAEAFKEWESDPDNEAAKSRVIDRFRILDGLIEANIPSFRIIGFEVPLLSVYVQAANLHLALLRDSVIFGERWGLTTKNVNDIYNRQIREIHEYSNHCVDTYNTELERLGFRSIAQWRIYNQFRRELTLTVLDIVALFPNYDSRLYPIQTFSQLTREIVTSPVSEFYYGVINSGNIIGTLTEQQIRRPHLMDFFNSMIMYTSDNRREHYWSGLEMTAYFTGFAGAQVSFPLVGTRGESAPPLTVRSVNDGIYRILSAPFYSAPFLGTIVLGSRGEKFDFALNNISPPPSTIYRHPGTVDSLVSIPPQDNSVPPHRGSSHRLSHVTMRASSPIFHWTHRSATTTNTINPNAIIQIPLVKAFNLHSGATVVRGPGFTGGDILRRTNTGTFADMRVNITGPLSQRYRVRIRYASTTDLQFFTRINGTSVNQGNFQRTMNRGDNLESGNFRTAGFSTPFSFSNAQSTFTLGTQAFSNQEVYIDRIEFVPAEVTFEAESDLERAQKAVNALFTSTNQLGLKTDVTDYQIDQVSNLVECLSDEFCLDEKRELSEKVKHAKRLSDKRNLLQDPNFTSINRQLDRGWRGSTDITIQGGNDVFKENYVTLPGTFDECYPTYLYQKIDESKLKAYTRYELRGYIEDSQDLEVYLIRYNAKHETVNVPGTGSLWPLSVESPIGRCGEPNRCVPHIEWNPDLDCSCRDGEKCAHHSHHFSLDIDVGCTDLNEDLGVWVIFKIKTQDGHARLGNLEFLEEKPLLGEALARVKRAEKKWRDKREQLQFETNIVYKEAKESVDALFVDSHYNRLQADTNITMIHAADKRVHRIREAYLPELSVIPGVNADIFEELEGLIFTAFSLYDARNIIKNGDFNNGLSCWNVKGHVDIQQNDHRSVLVVPEWESEVSQEVRVCPGRGYILRVTAYKEGYGEGCVTIHEIEDNTDELKFSNCIEEEVYPTDTGNDYTAHQGTTGCADACNSRNVGYEDGYEINTTASVNYKPTYEEEMYTDVRRDNHCEYDRGYGNHTPLPAGYVTKELEYFPETDTVWIEIGETEGTFIVDSVELLLMEE

SEQ ID NO: 43 - protein, 1170 aa, Bacillus thuringiensis
MEINNQNQCIPYNCLSNPEEVLLDGERILPDIDPLEVSMSLLQFLLNNFVPGGGFISGLFDKIWGALRPSDWELFLAQIEQLIDQRIEATVRAKAIAELEGLGRSFQLYVEAFKEWEETPDNTAARSRVTERFRIIDAQIEANIPSFRIPGFEVPLLSVYAQAANLHLALLRDSVIFGERWGLTTTNVNDIYNRQVKRIHEYSDHCVDTYKTELERLGFTSRAQWKIYNQFRRELTLTVLDIVAVFPNYDGKLYPIQTKSELTREIYTSPVSEYYYGAINNYNQNGIQTERQIRQPHLMDFFNTMTMYTSYNRREYYWSGLEMTAYFTGFAGPQVSFPLAGTRGDAAPPFNVRSVNDGIYRILSAPFYSAPFLGTSVLGSRGEEFMFALNNISPPPSARYRNPGTVDSLVSIPPQDNSVPPHRGSSHRLSHVTMRNSSPIFHWTHRSATTTNRINSDVITQIPMVKAYNLHAGATVVRGPGFTGGDILRRTSNGMVVTLRVDASAVRNQRYRIRFRYAATSNFYFVVRRGNLGVNGREIMKTMSTGEELKSASFVLGEFITPFNFFENQVPLQIEIQSLSPGGEVYLDKIEFIPADTTFEAEYDLERAQKAVNALFTSTNQRGLKTDVTDYHIDQVSNLVECLSDEFCLDEKRELSEKVKHAKRLSDERNLLQDPNFTSINGQLDRGWRGSTDITIQGGNDVFKENYVTLPGTFDECYPTYLYQKIDESKLKAYTRYELRGYIEDSQDLEVYLIRYNAKHETLNVPGTDSLRTLSVESQNGRCGELNRCMPHIKWNPDVDCSCRDGEKCAHHSHHFSLDIDVGCTDLQEDLGVWVVFKIKTQEGYARLGNLEFIEEKPLVGEALSRVKRAEKKWRDKREKLELETKRVYTEAKEAVDALFVDSQYDRLQADTNIGMIHAADKLVHRICETYLPELPFIPGINAIIFEELENRISTAFFLYEARNVINNGDFNNGLTCWNVKGHVDVQQSHHRSVLVIPEWEAEVSQKVRVCPGRGYILRVTAYKEGYGEGCVTIHEIEDNTDELKFRNCEEEGDYSNDTGTCNDYPASQGAAGCADVCNSRNVGYKDAYETNTSASVNYKPTYEEETYTDVREDNHCEYDRGYVNYPPLPAGYVTKELEYFPETDTVWIEIGETEGKFIVDSVELLLMEE

SEQ ID NO: 44 - protein, 1168 aa, Bacillus thuringiensis
MEINNQNQCIPYNCLSNPEEVFLDGERILPDIDPLEVSLSLLQFLLNNFVPGGGFISGLLDKIWGALRPSDWELFLAQIEQLIDRRIERTVRAKAIAELEGLGRSYQLYGEAFKEWEKTPDNTAARSRVTERFRIIDAQIEANIPSFRVSGFEVPLLLVYTQAANLHLALLRDSVVFGERWGLTTTNVNDIYNRQVNRIGEYSKHCVDTYKTELERLGFRSIAQWRIYNQFRRELTLTVLDIVAVFPNYDSRLYPIRTISQLTREIYTSPVSEFYYGVINSNNIIGTLTEQQIRRPHLMDFFNSMIMYTSDNRREHYWSGLEMTATNTEGHQRSFPLAGTIGNSAPPVTVRNNGEGIYRILSEPFYSAPFLGTSVLGSRGEEFAFASNTTTSLPSTIYRNRGTVDSLVSIPPQDYSVPPHRGYSHLLSHVTMRNSSPIFHWTHRSATPRNTIDPDSITQIPAVKGAYIFNSPVITGPGHTGGDIIRFNPNTQNNIRIPFQSNAVQRYRIRMRYAAEADCILESGVNIVTGAGVTFRPIPIKATMTPGSPLTYYSFQYADLNINLTAPIRPNNFVSIRRSNQPGNLYIDRIEFIPIDPIREAEHDLERAQKAVNALFTSSNQLGLKTDVTDYHIDQVSNLVACLSDEFCLDEKRELSEKVKHAKRLSDERNLLQDQNFTGINRQVDRGWRGSTDITIQGGNDVFKENYVTLPGTFDECYPTYLYQKIDESKLKPYTRYELRGYIEDSQDLEVYLIRYNAKHETLNVPGTGSLWPLAAESSIGRCGEPNRCAPHIEWNPELDCSCRDGEKCAHHSHHFSLDIDVGCTDLNEDLGVWVIFKIKTQDGYARLGNLEFLEEKPLLGEALARVKRAEKKWRDKRDKLEWETNIVYKEAKESVDALFVDSQYNRLQTDTNIAMIHVADKRVHRIREAYLPELSVIPGVNAAIFEELEGLIFTAFSLYDARNVIKNGDFNHGLSCWNVKGHVDVEEQNNHRSVLVVPEWEAEVSQEVRVCPGRGYILRVTAYKEGYGEGCVTIHEIEDHTDELKFRNCEEEEVYPNNTVTCNDYPANQEEYRAAETSRNRGYGESYESNSSIPAEYAPIYEKAYTDGRKENSCESNRGYGNYTPLPAGYVTKELEYFPETDKVWIEIGETEGTFIVDSVELLLMEE

SEQ ID NO: 45 - protein, 725 aa, Bacillus thuringiensis
  SITE 55: note = misc_feature - Xaa can be any naturally occurring amino acid
  SITE 76: note = misc_feature - Xaa can be any naturally occurring amino acid
  SITE 477: note = misc_feature - Xaa can be any naturally occurring amino acid
  SITE 600: note = misc_feature - Xaa can be any naturally occurring amino acid
  SITE 604: note = misc_feature - Xaa can be any naturally occurring amino acid
  SITE 619: note = misc_feature - Xaa can be any naturally occurring amino acid
  SITE 624: note = misc_feature - Xaa can be any naturally occurring amino acid
  SITE 637: note = misc_feature - Xaa can be any naturally occurring amino acid
  SITE 647: note = misc_feature - Xaa can be any naturally occurring amino acid
  SITE 653: note = misc_feature - Xaa can be any naturally occurring amino acid
  SITE 712: note = misc_feature - Xaa can be any naturally occurring amino acid
MCGFTPTRSREQVAEISLGLTRFLLENLFPGSTFGFGLIDIIWGIFGPDQWSMFXEQIEQLIDQRIETVERNRANXNINWVINSYDVYIEALKEWENNPDNSASQERVRNRFRTTDDALITGIPLLAIPNFEIATLSVYVQAANLHLSLLRDAVFFGERWGLTQINVDDLYRRLTNNIRTNSDHCARWYNEGLDNISGLSRSINFQREVTISVLDIVALFPNYDIRTYPISTTSQLTREIFTSPIVVPNDFSVAYEGVRRAPHLFEFLEKLVIYTGDRSGIRHWAGHEITSRRTDSYHGIIRYPLYGTAANAESPYTLALQPSGSIYRTLSEPIFSQTGGLSPHRRRVVEGVEFSIVNNNVNPSSFVYRRKGSLDSFTELPPEDESVPPYIGYSHQLCHVGFGRTNVIFEPSNFARVPVFSWTHRSATPTNTIDPDRITQIPSVKASSLRNSTVVSGPGFTGGDIVRMGAVHQIYAXDLSMNVRPSVALSRYLIRLRYACRGSSNIVIHGPSIRFVSLPSTMSNDEPLTYQSFRYASITTPITRPIYNMFNLSISRISGVQNLFIDRIEFIPVDANFEAERDLERAQKAVNALFTSTNQXGLKXDVTDYHIDQVSNLVXCLSDXFCLDEKRELSEKXKHAKRLSDEXNLLQDXNFTGINRQVDRGWRGSTDITIQGGNDVFKENYVTLPGTFDECYPTYLYQKIDESKLKPXTRYELRGYIEDSQ

SEQ ID NO: 46 - protein, 1170 aa, Bacillus thuringiensis; identical to SEQ ID NO: 43

SEQ ID NO: 47 - protein, 1167 aa, Bacillus thuringiensis
MTNPTILYPSYHNVLAHPIRLDSFFDPFVETFKDLKGAWEEFGKTGYMDPLKQHLQIAWDTSQNGTVDYLALTKASISLIGLIPGADAVVPFINMFVDFIFPKLFGRGSQQNAQAQFFELIIEKVKELVDEDFRNFTLNNLLNYLDGMQTALSHFQNDVQIAICQGEQPGLMLDQTPTACTPTTDHLISVRESFKDARTTIETALPHFKNPMLSTNDNTPDFNSDTVLLTLPMYTTGATLNLILHQGYIQFAERWKSVNYDESFINQTKVDLQRRIQDYSTTVSTTFEKFKPTLNPSNKESVNKYNRYVRSMTLQSLDIAATWPTLDNVNYPSNVDIQLDQTRLVFSDVAGPWEGNDNITSNIIDVLTPINTGIGFQESSDLRKFTYPRIELQSMQFHGQYVNSKSVEHCYSDGLKLNYKNKTITAGVSNIDESNQNNKHNYGPVINSPITDINVNSQNSQYLDLNSVMVNGGQKVTGCSPLSSNGNSNNAALPNQKINVIYSVQSNDKPEKHADTYRKWGYMSSHIPYDLVPENVIGDIDPDTKQPSLLLKGFPAEKGYGDSIAYVSEPLNGANAVKLTSYQVLQMEVTNQTTQKYRIRIRYATGGDTAASIWFHIIGPSGNDLTNEGHNFSSVSSRNKMFVQGNNGKYVLNILTDSIELPSGQQTILIQNTNSQDLFLDRIEFISLPSTSTPTSTNFVEPESLEKIINQVNQLFSSSSQTELAHTVSDYKIDQVVLKVNALSDDVFGVEKKALRKLVNQAKQLSKARNVLVGGNFEKGHEWALSREATMVANHELFKGDHLLLPPPTLYPSYAYQKIDESKLKSNTRYTVSGFIAQSEHLEVVVSRYGKEVHDMLDIPYEEALPISSDESPNCCKPAACQCSSCDGSQSDSHFFSYSIDVGSLQSDVNLGIEFGLRIAKPNGFAKISNLEIKEDRPLTEKEIKKVQRKEQKWKKAFNQEQAEVATTLQPTLDQINALYQNEDWNGSVHPASDYQHLSAVVVPTLPKQRHWFMEGREGEHVVLTQQFQQALDRAFQQIEEQNLIHNGNLANGLTDWTVTGDAQLTIFDEDPVLELAHWDASISQTIEIMDFEGRHRIQTACTWKRQRNSYRSTWRKRLETMTFNTTSFTTQEQTFYFEGDTVDVHVQSENNTFLIDSVELIEIIEE

SEQ ID NO: 48 - protein, 1167 aa, Bacillus thuringiensis
MTNPTILYPSYHNVLAHPIRLDSFFDPFVETFKDLKGAWEEFGKTGYMDPLKQHLQIAWDTSQNGTVDYLALTKASISLIGLIPGADAVVPFINMFVDFIFPKLFGRGSQQNAQAQFFELIIEKVKELVDEDFRNFTLNNLLNYLDGMQTALSHFQNDVQIAICQGEQPGLMLDQTPTACTPTTDHLISVRESFKDARTTIETALPHFKNPMLSTNDNTPDFNSDTVLLTLPMYTTAATLNLILHQGYIQFAERWKSVNYDESFINQTKVDLQRRIQDYSTTVSTTFEKFKPTLNPSNKESVNKYNRYVRSMTLQSLDIAATWPTLDNVNYPSNVDIQLDQTRLVFSDVAGPWEGNDNITSNIIDVLTPINTGIGFQESSDLRKFTYPRIELQSMQFHGQYVNSKSVEHCYSDGLKLNYKNKTITAGVSNIDESNQNNKHNYGPVINSPITDINVNSQNSQYLDLNSVMVNGGQKVAGCSPLSSNGNSNNAALPNQKINVIYSVQSNDKPEKHADTYRKWGYMSSHIPYDLVPENVIGDIDPDTKQPSLLLKGFPAEKGYGDSIAYVSEPLNGANAVKLTSYQVLKMEVTNQTTQKYRIRIRYATGGDTAASIWFHIIGPSGNDLTNEGHNFSSVSSRNKMFVQGNNGKYVLNILTDSIELPSGQQTILIQNTNSQDLFLDRIEFISLPSTSTPTSTNFVEPESLEKIINQVNQLFSSSSQTELAHTVSDYKIDQVVLKVNALSDDVFGVEKKALRKLVNQAKQLSKARNVLVGGNFEKGHEWALSREATMVANHELFKGDHLLLPPPTLYPSYAYQKIDESKLKSNTRYTVSGFIAQSEHLEVVVSRYGKEVHDMLDIPYEEALPISSDESPNCCKPAACQCSSCDGSQSDSHFFSYSIDVGSLQSDVNLGIEFGLRIAKPNGFAKISNLEIKEDRPLTEKEIKKVQRKEQKWKKAFNQEQAEVATTLQPTLDQINALYQNEDWNGSVHPHVTYQHLSAVVVPTLPKQRHWFMEDREGEHVVLTQQFQQALDRAFQQIEEQNLIHNGNFANGLTDWTVTGDAQLTIFDEDPVLELAHWDASISQTIEIMDFEEDTEYKLRVRGKGKGTVTVQHGEEELETMTFNTTSFTTQEQTFYFEGDTVDVHVQSENNTFLIDSVELIEIIEE

SEQ ID NO: 49 - protein, 633 aa, Bacillus thuringiensis
MNSVLNSRRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLSPLY

SEQ ID NO: 50 - protein, 633 aa, Bacillus thuringiensis
MNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSFWSVFKYQSLLVSSGANLNARGRGPQQAQSFTSPGWAFLNSLFQVNSNYVFNGFRGARLFNSFPNIIGLPGFTTTHPLLAAKVNYKGRISSGDIGASPVNQNFNCSKFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVARSNSDVSLDINVSLNSRTLIDLMNIMLVPTNISPLY

SEQ ID NO: 51 - protein, 633 aa, Bacillus thuringiensis
MNTVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNLPPLY

SEQ ID NO: 52 - protein, 633 aa, Bacillus thuringiensis
MNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAASLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNLPPLY

SEQ ID NO: 53 - protein, 633 aa, Bacillus thuringiensis
MNSVLNIGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSPGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNIPPLY

SEQ ID NO: 54 - protein, 633 aa, Bacillus thuringiensis
MNSVLNTGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 55 - protein, 633 aa, Bacillus thuringiensis
MNNVLNSRRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNLSPLY

SEQ ID NO: 56 - protein, 632 aa, Bacillus thuringiensis
MNTVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNLPPL

SEQ ID NO: 57 - protein, 633 aa, Bacillus thuringiensis; identical to SEQ ID NO: 51

SEQ ID NO: 58 - protein, 633 aa, Bacillus thuringiensis
MNSVLNNGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQSQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNIPPLY

SEQ ID NO: 59 - protein, 633 aa, Bacillus thuringiensis
MNNVLNSGRNITCHAHNVVAHDPFSFEHKSLNTIEKEWKEWKRTDHSLYVAPIVGTVGSFLLKKVGSLVGKRILSELQNLIFPSGSIDLMQEILRATEQFINQRLNADTLGRVNAELAGLQANVAEFNRQVDNFLNPNQNPVPLAIIDSVNTLQQLFLSRLPQFQIQGYQLLLLPLFAQAANLHLSFIRDVILNADEWGISAATVRTYRDHLRNFTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANEYGTMIHLAPEDYTGFTISPIHATQVNNQTRTFISEKFGNQGDSLRFEQSNTTARYTLRGNGNSYNLYLRASSIGNSTIRVTINGRAYTVSNVNTTTNNDGVNDNGARFSDINIGNIVASDNTNVTLDINVTLNSGTPFDLMNIMFVPTNLPPLY

SEQ ID NO: 60 - protein, 362 aa, Bacillus thuringiensis
MNKKSITHEEFIRQLKEYNLDNNLNYHDPAVLKKINELLPADQQYDLISPTQDWYQFKTLYPISKNGVIISSNLDDSSNVLVPELSENPYDPIPQSGKSTIQTAVRSPEALYIILTTNNSLSFGGGTNTMIATRIALLSVTRPELYQAITKVNYVYKSGQTAPRNAPVAYIELSPNNSYVQTLLNDSHMKRTSSYELVGSSIARRGIETKWSKSHTSGVSDTDSWSLAVSAGIDIEWDVGIPLTASAKEKLSLSITGTYGQSTTVSSQDTITQEYTFAKPGKDYKYDDYAYAVYQLKSNYQFIAGDAFNNLINSLSFGNQFSVHGDASYQYSTDTIFSTQTPDPTPTNEKSLIQVNFNPRFS

SEQ ID NO: 61 - protein, 609 aa, Bacillus thuringiensis
MGTWWPTDSASDTWGEMIGFAQELVGTALSEDLKIRTNQQIDSIRIALQAYYSSLEDWLNANKPLSGPLLNQVTEEFGNALRKSRDSIAYFKSDDSNVYTIILLPAYAQVANFHLALIHEGLKYATEWNLPRLQTFGYEEDLKHYTISYVNHCEYWYQKGLDILYPRNVIGMTQWMKRNFYRLNMTINVLDIISLFSLYDSKKYPNFFEDIYNAQKELISKFQLTRIVTTEPTLHQHKYLNDSSKKICQNESSCDPIDLDEYLTLPLMFQNWLRNINFQYLAPVISVGDEALYPFFVATQNINEYMNAEGNMIIGRQQGLWNFQFIIVHLFHLVYKKMTIFMVKWLSAYPLIDEDILNGVRTPYILQKIEFYNLNKSINSKQIRPISAGTTKSPLIDIYYGLPDVNGYNLDEPQFNFNAASHYFNSIQTCYYKENTSKNHYDIYQSYVFHWEHASVKRKDEVVSDRITIFPAIKSNPILSRGIQIISHQGHTGGNVIYFTPQSELHFKINFVSNRQKYKIRLRYVAFNPVVIQYHGSNSYASLSSITLPRTSSNQNVRDLRYEEFGYSDFEINMSSAGGLEDIKIISNNEFILDRIEFIPDTLFNYLSN

SEQ ID NO: 62 - protein, 837 aa, Bacillus thuringiensis
MKIYDIYPHYNVLSDAALITSYASDFRDSLKGLEEIWSKYATEGNRSAMTSYVSNIMQLYQTDSINYQALVKSAITLNLALTPLAPVSPFINMVIGFVRPFLFGSSKEVSMFDQIMDAVKKEMEHSFEQFTLNNLNDTINGLKTDLYDFTRLIDASTGVLQLPYESGEGCKPCKLGENSPCAPCYNDMIAINSKFQALTSSFNNALPHFKDPLANVIEKPNPGWNQEYITMCLPLYTLVATLELLTYQGYIQFAEKWRIPADSETSPITTTQIANMKANLRQRIQEHSNDIYTTFIKYLPKVNENATKGELNDYIRYHRVITLQCLDFVATWPFLDSRYYQSSGAATINFTRMTYLDIVGPVEDFYNDSKLDPSRDSGKLILSIKDINGKNDTNKISDVIAPLSLFYDNLELKSLKFNVYNSSWHCYINGYQGIYNSLSNDVSVSTNTNNLAKDLYLDFPLLNKLNMRSYAVGKASVDDSMVYVDDDSDVPSGNGHAIYGGCNGFYTGGPHSNNTLNYDNQIIQAIYEVTASNPNVYTYYYNADKTGYLATLSPIETTLSPIIGTDFQVINTVPAEQVISTSGTKFVEYITGSAVLELKPNQTAKYSILNQGLFQYKVRARVATTDSGSLSITMNNKTINLNIINTKNYTDPTTAFTNLKIQGKNGSYMVFPLSDAEHTTFDNVFNPNYGTVTMEVTNNSNTNIIIDRFEFIPYAPSKGTLLDTIPETIRTGGDSSILWKSTTGIGGQQVDLNIVIKDPSGIDVTTNYLLQYGSSYEFPLFSGQHSYPAVLNNEFTELGIDNCYGGNLGCNMNMPLKTIVPEGYTLTFSGNIYNNLK

SEQ ID NO: 63 - protein, 1220 aa, Bacillus thuringiensis
MAILNELYPSVPYNVLAYTPPSFLPDAGTQATPADLTAYEQLLKNLEKGINAGTYSKAIADVLKGIFIDDTINYQTYVNIGLSLITLAVPEIGIFTPFIGLFFAALNKHDAPPPPNAKDIFEAMKPAIQEMIDRTLTADEQTFLNGEISGLQNLAARYQSTMDDIQSHGGFNKVDSGLIKKFTDEVLSLNSFYTDRLPVFITDNTADRTLLGLPYYAILASMHLMLLRDIITKGPTWDSKINFTPDAIDSFKTDIKNNIKLYSKTIYDVFQKGLASYGTPSDLESFAKKKKYIEIMTTHCLDFARLFPTFDPDLYPTGSGDISLQKTRRILSPFIPIRTADGLTLNNTSIDTSNWPNYENGNGAFPNPKERILKQFKLYPSWRAGQYGGLLQPYLWAIEVQDSVETRLYGQLPAVDPQAGPNYVSIDSSNPIIQINMDTWKTPPQGASGWNTNLMRGSVSGLSFLQRDGTRLSAGMGGGFADTIYSLPATHYLSYLYGTPYQTSDNYSGHVGALVGVSTPQEATLPNIIGQPDEQGNVSTMGFPFEKASYGGTVVKEWLNGANAMKLSPGQSIGIPITNVTKHNYQVRCRYASNSDNPVFFNVDTGGANPIFQQINFASTVDSNMGVKEENGVYVVKSIKTVEIPAGSFYVHVTNQGSSDLFLDRIEFVPKIQFQFCDNNNLHCDCNNPVDTDCTFCCVCTSLTDCDCNNPRGIDCTLCCQVENQLPSFVTLTDLRNITSQVNGLFAPGTQNRLAQNISDHDIEEVVLKVDALSDEIFGTNKKALRKLVNQAKRLSRARNLLIGGSFENWDAWYKGRNVVTVSDHELFKSDHVLLPPPGLSPSYIFQKVEESKLKANTRYTVSGFIAHATDLEIVVSRYGQEIKKVVQVPYGEAFPLTSSGPVCCIPHSTSNGTLGNPHFFSYSIDVGALDVDTNPGIEFGLRIVNPTGMARVSNLEIREDRPLAANEIRQVQRVARNWRTEYEKERAEVTSLIQPVINRINGLYENENWNGSIRSDISYQNIDAIVLPTLPTLRHWFMSDRFSEQGDIMAKFQGALNRAYAQLEQSTLLHNGHFTKDAANWTIEGDAHQITLEDGRRVLRLPDWSSSVSQMIEIENFNPDKEYNLVFHGQGEGTVTLEHGEETKYIETHTHHFANFTTSQRQGLTFESNKVTVTISSEDGEFLVDNIALVEAPLPTDDQNSEGNTAFSTNSDTSMNNNQ

SEQ ID NO: 64 - protein, 1121 aa, Bacillus thuringiensis
MEGGNNQGFINIEGRLSLEQVLAVSAGVASYILSNLGPFGVFFSYIIGAFWPTAPSDTRVWEAFMELIEARIDQKIQESTRKDAIARLQGLGAASEVYQESLESWLENQNDARAMSVVRQQFVAFELDFVTAMPFFERSGDEILLLAVYAQAANLHLLLLRDASLYGADWGMEPYDIANYYNRQKERTATYSNHCMEWYKKGLDELWSAGGYGGDVWQDYNDFRREMTLSVLDFVALFPNYDTHLYPIEVKGELTREIYTPGINIDIDRGRLGKVTLENALVNSPRLFSWLKEIQLFTNINFNPRWKFLSATRIGYKLTGYRSVNFEPYEGFYFEGLFQSNFTLNDYQEIYIVDVTRFGSLYDTPTAITAMTFFKDDNTTFNYSSNTPSGEPMITSGFYLPGIDGSEEPTSNNFSHRLSTINTYNTTNQTFYHTFGWTHVSVDRNNTIVSNKITQIPFVKANTGNVVRGPGHTGGDLVVFKPAARPDIVLRVGNTRLQSYRVRVRYASNADISLELDTTYEKTNIHLQKTFNSSEAEDLTYDNFQYAEADNIVWLGQDLSQYIEFQNAITNDSTAIAYFERVEFLPVGATYEAEQDLETAKKVVNALFTNTKNALQMSVTDYEVTQAANLVECVSDELFPNEKRLLFDAVKEAKRLSGIRNLLQDSDFQEINGENGWVGSIEIEIREGDTLFKGYSLRLPSAREIYMEMFPTYLHQKIEESRLKPYTRYRLRGFVSSSQDLEIFSIRHETNRIVKNVSDDLLPNLSSYYTCGGVNRCSTQKYVYNRLEFQNSLSSGNRYSDAHSFSIPIDTGKIDLNDNTGIWIAFKLATTGGYATLGNLELVEEAPLIGDTLERVQREDQQWKSQMTRKREEAERKYMAAKQAIDRLYADYQDHQLNPNVEITDITATQHLVQSLPYVNNDVLQEIPGMNYTRFTELTNRLQQAWELYGLRNMIANGDFRNGLNDWDATSGVNIQQINHTSVLVISNWDAQISKQFTVQPNQKYVLRVTVRKEGSGDGYVTIRDGGNHTETLTFNTCDYERSNVYNEKVFQSNDYGTNNVYHTQTTNANRYTTNSLYNDGTGYVTKTVEFIPYTEQVWIEMSETEGVFYVESIEFILEEV

SEQ ID NO: 65 - protein, 1154 aa, Bacillus thuringiensis
MNRNNQNEYEIIDGTNCGCSSDEVVKYPLTDDPNAGLQNMNYKEYLQTYDGDYTGSLINPNLSINTRDVLQTGINIVGRVLGFLGVPFAGQLVTFYTFLLNQLWPTNNNAVWEAFMAQIEELIDQRISEQVVRNALDALTGIHDYYNEYLAALEEWLERPNGARANLAFQRFENLHQLFVSQMPSFGSGPGSERDAVALLTVYAQAANLHLLLLKDAEIYGARWGLNQGQINLYFNAQQDRTQIYTNHCVATYNRGLENLRGTNTESWYNYHQFRREMTLMAMDLVALFPYYNLRQYPNGANPQLTREIYTDPVVFNPPANQGLCRRWRNNPYMTFSELENTFIRPPHLFDRLNSLTINSHRFPISSNFMDYWAGHTLRRSYMNNSAVQEDSYGATTSTRVTINTGVNGTNRIESTAVDFRSGLLGVYGVHRASFVPGGLFNGTISPANAGCRNLHDTRDELPLEENNGSPSHRLSHVTFLSFLTDQAGSIRNSGAVPLYVWARQDIDLNNTITANRITQLPLVKASEIAAGTTVVRGPGFTGGDILRRTSAGTLGTIRVNVNSPLTQRYRVRFRYASTTDFNFFVIRGGTTVNNFTFPRTMNSGQESRYESYVTREFSTSFNFLQIQDTLRLTVQSFSSGQQVYVDRIEIIPVNPTREAEEDLEAAKKAVANLFTHTRDGLQVNVTDYQVDQAANLVSCLSDEQYSHDKKMLLEAVRAAKRLSRERNLLQDPDFNEINTIEENGWKASNGVTISEGGPFFKGRALQLASARENNPTYIYQKVDASVLKPYTRYRLDGFVASSQDLELDLIHQHKVHLVKNVPDNLVSDSYSDGSCSGINRCEEQHLVDMQLDAEHHPMDCCEAAETHEFSSYIDTGDLNPSVDQGIWVVLKVRTTDGYATLGNLELVEVGPLSGESLEREQKDNAKWNAELGRKRAETDRVYQAAKQAINHLFVDYQDQQLNPEIGLAEMNEASNLLESIPGVYSDTVLQIPGVNYEIYTELSNRLQQASYLFMSRNAVQNGDFNNGLDSWNATTDATVQQDGTMHFLVLSHWDAQVSQPLRVQPNCKYVLRVTARKVGGGDGYVTIQDGAHHQETLTFNACDYDVNGTYVNDNTYITKEVVFYPETKHMWIEVSESEGAFYIDSIEFIEIQE

SEQ ID NO: 66 - protein, 638 aa, Bacillus thuringiensis
EIIDGTNCGCSSDEVVKYPLTDDPNAGLQNMNYKEYLQTYDGDYTGSLINPNLSINTRDVLQTGINIVGRVLGFLGVPFAGQLVTFYTFLLNQLWPTNNNAVWEAFMAQIEELIDQRISEQVVRNALDALTGIHDYYNEYLAALEEWLERPNGARANLAFQRFENLHQLFVSQMPSFGSGPGSERDAVALLTVYAQAANLHLLLLKDAEIYGARWGLNQGQINLYFNAQQDRTQIYTNHCVATYNRGLENLRGTNTESWYNYHQFRREMTLMAMDLVALFPYYNLRQYPNGANPQLTREIYTDPVVFNPPANQGLCRRWRNNPYMTFSELENTFIRPPHLFDRLNSLTINSHRFPISSNFMDYWAGHTLRRSYMNNSAVQEDSYGATTSTRVTINTGVNGTNRIESTAVDFRSGLLGVYGVHRASFVPGGLFNGTISPANAGCRNLHDTRDELPLEENNGSPSHRLSHVTFLSFLTDQAGSIRNSGAVPLYVWARQDIDLNNTITANRITQLPLVKASEIAAGTTVVRGPGFTGGDILRRTSAGTLGTIRVNVNSPLTQRYRVRFRYASTTDFNFFVIRGGTTVNNFTFPRTMNSGQESRYESYVTREFSTSFNFLQIQDTLRLTVQSFSSGQQVYVD

SEQ ID NO: 67 - protein, 249 aa, Bacillus thuringiensis
MENLNHCPLEDIKVNPWKTPQSTARVITLRVEDPNEINNLLSINEIDNPNYILQAIMLANAFQNALVPTSTDFGDALRFSMAKGLEIANTITPMGAVVSYVDQNVTQTNNQVSVMINKVLEVLKTVLGVALSGSVIDQLTAAVTNTFTNLNTQKNEAWIFWGKETANQTNYTYNVLFAIQNAQTGGVMYCVPVGFEIKVSAVKEQVLFFTIQDSASYNVNIQSLKFAQPLVSSSQYPIADLTSAINGTL

SEQ ID NO: 68 - protein, 249 aa, Bacillus thuringiensis; identical to SEQ ID NO: 67

SEQ ID NO: 69 - protein, 249 aa, Bacillus thuringiensis
MENLNHCPLEDIKVNPWKTPQSTARVITLRVEDPNEINNLLSINEIDNPNYILQAIMLANAFQNALVPTSTDFGDALRFSMPKGLEIANTITPMGAVVSYVDQNVTQTNNQVSVMINKVLEVLKTVLGVALSGSVIDQLTAAVTNTFTNLNTQKNEAWIFWGKETANQTNYTYNVLFAIQNAQTGGVMYCVPVGFEIKVSAVKEQVLFFTIQDSASYNVNIQSLKFAQPLVSSSQYPIADLTSAINGTL

SEQ ID NO: 70 - protein, 250 aa, Bacillus thuringiensis
MENPNHCPLEDIQVNPWKTPQSKARVITLRIDDPNEINNLLSINEIENTNYLLQAIMLANAFQKALVPTSTEFAEDALQFSMTKGLEVANTISPPGAVVQYVDQNVSQTNNQVSAMINKVLDVLKSILGVALGQSVIEQLTSAVTNTFTNLNTQKNEAWIFWGRETSTQTNYTYNVLFAIQNGQTGGVMYCVPVGFEIKVSAVKERVLFLTIQDSASYNVNIQSLKFAQPLVSASEYPIADLTSAINGTL

SEQ ID NO: 71 - protein, 265 aa, Bacillus thuringiensis
MKESIYYNEENEIQISQGNCFPEELGHNPWRQPQSTARVIYLKVKDPIDTTQLLEITEIENPNYVLQAIQLAAAFQDALVPTETEFGEAIRFSMPKGLEVAKTIQPKGAVVAYTDQTLSQSNNQVSVMIDRVISVLKTVMGVALSGSIITQLTAAITDTFTNLNTQKDSAWVFWGKETSHQTNYTYNVMFAIQNETTGRVMMCVPIGFEIRVFTDKRTVLFLTTKDYANYSVNIQTLRFAQPLIDSRALSINDLSEALRSSKYLY

SEQ ID NO: 72 - protein, 525 aa, Bacillus thuringiensis
MAQSEFNQNLREQGQSRARVIILRVNNPGYNTNTLDIADIEDIIHLPQAIELANAFQSALVPTTSNFGEDTLRFDVERGLGIATHVYPRAINVNYVTRTLSQTNNQVQSMINKVIEELKSLLGINLANSVLQQLTTVITETFTNLYVQQQSAWLFWGRQTSSQTNYTYNIVFAIQNAQTGSFMKAIPIGFEISAYIARERLLFFNIQDYASYSVKIHAIQVMQPLIHESFQPLRGIFNIITSVNNRSAIQITEYYDENTHDYPVKLWDYNNIINQKWILVFNQTTRAYSIQNLIARYLVLTWDSTPGSNKVFASTNRWNDSQFWILESTADGSIFLTNMKDTQFVLEIENSSTTNGTNVIVNRKNNNAQQKFYLNKVNQEFQDGVYKIKTALNNSSVLQMSEDYFGYTSDYFVKLWTNNNNDINQKWIFEFDSTKSAYQIKSQRDPSLVLAWTWSVPTVKLPIPNNDDHLWFLQNAGSGTYYFVNMTDTRYVLEVASSSTTNGTILTINKRNGNLNQKFLLDMIN

SEQ ID NO: 73 - protein, 259 aa, Bacillus thuringiensis
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSNAPIVDIFNVNNYNLYHSNHKIIQNLNLSN

SEQ ID NO: 74 - protein, 259 aa, Bacillus thuringiensis; identical to SEQ ID NO: 73

SEQ ID NO: 75 - protein, 259 aa, Bacillus thuringiensis
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 76 - protein, 259 aa, Bacillus thuringiensis; identical to SEQ ID NO: 73

SEQ ID NO: 77 - protein, 263 aa, Bacillus thuringiensis
MHLNNLNNFNNLENNGEYHCSGPIIKKPFRHIALTVPSSDITNFNEIFYVEPQYIAQAIRLTNTFQGAIDPLTLNFNFEKALQIANGLPNAGVTGTINQSVIHQTIEVSVMISQIKEIIRSVLGLVINSANFWNSVVSAITNTFTNLEPQVDENWIVWRNLSATQTSYFYKILFSIQNEDTGRFMAILPIAFEITVDVQKQQLLFITIKDSARYEVKMKALTVVQALDSYNAPIIDVFNVRNYSLHRPNHNILQNLNVNPIKS

SEQ ID NO: 78 - protein, 263 aa, Bacillus thuringiensis; identical to SEQ ID NO: 77

SEQ ID NO: 79 - protein, 263 aa, Bacillus thuringiensis; identical to SEQ ID NO: 77

SEQ ID NO: 80 - protein, 263 aa, Bacillus thuringiensis; identical to SEQ ID NO: 77

SEQ ID NO: 81 - protein, 246 aa, Bacillus thuringiensis
YHCSGPIIKKPFRHIALTVPSSDITNFNEIFYVEPQYIAQAIRLTNTFQGAIDPLTLNFNFEKALQIANGLPNAGVTGTINQSVIHQTIEVSVMISQIKEIIRSVLGLVINSANFWNSVVSAITNTFTNLEPQVDENWIVWRNLSATQTSYFYKILFSIQNEDTGRFMAILPIAFEITVDVQKQQLLFITIKDSARYEVKMKALTVVQALDSYNAPIIDVFNVRNYSLHRPNHNILQNLNVNPIKS

SEQ ID NO: 82 - protein, 156 aa, Bacillus thuringiensis
NTFQGAIDPLTLNFNFEKALQIANGLPNAGVTGTINQSVIHQTIEVSVMISQIKEIIRSVLGLVINSANFWNSVVSAITNTFTNLEPQVDENWVVWRNLSATQTSYFYKILFSIQNEDTGRFMAILPIAFEITVDVQKQQLLFITIKDSARYEVKM

SEQ ID NO: 83 - protein, 157 aa, Bacillus thuringiensis
NTFQGAIDPLTLNFNFEKALQIANGLPNAGVTGTINQSVIHQTIEVSVMISQIKEIIRSVLGLVINSANFWNSVVSAITNTFTNLEPQVDENWIVWRNLSATQTSYFYKILFSIQNEDTGRFMAILPIAFEITVDVQKQQLLFITIKDSARYEVKMK

SEQ ID NO: 84 - protein, 263 aa, Bacillus thuringiensis; identical to SEQ ID NO: 77

SEQ ID NO: 85 - protein, 263 aa, Bacillus thuringiensis; identical to SEQ ID NO: 77

SEQ ID NO: 86 - protein, 263 aa, Bacillus thuringiensis
MYTKNLNSLEINEDYQYSRPIIKKPFRHITLTVPSSDIASFNEIFYLEPQYVAQALRLTNTFQAAIDPLTLNFDFEKALQIANGLPNAGITGTLNQSVIQQTIEISVMISQIKEIIRNVLGLVINSTNFWNSVLAAITNTFTNLEPQVDENWIVWRNLSATHTSYYYKILFSIQNEDTGAFMAVLPIAFEITVDVQKQQLLFITIRDSARYEVKMKALTVVQLLDSYNAPIIDVFNVHNYGLYQSNHPNHHILQNLNLNKIKG

SEQ ID NO: 87 - protein, 880 aa, Bacillus thuringiensis
MKNMKKKLASVVTCTLLAPMFLNGNVNAVYADSKTNQISTTQKNQQKEMDRKGLLGYYFKGKDFSNLTMFAPTRDSTLIYDQQTANKLLDKKQQEYQSIRWIGLIQSKETGDFTFNLSEDEQAIIEINGKIISNKGKEKQVVHLEKGKLVPIKIEYQSDTKFNIDSKTFKELKLFKIDSQNQPQQVQQDELRNPEFNKKESQEFLAKPSKINLFTQKMKREIDEDTDTDGDSIPDLWEENGYTIQNRIAVKWDDSLASKGYTKFVSNPLESHTVGDPYTDYEKAARDLDLSNAKETFNPLVAAFPSVNVSMEKVILSPNENLSNSVESHSSTNWSYTNTEGASVEAGIGPKGISFGVSVNYQHSETVAQEWGTSTGNTSQFNTASAGYLNANVRYNNVGTGAIYDVKPTTSFVLNNDTIATITAKSNSTALNISPGESYPKKGQNGIAITSMDDFNSHPITLNKKQVDNLLNNKPMMLETNQTDGVYKIKDTHGNIVTGGEWNGVIQQIKAKTASIIVDDGERVAEKRVAAKDYENPEDKTPSLTLKDALKLSYPDEIKEIEGLLYYKNKPIYESSVMTYLDENTAKEVTKQLNDTTGKFKDVSHLYDVKLTPKMNVTIKLSILYDNAESNDNSIGKWTNTNIVSGGNNGKKQYSSNNPDANLTLNTDAQEKLNKNRDYYISLYMKSEKNTQCEITIDGEIYPITTKTVNVNKDNYKRLDIIAHNIKSNPISSLHIKTNDEITLFWDDISITDVASIKPENLTDSEIKQIYSRYGIKLEDGILIDKKGGIHYGEFINEASFNIEPLQNYVTKYEVTYSSELGPNVSDTLESDKIYKDGTIKFDFTKYSKNEQGLFYDSGLNWDFKINAITYDGKEMNVFH

SEQ ID NO: 88 - protein, 881 aa, Bacillus thuringiensis
  SITE 846: note = misc_feature - Xaa can be any naturally occurring amino acid
MKKKLASVVTCTLLAPMFLNGNVNAVYADSKTNQISTTQKNQQKEMDRKGLLGYYFKGKDFSNLTMFAPTRDSTLIYDQQTANKLLDKKQQEYQSIRWIGLIQSKETGDFTFNLSEDEQAIIEINGKIISNKGKEKQVVHLEKGKLVPIKIEYQSDTKFNIDSKTFKELKLFKIDSQNQPQQVQQDELRNPEFNKKESQEFLAKPSKINLFTQKMKREIDEDTDTDGDSIPDLWEENGYTIQNRIAVKWDDSLASKGYTKFVSNPLESHTVGDPYTDYEKAARDLDLSNAKETFNPLVAAFPSVNVSMEKVILSPNENLSNSVESHSSTNWSYTNTEGASVEAGIGPKGISFGVSVNYQHSETVAQEWGTSTGNTSQFNTASAGYLNANVRYNNVGTGAIYDVKPTTSFVLNNDTIATITAKSNSTALNISPGESYPKKGQNGIAITSMDDFNSHPITLNKKQVDNLLNNKPMMLETNQTDGVYKIKDTHGNIVTGGEWNGVIQQIKAKTASIIVDDGERVAEKRVAAKDYENPEDKTPSLTLKDALKLSYPDEIKEIEGLLYYKNKPIYESSVMTYLDENTAKEVTKQLNDTTGKFKDVSHLYDVKLTPKMNVTIKLSILYDNAESNDNSIGKWTNTNIVSGGNNGKKQYSSNNPDANLTLNTDAQEKLNKNRDYYISLYMKSEKNTQCEITIDGEIYPITTKTVNVNKDNYKRLDIIAHNIKSNPISSIHIKTNDEITLFWDDISITDVASIKPENLTDSEIKQIYSRYGIKLEDGILIDKKGGIHYGEFINEASFNIEPLQNYVTKYKVTYSSELGQNVSDTLESDKIYKDGTIKFDFTKYSXNEQGLFYDSGLNWDFKINAITYDGKEMNVFHRYNK

SEQ ID NO: 89 - protein, 803 aa, Bacillus thuringiensis
MNMNNTKLNARALPSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLNIYLPKITSMLSDVMKQNYALSLQIEYLSRQLQEISDKLDVINLNVLINSTLTEITPSYQRIKYVNEKFDKLTFATESTLRAKQGIFNEDSFDNNTLENLTDLAELAKSITKNDVDSFEFYLHTFHDVLIGNNLFGRSALKTASELITKDEIKTSGSEIGKVYSFLIVLTSLQAKAFLTLTTCRKLLGLSDIDYTSIMNEHLNNEKNEFRDNILPALSNKFSNPSYAKTIGSDNYAKVILESEPGYALVGFEIINDPIPVLKAYKAKLKQNYQVDNQSLSEIVYLDIDKLFCPENSEQKYYTKNLTFPDGYVITKITFEKKLNNLIYEATANFYDPSTGDIDLNKKQVESTFPQTDYITMDIGDDDGIYMPLGVISETFLTPINSFGLEVDAKSKTLTLKCKSYLREYLLESDLKNKETGLIAPPNVFISNVVKNWDIEEDSLEPWVANNKNAYVDNTGGIERSKALFTQGDGEFSQFIGDKLKPNTDYIIQYTVKGKPAIYLKNKSTGYITYEDTNGNSEEFQTIAVKFTSETDLSQTHLVFKSQNGYEAWGDNFIILEAKLFETPESPELIKFNDWERFGTTYITGNELRIDHSRGGYFRQSLNIDSYSTYDLSFSFSGLWAKVIVKNSRGVVLFEKVKNNGSSYEDISESFTTASNKDGFFIELTAERTSSTFHSFRDISIKEKIE

SEQ ID NO: 90 - protein, 834 aa, Bacillus thuringiensis
MKNMKKKLASVVTCMLLAPMFLNGNVNAVNADSKINQISTTQENQQKEMDRKGLLGYYFKGKDFNNLTMFAPTRDNTLMYDQQTANALLDKKQQEYQSIRWIGLIQRKETGDFTFNLSKDEQAIIEIDGKIISNKGKEKQVVHLEKEKLVPIKIEYQSDTKFNIDSKTFKELKLFKIDSQNQSQQVQLRNPEFNKKESQEFLAKASKTNLFKQKMKRDIDEDTDTDGDSIPDLWEENGYTIQNKVAVKWDDSLASKGYTKFVSNPLDSHTVGDPYTDYEKAARDLDLSNAKETFNPLVAAFPSVNVSMEKVILSPNENLSNSVESHSSTNWSYTNTEGASIEAGGGPLGLSFGVSVTYQHSETVAQEWGTSTGNTSQFNTASAGYLNANVRYNNVGTGAIYDVKPTTSFVLNNNTIATITAKSNSTALRISPGDSYPEIGENAIAITSMDDFNSHPITLNKQQVNQLINNKPIMLETDQTDGVYKIRDTHGNIVTGGEWNGVTQQIKAKTASIIVDDGKQVAEKRVAAKDYGHPEDKTPPLTLKDTLKLSYPDEIKETNGLLYYDDKPIYESSVMTYLDENTAKEVKKQINDTTGKFKDVNHLYDVKLTPKMNFTIKMASLYDGAENNHNSLGTWYLTYNVAGGNTGKRQYRSAHSCAHVALSSEAKKKLNQNANYYLSMYMKADSTTEPTIEVAGEKSAITSKKVKLNNQNYQRVDILVKNSERNPMDKIYIRGNGTTNVYGDDVTIPEVSAINPASLSDEEIQEIFKDSTIEYGNPSFVADAVTFKNIKPLQNYVKEYEIYHKSHRYEKKTVFDIMGVHYEYSIAREQKKAA

SEQ ID NO: 91 - protein, 769 aa, Bacillus thuringiensis
MKNMKKKLASVVTCTLLAPMFFNGNVNTAYADNKINQDSSKQEYQQKEMDRKGLLGYYFKDKDFSNLTMFSPTRYNTLIYDQQTANKLLDKKQQEYQSIRWIGLIQSNKTGDFTFELSDDECAIIEMDGKVISNKGKEKQVVHLEKGKLVPIKIEYQLDEPLNIDDEKFKGFKLLKVDNQKQLHQVQQDELRNPEFNKKESQEFLAKASKINLFTKKIKRDIDEGTDTDGDSIPDMWEENGYTIQNRIAVKWNDSLASKGYTKFVSNPLDSHTVGDPYTDYEKASRDLDLSNAKETFNPLVAAFPSVNVSMEKVILSPNKNLSNSVESHSSTNWSYTNTEGASVEAGIGPKGFSFGVSANYQHSETVAQEWGASIGDTTQLNTASAGYLNANVRYNNVGTGAIYDVKPTTSFVLEKNTIATITAKSNSTALSISPGESYPKKGQNGIAITSMDDFNSHPITLNKKQLDQVLTNNPIMLETDQTDGIYKIKDTHGNIVTGGTWNGVTQQIKAKTASIIVDDGKQVAEKRVAAKDYAYPEDKTPSLTLKDALKLSFPEEIKETDGLLYYNNKPIYESSVMTYLDGNTAKEVKKQINDKTGEFKDVQHLYAVKLTPKMNFTIKVPVAYDTAKQAVNLGGDNPWGAKGLLGTWVNAMVVDNSGDKAYKRVEPGYLLSPTLEFSEGSLDNLKKNYSFYVSMYVKSDKPFTLRINAGPYSTKRTIEASNDFKRVDIPAFYIEGFPIDTIRLEGSDYPSAIWWKDVSITEVSAVKK

SEQ ID NO: 92 - protein, 784 aa, Bacillus thuringiensis
MTYMKKKLVSVVTCTLLAPMFLNGNVNPVYADNQTNQLSTAQENQEKEVDRKGLLGYYFKGKEFNHLTLFAPTRDNTLIYDQQTVDSLLDKKQQEYQSIRWIGLIQSKETGDFTFNLSDDKNAIMEIDTKTISHKGQNKQVVHLEKGKLVPIKIEYQPDQIVNRDSKIFKEFKLFKVDSKQQSHQVQLDELRNPEFNKKETQQFLEKASKTNLFTQNMKRDEDATDTDGDSIPDLWEENGYTIQNKVAVKWDDSFAAKGYTKFVSNPFDSHTVGDPYTDYEKAARDLDLANAKETFNPLVAAFPSVNVNLEKVILSPNEDLSNSVESHSSTNWSYTNTEGVSIEAGSGPLGISYGVSANYQHSETVAKEWGTSTGNTSQFNTASAGYLNANVRYNNVGTGAIYEVKPTTGFVLDNDTVATITAKSNSTALSISPGESYPKKGQNGIAINTMDDFNSHPITLNKQQLDQIFNNKPLMLETNQADGVYKIKDTSGNIVTGGEWNGVIQQIQAKTASIIVDTGEGVSEKRVAAKDYDNPEDKTPSLSLKEALKLGYPEEIKEKDGLLYYNDKPIYESSVMTYLDENTAKEVKEQLNDITGKFKDVKQLFDVKLTPKMNFTIKLATLYDGAEDGSSPTDVGISSPLGEWAFKPDINNVEGGNTGKRQYQLSKNKDGYYYGMLALSPEVSNKLKKNYQYYISMSIKADAGVEPTVTVMDNLNCIVDKKLKLSSNGYQRFDILVDNSESHPINVMVIDLGVSSQDYNNYSKNIYIDDITITEVSAMKVKN

SEQ ID NO: 93 - protein, 785 aa, Bacillus thuringiensis
MTYMKKKLVSVVTCALLAPMFLNGNVNPVYADNQTNQLSTAQENQEKEVDRKGLLGYYFKGKEFNHLTLFAPTRDNTLIYDQQTVDFLLDKKQQEYQSIRWIGLIQSKETGDFTFNLSDDKNAIIEIDGKTISHKGQNKQVVHLEKGKLVPIKIEYQSDQIVNRDSKIFKEFKLFKVDSQQQSHQVQLDELRNPEFNKKETQQFLEKASKTNIFTQNMKRDEDATDTDGDSIPDLWEENGYTIQNKVAVKWDDSFAAKGYTKFVSNPFDSHTVGDPYTDYEKAARDLDLANAKETFNPLVAAFPSVNVNLEKVILSPNEDLSNSVESHSSTNWSYTNTEGASIEAGSGPLGISYGVSANYQHSETVAKEWGTSTGNTSQFNTASAGYLNANVRYNNVGTGAIYEVKPTTGFVLDNDTVATITAKSNSTALSISPGESYPKKGQNGIAINTMDDFNSHPITLNKQQLDQIFNNKPLMLETNQADGVYKIKDTSGNIVTGGEWNGVIQQIQAKTASIIVDTGEGVSEKRVAAKDYDNPEDKTPSLSLKEALKLGYPEEIKEKDGLLYYNDKPIYESSVMTYLDENTAKEVKEQLNDITGKFKDVKQLFDVKLTPKMNFTIKLATLYDGAEDGSSPTDVGISSPLGEWAFKPDINNVEGGNTGKRQYQLSKNKDGYYYGMLALSPEVSNKLKKNYQYYISMSIKADAGVEPTVTVMDNLLNGIVDKKLKLSSNGYQRFDILVDNSESHPINVMVIDLGVSSQDYNNYSKNIYIDDITITEVSAMKVKN

SEQ ID NO: 94 - protein, 860 aa, Bacillus thuringiensis
MTYMKKKLVSVVTCTLLAPIFLTGNVHPVNADSKKSQPSTAQEKQEKPVDRKGLLGYFFKGKEFNHLTLFAPTRDNTLIYDQQTANSLLDTKQQEYQSIRWIGLIQSKETGDFTFNLSDDQNAIIEIDGKIISHKGQNKQVVHLEKGKLVPIKIEYQSDQILTRDSNIFKEFQLFKVDSQQHSHQVQLDELRNPDFNKKETQQFLEKAAKTNLFTQNMKRDTDDDDDTDTDGDSIPDLWEENGYTIQNKVAVKWDDSFAAKGYTKFVSNPLESHTVGDPYTDYEKAARDLDLANAKETFNPLVAAFPSVNVNLEKVILSPDENLSNSVESHSSTNWSYTNTEGASIEAGGGPLGISFGVSANYQHSETVAKEWGTSTGNTSQFNTASAGYLNANVRYNNVGTGAIYEVKPTTSFVLDKDTVATITAKSNSTALSISPGESYPKKGQNGIAINTMDDFNSHPITLNKQQLDQLLNNKPLMLETNQADGVYKIKDTSGNIVTGGEWNGVIQQIQAKTASIIVDTGESVSEKRVAAKDYDNPEDKTPSLSLKEALKLGYPEEIKEKDGLLYYKDKPIYESSVMTYLDENTAKEVEKQLQDTTGIYKDINHLYDVKLTPTMNFTIKLASLYDGAENNDVKNGPIGHWYYTYNTGGGNTGKHQYRSANPSANVVLSSEAKSKLDKNTNYYLSMYMKAESDTEPTIEVSGENSTITSKKVKLNSEGYQRVDILVPNSERNPINQIYVRGNNTTNVYWDDVSITNISAINPKTLTDEEIKEIYKDFSESKDWPWFNDVTFKNIKPLENYVKQYRVDFWNTNSDRSFNRIKDSYPVNEDGSVKVNMTEYNEGYPLRIESAYHLNISDL

SEQ ID NO: 95 - protein, 803 aa, Bacillus thuringiensis; identical to SEQ ID NO: 89

SEQ ID NO: 96 - protein, 775 aa, Bacillus thuringiensis
MKNMKKKLASVVTCTLLAPMFLNGNVNAVYADSKTNQISTTQKNQQKEMDRKGLLGYYFKGKDFSNLTMFAPTRDNTLIYDQQTANKLLDKKQQQYQSIRWIGLIQSKEKGDFTFNLSEDEQAIIEIDGKIISNKGKEKQVVHLEKEKLVPIKIEYQSDTKFNIDSKTFKEFKLFKIDSQNQSQQVKRDELRNPEFNKKESREFLAKASKTNFFMQKMKRDIDEDTDTDGDSIPDLWEENGYTIQNKVAVKWDDKFAQQGYVKYLSSPYQAHTVGDPYTDWEKAAGDIPKSNAAATRNPLVAAFPSINVDMRKMILSKDSNLSNSAEAHSNNSYTYANSEGASIEAGFGPKGFSFGVSANYQHTETVGSDWGNSKSNTEQFNSASAGYLNANVHYNNVGTGGIYDAQPTTSFILQDSTIATITAKSNATALSIPSGDRYPASKEGISLKTMDDFNSHPITLNKPQLDAVLNNEVIKINTDQTDGRYGIIGVDGKAEIGDRWSPIIDEIKGRTASIIIDPADGKALETRIAAKDYKNPEDKTPSLTIKEGLKIAYPESISEDKDGILFYEYKNDEGKVTKKQLSEENIMPYLDEDTSKEFERQLSDGSAKGLYDIKLTPKMNITIRLATVTLGFDDQFSAYPWENATWSDKFGNLRLGSLAIPQESKYTIPKDKVKPNYDYLITGYIKHDFTTDNESLGIVAFTKKDNFEMWNMGTSIFSQNSGGEFKKFTIKTQNISGDYILDSIQLMKRNNDVNKIDSYLDDISIIPIGPNKSR

SEQ ID NO: 97 - protein, 871 aa, Bacillus thuringiensis
MKYMKKGLSSVVIGTLFASMFLNGNVNAVYANSKTNQIATTTQASKDNQIDREGLLGYYFKGKDFNDLTLFAPTRDNTLIYDQQTANTLVDQKHQEYHSIRWIGLIQSSATGDFTFKLSDDENAIIELDGKVISEKGNNKQSVHLEKGQLVQIKIEYQSDDALHIDNKIFKELKLFKIDSQNHSQQVQQDELRNPEFNKKETQVFLKKASKTNLFTQKTKRDIDEDTDTDGDSIPDVWEENGYTIQNKVAVKWDDSLASKGYQKFTSNPLEAHTVGDPYSDYEKAARDMPLSNAKETFNPLVAAFPSVNVSLEKVILSKNEDLSHSVESSQSTNWSYTNTEGVNVNAGWSGLGPSFGVSVNYQHSETVANEWGSATNDGTHINGAESAYLNANVRYNNVGTGAIYETKPTTSFILDGTTIGTIKAKENTTALTILPDQSYPEKGKNGIAINTMDDFNSRPIPLNKEQLNTYLSNKKPILLETDQVEGKYAIKDTNGNITIAGDWNGITDEISAKTASIIVDNGNQMSEKRVAAKDYTNPEDKTPNLSVKEALKLAYPDEIEEKDGLLFYNDQPIFEASVQSYVDEYTAKQIRKQLNDSTGSFKDVKNLYDVKLEPKMNFTIKTSTLYDGGESDNTKIGNWYYTYVVNGGNTGKKQYRSANKGAFTELSTESKNKLKKNIDYYVSLYMKADSKVSVDIEIDGKQESIVTDNITLDHVGYQRINILVPNLEGNEINTISIKGDGQTNVYWDDVSFVEVGAEEIEYKDPVPQFDIIEGDFDFFGDPLAVKYHDATYFIDSPLITQTPGTFSFTYKVIGEQTKTVLDSGSGKNANRINLDFKNVKSDRSFLYTLSCKDDLWGSTRTAVVRIFAVD

SEQ ID NO: 98 - protein, 280 aa, Bacillus thuringiensis
MKRMEGKLFMVSKKLQVVTKTVLLSTVFSISLLNNEVIKAEQLNINSQSKYTNLQNLKITDKVEDFKEDKEKAKEWGKEKEKEWKLTATEKGKMNNFLDNKNDIKTNYKEITFSMAGSFEDEIKDLKEIDKMFDKTNLSNSIITYKNVEPTTIGFNKSLTEGNTINSDAMAQFKEQFLDRDIKFDSYLDTHLTAQQVSSKERVILKVTVPSGKGSTTPTKAGVILNNSEYKMLIDNGYMVHVDKVSKVVKKGVECLQIEGTLKKSLDFKNDINAEAHSWG

SEQ ID NO: 99 - protein, 446 aa, Bacillus thuringiensis
MFMVSKKLQVVTKTVLLSTVFSISLLNNEVIKAEQLNINSQSKYTNLQNLKITDKVEDFKEDKEKAKEWGKEKEKEWKLTATEKGKMNNFLDNKNDIKTNYKEITFSMAGSFEDEIKDLKEIDKMFDKTNLSNSIITYKNVEPTTIGFNKSLTEGNTINSDAMAQFKEQFLDRDIKFDSYLDTHLTAQQVSSKERVILKVTVPSGKGSTTPTKAGVILNNSEYKMLIDNGYMVHVDKVSKVVKKGVECLQIEGTLKKSLDFKNDINAEAHSWGMKNYEEWAKDLTDSQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKVTEVIIKVLSDM

SEQ ID NO: 100 - protein, 803 aa, Bacillus thuringiensis; identical to SEQ ID NO: 89

SEQ ID NO: 101 - protein, 462 aa, Bacillus thuringiensis
MQRMEGKLFVVSKTLQVVTRTVLLSTVYSITLLNNVVIKADQLNINSQSKYTNLQNLKIPDNAEDFKEDKGKAKEWGKEKGEEWRPPATEKGEMNNFLDNKNDIKTNYKEITFSMAGSCEDEIKDLEEIDKIFDKANLSSSIITYKNVEPATIGFNKSLTEGNTINSDAMAQFKEQFLGKDMKFDSYLDTHLTAQQVSSKKRVILKVTVPSGKGSTTPTKAGVILNNNEYKMLIDNGYVLHVDKVSKVVKKGMECLQVEGTLKKSLDFKNDINAEAHSWGMKIYEDWAKNLTASQREALDGYARQDYKEINNYLRNQGGSGNEKLDAQLKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKATEVIIKGVKRYVVDATLLTN

SEQ ID NO: 102 - protein, 280 aa, Bacillus thuringiensis
MKRMEGKLFMVSTKLQAVTKAVLLSTVLSISLLNNEVIKAEQLNMNSQNKYTNFENLKITDKVEDFKEDKEKAKEWGKEKEKEWKLTATEKGKMNNFLDNKNDIKTNYKEITFSMAGSFEDEIKDLKEIDKIFDKANLSSPIITYKNVEPATIGFNKSLTEGNTINSDAMAQFKEQFLDRDIKFDSYLDTHLTVQQVSSKERVILKVKVPSGKGSTTPTKAGIILNNSEYKMLIDNGYMVHVDKVSKVVKKGVECLQVEGTLKKSLDFKNDINAGAHSWG

SEQ ID NO: 103 - protein, 457 aa, Bacillus thuringiensis
MIVIIFTNVKGGNELKKNFYKNLICMSALLLAMPISSNVTYAYGSEKVDYLVKTTNNTEDFKEDKEKAKEWGKEKEKEWKLTVTEKTRMNNFLDNKNDIKKNYKEITFSMAGSFEDEIKDLKEIDKMFDKANLSSSIVTYKNVEPSTIGFNKPLTEGNTINTDVQAQFKEQFLGKDIKFDSYLDTHLTAQNVSSKERIILQVTVPSGKGSTIPTKAGVILNNNEYKMLIDNGYVLHVDNISKVVKKGYECLQIQGTLKKSLDFKNDINAEAHRWGMKNYEGWAKNLTDPQREALDGYARQDYKQINDYLRNQGGSGNEKLDTQIKNISEALEKQPIPENITVYRWCGMAEFGYQISDPLPSLKEMEEKFLNTMKEDKGYMSTSLSSERLSAFGSRKFILRLQVPKGSTGAYLSAIGGFASEKEILIDKDSNYHIDKITEVVIKGVKRYVVDATLLTK

SEQ ID NO: 104 - protein, 462 aa, Bacillus thuringiensis
MQRMEGKLFMVSKKLQAVTKTVLLSTVLSISLLNNEEVKAEQLNINSQNKYTNFQNLKITDNAEDFKEDKEKAKEWGEEKEKEWKLTATEKGKMNNFLDNKNDIKTNYKEITFSMAGSFEDEIKDLKEIDKIFDKANLSSSIITYKNVEPATIGFNKSLTEGNTINSDAMAQFKEQFLGKDMKFDSYLDTHLTAHQVSSKKRVILKVTVPSGKGSTTPTKAGVILTNNEYKMLIDNGYVLHVDKVSKVVKKGMECLQVEGTLKKSLDFKNDINAEAHSWGMKIYEDWAKNLTASQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKATEVIIKGVKRYVVDATLLTN

SEQ ID NO: 105 - protein, 280 aa, Bacillus thuringiensis
MKRMEGKLFMVSKKLQLVTKALLFSTVLSIPLLNNEEVKAEHLNLNSQSKYPSFQNQKITDNAEDFKEDKEKAKEWGEVKEKEWKLTATEKRKINDFLNDTNKIKTNYKEITFSMAGSFEDELKDLKEIDKMFDKANLSSSIITYKNVEPATIGFNKSLTEGNTINSDVMAQFKEQFLGKDIKFDSYLDTHLTVQQVSSKERVILKVTVPSGKGSTTPTKAGVILDGNEHKMLIDNGYVLHVDKVSKVVKRGLECLQVEGTLKKSLDFKNDISAKAHSWG

SEQ ID NO: 106 - protein, 462 aa, Bacillus thuringiensis
MKRMEGKLFMVSKKLQLVTKALLFSTVLSIPLLNNEEVKAEHLNLNSQSKYPSFQNQKITDNAEDFKEDKEKAKEWGEVKEKEWKLTATEKRKINDFLNDTNKIKTNYKEITFSMAGSFEDELKDLKEIDKMFDKANLSSSIITYKNVEPATIGFNKSLTEGNTINSDVMAQFKEQFLGKDIKFDSYLDTHLTVQQVSSKERVILKVTVPSGKGSTNPTKAGVILDGNEHKMLIDNGYVLHVDKVSKVVKKGLECLQVEGTLKKSLDFKNDISAKAHSWGMKNYEEWAANLTDSQRKALDGYARQDYKKINDYLRNQGGSGNEQLDAQIKNISETLNNKPIPENITVYRWCGMPEFGYQISEPLPALKDFEWEFLNTIKEDKGYISTSLSSERLAAFGSRKIILRLQIPKGSKGAYLSAIGGFANEKEILLDKDSKYHINKITEVVIKGIKRYVVDATLLTN

SEQ ID NO: 107 - protein, 280 aa, Bacillus thuringiensis
MKRMEGKLFMVSRKLQLVTKALLFSTVLSIPLLNNEEVKAEHLNLNSQSKYPSFQNQKITDNAEDFKEDKEKAKEWGEVKEKEWKLTATEKRKINDFLNDTNKIKTNYKEITFSMAGSFEDELKDLKEIDKMFDKANLSSSIITYKNVEPATIGFNKSLTEGNTINSDVMAQFKEQFLGKDIKFDSYLDTHLTVQQVSSKERVILKVTVPSGKGSTNPTKAGVILDGNEPKMLIDNGYVLHVDKVSKVVKKGLECLQVEGTLKKSLDFKNDISAKAHSWG

SEQ ID NO: 108 - protein, 462 aa, Bacillus thuringiensis
MKRMEGKLFMVSKKLQLVTKALLFSTVLSIPLLNNEEVKAEHLNLNSQSKYPSFQNQKITDNAEDFKEDKEKAKEWGEVKEKEWKLTATEKRKINDFLNDTNKIKTNYKEITFSMAGSFEDELKDLKEIDKMFDKANLSSSIITYKNVEPATIGFNKSLTEGNTINSDVMAQFKEQFLGKDIKFDSYLDTHLTVQQVSSKERVILKVTVPSGKGSTTPTKAGVILDGNEHKMLIDNGYVLHVDKVSKVVKRGLECLQVEGTLKKSLDFKNDISAKAHSWGMKNYEEWAANLTDSQRKALDGYARQDYKKINDYLRNQGGSGNEQLDAQIKNISETLNNKPIPENITVYRWCGMPEFGYQISEPLPALKDFEWEFLNTIKEDKGYISTSLSSERLAAFGSRKIILRLQIPKGSKGAYLSAIGGFANEKEILLDKDSKYHINKITEVVIKGIKRYVVDATLLTN

SEQ ID NO: 109 - protein, 451 aa, Bacillus thuringiensis
MVSKKLQLITKTLVFSTVLSIPLLNNSEIKAEQLNMNSQIKYPNFQNINIADKPVDFKEDKEKAREWGKEKEKEWKLTVTEKGKINDFLDDKDGLKTKYKEINFSKNFEYETELKELEKINTMLDKANLTNSIVTYKNVEPTTIGFNQSLIEGNQINAEAQQKFKEQFLGQDIKFDSYLDMHLTEQNVSSKERVILKVTVPSGKGSTPTKAGVVLNNNEYKMLIDNGYVLHVENITKVVKKGQECLQVEGTLKKSLDFKNDSDGKGDSWGKKNYKEWSDTLTTDQRKDLNDYGVRGYTEINKYLREGDTGNTELEEKIKNISDALEKNPIPENITVYRYCGMAEFGYPIKPEAPSVQDFEERFLDTIKEEKGYMSTSLSSDATSFGARKIILRLQVPKGSSGAYVAGLDGFKPAEKEILIDKGSKYRIDKVTEVVVKGTRKLVVDATLLTK

SEQ ID NO: 110 - protein, 350 aa, Bacillus thuringiensis
MKRMEERLFMVSKKLQLITKTLVFSTVLSIPLLNNSEIKAEQLNMNSQIKYPNFQNINIADKPVDFKEDKEKAREWGKEKEKEWKLTATEKGKINDFLDDKDGLKTKYKEINFSKNFEYETELKELEKINTMLDKANLTNSIVTYKNVEPTTIGFNQSLIEGNQINAEAQQKFKEQFLGQDIKFDSYLDMHLTEQNVSSKERVILKVTVPSGKGSTPTKAGVVLNNNEYKMLIDNGYVLHVENITKVVKKGQECLQVEGTLKKSLDFKNDSDGKGDSWGKKNYKEWSDTLTTDQRKDLNDYGARGYTEINKYLREGGTGNTELEEKIKNISDALEKNPIPENITVYRYCG

SEQ ID NO: 111 - protein, 860 aa, Bacillus thuringiensis; identical to SEQ ID NO: 94

SEQ ID NO: 112 - protein, 803 aa, Bacillus thuringiensis; identical to SEQ ID NO: 89

SEQ ID NO: 113 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 114 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADIRDELTELTELAKSVTKNDVDGFEFYLNTFHDVMVGNNLFGRSALKTASELITKENVKTSGSEVGNVYNFLIVLTALQAKAFLTLTPCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSGQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 115 - protein, 746 aa, Bacillus thuringiensis
MNKNNAKLSTRALPSFIDYFNGIYGFATGIKDIMNMIFKTDTGGDLALDEILENQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADIRDELSELTELAKSVTQNDVDGFEFYLNTFHDVMVGNNLFGRSALKTASELITKENVKTSGSEVGNVYNFLIVLTALQAQAFLTLTPCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSGQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKKDI

SEQ ID NO: 116 - protein, 790 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPANILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYKTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDNLKPKTEYVIQYTVKGKPSIHLIDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIKP

SEQ ID NO: 117 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVDNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFIKNIVENGSIEEDNLEPWKANNKNEYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEIFTTKFGKDNFYIELSQGNNLNGGPIVQFPDVSIK

SEQ ID NO: 118 - protein, 790 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIKP

SEQ ID NO: 119 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 120 - protein, 657 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLATCRKLLGLADIDYTSIMNEHLNKEKEEFRVNILPTLSNTFSNPNYAKVKGSDEDAKMIVEAKPGHALVGFEINNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFIKNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEA

SEQ ID NO: 121 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFGKRYMSGAKDVSEMFTTKFEKDNFYIELSQGNNLYGGPIVHFYDVSIK

SEQ ID NO: 122 - protein, 789 aa, Bacillus thuringiensis; identical to SEQ ID NO: 119

SEQ ID NO: 123 - protein, 789 aa, Bacillus thuringiensis; identical to SEQ ID NO: 119

SEQ ID NO: 124 - protein, 789 aa, Bacillus thuringiensis; identical to SEQ ID NO: 119

SEQ ID NO: 125 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEHRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 126 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTGLAKSVPKNDVDGFEFYLNTFHDVMVGNNLFGRSALKTASELITKENVKTSGSEVGNVYNSLIVLTLLQAKAFLTLTTCRKLLGLADIDYTSIMNEHLNKEKEEFRVNIPPTLSNTFSNPNYAKVKGSDEDAKMIVEAKPGHALVGFEISNDSITVLKVYEAKLKQNYQVDKDSLSEVIYGDMDKLLGPDQSGPIYYPNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFIKNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEIFTTKLGKDNFYIELSQGNNLNGGPIVKFYDVSIK

SEQ ID NO: 127 - protein, 789 aa, Bacillus thuringiensis; identical to SEQ ID NO: 119

SEQ ID NO: 128 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKLTSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 129 - protein, 790 aa, Bacillus thuringiensis; identical to SEQ ID NO: 118

SEQ ID NO: 130 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKTSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 131 - protein, 789 aa, Bacillus thuringiensis
MNKNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGYALVGFEMSNDSITVLKVYEAKLKQNYQVDKDSLSEVIYGDTDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADGNSRLITLTCKSYLRELLLATDLSNKETKLIVPPSGFISNIVENGSIEEDNLEPWKANNKNAYVDHTGGVNGTKALYVHKDGGFSQFIGDKLKPKTEYVIQYTVKGKPSIHLKDENTGYIHYEDTNNNLKDYQTITKRFTTGTDLKGVYLILKSQNGDEAWGDNFIILEISPSEKLLSPELINTNNWTSTGSTHISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 132 - protein, 789 aa, Bacillus thuringiensis; identical to SEQ ID NO: 113

SEQ ID NO: 133 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 134 - protein, 789 aa, Bacillus thuringiensis; identical to SEQ ID NO: 119

SEQ ID NO: 135 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNGYVITKIDFTKKMKTLRYEVTANFYDSSTGEIDLNKKKVESSEAEYRTLSANDDGVYMPLGVISETFLTPINGFGLQADENSRLITLTCKSHLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 136 - protein, 789 aa, Bacillus thuringiensis
MNKNNTKLSTRALPGFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELAFATETSSKVKKDGSPADILDELTELTELAKSVTKNDVDGFEFYLNTFHDVMVGNNLFGRSALKTASELITKENVKTSGSGVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 137 - protein, 803 aa, Bacillus thuringiensis; identical to SEQ ID NO: 89

SEQ ID NO: 138 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTKLSKEILKIANEQNQVLNDVNNKLDAINTMLRVYLPKITSMLSDVMKQNYALSLQIKYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQADKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIYHLHDVSIK

SEQ ID NO: 139 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILRIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSNEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINEFGLQADENSRLITLTCKSYLREPLLATDLSNKETKLIVPPSGFISNIVENGSIEEDNLEPWKANNKNAYVDHTGGVNGTKALYVHKDGGISQFIGDKLKPKTEYVIQYTVKGKPSIRLKDENTGYIHYEDTNNNLEDYQTINKRFTTGADLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYTELSQGNNLYGGPIVHFYDVSIK

SEQ ID NO: 140 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNGVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDELLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNLIILEISPSEKLLSPELINTNNWTSTGSTNISGNTLTLYQGGRGILKQNLQLDSLSTYRVYFSVSGDANVRIRNSREVLFEKRYMSGAKDVSEMFTTKFEKDNFYIELSQGNNLYGGPIVHFYDVSIK

SEQ ID NO: 141 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGHSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIS

SEQ ID NO: 142 - protein, 788 aa, Bacillus thuringiensis
MNKNNTKLSNFRCLYLVEYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMYTPKFEKDNFYIELLKAKNKNKLIALFYDVSIK

SEQ ID NO: 143 - protein, 787 aa, Bacillus thuringiensis
MNKNNTKLSHVVVISFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPETEYVIQYTVKGKPSIHLKDENTGYIPYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIEPLKGITIWVNSPFYDVSIK

SEQ ID NO: 144 - protein, 788 aa, Bacillus thuringiensis
MNKNNTKLSNVNELSSLSDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELLKGRTRIDLSPFYDVSIK

SEQ ID NO: 145 - protein, 788 aa, Bacillus thuringiensis
MNKNNTKLSKNELSRLSDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGKNKNRLNGPFYDVSIK

SEQ ID NO: 146 - protein, 789 aa, Bacillus thuringiensis; identical to SEQ ID NO: 119

SEQ ID NO: 147 - protein, 789 aa, Bacillus thuringiensis; identical to SEQ ID NO: 119

SEQ ID NO: 148 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFIKNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEIFTTKLGKDNFYIELSQGNNLNGGPIVKFSDVSIK

SEQ ID NO: 149 - protein, 789 aa, Bacillus thuringiensis; identical to SEQ ID NO: 119

SEQ ID NO: 150 - protein, 789 aa, Bacillus thuringiensis; identical to SEQ ID NO: 119

SEQ ID NO: 151 - protein, 789 aa, Bacillus thuringiensis
MNMNNTKLNARALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEVSNDSITVLKVYEAKLKQNYQVDKDFLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 152 - protein, 789 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQFIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGNTLTLYQGGGGNLKQNLQLDSFSTYRVNFSVTGDANVRIRNSREVLFEKRYMSGAKDVSEIFTTKLGKDNFYIELSQGNNLYGGPLVKFNDVSIK

SEQ ID NO: 153 - protein, 789 aa, Bacillus thuringiensis
MNKNNTKLSARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQFIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGNTLTLYQGGGGNLKQNLQLDSFSTYRVNFSVTGDANVRIRNSREVLFEKRYMSGAKDVSEIFTTKLGKDNFYIELSQGNNLYGGPLVHFYDVSIK

SEQ ID NO: 154 - protein, 789 aa, Bacillus thuringiensis
MNMNNTKLSARALPSLIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLLGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQSIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGYTLTLYQGGGGNLKQNLQLDSFSTYRVNFSVTGDANVRIRNSREVLFEKRYMSGAKDVSEIFTTKLGKDNFYIELSQGNNLYGGPLVHFYDVSIK

SEQ ID NO: 155 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISEAFLTPINGFGLQADENSRLITLTCKSYLRELLLATDLSNKETKLIVPPSGFIKNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWEDNFIILEISPSEKLLSPELINTNNWTSTGSTNISGNTLTLYQGGRGILKQNLQLDSFSTYRVYFSVSGDANVRIRNSREVLFEKRYMSGAKDVSEIFTTKLGKDNFYIELSQGNNLNGGPIVKFSDVSIK

SEQ ID NO: 156 - protein, 789 aa, Bacillus thuringiensis; identical to SEQ ID NO: 119

SEQ ID NO: 157 - protein, 789 aa, Bacillus thuringiensis; identical to SEQ ID NO: 119

SEQ ID NO: 158 - protein, 789 aa, Bacillus thuringiensis
MNKNNTKLSTRALPSFIDYFNGIYGFATGIKDIINMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 159 - protein, 803 aa, Bacillus thuringiensis; identical to SEQ ID NO: 89

SEQ ID NO: 160 - protein, 803 aa, Bacillus thuringiensis; identical to SEQ ID NO: 89

SEQ ID NO: 161 - protein, 788 aa, Bacillus thuringiensis
MNKNNTKLSTRALPSFIDYFNGVYGFATGIKDIMNMIFKTDTGGDLTLDEILKNQQLLNDISGKLDGVNGSLNDLIAQGNLNTELSKEILKIANEQNQVLNDVNNKLDAINTMLRVYPPKITSMLSDVMKQNYALSLQIEYLSKQLQEISDKLDIINVNVLINSTLTEITPAYQRIKYVNEKFEELTFATETSSKVKRDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANLYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTKYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSI

SEQ ID NO: 162 - protein, 788 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVRVGNNLFGRSALKTASELITKENVKTSGSEVGNVYNFLIVLTALQAKAFLTLTTCRKLLGLADIDYTSIMNEHLSKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVTQYTVKGKPSIHLKDENTGYIHYEDTNNNLEDYQTINKRFTTGTDLKGVYLILKSQNGDEAWGDNFIILEISPSEKSLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSI

SEQ ID NO: 163 - protein, 803 aa, Bacillus thuringiensis; identical to SEQ ID NO: 89

SEQ ID NO: 164 - protein, 803 aa, Bacillus thuringiensis; identical to SEQ ID NO: 89

SEQ ID NO: 165 - protein, 787 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQVEYLSKQLKEISDKLDVINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSMTVLKVYEAKLKQNYQVDKDSLSEVIYSDMDKLLCPDQSEQIYYTNNIVFPNEYVITKIDFTKKMKTLRYEVTANSYDSSTGEIDLNKKKVESSEAEYRTLSANNDGVYMPLGVISETFLTPINGFGLQADENSRLITLTCKSYLRELLLATDLSNKETKLIVPPISFISNIVENGNLEGENLEPWIANNKNAYVDHTGGINGTKVLYVHKDGEFSQFVGGKLKSKTEYVIQYIVKGKASIYLKDKKNENSIYEEINNDLEGFQTVTKRFITGTDSSGIHLIFTSQNGEGAFGGNFIISEIRTSEELLSPELIMSDAWVGSQGTWISGNSLTINSNVNGTFRQNLPLESYSTYSMNFTVNGFGKVTVRNSREVLFEKSYPQLSPKDISEKFTTAANNTGLYVELSRSTSGGAINFRDFSIK

SEQ ID NO: 166 - protein, 787 aa, Bacillus thuringiensis; identical to SEQ ID NO: 165

SEQ ID NO: 167 - protein, 786 aa, Bacillus thuringiensis
MQKNNKLSVKALPSFIDYFNGIYGFATGIKDIMNMIFKTNTGGDLTLDEILKNQQLLNEISGKLDGVNGSLNDLLAQGNLDTELSKEILKIANEQNKVLNDVNTKLDAINLMLNTYLPKITSMLSDVMKQNYALGLQIEYLSKQLKEISDKLDVINVNVLINSTLTEITPAYQRIKYVNEKFEALTSATETNLKTKQDSSHTDILDELTELTELAKSVTKNDVDGFEFYLNTFHDVMIGNNLFGRSALKTASELIAKENLKTSGSEVGNVYNFLIVLTALQAKAFLTLTTCRKLLGLADIDYTPIMNEHLNKEKEEFRVNILPTLSNTFSNPNYEKARGSDKDAKIIMEAKPGYALVGFEISKDSIAVLKVYQAKLKHNYQIDKDSLSEIVYGDIDKLLCPDQSEQMYYTNKIAFPNEYVITKIAFTKKLNSLRYEVTANFYDSSTGDIDLNKKKIESSEAEFSMLNANNDGVYMPIGTISETFLTPINGFGLVVDENSRLVTLTCKSYLRETLLATDLSNKETKLIVPPNGFISNIVENGNLEGENLEPWKANNKNAYVDHTGGVNGTKVLYVHEDGEFSQFIGDKLKLKTEYVIQYIVKGKAAIYLKDEKNGDYIYEETNNELEDFQAVTKRFITGTDSSRVHLIFTSQNGEEAFGGNFIISEIRPSEELLSPELIKSDAWVGSQGTWISGNSLNINSNVNGTFRQNLSLESYSTYSMNFNVNGFGKVTIRNSREVVFERSYLQFSSKYISEKFTTTTNNTGLYVELSRASSRGVINFGDFSIK

SEQ ID NO: 168 - protein, 786 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELAKQILKVANEQNQVLNDVNNKLDAINSMLKIYLPKITSMLSDVMKQNYVLSLQIEYLSKQLQEISDKLDIINVNVLINSTLTEITPAYQRIKYVNEKFDDLTFATENTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKAKGSNEDTKMIVEAKPGYVLVGFEMSNNSITVLKAYQAKLKKDYQIDKDSLSEIIYSDTDKLLCPDQSEQIYYTKNIAFPNEYVITKIAFTKKMNSLRYEATANFYDSSTGDIDLNKTKVESSEAEYSMLKASDDEVYMPLGLISETFLNPINGFRLAVDENSRLVTLTCRSYLRETLLATDLNNKETKLIVPPNVFISNIVENGNIEMDTLEPWKANNENANVDYSGGVNGTRALYVHKDGEFSHFIGDKLKSKTEYLIRYIVKGKASIFLKDEKNENYIYEDTNNNLEDYQTITKRFTTGTDSTGVYLIFNSQNGDEAWGDNFIILEISPCEKLLSPELIKTDKWISTGSTYISDDRLTLYQGGRGILKQNLQLDRFSTYRVNFSVNGDANVRIRNSREVLLEKRYLNRKGVSEMFTTKFDKDNFYVELSQGDNLGTVVHFYDFSIK

SEQ ID NO: 169 - protein, 786 aa, Bacillus thuringiensis
MNMNNAKLNARALPSFIDYFNGIYGFATGIKDIMNMIFKTDTGSNLTLDEILKNQQLLNEISGKLDGVNGSLNDLIAQGNLNTELAKQILKVANEQNQVLNDVNNKLDAINSMLKIYLPKITSMLSDVMKQNYVLSLQIEYLSKQLQEISDKLDIINVNVLINSTLTEITPAYQRMKYVNEKFEELTFATETTLKVKKDSPPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKAKGSNEDTKMIVEAKPGYVLVGFEMSNDSITVLKAYQAKLKKDYQIDKDSLSEIIYSDTDKLLCPDQSEQIYYTKNIAFPNEYVITKIAFTKKMNSLRYEATANFYDSSTGDIDLNKTKVESSEAEYSMLKASDDEVYMPLGLISETFLNPINGFRLAVDENSRLVTLTCRSYLRETLLATDLNNKETKLIVPPNVFISNIVENGNIEMDTLEPWKANNENANVDYSGGVNGTRALYVHKDGEFSHFIGDKLKSKTEYLIRYIVKGKASIFLKDEKNENYIYEDTNNNLEDYQTITKRFTTGTDSTGVYLIFNSQNGDEAWGDNFIILEISPCEKLLSPELIKTDKWNSTGSTYISDDRLTLYRGGRGILKQNLQLDGFSTYRVNFSVDGDANVRIRNSREVLLEKRYLNRKGVSEMFTTKFDKDNFYVELSQGDNLGTIVHFYDFSIK

SEQ ID NO: 170 - protein, 787 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKEDGSPADILDELTELTELAKSVTKNEVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYVKAKGSDRDAKMIVEAKPGYALVGFEMSNDSMTVLKAYQAKLKQDYQVDKDSLSEIVYGDMNKLLCPDQSEQIYYTNNIAFPREYVITKLTFTKKMNSLRYEATANFYDSSTGDMDLNKTKVESSEAEYSRLSASNDGVYMPLGLISETFLTPINGFGLVVDENSRLVTLTCKSYLREILLATDLSNKETKLIVPPNGFISNIVENGNLEGENLEPWKANNKNAYIDHTGGVKGTKVLYVHKDGEFSQFIGYKLKSKTEYVIQYIVKGKAVIYLKDEKNGDYIYEEINNELEDFQTVTKRFITGTDSSGVHLIFTSQNGEEAFGGNFIISEIRPSEELLSPELIKSDAWVGTQGAWNSGNSLTIYTNTNGTFRQNLPLESYSTYSMNFNITGFGKVTIRNSREVLFEKNFSQLSPKDYSEKFTTAANNTGFYVELSRGTQGGNITFRDFSIK

SEQ ID NO: 171 - protein, 788 aa, Bacillus thuringiensis
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDTDKLFCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGFSQFIGDKLKPKTEYVIQYTVKGKPSIHLKDENTGYIHYEDTNNNLKDYQTITKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSREVLFERSNLMSSTSHISGTFKTESNNTGLYVELSRRSGGGGHISFENVSIK

SEQ ID NO: 172 - protein, 787 aa, Bacillus thuringiensis
MNKNNTKLSTRALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLAGIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDTDKLFCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGFSQFIGDKLKPKTEYVIQYTVKGKPSIHLKDENTGYIHYEDTNNNLKDYQTITKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSRVLFERSNLMSSTSHISGTFKTESNNTGLYVELSRRSGGGGHISFENVSIK

SEQ ID NO: 173 - protein, 788 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSRGVLFERSNLMSSTSHISGTFKTESNNTGLYVELSRRSGGGGHISFENVSIK

SEQ ID NO: 174 - protein, 788 aa, Bacillus thuringiensis; identical to SEQ ID NO: 171

SEQ ID NO: 175 - protein, 787 aa, Bacillus thuringiensis
MNKNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLNAINTMLHIYLPKITSMLNDVMKQNYALSLQIEYLSKQLQEISDKLDVINVNVLINSTLTEITPAYQRMKYVNEKFEDLTFATETTLKVKKNSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTFIMNEHLDKEKEEFRVNILPTLSNTFSNPNYAKAKGSNEDAKIIVEAKPGYALVGFEMSNDSITVLKAYQAKLKQDYQVDKDSLSEIVYGDMDKLLCPDQSEQIYYTNNIAFPNEYVITKITFTKKMNSLRYEATANFYDSSTGDIDLNKTKVESSEAEYSTLSASTDGVYMPLGIISETFLTPINGFGIVVDENSKLVNLTCKSYLREVLLATDLSNKETKLIVPPIGFISNIVENGNLEGENLEPWKANNKNAYVDHTGGVNGTKALYVHKDGEFSQFIGDKLKSKTEYVIQYIVKGKASILLKDEKNGDCIYEDTNNGLEDFQTITKSFITGTDSSGVHLIFNSQNGDEAFGENFTISEIRLSEDLLSPELINSDAWVGSQGTWISGNSLTINSNVNGTFRQNLSLESYSTYSMNFNVNGFAKVTVRNSREVLFEKNYPQLSPKDISEKFTTAANNTGLYVELSRFTSGGAINFRNFSIK

SEQ ID NO: 176 - protein, 787 aa, Bacillus thuringiensis
MNKNNTKLSARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLNAINTMLHIYLPKITSMLNDVMKQNYALSLQIEYLSKQLQEISDKLDVINVNVLINSTLTEITPAYQRMKYVNEKFEDLTFATETTLKVKKNSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTFIMNEHLDKEKEEFRVNILPTLSNTFSNPNYAKAKGSNEDAKIIVEAKPGYALVGFEMSNDSITVLKAYQAKLKRDYQVDKDSLSEIVYGDMDKLLCPDQSEQIYYTNNIAFPNEYVITKITFTKKMNSLRYEATANFYDSSTGDIDLNKTKVESSEAEYSTLSASTDGVYTPLGIISETFLTPINGFGIVVDENSKLVNLTCKSYLREVLLATDLSNKETKLIVPPIGFISNIVENGNLEGENLEPWKANNKNAYVDHTGGVNGTKALYVHKDGEFSQFIGDKLKSKTEYVIQYIVKGKASILLKDEKNGDCIYEDTNNGLEDFQTITKSFITGTDSSGVHLIFNSQNGDEAFGENFTISEIRLSEDLLSPELINSDAWVGSQGTWISGNSLTINSNVNGTFRQNLSLESYSTYSMNFNVNGFAKVTVRNPREVLFEKNYPQLSPKDISEKFTTAANNTGLYVELSRFTSGGAINFRDFSIK

SEQ ID NO: 177 - protein, 840 aa, Bacillus thuringiensis
  SITE 58: note = misc_feature - Xaa can be any naturally occurring amino acid
MNMNNTKLNARALPSFIDYFNGIYGFATGIKDIMNMIFKTDTGGNLTLDEILKNQQLXNEISGKLDGVNGSLNDLIAQGNLNTELSKEILKIANEQNQVLNDVNNKLNAINTMLHIYLPKITSMLNDVMKQNYALSLQIEYLSKQLQEISDKLDVINVNVLINSTLTEITPAYQRMKYVNEKFEDLTFATETTLKVKKNSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTFIMNEHLDKEKEEFRVNILPTLSNTFSNPNYAKAKGSNEDAKIIVEAKPGYALVGFEMSNDSITVLKAYQAKLKQDYQVDKDSLSEIVYGDMDKLLCPDQSEQIYYTNNIAFPNEYVITKITFTKKMNSLRYEATANFYDSSTGDIDLNKTKVESSEAEYSTLSASTDGVYMPLGIISETFLTPINGFGIVVDENSKLVNLTCKSYLREVLLATDLSNKETKLIVPPIGFISNIVENGNLEGENLEPWKANNKNAYVDHTGGVNGTKALYVHKDGEFSQFIGDKLKSKTEYVIQYIVKGKASILLKDEKNGDCIYEDTNNGLEDFQTITKSFITGTDSSGVHLIFNSQNGDEAFGENFTISEIRLSEDLLSPELINSDAWVGSQGTWISGNSLTINSNVNGTFRQNLSLESYSTYSMNFNVNGFAKVTVRNSREVLFEKNYPQLSPKDISEKFTTAANNTGLYVELSRFTSGGAINFRDFSIKLVDPNSVQARLQDVDGTIDTRSKLAAAQLYTRASQPELAPEDPEDLEHHHHHH

SEQ ID NO: 178 - protein, 787 aa, Bacillus thuringiensis; identical to SEQ ID NO: 175

SEQ ID NO: 179 - protein, 787 aa, Bacillus thuringiensis
MNKNNTKLSARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLNAINTMLHIYLPKITSMLNDVMKQNYALSLQIEYLSKQLQEISDKLDVINVNVLINSTLTEITPAYQRMKYVNEKFEDLTFATETTLKVKKNSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTFIMNEHLDKEKEEFRVNILPTLSNTFSNPNYAKAKGSNEDAKIIVEAKPGYALVGFEMSNDSITVLKAYQAKLKQDYQVDKDSLSEIVYGDMDKLLCPDQSEQIYYTNNIAFPNEYVITKITFTKKMNSLRYEATANFYDSSTGDIDLNKTKVESSEAEYSTLSASTDGVYMPLGIISETFLTPINGFGIVVDENSKLVNLTCKSYLREVLLATDLSNKETKLIVPPIGFISNIVENGNLEGENLEPWKANNKNAYVDHTGGVNGTKALYVHKDGEFSQFIGDKLKSKTEYVIQYIVKGKASILLKDEKNGDCIYEDTNNGLEDFQTITKSFITGTDSSGVHLIFNSQNGDEAFGENFTISEIRLSEDLLSPELINSDAWVGSQGTWISGNSLTINSNVNGTFRQNLSLESYSTYSMNFNVNGFAKVTVRNSREVLFEKNYPQLSPKDISEKFTTAANNTGLYVELSRFTSGGAINFRDFSIK

SEQ ID NO: 180 - protein, 803 aa, Bacillus thuringiensis; identical to SEQ ID NO: 89

SEQ ID NO: 181 - protein, 795 aa, Bacillus thuringiensis
MNKNNTKLSTRALPSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETNLKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYIKTKGSDEDAEVIIQAEPGHALVGFEMINDPSPALKVYQAKLKPNYQVDKQSLSETVYGDMDKILCPDKSQQMYYLHNITFPNEYVITEIIFTKKKNSLRYEVIANYYEFSSGDIDLNKKLVKSSEAEYSTLSVSNDAIYMPLGVISETFLTPIKGFGLTVDESSRLVTLTCKSYLREILLATDLSNKATKLIVPPNGFISNLVENGDIEADNIEPWKGNNKNAYVDHTGGVNGTKALYTQDDGEFSQFIGDKLKSKTEYIIQYTVKGNTSIYLKDKKNENVIYEDKNNNLEAFQTITKRFTTELDSSDVYLVFKCKNGYKAWGDNFLITEIRPKEVVSPELIKVENWIGMGGSNHVNPDSLLLFTGGRSILKQNLQLDSYSTYRVRFSLMVIGKAKVIIRNSSEVLFEKSYVNDSEGVLEGVSETFTTKSIQDNFYVELSNEGTFGSKDVAYFYNFSIK

SEQ ID NO: 182 - protein, 803 aa, Bacillus thuringiensis; identical to SEQ ID NO: 89

SEQ ID NO: 183 - protein, 803 aa, Bacillus thuringiensis
MNMNNTKLNARALPSFIDYFNGIYGFATGIKDIMNMIFKTDTGGGNLTLDEILKNQDLLNQISDKLDGINGDLGDLIAQGNLNSELTKELLKIANEQNLMLNNVNAQLNSINATLNTYLPKITSMLNEVMKQNYVLSLQIEFLSKQLQEISDKLDIINLNVLINSTLTEITPAYQRIKYVNDKFDELTSTVEKNPKINQDNFTEDVIDNLTDLTELARSVTRNDMDSFEFYIKTFHDVMIGNNLFSRSALKTASELIAKENIHTRGSEIGNVYTFMIVLTSLQAKAFLTLTTCRKLLGLADIDYTQIMNENLDREKEEFRLNILPTLSNDFSNPNYTETLGSDLVDPIVTLEAEPGYALIGFEILNDPLPVLKVYQAKLKPNYQVDKESIMENIYGNIHKLLCPKQREQKYYIKDMTFPEGYVITKIVFEKKLNLLGYEVTANLYDPFTGSIDLNKTILESWKEDCCEEDCCEEDCCEEDCCEELYKIIEADTNGVYMPLGVISETFLTPIYSFKLIIDEKTKKISLAGKSYLRESLLATDLVNKETNLIPSPNGFISSIVQNWHITSDNIEPWKANNKNAYVDKTDAMVGFSSLYTHKDGEFLQFIGAKLKAKTEYIIQYTVKGNPEVYLKNNKDICYEDKTNNFDTFQTITKKFNSGVDPSEIYLVFKNQIGYEAWGNNFIILEIKSLETLPQILKPENWIPLGNAEIKEDGKIEISGNGSMNQYIQLEQNSKYHLRFSVKGKGRVTMQAQTSHINVPATNEEVSIMIETTRLYGEGIISLLNDEVENSGVIFSDVSIVKE

SEQ ID NO: 184 - protein, 803 aa, Bacillus thuringiensis; identical to SEQ ID NO: 89

SEQ ID NO: 185 - protein, 801 aa, Bacillus thuringiensis
MNNTKLNARALPSFIDYFNGIYGFATGIKDIMNMIFKTDTGGGNLTLDEILKNQDLLNQISDKLDGINGDLGDLIAQGNLNSELTKELLKIANEQNLMLNNVNAQLNSINSTLNTYLPKITSMLSEVMKQNYVLSLQIEFLSEQLQEISDKLDVINLNVLINSTLTEITPAYQRIKYVNDKFDELTSTVEKNPKINQDNFTEDVIDNLTDLTELARSVTRNDMDSFEFYIKTFHDVMIGNNLFSRSALKTASELIAKENIHTMGSEIGNVYTFMVVLTSLQAKAFLTLTACRKLLGLTDIDYTQIMNENLNREKEEFRLNILPTLSNDFSNPNYTETLGSDLVDPIVTLEADPGYALIGFEILNDPLPVLKVYQAKLKPNYQVDKESIMENIYGNIHKLLCPKQRHQKYYIKDITFPEGYVITKIVFEKKLNLLGYEVTANLYDPFTGSIDLNKTILESWKEECCEEECCEEECCEEECCEELYKIIEADTNGVYMPLGVISETFLTPIYSFKLIIDERTKRISLAGKSYLRESLLATDLVNKDTNLIPSPNGFINSIVENWNITSDNIEPWKANNKNAYVDKTDDMVGFNSLYTHKDGEFLQFIGAKLKAKTEYIIQYTVKGSPEVYLKNNKGIFYEDTTNKFDTFQTITKKFNSGVDPSEIYLVFKNQIGYEAWGNKFIILEIKSFETLPQILKPENWMPFGNAEIKEDGKIEISGNGTMTQNIQLEQNSKYHLRFSVKGKGRVAIQTQSSHINVPATNEEVSTMITTRNLYGEGMIYLFNDDVENSKVIFSDVSLVKE

SEQ ID NO: 186 - protein, 700 aa, Bacillus thuringiensis
MNNTKLNARALPSFIDYFNGIYGFATGIKDIMNMIFKTDTGGGNLTLDEILKNQDLLNQISDKLDGINGDLGDLIAQGNLNSELTKELLKIANEQNLMLNNVNAQLNSINSTLNTYLPKITSMLSEVMKQNYVLSLQIEFLSEQLQEISDKLDVINLNVLINSTLTEITPAYQRIKYVNDKFDELTSTVEKNPKINQDNFTEDVIDNLTDLTELARSVTRNDMDSFEFYIKTFHDVMIGNNLFSRSALKTASELIAKENIHTMGSEIGNVYTFMVVLTSLQAKAFLTLTACRKLLGLTDIDYTQIMNENLNREKEEFRLNILPTLSNDFSNPNYTETLGSDLVDPIVTLEADPGYALIGFEILNDPLPVLKVYQAKLKPNYQVDKESIMENIYGNIHKLLCPKQRQQKYYIKDITFPEGYVITKIVFEKKLNLLGYEVTANLYDPFTGSIDLNKTILESWKEECCEEECCEEECCEEECCEELYKIIEADTNGVYMPLGVISETFLTPIYSFKLIIDERTKRISLAGKSYLRESLLATDLVNKDTNLIPSPNGFISSIVENWNITSDNIEPWKANNKNAYVDKTDDMVGFNSLYTHKDGEFLQFIGAKLKAKTEYIIQYTVKGSPEVYLKNNKGIFYEDTTNKFDTFQTITKKFNSGVDPSEIYLVFKNQIGYEAWGNKFIILEIKSFETLPQILKPENW

SEQ ID NO: 187 - protein, 490 aa, Bacillus thuringiensis
MNKNNTKLSARALPSFIDYFNGIYGFATGIKDIMNMIFKTDTGGGNLTLDEILKNQDLLNQISDKLDGINGDLGDLIAQGNLNSELIKELLKIANEQNLMLNNVNAQLNSINSTLNTYLPKITSMLSEVMKQNYVLSLQIEFLSEQLQEISDKLDVINLNVLINSTLTEITPAYQRIKYVNDKFDELTSTVEKNPKINQDNFTEDVIDNLTDLTELARSVTRNDMDSFEFYIKTFHDAMIGNNLFSRSALKTASELIAKENIHTMGSEIGNVYTFMVVLTSLQAKAFLTLTACRKLLGLTDIDYTQIMNENLNREKEEFRLNVLPTLSNDFSNPNYTETLGSDLVDPIVTLEADPGYALIGFEILNDPLPVLKVYQAKLKPNYQVDKESIMENIYGNIHKLLCPKQRQQKYYIKDITFPEGYVITKIVFEKKLNLLGYEVTANLYDPFTGSIDLNKTILESWKEECCEEECCEEECCEEECCEELYKIIE

SEQ ID NO: 188 - protein, 803 aa, Bacillus thuringiensis
MNKNNTKLNARALPSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLNIYLPKITSMLSDVMKQNYALSLQIEYLSRQLQEISDKLDVINLNVLINSTLTEITPSYQRIKYVNEKFDKLTFATESTLRAKQGIFNEDSFDNNTLENLTDLAELAKSITKNDVDSFEFYLHTFHDVLIGNNLFGRSALKTASELITKDEIKTSGSEIGKVYSFLIVLTSLQAKAFLTLTTCRKLLGLSDIDYTSIMNEHLNNEKNEFRDNILPALSNKFSNPSYAKTIGSDNYAKVILESEPGYALVGFEIINDPIPVLKAYKAKLKQNYQVDNQSLSEIVYLDIDKLFCPENSEQKYYTKNLTFPDGYVITKITFEKKLNNLIYEATANFYDPSTGDIDLNKKQVESTFPQTDYITMDIGDDDGIYMPLGVISETFLTPINSFGLEVDAKSKTLTLKCKSYLREYLLESDLKNKETGLIAPPNVFISNVVKNWDIEEDSLEPWVANNKNAYVDNTGGIERSKALFTQGDGEFSQFIGDKLKPNTDYIIQYTVKGKPAIYLKNKSTGYITYEDTNGNSEEFQTIAVKFTSETDLSQTHLVFKSQNGYEAWGDNFIILEAKLFETPESPELIKFNDWERFGTTYITGNELRIDHSRGGYFRQSLNIDSYSTYDLSFSFSGLWAKVIVKNSRGVVLFEKVKNNGSSYEDISESFTTASNKDGFFIELTAERTSSTFHSFRDISIKEKIE

SEQ ID NO: 189 - protein, 803 aa, Bacillus thuringiensis; identical to SEQ ID NO: 89

SEQ ID NO: 190 - protein, 803 aa, Bacillus thuringiensis
MNKNNTKLNARALPSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLNIYLPKITSMLSDVMKQNYALSLQIEYLSRQLQEISDKLDVINLNVLINSTLTEITPSYQRIKYVNEKFDKLTFATESTLRAKQGIFNEDSFDNNTLENLADLAELAKSITKNDVDSFEFYLHTFHDVLIGNNLFGRSALKTASELITKDEIKTSGSEIGKVYSFLIVLTSLQAKAFLTLTTCRKLLGLSDIDYTSIMNEHLNNEKNEFRDNILPALSNKFSNPSYAKTIGSDNYAKVILESEPGYALVGFEIINDPIPVLKAYKAKLKQNYQVDNQSLSEIVYLDIDKLFCPENSEQKYYTKNLTFPDGYVITKITFEKKLNNLIYEATANFYDPSTGDIDLNKKQVESTFPQTDYITMDIGDDDGIYMPLGVISETFLTPINSFGLEVDAKSKTLTLKCKSYLREYLLESDLKNKETGLIAPPNVFISNVVKNWDIEEDSLEPWVANNKNAYVDNTGGIERSKALFTQGDGEFSQFIGDKLKPNTDYIIQYTVKGKPAIYLKNKSTGYITYEDTNGNSEEFQTIAVKFTSETDLSQTHLVFKSQNGYEAWGDNFIILEAKLFETPESPELIKFNDWERFGTTYITGNELRIDHSRGGYFRQSLNIDSYSTYDLSFSFSGLWAKVIVKNSRGVVLFEKVKNNGSSYEDISESFTTASNKDGFFIELTAERTSSTFHSFRDISIKEKIE

SEQ ID NO: 191 - protein, 803 aa, Bacillus thuringiensis; identical to SEQ ID NO: 89

SEQ ID NO: 192 - protein, 803 aa, Bacillus thuringiensis; identical to SEQ ID NO: 190

(19) United States
(12) Patent Application Publication
(10) Pub. No.: US 2025/0212890 A1
(43) Pub. Date: Jul. 3, 2025

(54) COMBINATIONS OF AV3 MUTANT POLYPEPTIDES AND Bt TOXINS FOR PEST CONTROL

(71) Applicant: Vestaron Corporation, Durham, NC (US)

(72) Inventor: Lin BAO, Portage, MI (US)

(73) Assignee: Vestaron Corporation, Durham, NC (US)

(21) Appl. No.: 18/852,355

(22) PCT Filed: Mar. 30, 2023
(86) PCT No.: PCT/US2023/065127

§ 371 Date: Sep. 27, 2024

Related U.S. Application Data
(60) Provisional application No. 63/325,603, filed on Mar. 30, 2022.

(51) Int. Cl. A01N 63/23; A01N 63/50; A01P 7/04

(52) U.S. Cl. CPC A01N 63/23; A01N 63/50; A01P 7/04

(57) ABSTRACT

New insecticidal peptides, polypeptides, proteins, and nucleotides; their expression in culture and plants; methods of producing the peptides, polypeptides, proteins, and nucleotides; new processes; new production techniques; new formulations; and new organisms, are disclosed. The present disclosure is also related to mutants named Av3 mutant polypeptides (AMPs) that are a non-naturally occurring, modified-form of the peptide, Av3, isolated from the sea anemone, Anemonia viridis. Here we describe: polynucleotides encoding AMPs; various formulations and combinations of both polynucleotides and peptides; and methods for using the same that are useful for the control of insects.

### CROSS REFERENCE TO RELATED APPLICATIONS

This application claims the benefit of, and priority to, U.S. Provisional Application Ser. No. 63/325,603 filed on Mar. 30, 2022, the disclosure of which is incorporated by reference herein in its entirety.

### SEQUENCE

This application incorporates by reference herein in its entirety the Sequence Listing entitled “225312-FAM GG.xml” (296 KB), which was created on Mar. 28, 2023, at 4:50 PM, and filed electronically herewith.

### TECHNICAL FIELD

New combinations of insecticidal proteins, new formulations, new agricultural compositions, and methods of making and using the same for the control of insects are described.

### BACKGROUND

Deleterious insects represent a worldwide threat to human health and food security. Insects pose a threat to human health because they are a vector for disease. One of the most notorious insect-vectors of disease is the mosquito. Mosquitoes in the genus Anopheles are the principal vectors of Zika virus, Chikungunva virus, and malaria—a disease caused by protozoa in the genus Trypanosoma. Another mosquito, Aedes aegypti, is the main vector of the viruses that cause Yellow fever and Dengue. And, Aedes spp. mosquitos are also the vectors for the viruses responsible for various types of encephalitis. Wuchereria bancrofti and Brugia malayi, parasitic roundworms that cause filariasis, are usually spread by mosquitoes in the genera Culex, Mansonia, and Anopheles.

Similar to the mosquito, other members of the Diptera order have likewise plagued humankind since time immemorial. In addition to producing painful bites, Horseflies and deerflies transmit the bacterial pathogens of tularemia (Pasteurella tularensis) and anthrax (Bacillus anthracis), as well as a parasitic roundworm (Loa loa) that causes loiasis in tropical Africa.

Blowflies (Chrysomya megacephala) and houseflies (Musca domestica) will in one moment take off from carrion and dung, and in the next moment alight in our homes and on our food-spreading dysentery, typhoid fever, cholera, poliomyelitis, yaws, leprosy, and tuberculosis in their wake.

Eye gnats in the genus Hippelates can carry the spirochaete pathogen that causes yaws (Treponema pertenue), and may also spread conjunctivitis (pinkeye). Tsetse flies in the genus Glossina transmit the protozoan pathogens that cause African sleeping sickness (Trypanosoma gambiense and T. rhodesiense). Sand flies in the genus Phlebotomus are vectors of a bacterium (Bartonella bacilliformis) that causes Carrion's disease (Oroyo fever) in South America. In parts of Asia and North Africa, they spread a viral agent that causes sand fly fever (Pappataci fever) as well as protozoan pathogens (Leishmania spp.) that cause Leishmaniasis.

Human food security is also threatened by insects. Insect pests indiscriminately target food crops earmarked for commercial purposes and personal use alike; indeed, the damage caused by insect pests can run the gamut from mere inconvenience to financial ruin in the former, to extremes such as malnutrition or starvation in the latter. Insect pests also cause stress and disease in domesticated animals. And, insect pests once limited by geographical and climate boundaries have expanded their range due to global travel and climate change.

### SUMMARY

The present disclosure describes an insecticidal combination comprising an Av3 mutant polypeptide (AMP), and a Bacillus thuringiensis ssp. kurstaki (Btk) toxin; wherein the AMP comprises an amino acid sequence that is at least 95%, 96%, 97%, 97%, 98%, 99%, or 100% identical to the amino acid sequence set forth in SEQ ID NO: 1, or a agriculturally acceptable salt thereof.

In addition, the present disclosure describes a combination comprising one or more fermentation solids, spores, or toxins isolated from a Bacillus thuringiensis ssp. kurstaki strain EVB-113-19, and an Av3 mutant polypeptide (AMP) having an amino acid sequence set forth in SEQ ID NO: 1.

In addition, the present disclosure describes a combination comprising one or more fermentation solids, spores, or toxins isolated from a Bacillus thuringiensis ssp. kurstaki strain ABTS-351, and an Av3 mutant polypeptide (AMP) having an amino acid sequence set forth in any one of SEQ ID NO: 1.

In addition, the present disclosure describes agricultural compositions comprising an Av3 mutant polypeptide (AMP); a Bacillus thuringiensis ssp. kurstaki (Btk) toxin; and at least one excipient; wherein the AMP comprises an amino acid sequence that is at least 95%, 96%, 97%, 97%, 98%, 99% or 100% identical to the amino acid sequence set forth in SEQ ID NO: 1, or a agriculturally acceptable salt thereof.

In addition, the present disclosure describes a method of combating, controlling, or inhibiting a pest comprising, applying a pesticidally effective amount of the combination comprising (1) an AMP and (2) a Btk toxin, and/or an agricultural composition thereof further comprising an excipient, to: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof.

### BRIEF DESCRIPTION OF THE DRAWINGS

FIG. 1 depicts the results of a randomized complete block design (RCBD) field experiment was performed to evaluate the effect of a combination of Av3b and Bacillus thuringiensis ssp. kurstaki (Btk) toxins on larvae number when used against diamondback moths (Plutella xylostella) on cabbage (Brassica oleraceae var. capitata f alba). The letters “a” and “b” denote significant differences between treatments (ANOVA) (e.g., “a” relative to “b” is significant). Here, plants treated with the med (18 g AI/acre) and high (36 g AI/acre) dose of Av3b had significantly fewer Plutella xylostella larvae as compared to the amount of larvae on plants treated with the low (9 g AI/acre) and control (UTC). Spray treatments were applied 4 times (treatments: A, B, C, and D), with each spray treatment separated by 7 days. 7 DA−D=7 days after treatment D.

FIG. 2 depicts the results of a randomized complete block design (RCBD) field experiment was performed to evaluate the effect of a combination of Av3bM170 and Bacillus thuringiensis ssp. kurstaki (Btk) toxins on larvae number when used against diamondback moths (Plutella xylostella) on cabbage (Brassica oleraceae var. capitata f. alba). The letters “a”, “b”, and “c” denote significant differences between treatments (ANOVA) (e.g., “a” relative to “c” is significant). Here, plants treated with the med (2 g AI/acre) and high (5 g AI/acre) dose of Av3bM170 had significantly fewer Plutella xylostella larvae as compared to the amount of larvae on plants treated with the low (1 g AI/acre) and control (UTC). Spray treatments were applied 3 times (treatments: 1, 2, and 3), with each spray treatment separated by 7 days. Larvae count per 10 plants was taken 7 days after treatment 3 (i.e., 7 DAT3).

FIG. 3 shows the logistic regression used to calculate the dose resulting in 50% larval control per plant relative to the UTC (EC50) when comparing Av3b to Av3bM170 in a randomized complete block design (RCBD) field experiment. Here, Av3b to Av3bM170 were both applied in combination with Bacillus thuringiensis ssp. kurstaki (Btk) toxins. When using Av3b+Btk toxins, the rate that caused 50% larval control per plant relative to the untreated control (UTC) was 13.17 grams of active ingredient per acre (g AI/acre). However, when using Av3bM170+Btk toxins, the rate that caused 50% larval control per plant relative to the UTC was 1.50 g AI/acre.

FIG. 4 shows the results of percent mortality in a field-to-lab experiment evaluating the effect of a combination of Av3b and Btk toxins, against Beet armyworm (Spodoptera exigua) on tomato (Solanum lycopersicum). Results show percent mortality 72-hours after application of Av3b+Btk toxins. Here, UTC=untreated control; g ai/A=grams of active ingredient per acre.

FIG. 5 depicts the results of randomized complete block design (RCBD) field experiment evaluating the effect of a combination of Av3bM170 and Bacillus thuringiensis ssp. kurstaki (Btk) toxins on percent almond (Prunus amygdalus) nut damage, when used against Navel Orangeworm (Amyelois transitella). The letters “a” and “b” denote significant differences between treatments (Tukey's HSD) (e.g., “a” relative to “b” is significant; “ab” is not significant from “a” or “b”). Here, plants treated with high (5 g AI/acre) dose of Av3bM170 had significantly less nut damage as compared to the untreated control (UTC), the low dose (1 g AI/acre), and medium dose (2 g AI/acre) of Av3bM170.

FIG. 6 shows the results of a foliar spray bioassay evaluating the effect of a combination of Av3b with Bacillus thuringiensis ssp. kurstaki (Btk) toxins, on fall armyworm (Spodoptera frugiperda) mortality. Here, the treatments were: (1) Av3b+Btk; (2) Btk alone; (3) Av3b alone; and (4) 0.25% Water/untreated control (UTC) (0.25% solution of non-ionic surfactant with water). Av3b was evaluated at concentrations of 1.4 mg/mL, 4.2 mg/mL, and 12.4 mg/mL. Btk was evaluated using a sublethal dose (LD₂₀) of 15 ppm. Here, the LC₅₀ for Av3b when combined with Bacillus thuringiensis ssp. kurstaki (Btk) toxins is 1.68 mg/mL.

FIG. 7 shows the results of a foliar spray bioassay evaluating the effect of a combination of Av3bM170 with Bacillus thuringiensis ssp. kurstaki (Btk) toxins, on fall armyworm (Spodoptera frugiperda) mortality. Here, the treatments were: (1) Av3bM170+Btk; (2) Btk alone; (3) Av3bM170 alone; and (4) 0.25% Water/untreated control (UTC) (0.25% solution of non-ionic surfactant with water). Av3bM170 was evaluated at concentrations of 0.3 mg/mL, 1 mg/mL, and 3 mg/mL. Btk was evaluated using a sublethal dose (LD₂₀) of 15 ppm. Here, the LC₅₀ for Av3bM170 when combined with Bacillus thuringiensis ssp. kurstaki (Btk) toxins is 1.51 mg/mL.

### DETAILED DESCRIPTION

### Definitions

“5′-end” and “3′-end” refers to the directionality, i.e., the end-to-end orientation of a nucleotide polymer (e.g., DNA). The 5′-end of a polynucleotide is the end of the polynucleotide that has the fifth carbon.

“5′- and 3′-homology arms” or “5′ and 3′ arms” or “left and right arms” refers to the polynucleotide sequences in a vector and/or targeting vector that homologously recombine with the target genome sequence and/or endogenous gene of interest in the host organism in order to achieve successful genetic modification of the host organism's chromosomal locus.

“Additive” refers to any agriculturally acceptable additive. Agriculturally acceptable additives include, without limitation, disintegrants, dispersing additives, coating additives, diluents, surfactants, absorption promoting additives, anti-caking additives, anti-microbial agents (e.g., preservatives), colorants, desiccants, plasticizers and dyes.

“Alignment” refers to a method of comparing two or more sequences (e.g., nucleotide, polynucleotide, amino acid, peptide, polypeptide, or protein sequences) for the purpose of determining their relationship to each other. Alignments are typically performed by computer programs that apply various algorithms, however, it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW (see Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV (see Larkin M. A., et al., CLUSTALW2, ClustalW and ClustalX version 2, Bioinformatics 23(21): 2947-2948, 2007); Mafft; Kalign; ProbCons; and T-Coffee (see Notredame et al., T-Coffee: A novel method for multiple sequence alignments, Journal of Molecular Biology 302: 205-217, 2000). Exemplary programs that implement one or more of the foregoing algorithms include, but are not limited to, MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In some embodiments, an alignment will introduce “phase shifts” and/or “gaps” into one or both of the sequences being compared in order to maximize the similarity between the two sequences, and scoring refers to the process of quantitatively expressing the relatedness of the aligned sequences.

“Agent” refers to one or more chemical substances, molecules, nucleotides, polynucleotides, peptides, polypeptides, proteins, poisons, insecticides, pesticides, organic compounds, inorganic compounds, prokaryote organisms, or eukaryote organisms, and agents produced therefrom.

“Agriculturally-acceptable carrier” covers all adjuvants, inert components, dispersants, surfactants, tackifiers, binders, etc. that are ordinarily used in pesticide formulation technology; these are well known to those skilled in pesticide formulation.

“Agriculturally acceptable salt” is synonymous with pharmaceutically acceptable salt, and as used herein refers to a compound that is modified by making acid or base salts thereof.

“Agroinfection” means a plant transformation method where DNA is introduced into a plant cell by using Agrobacteria A. tumefaciens or A. rhizogenes.

“Alpha-MF signal” or “αMF secretion signal” refers to a protein that directs nascent recombinant polypeptides to the secretory pathway.

“AMP” or “Av3 mutant polypeptide” or “Av3b mutant polypeptide” or “Av3b mutant peptide” or “Av3 mutant polypeptide” or “Av3b mutant polypeptide” or “Av3b mutant protein, as used herein, all refer to the Av3b mutant named “Av3bM170,” which has an amino acid sequence of: “KSCCPCYWGGCPWGQNCYPEGCGGPG” (SEQ ID NO: 1).

“AMP expression cassette” refers to one or more regulatory elements such as promoters; enhancer elements; mRNA stabilizing polyadenylation signal; an internal ribosome entry site (IRES); introns; post-transcriptional regulatory elements; and a polynucleotide operable to encode an AMP, e.g., an AMP ORF. For example, one example of an AMP expression cassette is one or more segments of DNA that contains a polynucleotide segment operable to express an AMP, a ADH1 promoter, a LAC4 terminator, and an alpha-MF secretory signal. An AMP expression cassette contains all of the nucleic acids necessary to encode an AMP or an AMP-insecticidal protein.

“AMP ORF” refers to a polynucleotide operable to encode an AMP, or an AMP-insecticidal protein.

“AMP ORF diagram” refers to the composition of one or more AMP ORFs, as written out in diagram or equation form. For example, a “AMP ORF diagram” can be written out as using acronyms or short-hand references to the DNA segments contained within the expression ORF. Accordingly, in one example, a “AMP ORF diagram” may describe the polynucleotide segments encoding the ERSP, LINKER, STA, and AMP, by diagramming in equation form the DNA segments as “ersp” (i.e., the polynucleotide sequence that encodes the ERSP polypeptide); “linker” or “L” (i.e., the polynucleotide sequence that encodes the LINKER polypeptide); “sta” (i.e., the polynucleotide sequence that encodes the STA polypeptide), and “amp” (i.e., the polynucleotide sequence encoding an AMP), respectively. An example of an AMP ORF diagram is “ersp-sta-(linkerᵢ-ampⱼ)_{N},” or “ersp-(ampⱼ-linkerᵢ)_{N}-sta” and/or any combination of the DNA segments thereof.

“AMP-insecticidal protein” or “AMP-insecticidal polypeptide” or “insecticidal protein” or “insecticidal polypeptide” refers to any protein, peptide, polypeptide, amino acid sequence, configuration, or arrangement, comprising: (1) at least one AMP, or two or more AMPs; and (2) additional peptide, polypeptide, or protein. For example, in some embodiments, these additional peptide, polypeptide, or protein have the ability to increase the mortality and/or inhibit the growth of insects when the insects are exposed to an AMP-insecticidal protein, relative to an AMP alone; increase the expression of said AMP-insecticidal protein, e.g., in a host cell or an expression system; and/or affect the post-translational processing of the AMP-insecticidal protein. In some embodiments, an AMP-insecticidal protein can be a polymer comprising two or more AMPs. In some embodiments, an AMP-insecticidal protein can be a polymer comprising two or more AMPs, wherein the AMPs are operably linked via a linker peptide, e.g., a cleavable and/or non-cleavable linker. In some embodiments, an AMP-insecticidal protein can refer to a one or more AMPs operably linked with one or more proteins such as a stabilizing domain (STA); an endoplasmic reticulum signaling protein (ERSP); an insect cleavable or insect non-cleavable linker (L); and/or any other combination thereof. In some embodiments, an AMP-insecticidal protein can be a non-naturally occurring protein comprising (1) an AMP; and (2) an additional peptide, polypeptide, or protein, e.g., an ERSP; a linker; a dipeptide, a STA; a UBI; or a histidine tag or similar marker.

“AMP construct” refers to the three-dimensional arrangement/orientation of peptides, polypeptides, and/or motifs of operably linked polypeptide segments (e.g., an AMP-insecticidal protein). For example, an AMP ORF can include one or more of the following components or motifs: an AMP; an endoplasmic reticulum signal peptide (ERSP); a linker peptide (L); a translational stabilizing protein (STA); or any combination thereof. And, as used herein, the term “AMP construct” is used to describe the designation and/or orientation of the structural motif. In other words, the AMP construct describes the arrangement and orientation of the components or motifs contained within a given AMP ORF. For example, in some embodiments, an AMP construct describes, without limitation, the orientation of one of the following AMP-insecticidal proteins: ERSP-AMP; ERSP-(AMP)_{N}; ERSP-AMP-L; ERSP-(AMP)_{N}-L; ERSP-(AMP-L)_{N}; ERSP-L-AMP; ERSP-L-(AMP)_{N}; ERSP-(L-AMP)_{N}; ERSP-STA-AMP; ERSP-STA-(AMP)_{N}; ERSP-AMP-STA; ERSP-(AMP)_{N}-STA; ERSP-(STA-AMP)_{N}; ERSP-(AMP-STA)_{N}; ERSP-L-AMP-STA; ERSP-L-STA-AMP; ERSP-L-(AMP-STA)_{N}; ERSP-L-(STA-AMP)_{N}; ERSP-L-(AMP)_{N}-STA; ERSP-(L-AMP)_{N}-STA; ERSP-(L-STA-AMP)_{N}; ERSP-(L-AMP-STA)_{N}; ERSP-(L-STA)_{N}-AMP; ERSP-(L-AMP)_{N}-STA; ERSP-STA-L-AMP; ERSP-STA-AMP-L; ERSP-STA-L-(AMP)_{N}; ERSP-(STA-L)_{N}-AMP; ERSP-STA-(L-AMP)_{N}; ERSP-(STA-L-AMP)_{N}; ERSP-STA-(AMP)_{N}-L; ERSP-STA-(AMP-L)_{N}; ERSP-(STA-AMP)_{N}-L; ERSP-(STA-AMP-L)_{N}; ERSP-AMP-L-STA; ERSP-AMP-STA-L; ERSP-(AMP)_{N}-STA-L ERSP-(AMP-L)_{N}-STA; ERSP-(AMP-STA)_{N}-L; ERSP-(AMP-L-STA)_{N}; or ERSP-(AMP-STA-L)_{N}; wherein N is an integer ranging from 1 to 200. See also “Structural motif.”

“Av3 mutant polynucleotide” refers to the polynucleotide sequence that encodes any AMP. The term “Av3 mutant polynucleotide” when used to describe the Av3 mutant polynucleotide sequence, e.g., such as one contained in an AMP open reading frame (ORF), its inclusion in a vector, and/or when describing the polynucleotides encoding an insecticidal protein, is written in lowercase and italicized, e.g., “amp” and/or “Amp”.

“Applying” or “application” or “apply” or “administering” or “administration” or “administer” means to dispense and/or otherwise provide, and refers to any method of application or route of administration. For example, applying can refer to, e.g., application of the combination of the present disclosure, e.g., an AMP or an agriculturally acceptable salt thereof and a Bt toxin; or application of the combination, and one or more excipients, e.g., a sprayable composition, a foam; a burning formulation; a fabric treatment; a surface-treatment; a dispersant; a microencapsulation, and the like. By “co-application” or “co-administer” it is meant that two or more components are applied or administered at the same time; or a one or more components are applied or administered just prior to, or just after the application the other one or more components. For example, in some embodiments, an AMP or agriculturally acceptable salt thereof and a Bt toxin, can be applied or administered simultaneously or sequentially.

“Av3b” refers to a peptide having an amino acid sequence of KSCCPCYWGGCPWGQNCYPEGCSGPK” (SEQ ID NO: 3). Here, Av3b has an N-terminal mutation and a C-terminal mutation relative to the wild type Av3 peptide (SEQ ID NO: 2), wherein the N-terminal mutation results in an amino acid substitution of R1K relative to SEQ ID NO:2, and the C-terminal mutation results in an amino acid deletion relative to SEQ ID NO:2; thus, in an Av3b peptide, the wild-type Av3 peptide amino acid sequence is changed from the wild-type Av3 amino acid sequence: “RSCCPCYWGGCPWGQNCYPEGCSGPKV” (SEQ ID NO: 2), to the Av3b amino acid sequence: “KSCCPCYWGGCPWGQNCYPEGCSGPK” (SEQ ID NO: 3).

“Binary vector” or “binary expression vector” means an expression vector which can replicate itself in both E. coli strains and Agrobacterium strains. Also, the vector contains a region of DNA (often referred to as t-DNA) bracketed by left and right border sequences that is recognized by virulence genes to be copied and delivered into a plant cell by Agrobacterium.

“bp” or “base pair” refers to a molecule comprising two chemical bases bonded to one another forming a. For example, a DNA molecule consists of two winding strands, wherein each strand has a backbone made of an alternating deoxyribose and phosphate groups. Attached to each deoxyribose is one of four bases, i.e., adenine (A), cytosine (C), guanine (G), or thymine (T), wherein adenine forms a base pair with thymine, and cytosine forms a base pair with guanine.

“Bt” refers Bacillus thuringiensis—a Gram positive, spore-forming bacterium, such as Bacillus thuringiensis ssp. kurstaki (Btk), Bacillus thuringiensis ssp. tenebrionis (Btt), and Bacillus thuringiensis ssp. israelensis (Bti).

“Bt toxin” or “Bacillus thuringiensis toxin” refers to any one or more fermentation solids, spores, insecticidal proteins, pesticidal proteins, or toxins produced by, isolated from, derived from, or otherwise originating in a Bacillus thuringiensis, or a subspecies thereof (e.g., Bacillus thuringiensis kurstaki). For example, in some embodiments, a Bt toxin can be any one or more fermentation solids, spores, insecticidal proteins, pesticidal proteins, or toxins produced by, isolated from, derived from, or otherwise originating in a Bacillus thuringiensis, or a subspecies thereof. In other embodiments, a Bt toxin can be a specific fermentation solid, spore, insecticidal protein, pesticidal protein, or toxin, belonging to a known class of Bt toxins. For example, in some embodiments, a Bt toxin can be an insecticidal protein, pesticidal protein, or toxin belonging to one of the following classes: Cry (e.g., such as proteins originally isolated from B. thuringiensis crystals in which the active form normally consists of three domains); Cyt (e.g., cytolytic proteins which normally comprise single domain proteins); Vip (multi-domain proteins originally identified as being Vegetative Insecticidal Proteins); Tpp (beta pore-forming pesticidal proteins containing the Toxin_10 (Bin-like) domain); Mpp (beta pore-forming pesticidal proteins from the ETX/Mtx2 family); Gpp (aegerolysin like pesticidal proteins); App (predominantly alpha helical pesticidal proteins); Spp (sphaericolysin like pesticidal proteins); Mcf (proteins related to the “Makes Caterpillars Floppy” toxins); Mix (proteins related to the Mtx1 toxin (2VSE) originally isolated from Lysinibacillus sphaericus); Vpa (proteins related to the ADP-ribosyltransferase active component of binary toxins); Vpb (proteins related to the binding component of binary toxins); Pra (proteins related to the Photorhabdus Insect-Related toxin A component); Prb (proteins related to the Photorhabdus Insect-Related toxin B component); Mpf (pesticidal proteins that are part of the Membrane Attack Complex/Perforin superfamily); or Xpp (a holding class for pesticidal proteins with currently uncharacterized structures).

“Btk toxin” or “Bacillus thuringiensis ssp. kurstaki toxin” refers to any one or more fermentation solids, spores, insecticidal proteins, pesticidal proteins, or toxins produced by, isolated from, derived from, or otherwise originating in a Bacillus thuringiensis ssp. kurstaki (Btk).

“C-terminus” or “C-terminal” refers to the free carboxyl group (i.e., —COOH) that is positioned on the terminal end of a polypeptide.

“cDNA” or “copy DNA” or “complementary DNA” refers to a molecule that is complementary to a molecule of RNA. In some embodiments, cDNA may be either single-stranded or double-stranded. In some embodiments, cDNA can be a double-stranded DNA synthesized from a single stranded RNA template in a reaction catalyzed by a reverse transcriptase. In yet other embodiments, “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein. In some embodiments, “cDNA” refers to a DNA that is complementary to and derived from an mRNA template.

“CEW” refers to Corn earworm.

“Cleavable Linker” see Linker.

“Cloning” refers to the process and/or methods concerning the insertion of a DNA segment (e.g., usually a gene of interest, for example amp) from one source and recombining it with a DNA segment from another source (e.g., usually a vector, for example, a plasmid) and directing the recombined DNA, or “recombinant DNA” to replicate, usually by transforming the recombined DNA into a bacteria or yeast host.

“Coding sequence” or “CDS” refers to a polynucleotide or nucleic acid sequence that can be transcribed (e.g., in the case of DNA) or translated (e.g., in the case of mRNA) into a peptide, polypeptide, or protein, when placed under the control of appropriate regulatory sequences and in the presence of the necessary transcriptional and/or translational molecular factors. The boundaries of the coding sequence are determined by a translation start codon at the 5′ (amino) terminus and a translation stop codon at the 3′ (carboxy) terminus. A transcription termination sequence will usually be located 3′ to the coding sequence. In some embodiments, a coding sequence may be flanked on the 5′ and/or 3′ ends by untranslated regions. In some embodiments, a coding sequence can be used to produce a peptide, a polypeptide, or a protein product. In some embodiments, the coding sequence may or may not be fused to another coding sequence or localization signal, such as a nuclear localization signal. In some embodiments, the coding sequence may be cloned into a vector or expression construct, may be integrated into a genome, or may be present as a DNA fragment.

“Codon optimization” refers to the production of a gene in which one or more endogenous, native, and/or wild-type codons are replaced with codons that ultimately still code for the same amino acid, but that are of preference in the corresponding host.

“Combination” refers to the result of combining two or more separate components. Thus, as used herein, a “combination” refers to an association of two or more separate components, e.g., an AMP and at least one Bt toxin. Accordingly, in some embodiments, a combination can refer to the association of an AMP and one or more Bt toxins. In some embodiments, the combination can be, e.g., a mixture, or as part of a composition further comprising one or more excipients. In some embodiments, a combination can refer to the simultaneous, separate, or sequential application of two or more separate components (e.g., an AMP and one or more Bt toxins). For example, in some embodiments, a “combination” refers to the result of a simultaneous application of both an AMP and one or more Bt toxins. In another embodiment, a “combination” refers to the result of a separate application of an AMP and one or more Bt toxins. In a further embodiments, a “combination” refers to the result of a sequential application of two or more separate components, e.g., a first application of an AMP, followed by a second application of one or more Bt toxins, or vice versa. Where the application is sequential or separate, the delay in applying the second component should not be such as to lose the beneficial effect of the combination. In some embodiments, the term combination can include separate application of two or more copmponents, (e.g., an AMP and one or more Bt toxins), wherein one of the components is expressed recombinantly in for example, a plant, a plant part or tissue, or a plant seed, and the other component is combined with the first component on the plant, or plant part, or plant tissue, or a plant seed in physical form, for example, in a separate composition or formulation applied to said plant, plant part or tissue, or a plant seed. In one illustrative example, a plant, a plant part or tissue, or a plant seed recombinantly expresses the one or more Bt toxins and the AMP is applied onto said plant, plant part or tissue, or plant seed in the form of a sprayable or spreadable composition or formulation. In other examples, the combination includes a plant, plant part or tissue, or a plant seed recombinantly expressing the AMP, and the one or more Bt toxins is/are applied onto said plant, plant part or tissue, or a plant seed in the form of a sprayable or spreadable composition or formulation. In still a further illustrative example, the combination of the two or more components can include a plant, a plant part or tissue, or a plant seed that recombinantly expresses both components, the AMP and the one or more Bt toxins. In each of these illustrative examples of combinations of the components (e.g. the AMP and the one or more Bt toxins), the components may be applied or expressed in the same part, or in different parts, of the plant, plant part or tissue, or a plant seed.

“Complementary” refers to the topological compatibility or matching together of interacting surfaces of two polynucleotides as understood by those of skill in the art. Thus, two sequences are “complementary” to one another if they are capable of hybridizing to one another to form a stable anti-parallel, double-stranded nucleic acid structure. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

“Conditioned medium” means the cell culture medium which has been used by cells and is enriched with cell derived materials but does not contain cells.

“Copy number” refers to the number of identical copies of a vector, an expression cassette, an amplification unit, a gene or indeed any defined nucleotide sequence, that are present in a host cell at any time. For example, in some embodiments, a gene or another defined chromosomal nucleotide sequence may be present in one, two, or more copies on the chromosome. An autonomously replicating vector may be present in one, or several hundred copies per host cell.

“Culture” or “cell culture” refers to the maintenance of cells in an artificial, in vitro environment.

“Culturing” refers to the propagation of organisms on or in various kinds of media. For example, the term “culturing” can mean growing a population of cells under suitable conditions in a liquid or solid medium. In some embodiments, culturing refers to fermentative recombinant production of a heterologous polypeptide of interest and/or other desired end products (typically in a vessel or reactor).

“Cystine” refers to an oxidized cysteine-dimer. Cystines are sulfur-containing amino acids obtained via the oxidation of two cysteine molecules, and are linked with a disulfide bond.

“Defined medium” means a medium that is composed of known chemical components but does not contain crude proteinaceous extracts or by-products such as yeast extract or peptone.

“Degeneracy” or “codon degeneracy” refers to the phenomenon that one amino acid can be encoded by different nucleotide codons. Thus, the nucleic acid sequence of a nucleic acid molecule that encodes a protein or polypeptide can vary due to degeneracies. As a result of the degeneracy of the genetic code, many nucleic acid sequences can encode a given polypeptide with a particular activity; such functionally equivalent variants are contemplated herein.

“Disulfide bond” or “disulfide bridges” refers to a covalent bond between two cysteine amino acids derived by the coupling of two thiol groups on their side chains. In some embodiments, a disulfide bond occurs via the oxidative folding of two different thiol groups (—SH) present in a polypeptide. In some embodiments, a polypeptide can comprise at least six different thiol groups (i.e., six cysteine residues each containing a thiol group); thus, in some embodiments, a polypeptide can form zero, one, two, three, or more intramolecular disulfide bonds.

“Double expression cassette” refers to two AMP expression cassettes contained on the same vector.

“Double transgene peptide expression vector” or “double transgene expression vector” means a yeast expression vector that contains two copies of the AMP expression cassette.

“DNA” refers to deoxyribonucleic acid, comprising a polymer of one or more deoxyribonucleotides or nucleotides (i.e., adenine [A], guanine [G], thymine [T], or cytosine [C]), which can be arranged in single-stranded or double-stranded form. For example, one or more nucleotides creates a polynucleotide.

“dNTPs” refers to the nucleoside triphosphates that compose DNA and RNA.

“Endogenous” refers to a polynucleotide, peptide, polypeptide, protein, or process that naturally occurs and/or exists in an organism, e.g., a molecule or activity that is already present in the host cell before a particular genetic manipulation.

“Enhancer element” refers to a DNA sequence operably linked to a promoter, which can exert increased transcription activity on the promoter relative to the transcription activity that results from the promoter in the absence of the enhancer element.

“ER” or “Endoplasmic reticulum” is a subcellular organelle common to all eukaryotes where some post translation modification processes occur.

“ERSP” or “Endoplasmic reticulum signal peptide” is an N-terminus sequence of amino acids that—during protein translation of the mRNA molecule encoding an AMP—is recognized and bound by a host cell signal-recognition particle, which moves the protein translation ribosome/mRNA complex to the ER in the cytoplasm. The result is the protein translation is paused until it docks with the ER where it continues and the resulting protein is injected into the ER.

“ersp” refers to a polynucleotide encoding the peptide, ERSP.

“ER trafficking” means transportation of a cell expressed protein into ER for post-translational modification, sorting and transportation.

“Excipient” refers to any agriculturally or pharmaceutically acceptable additive, carrier, surfactant, emulsifier, thickener, preservative, solvent, disintegrant, glidant, lubricant, diluent, filler, bulking agent, binder, emollient, stiffening agent, chelating agent, stabilizer, solubilizing agents, dispersing agent, suspending agent, antioxidant, antiseptic, wetting agent, humectant, fragrant, suspending agents, pigments, colorants, isotonic agents, viscosity enhancing agents, mucoadhesive agents, and/or any combination thereof, that can be added to an agricultural composition, preparation, and/or formulation, which may be useful in achieving a desired modification to the characteristics of the agricultural composition, preparation, and/or formulation. Such modifications include, but are not limited to, physical stability, chemical stability, pesticidal efficacy, and/or any combination thereof.

“Expression cassette” refers to (1) a DNA sequence of interest, e.g., a polynucleotide operable to encode an AMP; and one or more of the following: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements. The combination (1) with at least one of (2)-(6) is called an “expression cassette.” In some embodiments, there can be numerous expression cassettes cloned into a vector. For example, in some embodiments, there can be a first expression cassette comprising a polynucleotide operable to encode an AMP. In alternative embodiments, there are two expression cassettes, each comprising a polynucleotide operable to encode an AMP (i.e., a double expression cassette). In other embodiments, there are three expression cassettes operable to encode an AMP (i.e., a triple expression cassette). In some embodiments, a double expression cassette can be generated by subcloning a second expression cassette into a vector containing a first expression cassette. In some embodiments, a triple expression cassette can be generated by subcloning a third expression cassette into a vector containing a first and a second expression cassette. Methods concerning expression cassettes and cloning techniques are well-known in the art and described herein. See also AMP expression cassette.

“FECT” means a transient plant expression system using Foxtail mosaic virus with elimination of coating protein gene and triple gene block.

“Fermentation beer” refers to spent fermentation medium, i.e., fermentation medium supernatant after removal of organisms, that has been inoculated with and consumed by a transformed host cell (e.g., a yeast cell operable to express an AMP of the present disclosure). In some embodiments, fermentation beer refers to the solution that is recovered following the fermentation of the transformed host cell. The term “fermentation” refers broadly to the enzymatic and anaerobic or aerobic breakdown of organic substances (e.g., a carbon substrate) nutrient substances by microorganisms under controlled conditions (e.g., temperature, oxygen, pH, nutrients, and the like) to produce fermentation products (e.g., one or more peptides of the present disclosure). While fermentation typically describes processes that occur under anaerobic conditions, as used herein it is not intended that the term be solely limited to strict anaerobic conditions, as the term “fermentation” used herein may also occur processes that occur in the presence of oxygen.

“GFP” means green fluorescent protein from the jellyfish, Aequorea victoria.

“Growth medium” refers to a nutrient medium used for growing cells in vitro.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. Thus, in some embodiments, the term “homologous” refers to the sequence similarity between two polypeptide molecules, or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

There may be partial homology, or complete homology and thus identical. “Sequence identity” refers to a measure of relatedness between two or more nucleic acid sequences or two or more polypeptide sequences, and is given as a percentage with reference to the total comparison length. The identity calculation takes into account those nucleotide residues or amino acid residues that are identical and in the same relative positions in their respective larger sequences.

“Homologous recombination” refers to the event of substitution of a segment of DNA by another one that possesses identical regions (homologous) or nearly so. For example, in some embodiments, “homologous recombination” refers to a type of genetic recombination in which nucleotide sequences are exchanged between two similar or identical molecules of DNA. Briefly, homologous recombination is most widely used by cells to accurately repair harmful breaks that occur on both strands of DNA, known as double-strand breaks. Although homologous recombination varies widely among different organisms and cell types, most forms involve the same basic steps: after a double-strand break occurs, sections of DNA around the 5′ ends of the break are cut away in a process called resection. In the strand invasion step that follows, an overhanging 3′ end of the broken DNA molecule then “invades” a similar or identical DNA molecule that is not broken. After strand invasion, the further sequence of events may follow either of two main pathways, i.e., the double-strand break repair pathway, or the synthesis-dependent strand annealing pathway. Homologous recombination is conserved across all three domains of life as well as viruses, suggesting that it is a nearly universal biological mechanism. For example, in some embodiments, homologous recombination can occur using a site-specific integration (SSI) sequence, whereby there is a strand exchange crossover event between nucleic acid sequences substantially similar in nucleotide composition. These crossover events can take place between sequences contained in the targeting construct of the present disclosure (i.e., the SSI sequence) and endogenous genomic nucleic acid sequences (e.g., the polynucleotide encoding the peptide subunit). In addition, in some embodiments, it is possible that more than one site-specific homologous recombination event can occur, which would result in a replacement event in which nucleic acid sequences contained within the targeting construct have replaced specific sequences present within the endogenous genomic sequences.

“Hybridize” refers to the annealing of one single-stranded polynucleotide to another polynucleotide based on the well-understood principle of sequence complementarity. In some embodiments, the other polynucleotide is a single-stranded polynucleotide. The propensity for hybridization between polynucleotides depends on the temperature and ionic strength of their milieu, the length of the polynucleotides, and the degree of complementarity. The effect of these parameters on hybridization are well known in the art.

“Hybridization” refers to any process by which a strand of polynucleotide binds with a complementary strand through base pairing. Two single-stranded polynucleotides “hybridize” when they form a double-stranded duplex. Thus, as used herein, the term “hybridize” refers to the annealing of one single-stranded polynucleotide to another polynucleotide based on the well-understood principle of sequence complementarity. In some embodiments, the other polynucleotide is a single-stranded polynucleotide. The propensity for hybridization between polynucleotides depends on the temperature and ionic strength of their milieu, the length of the polynucleotides, and the degree of complementarity. The effect of these parameters on hybridization are well known in the art. When two single-stranded polynucleotides hybridize and form a double-stranded duplex, the region of double-strandedness can include the full-length of one or both of the single-stranded polynucleotides, or all of one single stranded polynucleotide and a subsequence of the other single stranded polynucleotide, or the region of double-strandedness can include a subsequence of each polynucleotide. Hybridization also includes the formation of duplexes which contain certain mismatches, provided that the two strands are still forming a double stranded helix. See “Stringent hybridization conditions” below.

“IC₅₀” or “IC50” refers to half-maximal inhibitory concentration, which is a measurement of how much of an agent is needed to inhibit a biological process by half, thus providing a measure of potency of said agent.

“Identity” refers to a relationship between two or more polypeptide sequences or two or more polynucleotide sequences, as determined by comparing said sequences. The term “identity” also means the degree of sequence relatedness between polypeptide or polynucleotide sequences, as the case may be, as determined by the match between strings of such sequences. “Identity” and “similarity” can be readily calculated by any one of the myriad methods known to those having ordinary skill in the art, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), the disclosures of which are incorporated herein by reference in their entireties. Furthermore, methods to determine identity and similarity are codified in publicly available computer programs. For example in some embodiments, methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), the disclosures of which are incorporated herein by reference in their entireties.

“in vivo” refers to in the living body of a plant or animal (e.g., an animal, plant or a cell) and to processes or reactions that occur within the living body of a plant or animal.

“Inactive” refers to a condition wherein something is not in a state of use, e.g., lying dormant and/or not working. For example, when used in the context of a gene or when referring to a gene, the term inactive means said gene is no longer actively synthesizing a gene product, having said gene product translated into a protein, or otherwise having the gene perform its normal function. For example, in some embodiments, the term inactive can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“Inhibiting” or “inhibit” or “combating” or “combat” or “controlling” or “control,” or any variation of these terms, refers to making something (e.g., the number of pests, the functions and/or activities of the pest, and/or the deleterious effect of the pest on a plant or animal susceptible to attack thereof) less in size, amount, intensity, or degree. For example, in some embodiments, the application of a pesticidally effective amount of a combination comprising an AMP or agriculturally acceptable salt thereof and a Bt toxin, or an agricultural composition comprising a combination of an AMP or agriculturally acceptable salt thereof, a Bt toxin, and at least one excipient, to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination thereof, results in the following effect: a decrease in the number of pests, or inhibition of the pest's activities (e.g., the pest dies stops or slows its movement; stops or slows its feeding; stops or slows its growth; becomes confused, e.g., with regard to navigation, locating food, sleeping behaviors, and/or mating; fails to pupate if applicable; interferes with reproduction of the pest; and/or precludes the pest from producing offspring and/or precludes the insect from producing fertile offspring) relative to the number of pests or activities thereof that had not been exposed to a pesticidally effective amount of a combination comprising an AMP or agriculturally acceptable salt thereof and a Bt toxin; or an agricultural composition comprising a combination of an AMP or agriculturally acceptable salt thereof, a Bt toxin, and at least one excipient.

In some embodiments, combating, controlling, or inhibiting a pest, includes any measurable decrease or complete inhibition to achieve a desired result. For example, there may be a decrease of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, in the number of pests or the activities thereof treated with peptides and/or compositions of the present disclosure, compared to untreated pests. About as used herein means within ±10%, preferably ±5% of a given value.

Thus, in some embodiments, the terms “combating, controlling, or inhibiting a pest,” refers to a decrease in the number of pests, or an inhibition of the activities of the pests (e.g., movement; feeding; growth; level of awareness or alertness, e.g., with regard to navigation, locating food, sleeping behaviors, and/or mating; pupation if applicable; reproduction; ability to produce offspring and/or ability to produce fertile offspring) that have received a pesticidally effective amount of a combination comprising an AMP or agriculturally acceptable salt thereof and a Bt toxin; or an agricultural composition comprising a combination of an AMP or agriculturally acceptable salt thereof, a Bt toxin, and at least one excipient, that is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 2.25%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.75%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8.75%, at least about 9%, at least about 9.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the number of pests, or the inhibition of activities of the pests (e.g., movement; feeding; growth; level of awareness or alertness, e.g., with regard to navigation, locating food, sleeping behaviors, and/or mating; pupation if applicable; reproduction; ability to produce offspring and/or ability to produce fertile offspring) that have not received a pesticidally effective amount of a combination comprising an AMP or agriculturally acceptable salt thereof and a Bt toxin; or an agricultural composition comprising a combination of an AMP or agriculturally acceptable salt thereof, a Bt toxin, and at least one excipient.

“Inoperable” refers to the condition of a thing not functioning, malfunctioning, or no longer able to function. For example, when used in the context of a gene or when referring to a gene, the term inoperable means said gene is no longer able to operate as it normally would, either permanently or transiently. For example, “inoperable,” in some embodiments, means that a gene is no longer able to synthesize a gene product, having said gene product translated into a protein, or is otherwise unable to gene perform its normal function. For example, in some embodiments, the term inoperable can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“Insect” includes all organisms in the class “Insecta.” The term “pre-adult” insects refers to any form of an organism prior to the adult stage, including, for example, eggs, larvae, and nymphs. As used herein, the term “insect refers to any arthropod and nematode, including acarids, and insects known to infest all crops, vegetables, and trees and includes insects that are considered pests in the fields of forestry, horticulture and agriculture. Examples of specific crops that might be protected with the methods disclosed herein are soybean, corn, cotton, alfalfa and the vegetable crops. A list of specific crops and insects is enclosed herein.

“Insect gut environment” or “gut environment” means the specific pH and proteinase conditions found within the fore, mid or hind gut of an insect or insect larva.

“Insect hemolymph environment” means the specific pH and proteinase conditions of found within an insect or insect larva.

“Insecticidal activity” means that upon or after exposing the insect to compounds, agents, or peptides, the insect either dies stops or slows its movement; stops or slows its feeding; stops or slows its growth; becomes confused (e.g., with regard to navigation, locating food, sleeping behaviors, and/or mating); fails to pupate; interferes with reproduction; and/or precludes the insect from producing offspring and/or precludes the insect from producing fertile offspring.

“Intervening linker” refers to a short peptide sequence in the protein separating different parts of the protein, or a short DNA sequence that is placed in the reading frame in the ORF to separate the upstream and downstream DNA sequences. For example, in some embodiments, an intervening linker may be used allowing proteins to achieve their independent secondary and tertiary structure formation during translation. In some embodiments, the intervening linker can be either resistant or susceptible to cleavage in plant cellular environments, in the insect and/or lepidopteran gut environment, and in the insect hemolymph and lepidopteran hemolymph environment.

“Isolated” refers to separating a thing and/or a component from its natural environment, e.g., a toxin isolated from a given genus or species means that toxin is separated from its natural environment.

“kb” refers to kilobase, i.e., 1000 bases. As used herein, the term “kb” means a length of nucleic acid molecules. For example, 1 kb refers to a nucleic acid molecule that is 1000 nucleotides long. A length of double-stranded DNA that is 1 kb long, contains two thousand nucleotides (i.e., one thousand on each strand). Alternatively, a length of single-stranded RNA that is 1 kb long, contains one thousand nucleotides.

“kDa” refers to kilodalton, a unit equaling 1,000 daltons; a “dalton” or “Da” is a unit of molecular weight (MW).

“KD₅₀” or “Knockdown dose 50” or “paralytic dose 50” or “PD₅₀” refers to the median dose required to cause paralysis or cessation of movement in 50% of a population, for example, and without limitation, a population of Musca domestica (common housefly), or a population of Aedes aegypti (mosquito).

“Knock in” or “knock-in” or “knocks-in” or “knocking-in” refers to the replacement of an endogenous gene with an exogenous or heterologous gene, or part thereof. For example, in some embodiments, the term “knock-in” refers to the introduction of a nucleic acid sequence encoding a desired protein to a target gene locus by homologous recombination, thereby causing the expression of the desired protein. In some embodiments, a “knock-in” mutation can modify a gene sequence to create a loss-of-function or gain-of-function mutation. The term “knock-in” can refer to the procedure by which a exogenous or heterologous polynucleotide sequence or fragment thereof is introduced into the genome, (e.g., “they performed a knock-in” or “they knocked-in the heterologous gene”), or the resulting cell and/or organism (e.g., “the cell is a “knock-in” or “the animal is a “knock-in”).

“Knock out” or “knockout” or “knock-out” or “knocks-out” or “knocking-out” refers to a partial or complete suppression of the expression gene product (e.g., mRNA) of a protein encoded by an endogenous DNA sequence in a cell. In some embodiments, the “knock-out” can be effectuated by targeted deletion of a whole gene, or part of a gene encoding a peptide, polypeptide, or protein. As a result, the deletion may render a gene inactive, partially inactive, inoperable, partly inoperable, or otherwise reduce the expression of the gene or its products in any cell in the whole organism and/or cell in which it is normally expressed. The term “knock-out” can refer to the procedure by which an endogenous gene is made completely or partially inactive or inoperable (e.g., “they performed a knock-out” or “they knocked-out the endogenous gene”), or the resulting cell and/or organism (e.g., “the cell is a “knock-out” or “the animal is a “knock-out”).

“l” or “linker” refers to a nucleotide encoding intervening linker peptide.

“L” or “LINKER” in the proper context refers to an intervening linker peptide, which links a translational stabilizing protein (STA) with an additional polypeptide, e.g., an AMP, and/or multiple AMP. When referring to amino acids, “L” can also mean leucine.

“LAC4 terminator” or “Lac4 terminator” refers to a DNA segment comprised of the transcriptional terminator sequence derived from the K. lactis β-galactosidase gene.

“Lepidopteran gut environment” means the specific pH and proteinase conditions of found within the fore, mid or hind gut of a lepidopteran insect or larva.

“Lepidopteran hemolymph environment” means the specific pH and proteinase conditions of found within lepidopteran insect or larva.

“LC₅₀” or “lethal concentration 50%” refers to the concentration of an agent required to kill 50% of a population.

“LD₂₀” refers to a dose required to kill 20% of a population.

“LD₅₀” refers to lethal dose 50 which means the dose required to kill 50% of a population.

“Linker” or “LINKER” or “peptide linker” or “L” or “intervening linker” refers to a short peptide sequence operable to link two peptides together. Linker can also refer to a short DNA sequence that is placed in the reading frame of an ORF to separate an upstream and downstream DNA sequences. In some embodiments, a linker can be cleavable by an insect protease. In some embodiments, a linker may allow proteins to achieve their independent secondary and tertiary structure formation during translation. In some embodiments, the linker can be either resistant or susceptible to cleavage in plant cellular environments, in the insect and/or lepidopteran gut environment, and/or in the insect hemolymph and lepidopteran hemolymph environment. In some embodiments, a linker can be cleaved by a protease, e.g., in some embodiments, a linker can be cleaved by a plant protease (e.g., papain, bromelain, ficin, actinidin, zingibain, and/or cardosins), an insect protease, a fungal protease, a vertebrate protease, an invertebrate protease, a bacteria protease, a mammal protease, a reptile protease, or an avian protease. In some embodiments, a linker can be cleavable or non-cleavable. In some embodiments, a linker comprises a binary or tertiary region, wherein each region is cleavable by at least two types of proteases: one of which is an insect and/or nematode protease and the other one of which is a human protease. In some embodiments, a linker can have one of (at least) three roles: to cleave in the insect gut environment, to cleave in the plant cell, or to be designed not to intentionally cleave.

“Locus of a pest” refers to the habitat of a pest; food supply of a pest; breeding ground of a pest; area traveled by or inhabited by a pest; material infested, eaten, used by a pest; and/or any environment in which a pest inhabits, uses, is present in, or is expected to be. In some embodiments, the locus of a pest includes, without limitation, a pest habitat; a pest food supply; a pest breeding ground; a pest area; a pest environment; any surface or location that may be frequented and/or infested by a pest; any plant or animal, or a locus of a plant or animal, susceptible to attack by a pest; and/or any surface or location where a pest may be found, may be expected to be found, or is likely to be attacked by a pest.

“Locus of a plant” refers to any place in which a plant is growing; any place where plant propagation materials of a plant are sown; any place where plant propagation materials of a plant will be placed into the soil; or any area where plants are stored, including without limitation, live plants and/or harvested plants, leaves, seeds, fruits, or parts thereof.

“Locus of an animal” refers to any place where animals live, eat, breed, sleep, or otherwise are present in.

“Medium” (plural “media”) refers to a nutritive solution for culturing cells in cell culture.

“MOA” refers to mechanism of action.

“Molecular weight (MW)” refers to the mass or weight of a molecule, and is typically measured in “daltons (Da)” or kilodaltons (kDa). In some embodiments, MW can be calculated using sodium dodecyl sulfate polyacrylamide gel electrophoresis (SDS-PAGE), analytical ultracentrifugation, or light scattering. In some embodiments, the SDS-PAGE method is as follows: the sample of interest is separated on a gel with a set of molecular weight standards. The sample is run, and the gel is then processed with a desired stain, followed by destaining for about 2 to 14 hours. The next step is to determine the relative migration distance (Rf) of the standards and protein of interest. The migration distance can be determined using the following equation:

$$Rf = \frac{Migration distance of the protein}{Migration distance of the dye front} \qquad Formula ( I )$$

Next, the logarithm of the MW can be determined based on the values obtained for the bands in the standard; e.g., in some embodiments, the logarithm of the molecular weight of an SDS-denatured polypeptide and its relative migration distance (Rf) is plotted into a graph. After plotting the graph, interpolating the value derived will provide the molecular weight of the unknown protein band.

“Motif” refers to a polynucleotide or polypeptide sequence that is implicated in having some biological significance and/or exerts some effect or is involved in some biological process.

“Multiple cloning site” or “MCS” refers to a segment of DNA found on a vector that contains numerous restriction sites in which a DNA sequence of interest can be inserted.

“Mutant” refers to an organism, DNA sequence, amino acid sequence, peptide, polypeptide, or protein, that has an alteration or variation (for example, in the nucleotide sequence or the amino acid sequence), which causes said organism and/or sequence to be different from the naturally occurring or wild-type organism, wild-type sequence, and/or reference sequence with which the mutant is being compared. In some embodiments, this alteration or variation can be one or more nucleotide and/or amino acid substitutions or modifications (e.g., deletion or addition). In some embodiments, the one or more amino acid substitutions or modifications can be conservative; here, such a conservative amino acid substitution and/or modification in a “mutant” does not substantially diminish the activity of the mutant in relation to its non-mutant form. For example, in some embodiments, a “mutant” possesses one or more conservative amino acid substitutions when compared to a peptide with a disclosed and/or claimed sequence, as indicated by a SEQ ID NO.

“N-terminus” or “N-terminal” refers to the free amine group (i.e., —NH₂) that is positioned on beginning or start of a polypeptide.

“NCBI” refers to the National Center for Biotechnology Information.

“nm” refers to nanometers.

“Non-Polar amino acid” is an amino acid that is weakly hydrophobic and includes glycine, alanine, proline, valine, leucine, isoleucine, phenylalanine and methionine. Glycine or gly is the most preferred non-polar amino acid for the dipeptides of this disclosure.

“Normalized peptide yield” means the peptide yield in the conditioned medium divided by the corresponding cell density at the point the peptide yield is measured. The peptide yield can be represented by the mass of the produced peptide in a unit of volume, for example, mg per liter or mg/L, or by the UV absorbance peak area of the produced peptide in the HPLC chromatograph, for example, mAu·sec. The cell density can be represented by visible light absorbance of the culture at wavelength of 600 nm (OD600).

“OD” refers to optical density. Typically, OD is measured using a spectrophotometer. When measuring growth over time of a cell population, OD600 is preferable to UV spectroscopy; this is because at a 600 nm wavelength, the cells will not be harmed as they would under too much UV light.

“OD660 nm” or “OD₆₆₀ₙₘ” refers to optical densities of a liquid sample measured (for example, yeast cell culture) when measured in a spectrophotometer at 660 nanometers (nm).

“One letter code” means the peptide sequence which is listed in its one letter code to distinguish the various amino acids in the primary structure of a protein: alanine=A, arginine=R, asparagine=N, aspartic acid=D, asparagine or aspartic acid=B, cysteine=C, glutamic acid=E, glutamine=Q, glutamine or glutamic acid=Z, glycine=G, histidine=H, isoleucine=I, leucine=L, lysine=K, methionine=M, phenylalanine=F, proline=P, serine=S, threonine=T, tryptophan=W, tyrosine=Y, and valine=V.

“Open reading frame” or “ORF” refers to a length of RNA or DNA sequence, between a translation start signal (e.g., AUG or ATG, respectively) and any one or more of the known termination codons, which encodes one or more polypeptide sequences. Put another way, the ORF describes the frame of reference as seen from the point of view of a ribosome translating the RNA code, insofar that the ribosome is able to keep reading (i.e., adding amino acids to the nascent protein) because it has not encountered a stop codon. Thus, “open reading frame” or “ORF” refers to the amino acid sequence encoded between translation initiation and termination codons of a coding sequence. Here, the terms “initiation codon” and “termination codon” refer to a unit of three adjacent nucleotides (i.e., a codon) in a coding sequence that specifies initiation and chain termination, respectively, of protein synthesis (mRNA translation).

In some embodiments, an ORF is a continuous stretch of codons that begins with a start codon (usually ATG for DNA, and AUG for RNA) and ends at a stop codon (usually UAA, UAG or UGA). In other embodiments, an ORF can be length of RNA or DNA sequence, between a translation start signal (e.g., AUG or ATG) and any one or more of the known termination codons, wherein said length of RNA or DNA sequence encodes one or more polypeptide sequences. In some other embodiments, an ORF can be a DNA sequence encoding a protein which begins with an ATG start codon and ends with a TGA, TAA or TAG stop codon. ORF can also mean the translated protein that the DNA encodes. Generally, those having ordinary skill in the art distinguish the terms “open reading frame” and “ORF,” from the term “coding sequence,” based upon the fact that the broadest definition of “open reading frame” simply contemplates a series of codons that does not contain a stop codon. Accordingly, while an ORF may contain introns, the coding sequence is distinguished by referring to those nucleotides (e.g., concatenated exons) that can be divided into codons that are actually translated into amino acids by the ribosomal translation machinery (i.e., a coding sequence does not contain introns); however, as used herein, the terms “coding sequence”; “CDS”; “open reading frame”; and “ORF,’ are used interchangeably.

“Operable” refers to the ability to be used, the ability to do something, and/or the ability to accomplish some function or result. For example, in some embodiments, “operable” refers to the ability of a polynucleotide, DNA sequence, RNA sequence, or other nucleotide sequence or gene to encode a peptide, polypeptide, and/or protein. For example, in some embodiments, a polynucleotide may be operable to encode a protein, which means that the polynucleotide contains information that imbues it with the ability to create a protein (e.g., by transcribing mRNA, which is in turn translated to protein).

“Operably linked” refers to a juxtaposition wherein the components so described are in a relationship permitting them to function in their intended manner. For example, in some embodiments, operably linked can refer to two or more DNA, peptide, or polypeptide sequences. In other embodiments, operably linked can mean that the two adjacent DNA sequences are placed together such that the transcriptional activation of one DNA sequence can act on the other DNA sequence. In yet other embodiments, the term “operably linked” can refer to two or more peptides and/or polypeptides, wherein said two or more peptides and/or polypeptides are connected in such a way as to yield a single polypeptide chain; alternatively, the term operably linked can refer to two or more peptides that are connected in such a way that one peptide exerts some effect on the other. In yet other embodiments, operably linked can refer to two adjacent DNA sequences are placed together such that the transcriptional activation of one can act on the other.

“Out-recombined” or “out-recombination” refers to the removal of a gene and/or polynucleotide sequence (e.g., an endogenous gene, a transgene, a heterologous polynucleotide, etc.) that is flanked by two site-specific recombination sites (e.g., the 5′- and 3′-nucleotide sequence of a target gene that is homologous to the homology arms of a target vector) during in vivo homologous recombination. In some embodiments, the term “out-recombined” refers to the process wherein an endogenous gene is removed, e.g., during homologous recombination. In other embodiments, the term “out-recombined” refers to the process wherein a heterologous polynucleotide is removed via molecular mechanisms intrinsic to the host cell.

“Pest” includes, but is not limited to: insects, fungi, bacteria, nematodes, mites, ticks, and the like.

“Pesticidally-effective amount” refers to an amount of the pesticide that is able to do one or more of the following: bring about death to at least one pest; or to noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. The formulations may also vary with respect to climatic conditions, environmental considerations, and/or frequency of application and/or severity of pest infestation.

“Pharmaceutically acceptable salt” is synonymous with agriculturally acceptable salt, and as used herein refers to a compound that is modified by making acid or base salts thereof.

“Plant” shall mean whole plants, plant tissues, plant cells, plant parts, plant organs (e.g., leaves, stems, roots, etc.), seeds, propagules, embryos and progeny of the same. Plant cells can be differentiated or undifferentiated (e.g. callus, suspension culture cells, protoplasts, leaf cells, root cells, phloem cells, and pollen).

“Plant transgenic protein” means a protein from a heterologous species that is expressed in a plant after the DNA or RNA encoding it was delivered into one or more of the plant cells.

“Plant-incorporated protectant” or “PIP” means an insecticidal protein produced by transgenic plants, and the genetic material necessary for the plant to produce the protein.

“Plant cleavable linker” means a cleavable linker peptide, or a nucleotide encoding a cleavable linker peptide, which contains a plant protease recognition site and can be cleaved during the protein expression process in the plant cell.

“Plant regeneration media” means any media that contains the necessary elements and vitamins for plant growth and plant hormones necessary to promote regeneration of a cell into an embryo which can germinate and generate a plantlet derived from tissue culture. Often the media contains a selectable agent to which the transgenic cells express a selection gene that confers resistance to the agent.

“Plasmid” refers to a DNA segment that acts as a carrier for a gene of interest, and, when transformed or transfected into an organism, can replicate and express the DNA sequence contained within the plasmid independently of the host organism. Plasmids are a type of vector, and can be “cloning vectors” (i.e., simple plasmids used to clone a DNA fragment and/or select a host population carrying the plasmid via some selection indicator) or “expression plasmids” (i.e., plasmids used to produce large amounts of polynucleotides and/or polypeptides).

“Polar amino acid” is an amino acid that is polar and includes serine, threonine, cysteine, asparagine, glutamine, histidine, tryptophan and tyrosine; preferred polar amino acids are serine, threonine, cysteine, asparagine and glutamine; with serine being most highly preferred.

“Polynucleotide” refers to a polymeric-form of nucleotides (e.g., ribonucleotides, deoxyribonucleotides, or analogs thereof) of any length; e.g., a sequence of two or more ribonucleotides or deoxyribonucleotides. As used herein, the term “polynucleotide” includes double- and single-stranded DNA, as well as double- and single-stranded RNA; it also includes modified and unmodified forms of a polynucleotide (modifications to and of a polynucleotide, for example, can include methylation, phosphorylation, and/or capping). In some embodiments, a polynucleotide can be one of the following: a gene or gene fragment (for example, a probe, primer, EST, or SAGE tag); genomic DNA; genomic DNA fragment; exon; intron; messenger RNA (mRNA); transfer RNA; ribosomal RNA; ribozyme; cDNA; recombinant polynucleotide; branched polynucleotide; plasmid; vector; isolated DNA of any sequence; isolated RNA of any sequence; nucleic acid probe; primer or amplified copy of any of the foregoing.

In yet other embodiments, a polynucleotide can refer to a polymeric-form of nucleotides operable to encode the open reading frame of a gene.

In some embodiments, a polynucleotide can refer to cDNA.

In some embodiments, polynucleotides can have any three-dimensional structure and may perform any function, known or unknown. The structure of a polynucleotide can also be referenced to by its 5′- or 3′-end or terminus, which indicates the directionality of the polynucleotide. Adjacent nucleotides in a single-strand of polynucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the polynucleotide, which may be called the 5′ and 3′ ends or termini. The 5′ and 3′ ends can also be called the phosphoryl (PO₄) and hydroxyl (OH) ends, respectively, because of the chemical groups attached to those ends. The term polynucleotide also refers to both double- and single-stranded molecules. Unless otherwise specified or required, any embodiment that makes or uses a polynucleotide encompasses both the double-stranded form and each of two complementary single-stranded forms known or predicted to make up the double-stranded form.

In some embodiments, a polynucleotide can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the polynucleotide.

In some embodiments, a polynucleotide can also be further modified after polymerization, such as by conjugation with a labeling component. Additionally, the sequence of nucleotides in a polynucleotide can be interrupted by non-nucleotide components. One or more ends of the polynucleotide can be protected or otherwise modified to prevent that end from interacting in a particular way (e.g. forming a covalent bond) with other polynucleotides.

In some embodiments, a polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term “sequence” refers to the alphabetical representation of a polynucleotide or any nucleic acid molecule, including natural and non-natural bases.

The term “RNA molecule” or ribonucleic acid molecule refers to a polynucleotide having a ribose sugar rather than deoxyribose sugar and typically uracil rather than thymine as one of the pyrimidine bases. An RNA molecule of the disclosure is generally single-stranded, but can also be double-stranded. In the context of an RNA molecule from an RNA sample, the RNA molecule can include the single-stranded molecules transcribed from DNA in the cell nucleus, mitochondrion or chloroplast, which have a linear sequence of nucleotide bases that is complementary to the DNA strand from which it is transcribed.

In some embodiments, a polynucleotide can further comprise one or more heterologous regulatory elements. For example, in some embodiments, the regulatory element is one or more promoters; enhancers; silencers; operators; splicing signals; polyadenylation signals; termination signals; RNA export elements, internal ribosomal entry sites (IRES); poly-U sequences; or combinations thereof.

“Post-transcriptional regulatory elements” are DNA segments and/or mechanisms that affect mRNA after it has been transcribed. Mechanisms of post-transcriptional mechanisms include splicing events; capping, splicing, and addition of a Poly (A) tail, and other mechanisms known to those having ordinary skill in the art.

“Promoter” refers to a region of DNA to which RNA polymerase binds and initiates the transcription of a gene.

“Protein” has the same meaning as “peptide” and/or “polypeptide” in this document.

“Ratio” refers to the quantitative relation between two amounts showing the number of times one value contains or is contained within the other.

“Reading frame” refers to one of the six possible reading frames, three in each direction, of the double stranded DNA molecule. The reading frame that is used determines which codons are used to encode amino acids within the coding sequence of a DNA molecule. In some embodiments, a reading frame is a way of dividing the sequence of nucleotides in a polynucleotide and/or nucleic acid (e.g., DNA or RNA) into a set of consecutive, non-overlapping triplets.

“Recombinant DNA” or “rDNA” refers to DNA that is comprised of two or more different DNA segments.

“Recombinant vector” means a DNA plasmid vector into which foreign DNA has been inserted.

“Regulatory elements” refers to a genetic element that controls some aspect of the expression and/or processing of nucleic acid sequences. For example, in some embodiments, a regulatory element can be found at the transcriptional and post-transcriptional level. Regulatory elements can be cis-regulatory elements (CREs), or trans-regulatory elements (TREs). In some embodiments, a regulatory element can be one or more promoters; enhancers; silencers; operators; splicing signals; polyadenylation signals; termination signals; RNA export elements, internal ribosomal entry sites (IRES); poly-U sequences; and/or other elements that influence gene expression, for example, in a tissue-specific manner; temporal-dependent manner; to increase or decrease expression; and/or to cause constitutive expression.

“Restriction enzyme” or “restriction endonuclease” refers to an enzyme that cleaves DNA at a specified restriction site. For example, a restriction enzyme can cleave a plasmid at an EcoRI, SacII or BstXI restriction site allowing the plasmid to be linearized, and the DNA of interest to be ligated.

“Restriction site” refers to a location on DNA comprising a sequence of 4 to 8 nucleotides, and whose sequence is recognized by a particular restriction enzyme.

“Selection gene” means a gene which confers an advantage for a genetically modified organism to grow under the selective pressure.

“sp.” or “sp.” refers to species.

“ssp.” or “subsp.” or “ssp.” or “subsp.” refers to subspecies.

“Subcloning” or “subcloned” refers to the process of transferring DNA from one vector to another, usually advantageous vector. For example, polynucleotide encoding a mutant AMP can be subcloned into a pLB102 plasmid subsequent to selection of yeast colonies transformed with pKLAC1 plasmids.

“SSI” is an acronym that is context dependent. In some contexts, it can refer to “site-specific integration,” which is used to refer to a sequence that will permit in vivo homologous recombination to occur at a specific site within a host organism's genome. Thus, in some embodiments, the term “site-specific integration” refers to the process directing a transgene to a target site in a host-organism's genome, allowing the integration of genes of interest into pre-selected genome locations of a host-organism. However, in other contexts, SSI can refer to “surface spraying indoors,” which is a technique of applying a variable volume sprayable volume of an insecticide onto surfaces where vectors rest, such as on walls, windows, floors and ceilings.

“STA” or “Translational stabilizing protein” or “stabilizing domain” or “stabilizing protein” (used interchangeably herein) means a peptide or protein with sufficient tertiary structure that it can accumulate in a cell without being targeted by the cellular process of protein degradation. The protein can be between 5 and 50 amino acids long. The translational stabilizing protein is coded by a DNA sequence for a protein that is operably linked with a sequence encoding an insecticidal protein or an AMP in the ORF. The operably-linked STA can either be upstream or downstream of the AMP and can have any intervening sequence between the two sequences (STA and AMP) as long as the intervening sequence does not result in a frame shift of either DNA sequence. The translational stabilizing protein can also have an activity which increases delivery of the AMP across the gut wall and into the hemolymph of the insect.

“sta” means a nucleotide encoding a translational stabilizing protein.

“Stringent hybridization” or “stringent hybridization conditions” refers to conditions under which a polynucleotide (e.g., a nucleic acid probe, primer or oligonucleotide) will hybridize to its target sequence, typically in a complex mixture of nucleic acids, but not to other sequences. Stringent hybridization conditions are sequence- and length-dependent, and depend on % (percent)-identity (or %-mismatch) over a certain length of nucleotide residues. Longer sequences hybridize specifically at higher temperatures than shorter sequences. Generally, stringent conditions are selected to be about 5° C. lower than the thermal melting point (Tm) for the specific sequence at a defined ionic strength and pH. Stringent conditions may also be achieved with the addition of destabilizing agents, such as formamide. In some embodiments, a polynucleotide of the present disclosure can stringently hybridize to a polynucleotide encoding an AMP, an Av3b, or a complementary nucleotide sequence thereof. For example, in some embodiments, a polynucleotide of the present disclosure can stringently hybridize to a polynucleotide operable to encode an amino acid sequence as set forth in SEQ ID NO: 1 or 3, or a complementary nucleotide sequence thereof.

“Structural motif” refers to the three-dimensional arrangement of peptides and/or polypeptides, and/or the arrangement of operably linked polypeptide segments. For example, the polypeptide comprising ERSP-STA-L-AMP has an ERSP motif, an STA motif, a LINKER motif, and an AMP polypeptide motif.

“Susceptible to attack by a pest(s),” refer to plants, or human or animal patients or subjects, susceptible to a pest or a pest infections.

“Toxin” refers to a venom and/or a poison, especially a protein or conjugated protein produced by certain animals, higher plants, and pathogenic bacteria. Generally, the term “toxin” is reserved natural products, e.g., molecules and peptides found in scorpions, spiders, snakes, poisonous mushrooms, etc., whereas the term “toxicant” is reserved for man-made products and/or artificial products e.g., man-made chemical pesticides. However, as used herein, the terms “toxin” and “toxicant” are used synonymously

“Transfection” and “transformation” both refer to the process of introducing exogenous and/or heterologous DNA or RNA (e.g., a vector containing a polynucleotide that encodes a CRIP) into a host organism (e.g., a prokaryote or a eukaryote). Generally, those having ordinary skill in the art sometimes reserve the term “transformation” to describe processes where exogenous and/or heterologous DNA or RNA are introduced into a bacterial cell; and reserve the term “transfection” for processes that describe the introduction of exogenous and/or heterologous DNA or RNA into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

“Transgene” means a heterologous and/or exogenous polynucleotide sequence that is transformed into an organism and/or a cell therefrom.

“Transgenic host cell” or “host cell” means a cell which is transformed with a gene and has been selected for its transgenic status via an additional selection gene.

“Transgenic plant” means a plant that has been derived from a single cell that was transformed with foreign DNA such that every cell in the plant contains that transgene.

“Transient expression system” means an Agrobacterium tumefaciens-based system which delivers DNA encoding a disarmed plant virus into a plant cell where it is expressed. The plant virus has been engineered to express a protein of interest at high concentrations, up to 40% of the total soluble protein (TSP).

“Triple expression cassette refers to three AMP expression cassettes contained on the same vector.

“TRBO” means a transient plant expression system using Tobacco mosaic virus with removal of the viral coating protein gene.

“Trypsin cleavage” means an in vitro assay that uses the protease enzyme trypsin (which recognizes exposed lysine and arginine amino acid residues) to separate a cleavable linker at that cleavage site. It also means the act of the trypsin enzyme cleaving that site.

“TSP” or “total soluble protein” means the total amount of protein that can be extracted from a plant tissue sample and solubilized into the extraction buffer.

“var.” refers to varietas or variety. The term “var.” is used to indicate a taxonomic category that ranks below the species level and/or subspecies (where present). In some embodiments, the term “var.” represents members differing from others of the same subspecies or species in minor but permanent or heritable characteristics.

“Vector” refers to the DNA segment that accepts a heterologous polynucleotide operable to encode a peptide of interest (e.g., amp). The heterologous polynucleotide is known as an “insert” or “transgene.”

“Wild type” or “WT” or “wild-type” or “wildtype” refer to the phenotype and/or genotype (i.e., the appearance or sequence) of an organism, polynucleotide sequence, and/or polypeptide sequence, as it is found and/or observed in its naturally occurring state or condition.

“Yield” refers to the production of a peptide, and increased yields can mean increased amounts of production, increased rates of production, and an increased average or median yield and increased frequency at higher yields. The term “yield” when used in reference to plant crop growth and/or production, as in “yield of the plant” refers to the quality and/or quantity of biomass produced by the plant.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed. 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

Although the disclosure of the invention has been described in detail for purposes of clarity and understanding, it will be obvious to those with skill in the art that certain modifications can be practiced within the scope of the appended claims. All publications and patent documents cited herein are hereby incorporated by reference in their entirety for all purposes to the same extent as if each were so individually denoted.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers, or disavowals, and except to the extent that the incorporated material is inconsistent with the express disclosure herein, in which case the language in this disclosure controls.

### Av3b Mutant Peptides (AMPs)

The sea anemone, Anemonia viridis, possesses a variety of toxins that it uses to defend itself: one of these toxins is the neurotoxin, “Av3.” Av3 is a type III sea anemone toxin that inhibits the inactivation of voltage-gated sodium (Na⁺) channels at receptor site 3, resulting in contractile paralysis. The binding of an Av3 toxin to site 3 results in the inactivated state of the sodium channel to become destabilized, which in turn causes the channel to remain in the open position (see Blumenthal et al., Voltage-gated sodium channel toxins: poisons, probes, and future promise. Cell Biochem Biophys. 2003; 38(2):215-38). Av3 shows high selectivity for crustacean and insect sodium channels, and low selectivity for mammalian sodium channels (see Moran et al., Sea anemone toxins affecting voltage-gated sodium channels—molecular and evolutionary features, Toxicon. 2009 Dec. 15; 54(8): 1089-1101). An exemplary Av3 polypeptide from Anemonia viridis is provided having the amino acid sequence of “RSCCPCYWGGCPWGQNCYPEGCSGPKV” (SEQ ID NO:2) (NCBI Accession No. P01535.1).

In some embodiments, wild-type Av3 can be mutated, e.g., a wild-type Av3 can have an N-terminal mutation and a C-terminal mutation, wherein the N-terminal mutation results in an amino acid substitution of R1K relative to SEQ ID NO:2, and the C-terminal mutation results in an amino acid deletion relative to SEQ ID NO:2; thus, the wild-type Av3 peptide amino acid sequence is changed from “RSCCPCYWGGCPWGQNCYPEGCSGPKV” (SEQ ID NO: 2), to the amino acid sequence: “KSCCPCYWGGCPWGQNCYPEGCSGPK” (SEQ ID NO:3).

When wild-type Av3 has an R1K mutation and a C-terminal deletion, resulting in the peptide having an amino acid sequence of SEQ ID NO: 3, the resulting peptide is called, “Av3b.” An exemplary method of obtaining Av3b is disclosed in PCT Application No. PCT/US2019/051093, the disclosure of which is incorporated herein by reference in its entirety.

The Av3b peptide has characteristics that make it superior to wild-type Av3. See PCT/US2019/051093. However, the present disclosure describes a novel and inventive variant of Av3b, called an Av3b mutant polypeptide (AMP). As used herein, the term “AMP” refers to the Av3b mutant polypeptide, “Av3bM170,” which has an amino acid sequence of: “KSCCPCYWGGCPWGQNCYPEGCGGPG” (SEQ ID NO: 1).

### Exemplary AMPs

In some embodiments, the present disclosure comprises, consists essentially of, or consists of, a combination comprising an Av3 mutant polypeptide (AMP), and a Bacillus thuringiensis (Bt) toxin; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1); or an agriculturally acceptable salt thereof.

In some embodiments, the present disclosure comprises, consists essentially of, or consists of, a combination comprising an Av3 mutant polypeptide (AMP), and a Bacillus thuringiensis (Bt) toxin; wherein the AMP comprises the amino acid sequence: “KSCCPCYWGGCPWGQNCYPEGCGGPG” (SEQ ID NO: 1); or an agriculturally acceptable salt thereof.

In some embodiments, the present disclosure comprises, consists essentially of, or consists of, a combination comprising an Av3 mutant polypeptide (AMP), and a Bacillus thuringiensis (Bt) toxin; wherein the AMP consists essentially of the amino acid sequence: “KSCCPCYWGGCPWGQNCYPEGCGGPG” (SEQ ID NO: 1); or an agriculturally acceptable salt thereof.

In some embodiments, the present disclosure comprises, consists essentially of, or consists of, a combination comprising an Av3 mutant polypeptide (AMP), and a Bacillus thuringiensis (Bt) toxin; wherein the AMP consists of the amino acid sequence: “KSCCPCYWGGCPWGQNCYPEGCGGPG” (SEQ ID NO: 1); or an agriculturally acceptable salt thereof.

In some embodiments, an AMP of the present disclosure can comprise, consist essentially of, or consist of, a homopolymer or heteropolymer of two or more AMPs, wherein the amino acid sequence of each AMP is the same.

In some embodiments, an AMP of the present disclosure can comprise, consist essentially of, or consist of, an AMP that is a fused protein comprising two or more AMPs separated by a cleavable or non-cleavable linker, and wherein the amino acid sequence of each AMP is the same.

In some embodiments, the linker is a cleavable linker.

In some embodiments, the linker has an amino acid sequence as set forth in any one of SEQ ID NOs: 4-16.

In some embodiments, the linker is cleavable inside at least one of (i) the gut or hemolymph of an insect, and (ii) cleavable inside the gut of a mammal.

### AMP-Insecticidal Proteins

In some embodiments, a combination of the present disclosure can comprise an AMP-insecticidal protein, and a Bacillus thuringiensis (Bt) toxin; wherein the AMP-insecticidal protein is any protein, peptide, polypeptide, amino acid sequence, configuration, construct, or arrangement, comprising: (1) at least one AMP, or two or more AMPs (wherein the amino acid sequence of each AMP is the same); and (2) one or more additional non-AMP peptides, polypeptides, or proteins. For example, in some embodiments, these additional non-AMP peptides, polypeptides, or proteins may have the ability to increase the mortality and/or inhibit the growth of insects exposed to the AMP-insecticidal protein, relative to the AMP alone; increase the expression of the AMP-insecticidal protein, e.g., in a host cell; and/or affect the post-translational processing of the AMP-insecticidal protein.

In some embodiments, a combination of the present disclosure can comprise an AMP-insecticidal protein, and a Bacillus thuringiensis (Bt) toxin; wherein the AMP-insecticidal comprises an AMP having an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1); or an agriculturally acceptable salt thereof.

In some embodiments, a combination of the present disclosure can comprise an AMP-insecticidal protein, and a Bacillus thuringiensis (Bt) toxin; wherein the AMP-insecticidal comprises an AMP having the amino acid sequence: “KSCCPCYWGGCPWGQNCYPEGCGGPG” (SEQ ID NO: 1); or an agriculturally acceptable salt thereof.

In some embodiments, a combination of the present disclosure can comprise an AMP-insecticidal protein, and a Bacillus thuringiensis (Bt) toxin; wherein the AMP-insecticidal comprises an AMP having an amino acid sequence that consists essentially of the amino acid sequence: “KSCCPCYWGGCPWGQNCYPEGCGGPG” (SEQ ID NO: 1); or an agriculturally acceptable salt thereof.

In some embodiments, a combination of the present disclosure can comprise an AMP-insecticidal protein, and a Bacillus thuringiensis (Bt) toxin; wherein the AMP-insecticidal comprises an AMP having an amino acid sequence that consists of the amino acid sequence: “KSCCPCYWGGCPWGQNCYPEGCGGPG” (SEQ ID NO: 1); or an agriculturally acceptable salt thereof.

In some embodiments, an AMP-insecticidal protein can be a polymer comprising two or more AMPs. In yet other embodiments, an AMP-insecticidal protein can be a polymer comprising two or more AMPs, wherein the AMPs are operably linked via a linker peptide, e.g., a cleavable and/or a non-cleavable linker. Here, the linker peptide falls under the category of the additional non-AMP peptide described above.

In some embodiments, an AMP-insecticidal protein can refer to a one or more AMPs operably linked with one or more proteins such as a stabilizing domain (STA); an endoplasmic reticulum signaling protein (ERSP); an insect cleavable or insect non-cleavable linker (L); and/or any other combination thereof.

In some embodiments, an AMP-insecticidal protein can be a polymer of amino acids that, when properly folded or in its most natural thermodynamic state, exerts an insecticidal activity against one or more insects.

In other embodiments, an insecticidal protein can be a polymer of two or more AMPs that are the same.

In yet other embodiments, an AMP-insecticidal protein can comprise one or more AMPs, and one or more peptides, polypeptides, or proteins, that may assist in the AMP-insecticidal protein's folding.

In some embodiments, an AMP-insecticidal protein can comprise one or more AMPs, and one or more peptides, polypeptides, or proteins, wherein the one or more peptides, polypeptides, or proteins are protein tags that help stability or solubility. In other embodiments, the peptides, polypeptides, or proteins can be protein tags that aid in affinity purification.

In some embodiments, an AMP-insecticidal protein can refer to a one or more AMPs operably linked with one or more proteins such as a stabilizing domain (STA); an endoplasmic reticulum signaling protein (ERSP); an insect cleavable or insect non-cleavable linker; one or more heterologous peptides; one or more additional polypeptides; and/or any other combination thereof. In some embodiments, an insecticidal protein can comprise a one or more AMPs as disclosed herein.

In some embodiments, an AMP-insecticidal protein can comprise an AMP homopolymer, e.g., two or more AMP monomers that are the same AMP.

In some embodiments, an AMP-insecticidal protein can comprise, consist essentially of, or consist of one or more AMPs having an amino acid sequence set forth in SEQ ID NO: 1, or an agriculturally acceptable salt thereof. In some embodiments, the AMP-insecticidal protein may comprise an AMP having an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% amino acid sequence identity to of SEQ ID NO: 1, or an agriculturally acceptable salt thereof.

Examples of linkers include, but not limited to, the following sequences: IGER (SEQ ID NO:4), EEKKN, (SEQ ID NO:5), and ETMFKHGL (SEQ ID NO:6), or combinations thereof.

In some embodiments, the linker can be one or more of the following:

(SEQ ID NO: 7)
ALKFLV,
(SEQ ID NO: 8)
ALKLFV,
(SEQ ID NO: 9)
IFVRLR,
(SEQ ID NO: 10)
LFAAPF,
(SEQ ID NO: 11)
ALKFLVGS,
(SEQ ID NO: 12)
ALKLFVGS,
(SEQ ID NO: 13)
IFVRLRGS,
(SEQ ID NO: 14)
LFAAPFGS,
(SEQ ID NO: 15)
LFVRLRGS,
and/or
(SEQ ID NO: 16)
LGERGS.

Exemplary methods for the generation of cleavable and non-cleavable linkers can be found in U.S. patent application Ser. No. 15/727,277; and PCT Application No. PCT/US2013/030042, the disclosure of which are incorporated herein by reference in their entireties.

Exemplary ERSPs and STAs and their methods of use are provided in U.S. Pat. No. 9,567,381, the disclosure of which is incorporated herein by reference in its entirety.

Bacillus thuringiensis Organisms, and Toxins Therefrom

“Bt” are the initials for a bacterium called Bacillus thuringiensis. The Bt bacteria produce a family of peptides that are toxic to many insects. Indeed, strains of Bacillus thuringiensis (Bt) have been used as a source for insecticidal proteins since the discovery that Bt strains and the toxins derived therefrom demonstrate a high toxicity against specific insects. Bt strains are known to produce delta-endotoxins that are localized within parasporal crystalline inclusion bodies at the onset of sporulation and during the stationary growth phase (e.g., Cry proteins), and are also known to produce secreted insecticidal proteins. Upon ingestion by a susceptible insect, delta-endotoxins as well as secreted toxins exert their effects at the surface of the midgut epithelium, disrupting the cell membrane, leading to cell disruption and death. Genes encoding insecticidal proteins have also been identified in bacterial species other than Bt, including other Bacillus and a diversity of other bacterial species, such as Brevibacillus laterosporus, Lysinibacillus sphaericus (“Ls” formerly known as Bacillus sphaericus) and Paenibacillus popilliae.

The parasporal crystalline protein inclusions (usually referred to as crystals) typically fall under two major classes of toxins: crystal Bt proteins (Cry), and cytolysins (Cyt). Since the cloning and sequencing of the first crystal proteins genes in the early-1980s, many other toxins have been characterized and are now classified according to the nomenclature of Crickmore et al. (1998). Generally, Cyt proteins are toxic towards the insect orders Coleoptera (beetles) and Diptera (flies), and Cry proteins target Lepidopterans (moths and butterflies). Cry proteins bind to specific receptors on the membranes of mid-gut (epithelial) cells resulting in rupture of those cells. If a Cry protein cannot find a specific receptor on the epithelial cell to which it can bind, then it is not toxic. Bt strains can have different complements of Cyt and Cry proteins, thus defining their host ranges. The genes encoding many Cry proteins have been identified.

Currently there are four main pathotypes of insecticidal Bt parasporal peptides based on order specificity: Lepidoptera-specific (CryI, now Cry1), Coleoptera-specific (CryIII, now Cry3), Diptera-specific (CryIV, now Cry4, Cry 10, Cry11; and CytA, now Cyt1A), and CryII (Now Cry2), the only family known at that time to have dual (Lepidoptera and Diptera) specificity. Cross-order activity is now apparent in many cases.

The nomenclature assigns holotype sequences a unique name which incorporates ranks based on the degree of divergence, with the boundaries between the primary (Arabic numeral), secondary (uppercase letter), and tertiary (lower case letter) rank representing approximately 95%, 78% and 45% identities. A fourth rank (another Arabic number) is used to indicate independent isolations of holotype toxin genes with sequences that are identical or differ only slightly. Currently, the nomenclature distinguishes 174 holotype sequences that are grouping in 55 cry and 2 cyt families. An exemplary description of Bt toxins and their nomenclature is provided in Crickmore et al., Revision of the Nomenclature for the Bacillus thuringiensis Pesticidal Crystal Proteins. Microbiol Mol Biol Rev. 1998 September; 62(3): 807-813; and Crickmore et al., A structure-based nomenclature for Bacillus thuringiensis and other bacteria-derived pesticidal proteins. J Invertebr Pathol. 2021 November; 186:107438; the disclosures of which are incorporated herein by reference in their entireties.

Also included in the descriptions of the present disclosure are families of highly related crystal proteins produced by other bacteria: Cry16 and Cry17 from Clostridium bifermentans (Barloy et al., 1996, 1998), Cry 18 from Bacillus popilliae (Zhang et al., 1997), Cry43 from Paenibacillus lentimorbis (Yokoyama et al., 2004) and the binary Cry48/Cry49 produced by Bacillus sphaericus (Jones et al., 2008). Other crystalline or secreted pesticidal proteins, such as the S-layer proteins (Peña et al., 2006) that are included here are, genetically altered crystal proteins, except those that were modified through single amino acid substitutions (e.g., Lambert et al., 1996). Any of these genes may be used to produce a suitable Bt related toxin for this invention.

Naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the Bt protein proteins disclosed in the present disclosure as discussed below. Variant proteins encompassed by the present disclosure are biologically active, that is they continue to possess the desired biological activity of the native protein, i.e., retaining pesticidal activity. By “retains activity” is intended that the variant will have at least about 30%, at least about 50%, at least about 70%, or at least about 80% of the pesticidal activity of the native protein. Methods for measuring pesticidal activity are well known in the art. See, for example, Czapla and Lang (1990) J. Econ. Entomol. 83: 2480-2485; Andrews et al. (1988) Biochem. J. 252:199-206; Marrone et al. (1985) J. of Economic Entomology 78:290-293; and U.S. Pat. No. 5,743,477, all of which are herein incorporated by reference in their entirety, and all sequences identified by number specifically incorporated by reference.

In some embodiments, a combination of the present disclosure comprises an AMP and a Bt toxin, wherein the Bt toxin is any one or more known fermentation solids, spores, toxins, pesticidal proteins, or a variant thereof, produced by any species belonging to the genus, Bacillus.

In some embodiments, a combination of the present disclosure comprises an AMP and a Bt toxin, wherein the Bt toxin is one or more fermentation solids, spores, toxins, pesticidal proteins, or variant thereof, isolated or originating from a Bacillus thuringiensis subspecies. For example, in some embodiments, the Bacillus thuringiensis subspecies can be one of the following subspecies: aizawai; aizawai/pacificus; alesti; amagiensis; andalousiensis; argentinensis; asturiensis; azorensis; balearica; berliner; bolivia; brasilensis; cameroun; canadensis; chanpaisis; chinensis; colmeri; coreanensis; dakota; darmstadiensis; dendrolimus; entomocidus; entomocidus/subtoxicus; finitimus; fukuokaensis; galechiae; galleriae; graciosensis; guiyangiensis; higo; huazhongensis; iberica; indiana; israelensis; israelensis/tochigiensis; japonensis; jegathesan; jinghongiensis; kenyae; kim; kumamtoensis; kurstaki; kyushuensis; leesis; londrina; malayensis; medellin; mexicanensis; mogi; monterrey; morrisoni; muju; navarrensis; neoleonensis; nigeriensis; novosibirsk; ostriniae; oswaldocruzi; pahangi; pakistani; palmanyolensis; pingluonsis; pirenaica; poloniensis; pondicheriensis; pulsiensis; rongseni; roskildiensis; san diego; seoulensis; shandongiensis; silo; sinensis; sooncheon; sotto; sotto/dendrolimus; subtoxicus; sumiyoshiensis; sylvestriensis; tenebrionis; thailandensis; thompsoni; thuringiensis; tochigiensis; toguchini; tohokuensis; tolworthi; toumanoffi; vazensis; wratislaviensis; wuhanensis; xiaguangiensis; yosoo; yunnanensis; zhaodongensis; str. Al Hakam; or konkukian.

In some embodiments, a combination of the present disclosure comprises an AMP and a Bt toxin, wherein the Bt toxin is one or more fermentation solids, spores, toxins, pesticidal proteins, or variant thereof, isolated or originating from a Bacillus thuringiensis ssp. or varietas. For example, in some embodiments, an Bt toxin can isolated from a Bacillus thuringiensis ssp. selected from the following group: Bacillus thuringiensis ssp. aizawai; Bacillus thuringiensis ssp. aizawai/pacificus; Bacillus thuringiensis ssp. alesti; Bacillus thuringiensis ssp. amagiensis; Bacillus thuringiensis ssp. andalousiensis; Bacillus thuringiensis ssp. argentinensis; Bacillus thuringiensis ssp. asturiensis; Bacillus thuringiensis ssp. azorensis; Bacillus thuringiensis ssp. balearica; Bacillus thuringiensis ssp. berliner; Bacillus thuringiensis ssp. bolivia; Bacillus thuringiensis ssp. brasilensis; Bacillus thuringiensis ssp. cameroun; Bacillus thuringiensis ssp. canadensis; Bacillus thuringiensis ssp. chanpaisis; Bacillus thuringiensis ssp. chinensis; Bacillus thuringiensis ssp. colmeri; Bacillus thuringiensis ssp. coreanensis; Bacillus thuringiensis ssp. dakota; Bacillus thuringiensis ssp. darmstadiensis; Bacillus thuringiensis ssp. dendrolimus; Bacillus thuringiensis ssp. entomocidus; Bacillus thuringiensis ssp. entomocidus/subtoxicus; Bacillus thuringiensis ssp. finitimus; Bacillus thuringiensis ssp. fukuokaensis; Bacillus thuringiensis ssp. galechiae; Bacillus thuringiensis ssp. galleriae; Bacillus thuringiensis ssp. graciosensis; Bacillus thuringiensis ssp. guiyangiensis; Bacillus thuringiensis ssp. higo; Bacillus thuringiensis ssp. huazhongensis; Bacillus thuringiensis ssp. iberica; Bacillus thuringiensis ssp. indiana; Bacillus thuringiensis ssp. israelensis; Bacillus thuringiensis ssp. israelensis/tochigiensis; Bacillus thuringiensis ssp. japonensis; Bacillus thuringiensis ssp. jegathesan; Bacillus thuringiensis ssp. jinghongiensis; Bacillus thuringiensis ssp. kenyae; Bacillus thuringiensis ssp. kim; Bacillus thuringiensis ssp. kumamtoensis; Bacillus thuringiensis ssp. kunthalanags3; Bacillus thuringiensis ssp. kunthalaRX24; Bacillus thuringiensis ssp. kunthalaRX27: Bacillus thuringiensis ssp. kunthalaRX28; Bacillus thuringiensis ssp. kurstaki; Bacillus thuringiensis ssp. kyushuensis; Bacillus thuringiensis ssp. leesis; Bacillus thuringiensis ssp. londrina; Bacillus thuringiensis ssp. malayensis; Bacillus thuringiensis ssp. medellin; Bacillus thuringiensis ssp. mexicanensis; Bacillus thuringiensis ssp. mogi; Bacillus thuringiensis ssp. monterrey; Bacillus thuringiensis ssp. morrisoni; Bacillus thuringiensis ssp. muju; Bacillus thuringiensis ssp. navarrensis; Bacillus thuringiensis ssp. neoleonensis; Bacillus thuringiensis ssp. nigeriensis; Bacillus thuringiensis ssp. novosibirsk; Bacillus thuringiensis ssp. ostriniae; Bacillus thuringiensis ssp. oswaldocruzi; Bacillus thuringiensis ssp. pahangi; Bacillus thuringiensis ssp. pakistani; Bacillus thuringiensis ssp. palmanyolensis; Bacillus thuringiensis ssp. pingluonsis; Bacillus thuringiensis ssp. pirenaica; Bacillus thuringiensis ssp. poloniensis; Bacillus thuringiensis ssp. pondicheriensis; Bacillus thuringiensis ssp. pulsiensis; Bacillus thuringiensis ssp. rongseni; Bacillus thuringiensis ssp. roskildiensis; Bacillus thuringiensis ssp. san diego; Bacillus thuringiensis ssp. seoulensis; Bacillus thuringiensis ssp. shandongiensis; Bacillus thuringiensis ssp. silo; Bacillus thuringiensis ssp. sinensis; Bacillus thuringiensis ssp. sooncheon; Bacillus thuringiensis ssp. sotto; Bacillus thuringiensis ssp. sotto/dendrolimus; Bacillus thuringiensis ssp. subtoxicus; Bacillus thuringiensis ssp. sumiyoshiensis; Bacillus thuringiensis ssp. sylvestriensis; Bacillus thuringiensis ssp. tenebrionis; Bacillus thuringiensis ssp. thailandensis; Bacillus thuringiensis ssp. thompsoni; Bacillus thuringiensis ssp. thuringiensis; Bacillus thuringiensis ssp. tochigiensis; Bacillus thuringiensis ssp. toguchini; Bacillus thuringiensis ssp. tohokuensis; Bacillus thuringiensis ssp. tolworthi; Bacillus thuringiensis ssp. toumanoffi; Bacillus thuringiensis ssp. vazensis; Bacillus thuringiensis ssp. wratislaviensis; Bacillus thuringiensis ssp. wuhanensis; Bacillus thuringiensis ssp. xiaguangiensis; Bacillus thuringiensis ssp. yosoo; Bacillus thuringiensis ssp. yunnanensis; Bacillus thuringiensis ssp. zhaodongensis; Bacillus thuringiensis str. Al Hakam; Bacillus thuringiensis T01-328; Bacillus thuringiensis YBT-1518; or Bacillus thuringiensis ssp. konkukian.

In some embodiments, a combination of the present disclosure comprises an AMP and a Bt toxin, wherein the Bt toxin is one or more fermentation solids, spores, toxins, pesticidal proteins, or variant thereof, isolated or originating from a Bacillus thuringiensis serovar. For example, in some embodiments, the Bt toxin can be produced by a Bacillus thuringiensis serovar selected from the following group: Bacillus thuringiensis AKS-7; Bacillus thuringiensis Bt18247; Bacillus thuringiensis Bt18679: Bacillus thuringiensis Bt407; Bacillus thuringiensis DAR 81934; Bacillus thuringiensis DB27; Bacillus thuringiensis F14-1; Bacillus thuringiensis FC1; Bacillus thuringiensis FC10; Bacillus thuringiensis FC2; Bacillus thuringiensis FC6; Bacillus thuringiensis FC7; Bacillus thuringiensis FC8; Bacillus thuringiensis FC9; Bacillus thuringiensis HD-771; Bacillus thuringiensis HD-789; Bacillus thuringiensis HD1002; Bacillus thuringiensis IBL 200; Bacillus thuringiensis IBL 4222; Bacillus thuringiensis JM-Mgvxx-63; Bacillus thuringiensis LDC 391; Bacillus thuringiensis LM1212; Bacillus thuringiensis MC28; Bacillus thuringiensis Sbt003; Bacillus thuringiensis serovar aizawai; Bacillus thuringiensis serovar aizawai/pacificus; Bacillus thuringiensis serovar alesti; Bacillus thuringiensis serovar amagiensis; Bacillus thuringiensis serovar andalousiensis; Bacillus thuringiensis serovar argentinensis; Bacillus thuringiensis serovar asturiensis; Bacillus thuringiensis serovar azorensis; Bacillus thuringiensis serovar balearica; Bacillus thuringiensis serovar berliner; Bacillus thuringiensis serovar bolivia; Bacillus thuringiensis serovar brasilensis; Bacillus thuringiensis serovar cameroun; Bacillus thuringiensis serovar canadensis; Bacillus thuringiensis serovar chanpaisis; Bacillus thuringiensis serovar chinensis; Bacillus thuringiensis serovar colmeri; Bacillus thuringiensis serovar coreanensis; Bacillus thuringiensis serovar dakota; Bacillus thuringiensis serovar darmstadiensis; Bacillus thuringiensis serovar dendrolimus; Bacillus thuringiensis serovar entomocidus; Bacillus thuringiensis serovar entomocidus/subtoxicus; Bacillus thuringiensis serovar finitimus; Bacillus thuringiensis serovar fukuokaensis; Bacillus thuringiensis serovar galechiae; Bacillus thuringiensis serovar galleriae; Bacillus thuringiensis serovar graciosensis; Bacillus thuringiensis serovar guiyangiensis; Bacillus thuringiensis serovar higo; Bacillus thuringiensis serovar huazhongensis; Bacillus thuringiensis serovar iberica; Bacillus thuringiensis serovar indiana; Bacillus thuringiensis serovar israelensis; Bacillus thuringiensis serovar israelensis/tochigiensis; Bacillus thuringiensis serovar japonensis; Bacillus thuringiensis serovar jegathesan; Bacillus thuringiensis serovar jinghongiensis; Bacillus thuringiensis serovar kenyae; Bacillus thuringiensis serovar kim; Bacillus thuringiensis serovar kumamtoensis; Bacillus thuringiensis serovar kunthalanags3; Bacillus thuringiensis serovar kunthalaRX24: Bacillus thuringiensis serovar kunthalaRX27: Bacillus thuringiensis serovar kunthalaRX28: Bacillus thuringiensis serovar kurstaki; Bacillus thuringiensis serovar kyushuensis; Bacillus thuringiensis serovar leesis; Bacillus thuringiensis serovar londrina; Bacillus thuringiensis serovar malayensis; Bacillus thuringiensis serovar medellin; Bacillus thuringiensis serovar mexicanensis; Bacillus thuringiensis serovar mogi; Bacillus thuringiensis serovar monterrey; Bacillus thuringiensis serovar morrisoni; Bacillus thuringiensis serovar muju; Bacillus thuringiensis serovar navarrensis; Bacillus thuringiensis serovar neoleonensis; Bacillus thuringiensis serovar nigeriensis; Bacillus thuringiensis serovar novosibirsk; Bacillus thuringiensis serovar ostriniae; Bacillus thuringiensis serovar oswaldocruzi; Bacillus thuringiensis serovar pahangi; Bacillus thuringiensis serovar pakistani; Bacillus thuringiensis serovar palmanyolensis; Bacillus thuringiensis serovar pingluonsis; Bacillus thuringiensis serovar pirenaica; Bacillus thuringiensis serovar poloniensis; Bacillus thuringiensis serovar pondicheriensis; Bacillus thuringiensis serovar pulsiensis; Bacillus thuringiensis serovar rongseni; Bacillus thuringiensis serovar roskildiensis; Bacillus thuringiensis serovar san diego; Bacillus thuringiensis serovar seoulensis; Bacillus thuringiensis serovar shandongiensis; Bacillus thuringiensis serovar silo; Bacillus thuringiensis serovar sinensis; Bacillus thuringiensis serovar sooncheon; Bacillus thuringiensis serovar sotto; Bacillus thuringiensis serovar sotto/dendrolimus; Bacillus thuringiensis serovar subtoxicus: Bacillus thuringiensis serovar sumiyoshiensis; Bacillus thuringiensis serovar sylvestriensis; Bacillus thuringiensis serovar tenebrionis; Bacillus thuringiensis serovar thailandensis; Bacillus thuringiensis serovar thompsoni; Bacillus thuringiensis serovar thuringiensis; Bacillus thuringiensis serovar tochigiensis; Bacillus thuringiensis serovar toguchini; Bacillus thuringiensis serovar tohokuensis; Bacillus thuringiensis serovar tolworthi; Bacillus thuringiensis serovar toumanoffi; Bacillus thuringiensis serovar vazensis; Bacillus thuringiensis serovar wratislaviensis; Bacillus thuringiensis serovar wuhanensis: Bacillus thuringiensis serovar xiaguangiensis; Bacillus thuringiensis serovar yosoo; Bacillus thuringiensis serovar yunnanensis: Bacillus thuringiensis serovar zhaodongensis; Bacillus thuringiensis str. Al Hakam; Bacillus thuringiensis T01-328; Bacillus thuringiensis YBT-1518; and Bacillus thuringiensis serovar konkukian.

In some embodiments, a combination of the present disclosure comprises an AMP and a Bt toxin, wherein the Bt toxin is one or more fermentation solids. spores, toxins, pesticidal proteins, or variant thereof, isolated or originating from Bacillus thuringiensis var. israelensis, Bacillus thuringiensis var. aizawai, Bacillus thuringiensis var. kurstaki, or Bacillus thuringiensis var. tenebrionensis.

In some embodiments, a combination of the present disclosure comprises an AMP and a Bt toxin, wherein the Bt toxin is a toxin or insecticidal protein belonging to the following class: Cry (Proteins originally isolated from B. thuringiensis crystals in which the active form normally consists of three domains); Cyt (Cytolytic, normally single domain proteins); Vip (Multi-domain proteins originally identified as being Vegetative Insecticidal Proteins); Tpp (Beta pore-forming pesticidal proteins containing the Toxin_10 (Bin-like) domain); Mpp (Beta pore-forming pesticidal proteins from the ETX/Mtx2 family); Gpp (Aegerolysin like pesticidal proteins); App (Predominantly alpha helical pesticidal proteins); Spp (Sphaericolysin like pesticidal proteins); Mcf (Proteins related to the “Makes Caterpillars Floppy” toxins); Mtx (Proteins related to the Mtx1 toxin (2VSE) originally isolated from Lysinibacillus sphaericus); Vpa (Proteins related to the ADP-ribosyltransferase active component of binary toxins); Vpb (Proteins related to the binding component of binary toxins); Pra (Proteins related to the Photorhabdus Insect-Related toxin A component); Prb (Proteins related to the Photorhabdus Insect-Related toxin B component); Mpf (Pesticidal proteins that are part of the Membrane Attack Complex/Perforin superfamily); or Xpp (A holding class for pesticidal proteins with currently uncharacterized structures).

In some embodiments, a combination of the present disclosure comprises an AMP and a Bt toxin, wherein the Bt toxin is a parasporal crystal toxin, a secreted protein, a β-exotoxin, a 41.9-kDa insecticidal toxin, a sphaericolysin, an alveolysin, or an enhancin-like protein.

In some embodiments, a combination of the present disclosure comprises an AMP and a Bt toxin, wherein the Bt toxin

In some embodiments, a combination of the present disclosure comprises an AMP and a Bt toxin, wherein the Bt toxin

In some embodiments, a combination of the present disclosure comprises an AMP and a Bt toxin, wherein the Bt toxin is a δ-endotoxin.

In some embodiments, a combination of the present disclosure comprises an AMP and a Bt toxin, wherein the Bt toxin a Three-domain (3D) Cry family protein, a binary Bin-like family toxin, an ETX_MTX2-like family toxin, a Toxin-10 family toxin, an Aerolysin family toxin, or a cytolysin.

In some embodiments, a combination of the present disclosure comprises an AMP and a Bt toxin, wherein the Bt toxin a Three-domain (3D) Cry toxin, a mosquitocidal Cry toxin (Mtx), a binary-like (Bin) toxin, or a Cyt toxin.

In some embodiments, a combination of the present disclosure comprises an AMP and a Bt toxin, wherein the Bt toxin is a Three-domain (3D) Cry toxin or a Cyt toxin.

In some embodiments, a combination of the present disclosure comprises an AMP and a Bt toxin, wherein the Bt toxin can be a MTX2 toxin, e.g., a MTX2 toxin isolated from Lysinibacillus sphaericus.

In some embodiments, a combination of the present disclosure comprises an AMP and a Bt toxin, wherein the Bt toxin can be a Bin-like toxin, e.g., a Bin-like toxin isolated from Lysinibacillus sphaericus.

In some embodiments, a combination of the present disclosure comprises an AMP and a Bt toxin, wherein the Bt toxin can be a Bacillus thuringiensis var. israelensis (Bti) toxin.

In some embodiments, a combination of the present disclosure comprises an AMP and a Bt toxin, wherein the Bt toxin can be a Bacillus thuringiensis ssp. israelensis Strain BMP 144 Bti toxin.

In some embodiments, a combination of the present disclosure comprises an AMP and a Bt toxin, wherein the Bt toxin can be a Bacillus thuringiensis var. kurstaki (Btk) toxin.

In some embodiments, a combination of the present disclosure comprises an AMP and a Bt toxin, wherein the Bt toxin can be a Bacillus thuringiensis ssp. kurstaki strain EVB-113-19 Btk toxin.

In some embodiments, a combination of the present disclosure comprises an AMP and a Bt toxin, wherein the Bt toxin can be a Bacillus thuringiensis var. tenebrionis (Btt) toxin.

In some embodiments, a combination of the present disclosure comprises an AMP and a Bt toxin, wherein the Bt toxin can be a Bacillus thuringiensis ssp. tenebrionis strain NB-176 Btt toxin.

In some embodiments, a combination of the present disclosure comprises an AMP and a Bt toxin, wherein the Bt toxin can be contained in a commercially available product. For example, in some embodiments, the commercially available product comprising an IA can be AQUABAC XT® from Becker Microbial Products, Inc.; NOVODOR® FC from VALENT® U.S.A. LLC Agricultural Products; and/or BioProtec Plus™ from AEF Global Inc.

In some embodiments, a combination of the present disclosure comprises an AMP and a Bt toxin, wherein the Bt toxin can be one or more Bacillus thuringiensis ssp. kurstaki strain EVB-113-19 cells.

In some embodiments, a combination of the present disclosure comprises an AMP and a Bt toxin, wherein the Bt toxin can be one or more fermentation solids, spores, and/or insecticidal toxins isolated from Bacillus thuringiensis ssp. kurstaki strain EVB-113-19 cells.

In some embodiments, a combination of the present disclosure comprises an AMP and a Bt toxin, wherein the Bt toxin can be one or more Bacillus thuringiensis ssp. tenebrionis strain NB-176 cells.

In some embodiments, a combination of the present disclosure comprises an AMP and a Bt toxin, wherein the Bt toxin can be one or more fermentation solids, spores, and/or insecticidal toxins isolated from Bacillus thuringiensis ssp. tenebrionis strain NB-176 cells.

In some embodiments, a combination of the present disclosure comprises an AMP and a Bt toxin, wherein the Bt toxin can be one or more Bacillus thuringiensis ssp. israelensis Strain BMP 144 cells.

In some embodiments, a combination of the present disclosure comprises an AMP and a Bt toxin, wherein the Bt toxin can be one or more fermentation solids, spores, and/or insecticidal toxins isolated from Bacillus thuringiensis ssp. israelensis Strain BMP 144 cells.

In some embodiments, a combination of the present disclosure comprises an AMP and a Bt toxin, wherein the Bt toxin can be obtained from AQUABAC XT®. consisting of the following ingredients: 6-10% (˜8%) Bacillus thuringiensis ssp. israelensis Strain BMP 144 solids, spores & insecticidal toxins, wherein said insecticidal toxins are δ-endotoxins, and equivalent to 1,200 International Toxic Units (ITU/mg) (4.84 Billion ITU/gallon or 1.2 Billion ITU/Liter); and ˜92% other/inactive ingredients.

In some embodiments, a combination of the present disclosure comprises an AMP and a Bt toxin, wherein the Bt toxin can be obtained from NOVODOR® FC (or flowable concentrate), consisting of 10% Bacillus thuringiensis ssp. tenebrionis strain NB-176 fermentation solids and solubles, with a potency of 15,000 Leptinotarsa Units (LTU) per gram of product (equivalent to 16.3 Million LTU's per quart of product); and 90% other/inactive ingredients.

In some embodiments, a combination of the present disclosure comprises an AMP and a Bt toxin, wherein the Bt toxin can be obtained from BioProtec Plus™, consisting of 14.49% Bacillus thuringiensis ssp. kurstaki strain EVB-113-19 fermentation solids, spores, and insecticidal toxins with a potency of 17,500 Cabbage Looper Units (CLU) per mg of product (equivalent to 76 billion CLU per gallon of product); and 85.51% other/inactive ingredients.

In some embodiments, a combination of the present disclosure comprises an AMP and a Bt toxin, wherein the Bt toxin can be obtained from Leprotec®, which consists of 14.49% Bacillus thuringiensis ssp. kurstaki (Btk) strain EVB-113-19 fermentation solids, spores, and insecticidal toxins, with a potency of 17,500 Cabbage Looper Units (CLU) per mg of product (equivalent to 76 billion CLU per gallon of product); and 85.51% other/inactive ingredients. Leprotec® is available from Vestaron Corporation (4717 Campus Dr. Suite 1200, Kalamazoo, MI 49008; website: https://www.vestaron.com/leprotec/; CAS number: 68038-71-1; lot number: 23J19M.).

In some embodiments, a combination of the present disclosure comprises an AMP and a Bt toxin, wherein the Bt toxin has amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences set forth in any one of SEQ ID NOs: 17-192.

In some embodiments, a combination of the present disclosure comprises an AMP and a Bt toxin, wherein the Bt toxin can be a Cry protein having amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical. at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences set forth in any one of SEQ ID NOs: 17-66.

In some embodiments, a combination of the present disclosure comprises an AMP and a Bt toxin, wherein the Bt toxin can be a Cyt protein having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences set forth in any one of SEQ ID NOs: 67-86.

In some embodiments, a combination of the present disclosure comprises an AMP and a Bt toxin, wherein the Bt toxin can be a Vip having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to any one of the amino acid sequences set forth in any one of SEQ ID NOs: 87-192.

### Cry Proteins

In some embodiments, a combination of the present disclosure comprises an AMP and a Bt toxin, wherein the Bt toxin can be one or more of the following Cry proteins: Cry1Aa1, Cry1Aa2, Cry1Aa3, Cry1Aa4, Cry1Aa5, Cry1Aa6, Cry1Aa7, Cry1Aa8, Cry1Aa9, Cry1Aa10, Cry1Aa11, Cry1Aa12, Cry1Aa13, Cry1Aa14, Cry1Aa15, Cry1Aa16, Cry1Aa17, Cry1Aa18, Cry1Aa19, Cry1Aa20, Cry1Aa21, Cry1Aa22, Cry1Aa23, Cry1Aa24, Cry1Aa25, Cry1Ab1, Cry1Ab2, Cry1Ab3, Cry1Ab4, Cry1Ab5, Cry1Ab6, Cry1Ab7, Cry1Ab8, Cry1Ab9, Cry1Ab10, Cry1Ab11, Cry1Ab12, Cry1Ab13, Cry1Ab14, Cry1Ab15, Cry1Ab16, Cry1Ab17, Cry1Ab18, Cry1Ab19, Cry1Ab20, Cry1Ab21, Cry1Ab22, Cry1Ab23, Cry1Ab24, Cry1Ab25, Cry1Ab26, Cry1Ab27, Cry1Ab28, Cry1Ab29, Cry1Ab30, Cry1Ab31, Cry1Ab32, Cry1Ab33, Cry1Ab34, Cry1Ab35, Cry1Ab36, Cry1Ab-like, Cry1Ab-like, Cry1Ab-like, Cry1Ab-like, Cry1Ac1, Cry1Ac2, Cry1Ac3, Cry1Ac4, Cry1Ac5, Cry1Ac6, Cry1Ac7, Cry1Ac8, Cry1Ac9, Cry1Ac10, Cry1Ac11, Cry1Ac12, Cry1Ac13, Cry1Ac14, Cry1Ac15, Cry1Ac16, Cry1Ac17, Cry1Ac18, Cry1Ac19, Cry1Ac20, Cry1Ac21, Cry1Ac22, Cry1Ac23, Cry1Ac24, Cry1Ac25, Cry1Ac26, Cry1Ac27, Cry1Ac28, Cry1Ac29, Cry1Ac30, Cry1Ac31, Cry1Ac32, Cry1Ac33, Cry1Ac34, Cry1Ac35, Cry1Ac36, Cry1Ac37, Cry1Ac38, Cry1Ac39, Cry1Ad1, Cry1Ad2, Cry1Ae1, Cry1Af1, Cry1Ag1, Cry1Ah1, Cry1Ah2, Cry1Ah3, Cry1Ai1, Cry1Ai2, Cry1Aj1, Cry1A-like, Cry1Ba1, Cry1Ba2, Cry1Ba3, Cry1Ba4, Cry1Ba5, Cry1Ba6, Cry1Ba7, Cry1Ba8, Cry1Bb1, Cry1Bb2, Cry1Bb3, Cry1Be1, Cry1Bd1, Cry1Bd2, Cry1Bd3, Cry1Be1, Cry1Be2, Cry1Be3, Cry1Be4, Cry1Be5, Cry1Bf1, Cry1Bf2, Cry1Bg1, Cry1Bh1, Cry1Bi1, Cry1Bj1, Cry1Ca1, Cry1Ca2, Cry1Ca3, Cry1Ca4, Cry1Ca5, Cry1Ca6, Cry1Ca7, Cry1Ca8, Cry1Ca9, Cry1Ca10, Cry1Ca11, Cry1Ca12, Cry1Ca13, Cry1Ca14, Cry1Ca15, Cry1Cb1, Cry1Cb2, Cry1Cb3, Cry1Cb-like, Cry1Da1, Cry1Da2, Cry1Da3, Cry1Da4, Cry1Da5, Cry1Db1, Cry1Db2, Cry1Dc1, Cry1Dd1, Cry1Ea1, Cry1Ea2, Cry1Ea3, Cry1Ea4, Cry1Ea5, Cry1Ea6, Cry1Ea7, Cry1Ea8, Cry1Ea9, Cry1Ea10, Cry1Ea11, Cry1Ea12, Cry1Eb1, Cry1Fa1, Cry1Fa2, Cry1Fa3, Cry1Fa4, Cry1Fb1, Cry1Fb2, Cry1Fb3, Cry1Fb4, Cry1Fb5, Cry1Fb6, Cry1Fb7, Cry1Ga1, Cry1Ga2, Cry1Gb1, Cry1Gb2, Cry1Gc1, Cry1Ha1, Cry1Hb1, Cry1Hb2, Cry1He1, Cry1H-like, Cry1Ia1, Cry1Ia2, Cry1Ia3, Cry1Ia4, Cry1Ia5, Cry1Ia6, Cry1Ia7, Cry1Ia8, Cry1Ia9, Cry1Ia10, Cry1Ia11, Cry1Ia12, Cry1Ia13, Cry1Ia14, Cry1Ia15, Cry1Ia16, Cry1Ia17, Cry1Ia18, Cry1Ia19, Cry1Ia20, Cry1Ia2l, Cry1Ia22, Cry1Ia23, Cry1Ia24, Cry1Ia25, Cry1Ia26, Cry1Ia27, Cry1Ia28, Cry1Ia29, Cry1Ia30, Cry1Ia3l, Cry1Ia32, Cry1Ia33, Cry1Ia34, Cry1Ia35, Cry1Ia36, Cry1Ia37, Cry1Ia38, Cry1Ia39, Cry1Ia40, Cry1Ib1, Cry1Ib2, Cry1Ib3, Cry1Ib4, Cry1Ib5, Cry1Ib6, Cry1Ib7, Cry1Ib8, Cry1Ib9, Cry1Ib10, Cry1Ib11, Cry1Ic1, Cry1Ic2, Cry1Id1, Cry1Id2, Cry1Id3, Cry1Ie1, Cry1Ie2, Cry1Ie3, Cry1Ie4, Cry1Ie5, Cry1If1, Cry1Ig1, Cry1I-like, Cry1I-like, Cry1Ja1, Cry1Ja2, Cry1Ja3, Cry1Jb1, Cry1Jc1, Cry1Jc2, Cry1Jd1, Cry1Ka1, Cry1Ka2, Cry1La1, Cry1La2, Cry1La3, Cry1Ma1, Cry1Ma2, Cry1Na1, Cry1Na2, Cry1Na3, Cry1Nb1, Cry1-like, Cry2Aa1, Cry2Aa2, Cry2Aa3, Cry2Aa4, Cry2Aa5, Cry2Aa6, Cry2Aa7, Cry2Aa8, Cry2Aa9, Cry2Aa10, Cry2Aa11, Cry2Aa12, Cry2Aa13, Cry2Aa14, Cry2Aa15, Cry2Aa16, Cry2Aa17, Cry2Aa18, Cry2Aa19, Cry2Aa20, Cry2Aa21, Cry2Aa22, Cry2Aa23, Cry2Aa23, Cry2Aa25, Cry2Ab1, Cry2Ab2, Cry2Ab3, Cry2Ab4, Cry2Ab5, Cry2Ab6, Cry2Ab7, Cry2Ab8, Cry2Ab9, Cry2Ab10, Cry2Ab11, Cry2Ab12, Cry2Ab13, Cry2Ab14, Cry2Ab15, Cry2Ab16, Cry2Ab17, Cry2Ab18, Cry2Ab19, Cry2Ab20, Cry2Ab21, Cry2Ab22, Cry2Ab23, Cry2Ab24, Cry2Ab25, Cry2Ab26, Cry2Ab27, Cry2Ab28, Cry2Ab29, Cry2Ab30, Cry2Ab31, Cry2Ab32, Cry2Ab33, Cry2Ab34, Cry2Ab35, Cry2Ab36, Cry2Ac1, Cry2Ac2, Cry2Ac3, Cry2Ac4, Cry2Ac5, Cry2Ac6, Cry2Ac7, Cry2Ac8, Cry2Ac9, Cry2Ac10, Cry2Ac11, Cry2Ac12, Cry2Ad1, Cry2Ad2, Cry2Ad3, Cry2Ad4, Cry2Ad5, Cry2Ae1, Cry2Af1, Cry2Af2, Cry2Ag1, Cry2Ah1, Cry2Ah2, Cry2Ah3, Cry2Ah4, Cry2Ah5, Cry2Ah6, Cry2Ai1, Cry2Aj1, Cry2Ak1, Cry2Al1, Cry2Ba1, Cry2Ba2, Cry3Aa1, Cry3Aa2, Cry3Aa3, Cry3Aa4, Cry3Aa5, Cry3Aa6, Cry3Aa7, Cry3Aa8, Cry3Aa9, Cry3Aa10, Cry3Aa11, Cry3Aa12, Cry3Ba1, Cry3Ba2, Cry3Ba3, Cry3Bb1, Cry3Bb2, Cry3Bb3, Cry3Ca1, Cry4Aa1, Cry4Aa2, Cry4Aa3, Cry4Aa4, Cry4A-like, Cry4Ba1, Cry4Ba2, Cry4Ba3, Cry4Ba4, Cry4Ba5, Cry4Ba-like, Cry4Ca1, Cry4Ca2, Cry4Cb1, Cry4Cb2, Cry4Cb3, Cry4Cc1, Cry5Aa1, Cry5Ab1, Cry5Ac1, Cry5Ad1, Cry5Ba1, Cry5Ba2, Cry5Ba3, Cry5Ca1, Cry5Ca2, Cry5Da1, Cry5Da2, Cry5Ea1, Cry5Ea2, Cry6Aa1, Cry6Aa2, Cry6Aa3, Cry6Ba1, Cry7Aa1, Cry7Aa2, Cry7Ab1, Cry7Ab2, Cry7Ab3, Cry7Ab4, Cry7Ab5, Cry7Ab6, Cry7Ab7, Cry7Ab8, Cry7Ab9, Cry7Ac1, Cry7Ba1, Cry7Bb1, Cry7Ca1, Cry7Cb1, Cry7Da1, Cry7Da2, Cry7Da3, Cry7Ea1, Cry7Ea2, Cry7Ea3, Cry7Fa1, Cry7Fa2, Cry7Fb1, Cry7Fb2, Cry7Fb3, Cry7Ga1, Cry7Ga2, Cry7Gb1, Cry7Gc1, Cry7Gd1, Cry7Ha1, Cry7Ia1, Cry7Ja1, Cry7Ka1, Cry7Kb1, Cry7La1, Cry8Aa1, Cry8Ab1, Cry8Ac1, Cry8Ad1, Cry8Ba1, Cry8Bb1, Cry8Bc1, Cry8Ca1, Cry8Ca2, Cry8Ca3, Cry8Ca4, Cry8Ca5, Cry8Da1, Cry8Da2, Cry8Da3, Cry8Db1, Cry8Ea1, Cry8Ea2, Cry8Ea3, Cry8Ea4, Cry8Ea5, Cry8Ea6, Cry8Fa1, Cry8Fa2, Cry8Fa3, Cry8Fa4, Cry8Ga1, Cry8Ga2, Cry8Ga3, Cry8Ha1, Cry8Hb1, Cry8Ia1, Cry8Ia2, Cry8Ia3, Cry8Ia4, Cry8Ib1, Cry8Ib2, Cry8Ib3, Cry8Ja1, Cry8Ka1, Cry8Ka2, Cry8Ka3, Cry8Kb1, Cry8Kb2, Cry8Kb3, Cry8La1, Cry8Ma1, Cry8Ma2, Cry8Ma3, Cry8Na1, Cry8Pa1, Cry8Pa2, Cry8Pa3, Cry8Qa1, Cry8Qa2, Cry8Ra1, Cry8Sa1, Cry8Ta1, Cry8-like, Cry8-like, Cry9Aa1, Cry9Aa2, Cry9Aa3, Cry9Aa4, Cry9Aa5, Cry9Aa, like, Cry9Ba1, Cry9Ba2, Cry9Bb1, Cry9Ca1, Cry9Ca2, Cry9Cb1, Cry9Da1, Cry9Da2, Cry9Da3, Cry9Da4, Cry9Db1, Cry9Dc1, Cry9Ea1, Cry9Ea2, Cry9Ea3, Cry9Ea4, Cry9Ea5, Cry9Ea6, Cry9Ea7, Cry9Ea8, Cry9Ea9, Cry9Ea10, Cry9Ea11, Cry9Eb1, Cry9Eb2, Cry9Eb3, Cry9Ec1, Cry9Ed1, Cry9Ee1, Cry9Ee2, Cry9Fa1, Cry9Ga1, Cry9-like, Cry10Aa1, Cry10Aa2, Cry10Aa3, Cry10Aa4, Cry10A-like, Cry11Aa1, Cry11Aa2, Cry11Aa3, Cry11Aa4, Cry11Aa5, Cry11Aa-like, Cry11Ba1, Cry11Bb1, Cry11Bb2, Cry12Aa1, Cry13Aa1, Cry13Aa2, Cry14Aa1, Cry14Ab1, Cry15Aa1, Cry16Aa1, Cry17Aa1, Cry18Aa1, Cry18Ba1, Cry18Ca1, Cry19Aa1, Cry19Ba1, Cry19Ca1, Cry20Aa1, Cry20Ba1, Cry20Ba2, Cry20-like, Cry21Aa1, Cry21Aa2, Cry21Aa3, Cry21Ba1, Cry21Ca1, Cry21Ca2, Cry21Da1, Cry21Ea1, Cry21Fa1, Cry21Ga1, Cry21Ha1, Cry22Aa1, Cry22Aa2, Cry22Aa3, Cry22Ab1, Cry22Ab2, Cry22Ba1, Cry22Bb1, Cry23Aa1, Cry24Aa1, Cry24Ba1, Cry24Ca1, Cry24Da1, Cry25Aa1, Cry26Aa1, Cry27Aa1, Cry28Aa1, Cry28Aa2, Cry29Aa1, Cry29Ba1, Cry30Aa1, Cry30Ba1, Cry30Ca1, Cry30Ca2, Cry30Da1, Cry30Db1, Cry30Ea1, Cry30Ea2, Cry30Ea3, Cry30Ea4, Cry30Fa1, Cry30Ga1, Cry30Ga2, Cry31Aa1, Cry31Aa2, Cry31Aa3, Cry31Aa4, Cry31Aa5, Cry31Aa6, Cry31Ab1, Cry31Ab2, Cry31Ac1, Cry31Ac2, Cry31Ad1, Cry31Ad2, Cry32Aa1, Cry32Aa2, Cry32Ab1, Cry32Ba1, Cry32Ca1, Cry32Cb1, Cry32Da1, Cry32Ea1, Cry32Ea2, Cry32Eb1, Cry32Fa1, Cry32Ga1, Cry32Ha1, Cry32Hb1, Cry32Ia1, Cry32Ja1, Cry32Ka1, Cry32La1, Cry32Ma1, Cry32Mb1, Cry32Na1, Cry32Oa1, Cry32Pa1, Cry32Qa1, Cry32Ra1, Cry32Sa1, Cry32Ta1, Cry32Ua1, Cry32Va1, Cry32Wa1, Cry32Wa2, Cry32Xa1, Cry32Ya1, Cry33Aa1, Cry34Aa1, Cry34Aa2, Cry34Aa3, Cry34Aa4, Cry34Ab1, Cry34Ac1, Cry34Ac2, Cry34Ac3, Cry34Ba1, Cry34Ba2, Cry34Ba3, Cry35Aa1, Cry35Aa2, Cry35Aa3, Cry35Aa4, Cry35Ab1, Cry35Ab2, Cry35Ab3, Cry35Ac1, Cry35Ba1, Cry35Ba2, Cry35Ba3, Cry36Aa1, Cry37Aa1, Cry38Aa1, Cry39Aa1, Cry40Aa1, Cry40Ba1, Cry40Ca1, Cry40Da1, Cry41Aa1, Cry41Ab1, Cry41Ba1, Cry41Ba2, Cry41Ca1, Cry42Aa1, Cry43Aa1, Cry43Aa2, Cry43Ba1, Cry43Ca1, Cry43Cb1, Cry43Cc1, Cry43-like, Cry44Aa1, Cry45Aa1, Cry45Ba1, Cry46Aa1, Cry46Aa2, Cry46Ab1, Cry47Aa1, Cry48Aa1, Cry48Aa2, Cry48Aa3, Cry48Ab1, Cry48Ab2, Cry49Aa1, Cry49Aa2, Cry49Aa3, Cry49Aa4, Cry49Ab1, Cry50Aa1, Cry50Ba1, Cry50Ba2, Cry51Aa1, Cry51Aa2, Cry52Aa1, Cry52Ba1, Cry52Ca1, Cry53Aa1, Cry53Ab1, Cry54Aa1, Cry54Aa2, Cry54Ab1, Cry54Ba1, Cry54Ba2, Cry55Aa1, Cry55Aa2, Cry55Aa3, Cry56Aa1, Cry56Aa2, Cry56Aa3, Cry56Aa4, Cry57Aa1, Cry57Ab1, Cry58Aa1, Cry59Ba1, Cry59Aa1, Cry60Aa1, Cry60Aa2, Cry60Aa3, Cry60Ba1, Cry60Ba2, Cry60Ba3, Cry61Aa1, Cry61Aa2, Cry61Aa3, Cry62Aa1, Cry63Aa1, Cry64Aa1, Cry64Ba1, Cry64Ca1, Cry65Aa1, Cry65Aa2, Cry66Aa1, Cry66Aa2, Cry67Aa1, Cry67Aa2, Cry68Aa1, Cry69Aa1, Cry69Aa2, Cry69Ab1, Cry70Aa1, Cry70Ba1, Cry70Bb1, Cry71Aa1, Cry72Aa1, Cry72Aa2, Cry73Aa1, Cry74Aa, Cry75Aa1, Cry75Aa2, Cry75Aa3, Cry76Aa1, Cry77Aa1, and/or Cry78Aa1.

In some embodiments, a combination of the present disclosure comprises an AMP and a Bt toxin, wherein the Bt toxin can be any of the Cry toxins as described herein, or presented in Table 1.

TABLE 1
Non-limiting examples of Cry toxins, their accession numbers
on NCBI, and strain. Here, if a cell is left blank, then
the accession number and/or strain is not applicable.

| Name | NCBI Accession No. | Strain/Other ID |
|---|---|---|
| Cry1Aa1 | AAA22353 | Bt kurstaki HD1 |
| Cry1Aa2 | AAA22552 | Bt sotto |
| Cry1Aa3 | BAA00257 | Bt aizawai IPL7 |
| Cry1Aa4 | CAA31886 | Bt entomocidus |
| Cry1Aa5 | BAA04468 | Bt Fu-2-7 |
| Cry1Aa6 | AAA86265 | Bt kurstaki NRD-12 |
| Cry1Aa7 | AAD46139 | Bt C12 |
| Cry1Aa8 | I26149 | |
| Cry1Aa9 | BAA77213 | Bt dendrolimus T84A1 |
| Cry1Aa10 | AAD55382 | Bt kurstaki HD-1-02 |
| Cry1Aa11 | CAA70856 | Bt kurstaki |
| Cry1Aa12 | AAP80146 | Bt Ly30 |
| Cry1Aa13 | AAM44305 | Bt sotto |
| Cry1Aa14 | AAP40639 | unpublished |
| Cry1Aa15 | AAY66993 | Bt INTA Mol-12 |
| Cry1Aa16 | HQ439776 | Bt Ps9-E2 |
| Cry1Aa17 | HQ439788 | Bt PS9-C12 |
| Cry1Aa18 | HQ439790 | Bt PS9-D12 |
| Cry1Aa19 | HQ685121 | Bt LS-R-21 |
| Cry1Aa20 | JF340156 | Bt SK-798 |
| Cry1Aa21 | JN651496 | Bt LTS-209 |
| Cry1Aa22 | KC158223 | Bt Lip |
| Cry1Aa23 | KJ125392 | Bt |
| Cry1Aa24 | AGH68331 | Btk NAIMCC-B-00167 |
| Cry1Aa25 | MK391629 | Bt MPUB5 |
| Cry1Ab1 | AAA22330 | Bt berliner 1715 |
| Cry1Ab2 | AAA22613 | Bt kurstaki |
| Cry1Ab3 | AAA22561 | Bt kurstaki HD1 |
| Cry1Ab4 | BAA00071 | Bt kurstaki HD1 |
| Cry1Ab5 | CAA28405 | Bt berliner 1715 |
| Cry1Ab6 | AAA22420 | Bt kurstaki NRD-12 |
| Cry1Ab7 | CAA31620 | Bt aizawai IC1 |
| Cry1Ab8 | AAA22551 | Bt aizawai IPL7 |
| Cry1Ab9 | CAA38701 | Bt aizawai HD133 |
| Cry1Ab10 | A29125 | Bt kurstaki HD1 |
| Cry1Ab11 | I12419 | Bt A20 |
| Cry1Ab12 | AAC64003 | Bt kurstaki S93 |
| Cry1Ab13 | AAN76494 | Bt c005 |
| Cry1Ab14 | AAG16877 | Native Chilean Bt |
| Cry1Ab15 | AAO13302 | Bt B-Hm-16 |
| Cry1Ab16 | AAK55546 | Bt AC-11 |
| Cry1Ab17 | AAT46415 | Bt WB9 |
| Cry1Ab18 | AAQ88259 | Bt |
| Cry1Ab19 | AAW31761 | Bt X-2 |
| Cry1Ab20 | ABB72460 | BtC008 |
| Cry1Ab21 | ABS18384 | Bt IS5056 |
| Cry1Ab22 | ABW87320 | BtS2491Ab |
| Cry1Ab23 | HQ439777 | Bt N32-2-2 |
| Cry1Ab24 | HQ439778 | Bt HD12 |
| Cry1Ab25 | HQ685122 | Bt LS-R-30 |
| Cry1Ab26 | HQ847729 | DOR BT-1 |
| Cry1Ab27 | JN135249 | |
| Cry1Ab28 | JN135250 | |
| Cry1Ab29 | JN135251 | |
| Cry1Ab30 | JN135252 | |
| Cry1Ab31 | JN135253 | |
| Cry1Ab32 | JN135254 | |
| Cry1Ab33 | AAS93798 | Bt kenyae K3 |
| Cry1Ab34 | KC156668 | ARP102 |
| Cry1Ab35 | KT692985 | Bt GS36 |
| Cry1Ab36 | KY440260 | Bt NEAU B-X5 |
| Cry1Ab-like | AAK14336 | Bt kunthala RX24 |
| Cry1Ab-like | AAK14337 | Bt kunthala RX28 |
| Cry1Ab-like | AAK14338 | Bt kunthala RX27 |
| Cry1Ab-like | ABG88858 | Bt ly4a3 |
| Cry1Ac1 | AAA22331 | Bt kurstaki HD73 |
| Cry1Ac2 | AAA22338 | Bt kenyae |
| Cry1Ac3 | CAA38098 | Bt BTS89A |
| Cry1Ac4 | AAA73077 | Bt kurstaki PS85A1 |
| Cry1Ac5 | AAA22339 | Bt kurstaki PS81GG |
| Cry1Ac6 | AAA86266 | Bt kurstaki NRD-12 |
| Cry1Ac7 | AAB46989 | Bt kurstaki HD73 |
| Cry1Ac8 | AAC44841 | Bt kurstaki HD73 |
| Cry1Ac9 | AAB49768 | Bt DSIR732 |
| Cry1Ac10 | CAA05505 | Bt kurstaki YBT-1520 |
| Cry1Ac11 | CAA10270 | |
| Cry1Ac12 | I12418 | Bt A20 |
| Cry1Ac13 | AAD38701 | Bt kurstaki HD1 |
| Cry1Ac14 | AAQ06607 | Bt Ly30 |
| Cry1Ac15 | AAN07788 | Bt from Taiwan |
| Cry1Ac16 | AAU87037 | Bt H3 |
| Cry1Ac17 | AAX18704 | Bt kenyae HD549 |
| Cry1Ac18 | AAY88347 | Bt SK-729 |
| Cry1Ac19 | ABD37053 | Bt C-33 |
| Cry1Ac20 | ABB89046 | |
| Cry1Ac21 | AAY66992 | INTA Mol-12 |
| Cry1Ac22 | ABZ01836 | Bt W015-1 |
| Cry1Ac23 | CAQ30431 | Bt |
| Cry1Ac24 | ABL01535 | Bt 146-158-01 |
| Cry1Ac25 | FJ513324 | Bt Tm37-6 |
| Cry1Ac26 | FJ617446 | Bt Tm41-4 |
| Cry1Ac27 | FJ617447 | Bt Tm44-1B |
| Cry1Ac28 | ACM90319 | Bt Q-12 |
| Cry1Ac29 | DQ438941 | INTA TA24-6 |
| Cry1Ac30 | GQ227507 | Bt S1478-1 |
| Cry1Ac31 | GU446674 | Bt S3299-1 |
| Cry1Ac32 | HM061081 | Bt ZQ-89 |
| Cry1Ac33 | GQ866913 | Bt SK-711 |
| Cry1Ac34 | HQ230364 | Bt SK-783 |
| Cry1Ac35 | JF340157 | Bt SK-784 |
| Cry1Ac36 | JN387137 | Bt SK-958 |
| Cry1Ac37 | JQ317685 | Bt SK-793 |
| Cry1Ac38 | ACC86135 | Bt LSZ9408 |
| Cry1Ac39 | ALT07695 | LBIT1200 |
| Cry1Ad1 | AAA22340 | Bt aizawai PS81I |
| Cry1Ad2 | CAA01880 | Bt PS81RR1 |
| Cry1Ae1 | AAA22410 | Bt a1esti |
| Cry1Af1 | AAB82749 | Bt NT0423 |
| Cry1Ag1 | AAD46137 | |
| Cry1Ah1 | AAQ14326 | |
| Cry1Ah2 | ABB76664 | Bt a1esti |
| Cry1Ah3 | HQ439779 | Bt S6 |
| Cry1Ai1 | AAO39719 | |
| Cry1Ai2 | HQ439780 | Bt SC6H8 |
| Cry1Aj1 | KJ28846 | |
| Cry1A-like | AAK14339 | Bt kunthala nags3 |
| Cry1Ba1 | CAA29898 | Bt thuringiensis HD2 |
| Cry1Ba2 | CAA65003 | Bt entomocidus HD110 |
| Cry1Ba3 | AAK63251 | |
| Cry1Ba4 | AAK51084 | Bt entomocidus HD9 |
| Cry1Ba5 | ABO20894 | Bt sfw-12 |
| Cry1Ba6 | ABL60921 | Bt S601 |
| Cry1Ba7 | HQ439781 | Bt N17-37 |
| Cry1Ba8 | KJ868173 | Bt Na205-3 |
| Cry1Bb1 | AAA22344 | Bt EG5847 |
| Cry1Bb2 | HQ439782 | Bt WBT-2 |
| Cry1Bb3 | KJ619659 | Bt FH21 |
| Cry1Bc1 | CAA86568 | Bt morrisoni |
| Cry1Bd1 | AAD10292 | Bt wuhanensis HD525 |
| Cry1Bd2 | AAM93496 | Bt 834 |
| Cry1Bd3 | KX398132 | Bt K4 |
| Cry1Be1 | AAC32850 | Bt PS158C2 |
| Cry1Be2 | AAQ52387 | |
| Cry1Be3 | ACV96720 | Bt g9 |
| Cry1Be4 | HM070026 | |
| Cry1Be5 | KU761578 | Bt LBR2 |
| Cry1Bf1 | CAC50778 | |
| Cry1Bf2 | AAQ52380 | |
| Cry1Bg1 | AAO39720 | |
| Cry1Bh1 | HQ589331 | Bt PS46L |
| Cry1Bi1 | KC156700 | ARP260 |
| Cry1Bj1 | KT952325 | Bt |
| Cry1Ca1 | CAA30396 | Bt entomocidus 60.5 |
| Cry1Ca2 | CAA31951 | Bt aizawai 7.29 |
| Cry1Ca3 | AAA22343 | Bt aizawai PS81I |
| Cry1Ca4 | CAA01886 | Bt entomocidus HD110 |
| Cry1Ca5 | CAA65457 | Bt aizawai 7.29 |
| Cry1Ca6 [1] | AAF37224 | Bt AF-2 |
| Cry1Ca7 | AAG50438 | Bt J8 |
| Cry1Ca8 | AAM00264 | Bt c002 |
| Cry1Ca9 | AAL79362 | Bt G10-01A |
| Cry1Ca10 | AAN16462 | Bt E05-20a |
| Cry1Ca11 | AAX53094 | Bt C-33 |
| Cry1Ca12 | HM070027 | mo3-E7 |
| Cry1Ca13 | HQ412621 | Bt LB-R-78 |
| Cry1Ca14 | JN651493 | Bt LTS-38 |
| Cry1Ca15 | MK391630 | Bt MPU B5 |
| Cry1Cb1 | M97880 | Bt galleriae HD29 |
| Cry1Cb2 | AAG35409 | Bt c001 |
| Cry1Cb3 | ACD50894 | Bt 087 |
| Cry1Cb-like | AAX63901 | Bt TA476-1 |
| Cry1Da1 | CAA38099 | Bt aizawai HD68 |
| Cry1Da2 | I76415 | |
| Cry1Da3 | HQ439784 | Bt HD12 |
| Cry1Da4 | KJ619660 | Bt FH21 |
| Cry1Da5 | MG181949 | QL75-2 |
| Cry1Db1 | CAA80234 | Bt BTS00349A |
| Cry1Db2 | AAK48937 | Bt B-Pr-88 |
| Cry1Dc1 | ABK35074 | Bt JC291 |
| Cry1Dd1 | KJ28844 | |
| Cry1Ea1 | CAA37933 | Bt kenyae 4F1 |
| Cry1Ea2 | CAA39609 | Bt kenyae |
| Cry1Ea3 | AAA22345 | Bt kenyae PS81F |
| Cry1Ea4 | AAD04732 | Bt kenyae LBIT-147 |
| Cry1Ea5 | A15535 | |
| Cry1Ea6 | AAL50330 | Bt YBT-032 |
| Cry1Ea7 | AAW72936 | Bt JC190 |
| Cry1Ea8 | ABX11258 | Bt HZM2 |
| Cry1Ea9 | HQ439785 | Bt S6 |
| Cry1Ea10 | ADR00398 | Bt BR64 |
| Cry1Ea11 | JQ652456 | Bt |
| Cry1Ea12 | KF601559 | Bt strain V4 |
| Cry1Eb1 | AAA22346 | Bt aizawai PS81A2 |
| Cry1Fa1 | AAA22348 | Bt aizawai EG6346 |
| Cry1Fa2 | AAA22347 | Bt aizawai PS81I |
| Cry1Fa3 | HM070028 | Bt mo3-D8 |
| Cry1Fa4 | HM439638 | Bt mo3-D10 |
| Cry1Fb1 | CAA80235 | Bt BTS00349A |
| Cry1Fb2 | BAA25298 | Bt morrisoni INA67 |
| Cry1Fb3 | AAF21767 | Bt morrisoni |
| Cry1Fb4 | AAC10641 | |
| Cry1Fb5 | AAO13295 | Bt B-Pr-88 |
| Cry1Fb6 | ACD50892 | Bt 012 |
| Cry1Fb7 | ACD50893 | Bt 087 |
| Cry1Ga1 | CAA80233 | Bt BTS0349A |
| Cry1Ga2 | CAA70506 | Bt wuhanensis |
| Cry1Gb1 | AAD10291 | Bt wuhanensis HD525 |
| Cry1Gb2 | AAO13756 | Bt B-Pr-88 |
| Cry1Gc1 | AAQ52381 | |
| Cry1Ha1 | CAA80236 | Bt BTS02069AA |
| Cry1Hb1 | AAA79694 | Bt morrisoni BF190 |
| Cry1Hb2 | HQ439786 | Bt WBT-2 |
| Cry1Hc1 | KJ28845 | |
| Cry1H-like | AAF01213 | Bt JC291 |
| Cry1Ia1 | CAA44633 | Bt kurstaki |
| Cry1Ia2 | AAA22354 | Bt kurstaki |
| Cry1Ia3 | AAC36999 | Bt kurstaki HD1 |
| Cry1Ia4 | AAB00958 | Bt AB88 |
| Cry1Ia5 | CAA70124 | Bt 61 |
| Cry1Ia6 | AAC26910 | Bt kurstaki S101 |
| Cry1Ia7 | AAM73516 | Bt |
| Cry1Ia8 | AAK66742 | |
| Cry1Ia9 | AAQ08616 | Bt Ly30 |
| Cry1Ia10 | AAP86782 | Bt thuringiensis |
| Cry1Ia11 | CAC85964 | Bt kurstaki BNS3 |
| Cry1Ia12 | AAV53390 | Bt |
| Cry1Ia13 | ABF83202 | Bt |
| Cry1Ia14 | ACG63871 | Bt11 |
| Cry1Ia15 | FJ617445 | Bt E-1B |
| Cry1Ia16 | FJ617448 | Bt E-1A |
| Cry1Ia17 | GU989199 | Bt MX2 |
| Cry1Ia18 | ADK23801 | Bt MX9 |
| Cry1Ia19 | HQ439787 | Bt SC6H6 |
| Cry1Ia20 | JQ228426 | Bt wu1H-3 |
| Cry1Ia21 | JQ228424 | Bt you1D-9 |
| Cry1Ia22 | JQ228427 | Bt wu1E-3 |
| Cry1Ia23 | JQ228428 | Bt wu1E-4 |
| Cry1Ia24 | JQ228429 | Bt wu2B-6 |
| Cry1Ia25 | JQ228430 | Bt wu2G-11 |
| Cry1Ia26 | JQ228431 | Bt wu2G-12 |
| Cry1Ia27 | JQ228432 | Bt you2D-3 |
| Cry1Ia28 | JQ228433 | Bt you2E-3 |
| Cry1Ia29 | JQ228434 | Bt you2F-3 |
| Cry1Ia30 | JQ317686 | Bt 4J4 |
| Cry1Ia31 | JX944038 | Bt SC-7 |
| Cry1Ia32 | JX944039 | Bt SC-13 |
| Cry1Ia33 | JX944040 | Bt SC-51 |
| Cry1Ia34 | KJ868171 | Bt Na205-3 |
| Cry1Ia35 | AIF79803 | Bt V4 |
| Cry1Ia36 | KY212747 | Bt YC-10 |
| Cry1Ia37 | MG674828 | Bt SY80 |
| Cry1Ia38 | MG584186 | |
| Cry1Ia39 | MK393238 | Bt INTA H4-3 |
| Cry1Ia40 | MK391631 | Bt MPU B9 |
| Cry1Ib1 | AAA82114 | Bt entomocidus BP465 |
| Cry1Ib2 | ABW88019 | Bt PP61 |
| Cry1Ib3 | ACD75515 | Bt GS8 |
| Cry1Ib4 | HM051227 | Bt BF-4 |
| Cry1Ib5 | HM070028 | Bt mo3-D8 |
| Cry1Ib6 | ADK38579 | Bt LB52 |
| Cry1Ib7 | JN571740 | Bt SK-935 |
| Cry1Ib8 | JN675714 | |
| Cry1Ib9 | JN675715 | |
| Cry1Ib10 | JN675716 | |
| Cry1Ib11 | JQ228423 | Bt HD12 |
| Cry1Ic1 | AAC62933 | Bt C18 |
| Cry1Ic2 | AAE71691 | |
| Cry1Id1 | AAD44366 | |
| Cry1Id2 | JQ228422 | Bt HD12 |
| Cry1Id3 | KJ619661 | Bt FH21 |
| Cry1Ie1 | AAG43526 | Bt BTC007 |
| Cry1Ie2 | HM439636 | Bt T03B001 |
| Cry1Ie3 | KC156647 | ARP058 |
| Cry1Ie4 | KC156681 | ARP131 |
| Cry1Ie5 | KJ710646 | BN23-5 |
| Cry1If1 | AAQ52382 | |
| Cry1Ig1 | KC156701 | ARP166 |
| Cry1I-like | AAC31094 | |
| Cry1I-like | ABG88859 | Bt ly4a3 |
| Cry1Ja1 | AAA22341 | Bt EG5847 |
| Cry1Ja2 | HM070030 | WBT-1 |
| Cry1Ja3 | JQ228425 | Bt FH21 |
| Cry1Jb1 | AAA98959 | Bt EG5092 |
| Cry1Jc1 | AAC31092 | |
| Cry1Jc2 | AAQ52372 | |
| Cry1Jd1 | CAC50779 | Bt |
| Cry1Ka1 | AAB00376 | Bt morrisoni BF190 |
| Cry1Ka2 | HQ439783 | Bt WBT-2 |
| Cry1La1 | AAS60191 | Bt kurstaki K1 |
| Cry1La2 | HM070031 | Bt SC6H8 |
| Cry1La3 | KT692983 | Bt GS27 |
| Cry1Ma1 | FJ884067 | LBIT 1189 |
| Cry1Ma2 | KC156659 | ARP080 |
| Cry1Na1 | KC156648 | ARP009 |
| Cry1Na2 | AEH31422 | Bt T03B001 |
| Cry1Na3 | AKQ08661 | Bt BRC-ZYR2 |
| Cry1Nb1 | KC156678 | ARP146 |
| Cry1-like | AAC31091 | |
| Cry2Aa1 | AAA22335 | Bt kurstaki |
| Cry2Aa2 | AAA83516 | Bt kurstaki HD1 |
| Cry2Aa3 | D86064 | Bt sotto |
| Cry2Aa4 | AAC04867 | Bt kenyae HD549 |
| Cry2Aa5 | CAA10671 | Bt SL39 |
| Cry2Aa6 | CAA10672 | Bt YZ71 |
| Cry2Aa7 | CAA10670 | Bt CY29 |
| Cry2Aa8 | AAO13734 | Bt Dongbei 66 |
| Cry2Aa9 | AAO13750 | |
| Cry2Aa10 | AAQ04263 | |
| Cry2Aa11 | AAQ52384 | |
| Cry2Aa12 | ABI83671 | Bt Rpp39 |
| Cry2Aa13 | ABL01536 | Bt 146-158-01 |
| Cry2Aa14 | ACF04939 | Bt HD-550 |
| Cry2Aa15 | JN426947 | Bt SSy77 |
| Cry2Aa16 | KF667522 | Bt V4 |
| Cry2Aa17 | KF860848 | |
| Cry2Aa18 | ANF99565 | Bt SY49.1 |
| Cry2Aa19 | MG983752 | Bt-T32 |
| Cry2Aa20 | MG983753 | Bt-T405 |
| Cry2Aa21 | MG983754 | Bt-T414 |
| Cry2Aa22 | MH475904 | Bt-T527 |
| Cry2Aa23 | MH475905 | Bt-T532 |
| Cry2Aa23 | MH475906 | Bt-T536 |
| Cry2Aa25 | MH475907 | Bt-T543 |
| Cry2Ab1 | AAA22342 | Bt kurstaki HD1 |
| Cry2Ab2 | CAA39075 | Bt kurstaki HD1 |
| Cry2Ab3 | AAG36762 | Bt BTC002 |
| Cry2Ab4 | AAO13296 | Bt B-Pr-88 |
| Cry2Ab5 | AAQ04609 | Bt ly30 |
| Cry2Ab6 | AAP59457 | Bt WZ-7 |
| Cry2Ab7 | AAZ66347 | Bt 14-1 |
| Cry2Ab8 | ABC95996 | Bt WB2 |
| Cry2Ab9 | ABC74968 | Bt LLB6 |
| Cry2Ab10 | ABM21766 | Bt LyL |
| Cry2Ab11 | CAM84575 | Bt CMBL-BT1 |
| Cry2Ab12 | ABM21764 | Bt LyD |
| Cry2Ab13 | ACG76120 | Bt ywc5-4 |
| Cry2Ab14 | ACG76121 | Bt Bts |
| Cry2Ab15 | HM037126 | Bt BF-4 |
| Cry2Ab16 | GQ866914 | SK-793 |
| Cry2Ab17 | HQ439789 | Bt PS9-C12 |
| Cry2Ab18 | JN135255 | |
| Cry2Ab19 | JN135256 | |
| Cry2Ab20 | JN135257 | |
| Cry2Ab21 | JN135258 | |
| Cry2Ab22 | JN135259 | |
| Cry2Ab23 | JN135260 | |
| Cry2Ab24 | JN135261 | |
| Cry2Ab25 | JN415485 | Btk MnD |
| Cry2Ab26 | JN426946 | Bt SSy77 |
| Cry2Ab27 | JN415764 | |
| Cry2Ab28 | JN651494 | Bt LTS-7 |
| Cry2Ab29 | KF860847 | |
| Cry2Ab30 | EU623976 | Bt LSZ9408 |
| Cry2Ab31 | AHM93475 | Bt HTS-S-38 |
| Cry2Ab32 | KJ710647 | BN23-5 |
| Cry2Ab33 | KP053646 | Bt CYZ-4 |
| Cry2Ab34 | KX236449 | Bt BJH406 |
| Cry2Ab35 | KY212748 | Bt YC-10 |
| Cry2Ab36 | MK391632 | MPU B5 |
| Cry2Ac1 | CAA40536 | Bt shanghai S1 |
| Cry2Ac2 | AAG35410 | |
| Cry2Ac3 | AAQ52385 | |
| Cry2Ac4 | ABC95997 | Bt WB9 |
| Cry2Ac5 | ABC74969 | |
| Cry2Ac6 | ABC74793 | Bt wuhanensis |
| Cry2Ac7 | CAL18690 | Bt SBSBT-1 |
| Cry2Ac8 | CAM09325 | Bt CMBL-BT1 |
| Cry2Ac9 | CAM09326 | Bt CMBL-BT2 |
| Cry2Ac10 | ABN15104 | Bt QCL-1 |
| Cry2Ac11 | CAM83895 | Bt HD29 |
| Cry2Ac12 | CAM83896 | Bt CMBL-BT3 |
| Cry2Ad1 | AAF09583 | Bt BR30 |
| Cry2Ad2 | ABC86927 | Bt WB10 |
| Cry2Ad3 | CAK29504 | Bt 5_2AcT(1) |
| Cry2Ad4 | CAM32331 | Bt CMBL-BT2 |
| Cry2Ad5 | CAO78739 | Bt HD29 |
| Cry2Ae1 | AAQ52362 | |
| Cry2Af1 | ABO30519 | Bt C81 |
| Cry2Af2 | GQ866915 | SK-758 |
| Cry2Ag1 | ACH91610 | Bt JF19-2 |
| Cry2Ah1 | EU939453 | Bt SC6H8 |
| Cry2Ah2 | ACL80665 | Bt BRC-ZQL3 |
| Cry2Ah3 | GU073380 | HYW-8 |
| Cry2Ah4 | KC156702 | ARP193 |
| Cry2Ah5 | KT692984 | Bt GS3 |
| Cry2Ah6 | KX034204 | |
| Cry2Ai1 | FJ788388 | Bt |
| Cry2Aj1 | | |
| Cry2Ak1 | KC156660 | ARP067 |
| Cry2Al1 | KJ149819 | Bt SWK1 |
| Cry2Ba1 | KC156658 | ARP026 |
| Cry2Ba2 | KF014123 | HD395 |
| Cry3Aa1 | AAA22336 | Bt san diego |
| Cry3Aa2 | AAA22541 | Bt tenebrionis |
| Cry3Aa3 | CAA68482 | |
| Cry3Aa4 | AAA22542 | Bt tenebrionis |
| Cry3Aa5 | AAA50255 | Bt morrisoni EG2158 |
| Cry3Aa6 | AAC43266 | Bt tenebrionis |
| Cry3Aa7 | CAB41411 | Bt 22 |
| Cry3Aa8 | AAS79487 | Bt YM-03 |
| Cry3Aa9 | AAW05659 | Bt UTD-001 |
| Cry3Aa10 | AAU29411 | Bt 886 |
| Cry3Aa11 | AAW82872 | Bt tenebrionis Mm2 |
| Cry3Aa12 | ABY49136 | Bt tenebrionis |
| Cry3Ba1 | CAA34983 | Bt tolworthi 43F |
| Cry3Ba2 | CAA00645 | Bt PGSI208 |
| Cry3Ba3 | JQ397327 | Bt ML090 |
| Cry3Bb1 | AAA22334 | Bt EG4961 |
| Cry3Bb2 | AAA74198 | Bt EG5144 |
| Cry3Bb3 | I15475 | |
| Cry3Ca1 | CAA42469 | Bt kurstaki BtI109P |
| Cry4Aa1 | CAA68485 | Bt israelensis |
| Cry4Aa2 | BAA00179 | Bt israelensis HD522 |
| Cry4Aa3 | CAD30148 | Bt israelensis |
| Cry4Aa4 | AFB18317 | Bti BRC-LLP29 |
| Cry4A-like | AAY96321 | Bt LDC-9 |
| Cry4Ba1 | CAA30312 | Bt israelensis 4Q2-72 |
| Cry4Ba2 | CAA30114 | Bt israelensis |
| Cry4Ba3 | AAA22337 | Bt israelensis |
| Cry4Ba4 | BAA00178 | Bt israelensis HD522 |
| Cry4Ba5 | CAD30095 | Bt israelensis |
| Cry4Ba-like | ABC47686 | Bt LDC-9 |
| Cry4Ca1 | EU646202 | Bt Y41 |
| Cry4Ca2 | KM053252 | Bt SK700 |
| Cry4Cb1 | FJ403208 | Bt HS18-1 |
| Cry4Cb2 | FJ597622 | Bt Ywc2-8 |
| Cry4Cb3 | AHG25301 | Bt S2160-1 |
| Cry4Cc1 | FJ403207 | Bt MC28 |
| Cry5Aa1 | AAA67694 | Bt darmstadiensis PS17 |
| Cry5Ab1 | AAA67693 | Bt darmstadiensis PS17 |
| Cry5Ac1 | I34543 | |
| Cry5Ad1 | ABQ82087 | Bt L366 |
| Cry5Ba1 | AAA68598 | Bt PS86Q3 |
| Cry5Ba2 | ABW88931 | YBT 1518 |
| Cry5Ba3 | AFJ04417 | Bt zjfc85 |
| Cry5Ca1 | HM461869 | Sbt003 |
| Cry5Ca2 | ZP_04123426 | Bt T13001 |
| Cry5Da1 | HM461870 | Sbt003 |
| Cry5Da2 | ZP_04123980 | Bt T13001 |
| Cry5Ea1 | HM485580 | Sbt003 |
| Cry5Ea2 | ZP_04124038 | Bt T13001 |
| Cry6Aa1 | AAA22357 | Bt PS52A1 |
| Cry6Aa2 | AAM46849 | YBT 1518 |
| Cry6Aa3 | ABH03377 | Bt 96418 |
| Cry6Ba1 | AAA22358 | Bt PS69D1 |
| Cry7Aa1 | AAA22351 | Bt ga1leriae PGSI245 |
| Cry7Aa2 | MK840959 | Bt BM311.1 |
| Cry7Ab1 | AAA21120 | Bt dakota HD511 |
| Cry7Ab2 | AAA21121 | Bt kumamotoensis 867 |
| Cry7Ab3 | ABX24522 | Bt WZ-9 |
| Cry7Ab4 | EU380678 | Bt HQ122 |
| Cry7Ab5 | ABX79555 | Bt monterrey GM-33 |
| Cry7Ab6 | ACI44005 | Bt HQ122 |
| Cry7Ab7 | ADB89216 | Bt GW6 |
| Cry7Ab8 | GU145299 | |
| Cry7Ab9 | ADD92572 | Bt QG-121 |
| Cry7Ac1 | KJ789922 | Bt QZL20-1 |
| Cry7Ba1 | ABB70817 | Bt huazhongensis |
| Cry7Bb1 | KC156653 | ARP013 |
| Cry7Ca1 | ABR67863 | Bt BTH-13 |
| Cry7Cb1 | KC156698 | ARP269 |
| Cry7Da1 | ACQ99547 | Bt LH-2 |
| Cry7Da2 | HM572236 | |
| Cry7Da3 | KC156679 | ARP140 |
| Cry7Ea1 | HM035086 | Sbt009 |
| Cry7Ea2 | HM132124 | HD868(D8) |
| Cry7Ea3 | EEM19403 | BGSC 4Y1 |
| Cry7Fa1 | HM035088 | SBt009 |
| Cry7Fa2 | EEM19090 | BGSC 4Y1 |
| Cry7Fb1 | HM572235 | Bt |
| Cry7Fb2 | KC156682 | ARP162 |
| Cry7Fb3 | HM572235 | |
| Cry7Ga1 | HM572237 | Bt |
| Cry7Ga2 | KC156669 | ARP103 |
| Cry7Gb1 | KC156650 | ARP011 |
| Cry7Gc1 | KC156654 | ARP012 |
| Cry7Gd1 | KC156697 | ARP271 |
| Cry7Ha1 | KC156651 | ARP021 |
| Cry7Ia1 | KC156665 | ARP112 |
| Cry7Ja1 | KC156671 | ARP114 |
| Cry7Ka1 | KC156680 | ARP171 |
| Cry7Kb1 | BAM99306 | Bt dakota |
| Cry7La1 | BAM99307 | Bt dakota |
| Cry8Aa1 | AAA21117 | Bt kumamotoensis |
| Cry8Ab1 | EU044830 | Bt B-JJX |
| Cry8Ac1 | KC156662 | ARP068 |
| Cry8Ad1 | KC156684 | ARP215 |
| Cry8Ba1 | AAA21118 | Bt kumamotoensis |
| Cry8Bb1 | CAD57542 | |
| Cry8Bc1 | CAD57543 | |
| Cry8Ca1 | AAA21119 | Bt japonensis Buibui |
| Cry8Ca2 | AAR98783 | Bt HBF-1 |
| Cry8Ca3 | EU625349 | Bt FTL-23 |
| Cry8Ca4 | ADB54826 | Bt S185 |
| Cry8Ca5 | MK167020 | Bt BJH500 |
| Cry8Da1 | BAC07226 | Bt galleriae |
| Cry8Da2 | BD133574 | Bt |
| Cry8Da3 | BD133575 | Bt |
| Cry8Db1 | BAF93483 | Bt BBT2-5 |
| Cry8Ea1 | AAQ73470 | Bt 185 |
| Cry8Ea2 | EU047597 | Bt B-DLL |
| Cry8Ea3 | KC855216 | Bt GWL |
| Cry8Ea4 | AGM16383 | QZL144-1 |
| Cry8Ea5 | AGM16384 | QZL144-4 |
| Cry8Ea6 | KT692742 | ZK1 |
| Cry8Fa1 | AAT48690 | Bt 185 |
| Cry8Fa2 | HQ174208 | Bt DLL |
| Cry8Fa3 | AFH78109 | Bt L-27 |
| Cry8Fa4 | AGM16382 | QHW7-2 |
| Cry8Ga1 | AAT46073 | Bt HBF-18 |
| Cry8Ga2 | ABC42043 | Bt 145 |
| Cry8Ga3 | FJ198072 | Bt FCD114 |
| Cry8Ha1 | AAW81032 | Bt 185 |
| Cry8Hb1 | KP713881 | Bt |
| Cry8Ia1 | EU381044 | Bt su4 |
| Cry8Ia2 | GU073381 | Bt HW-11 |
| Cry8Ia3 | HM044664 | Sbt030 |
| Cry8Ia4 | KC156674 | ARP124 |
| Cry8Ib1 | GU325772 | Bt F4 |
| Cry8Ib2 | KC156677 | ARP135 |
| Cry8Ib3 | AHG25076 | Bt TS3 |
| Cry8Ja1 | EU625348 | Bt FPT-2 |
| Cry8Ka1 | FJ422558 | |
| Cry8Ka2 | ACN87262 | Bt kenyae |
| Cry8Ka3 | AGM16381 | QHW7-2 |
| Cry8Kb1 | HM123758 | ST8 |
| Cry8Kb2 | KC156675 | ARP158 |
| Cry8Kb3 | KJ123823 | INTA Fr7-4 |
| Cry8La1 | GU325771 | Bt F4 |
| Cry8Ma1 | | Sbt016 |
| Cry8Ma2 | EEM86551 | BGSC 4CC1 |
| Cry8Ma3 | HM210574 | NARC Bt17 (C6) |
| Cry8Na1 | HM640939 | BtQ52-7 |
| Cry8Pa1 | HQ388415 | Bt ST8 |
| Cry8Pa2 | HQ413324 | Bt QCM(T1) |
| Cry8Pa3 | KJ123823 | INTA Fr7-4 |
| Cry8Qa1 | HQ441166 | Bt ST8 |
| Cry8Qa2 | KC152468 | Bt INTA Fr7-4 |
| Cry8Ra1 | AFP87548 | Bt R36 |
| Cry8Sa1 | JQ740599 | Bt Strain 62 |
| Cry8Ta1 | KC156673 | ARP110 |
| Cry8-like | FJ770571 | Bt canadensis |
| Cry8-like | ABS53003 | Bt |
| Cry9Aa1 | CAA41122 | Bt galleriae |
| Cry9Aa2 | CAA41425 | Bt DSIR517 |
| Cry9Aa3 | GQ249293 | Bt SC5(D2) |
| Cry9Aa4 | GQ249294 | Bt T03C001 |
| Cry9Aa5 | JX174110 | BGSN1 |
| Cry9Aa like | AAQ52376 | |
| Cry9Ba1 | CAA52927 | Bt galleriae |
| Cry9Ba2 | GU299522 | Bt B-SC5 |
| Cry9Bb1 | AAV28716 | Bt japonensis |
| Cry9Ca1 | CAA85764 | Bt tolworthi |
| Cry9Ca2 | AAQ52375 | |
| Cry9Cb1 | MK005301 | |
| Cry9Da1 | BAA19948 | Bt japonensis N141 |
| Cry9Da2 | AAB97923 | Bt japonensis |
| Cry9Da3 | GQ249293 | Bt SC5 (D2) |
| Cry9Da4 | GQ249297 | Bt T03B001 |
| Cry9Db1 | AAX78439 | Bt kurstaki DP1019 |
| Cry9Dc1 | KC156683 | ARP168 |
| Cry9Ea1 | BAA34908 | Bt aizawai SSK-10 |
| Cry9Ea2 | AAO12908 | Bt B-Hm-16 |
| Cry9Ea3 | ABM21765 | Bt lyA |
| Cry9Ea4 | ACE88267 | Bt ywc5-4 |
| Cry9Ea5 | ACF04743 | Bts |
| Cry9Ea6 | ACG63872 | Bt 11 |
| Cry9Ea7 | FJ380927 | Bt 4 |
| Cry9Ea8 | GQ249292 | Bt SC5(E8) |
| Cry9Ea9 | JN651495 | Bt LTS-7 |
| Cry9Ea10 | KT692743 | ZK2 |
| Cry9Ea11 | MK391633 | Bt MPU B9 |
| Cry9Eb1 | CAC50780 | |
| Cry9Eb2 | GQ249298 | Bt T23001 |
| Cry9Eb3 | KC156646 | ARP057 |
| Cry9Ec1 | AAC63366 | Bt galleriae |
| Cry9Ed1 | AAX78440 | Bt kurstaki DP1019 |
| Cry9Ee1 | GQ249296 | Bt T03B001 |
| Cry9Ee2 | KC156664 | ARP095 |
| Cry9Fa1 | KC156692 | ARP212 |
| Cry9Ga1 | KC156699 | ARP188 |
| Cry9-like | AAC63366 | Bt galleriae |
| Cry10Aa1 | AAA22614 | Bt israelensis |
| Cry10Aa2 | E00614 | Bt israelensis ONR-60A |
| Cry10Aa3 | CAD30098 | Bt israelensis |
| Cry10Aa4 | AFB18318 | Bti BRC-LLP29 |
| Cry10A-like | DQ167578 | Bt LDC-9 |
| Cry11Aa1 | AAA22352 | Bt israelensis |
| Cry11Aa2 | AAA22611 | Bt israelensis |
| Cry11Aa3 | CAD30081 | Bt israelensis |
| Cry11Aa4 | AFB18319 | Bti BRC-LLP29 |
| Cry11Aa5 | MH253686 | |
| Cry11Aa-like | DQ166531 | Bt LDC-9 |
| Cry11Ba1 | CAA60504 | Bt jegathesan 367 |
| Cry11Bb1 | AAC97162 | Bt medellin |
| Cry11Bb2 | HM068615 | Bt K34 |
| Cry12Aa1 | AAA22355 | Bt PS33F2 |
| Cry13Aa1 | AAA22356 | Bt PS63B |
| Cry13Aa2 | CP015350 | Bt MYBT18246 |
| Cry14Aa1 | AAA21516 | Bt sotto PS80JJ1 |
| Cry14Ab1 | KC156652 | ARP001 |
| Cry15Aa1 | AAA22333 | Bt thompsoni |
| Cry16Aa1 | CAA63860 | Cb malaysia CH18 |
| Cry17Aa1 | CAA67841 | Cb malaysia CH18 |
| Cry18Aa1 | CAA67506 | Paenibacillus popilliae |
| Cry18Ba1 | AAF89667 | Paenibacillus popilliae |
| Cry18Ca1 | AAF89668 | Paenibacillus popilliae |
| Cry19Aa1 | CAA68875 | Bt jegathesan 367 |
| Cry19Ba1 | BAA32397 | Bt higo |
| Cry19Ca1 | AFM37572 | BGSC 4CE1 |
| Cry20Aa1 | AAB93476 | Bt fukuokaensis |
| Cry20Ba1 | ACS93601 | Bt higo LBIT-976 |
| Cry20Ba2 | KC156694 | ARP192 |
| Cry20-like | GQ144333 | Bt Y-5 |
| Cry21Aa1 | I32932 | |
| Cry21Aa2 | I66477 | |
| Cry21Aa3 | MF893204 | |
| Cry21Ba1 | BAC06484 | Bt roskildiensis |
| Cry21Ca1 | JF521577 | |
| Cry21Ca2 | KC156687 | ARP258 |
| Cry21Da1 | JF521578 | Sbt072 |
| Cry21Ea1 | KC865049 | |
| Cry21Fa1 | KF701307 | DB27 |
| Cry21Ga1 | KF771885 | DB27 |
| Cry21Ha1 | KF771886 | DB27 |
| Cry22Aa1 | I34547 | |
| Cry22Aa2 | CAD43579 | Bt |
| Cry22Aa3 | ACD93211 | Bt FZ-4 |
| Cry22Ab1 | AAK50456 | Bt EG4140 |
| Cry22Ab2 | CAD43577 | Bt |
| Cry22Ba1 | CAD43578 | Bt |
| Cry22Bb1 | KC156672 | ARP148 |
| Cry23Aa1 | AAF76375 | Bt |
| Cry24Aa1 | AAC61891 | Bt jegathesan |
| Cry24Ba1 | BAD32657 | Bt sotto |
| Cry24Ca1 | CAJ43600 | Bt FCC-41 |
| Cry24Da1 | KJ439561 | BLB32 |
| Cry25Aa1 | AAC61892 | Bt jegathesan |
| Cry26Aa1 | AAD25075 | Bt finitimus B-1166 |
| Cry27Aa1 | BAA82796 | Bt higo |
| Cry28Aa1 | AAD24189 | Bt finitimus B-1161 |
| Cry28Aa2 | AAG00235 | Bt finitimus |
| Cry29Aa1 | CAC80985 | Bt medellin |
| Cry29Ba1 | KC865046 | |
| Cry30Aa1 | CAC80986 | Bt medellin |
| Cry30Ba1 | BAD00052 | Bt entomocidus |
| Cry30Ca1 | BAD67157 | Bt sotto |
| Cry30Ca2 | ACU24781 | Bt jegathesan 367 |
| Cry30Da1 | EF095955 | Bt Y41 |
| Cry30Db1 | BAE80088 | Bt aizawai BUN1-14 |
| Cry30Ea1 | ACC95445 | Bt S2160-1 |
| Cry30Ea2 | FJ499389 | Bt Ywc2-8 |
| Cry30Ea3 | FJ527836 | Bt Hs18-1 |
| Cry30Ea4 | KJ740649 | BN15-6 |
| Cry30Fa1 | ACI22625 | Bt MC28 |
| Cry30Ga1 | ACG60020 | Bt HS18-1 |
| Cry30Ga2 | HQ638217 | Bt S2160-1 |
| Cry31Aa1 | BAB11757 | Bt 84-HS-1-11 |
| Cry31Aa2 | AA187458 | Bt M15 |
| Cry31Aa3 | BAE79808 | Bt B0195 |
| Cry31Aa4 | BAF32571 | Bt 79-25 |
| Cry31Aa5 | BAF32572 | Bt 92-10 |
| Cry31Aa6 | BAI44026 | M019 |
| Cry31Ab1 | BAE79809 | Bt B0195 |
| Cry31Ab2 | BAF32570 | Bt 31-5 |
| Cry31Ac1 | BAF34368 | Bt 87-29 |
| Cry31Ac2 | AB731600 | Bt B0462 |
| Cry31Ad1 | BAI44022 | Bt MO19 |
| Cry31Ad2 | AGO57767 | Bt 64-1-94 |
| Cry32Aa1 | AAG36711 | Bt yunnanensis |
| Cry32Aa2 | GU063849 | Bt FBG-1 |
| Cry32Ab1 | GU063850 | Bt FZ-2 |
| Cry32Ba1 | BAB78601 | Bt |
| Cry32Ca1 | BAB78602 | Bt |
| Cry32Cb1 | KC156708 | ARP227 |
| Cry32Da1 | BAB78603 | Bt |
| Cry32Ea1 | GU324274 | Bt HYD-3 |
| Cry32Ea2 | KC156686 | ARP239 |
| Cry32Eb1 | KC156663 | ARP092 |
| Cry32Fa1 | KC156656 | ARP055 |
| Cry32Ga1 | KC156657 | ARP052 |
| Cry32Ha1 | KC156661 | ARP076 |
| Cry32Hb1 | KC156666 | ARP096 |
| Cry32Ia1 | KC156667 | ARP104 |
| Cry32Ja1 | KC156685 | ARP262 |
| Cry32Ka1 | KC156688 | ARP259 |
| Cry32La1 | KC156689 | ARP203 |
| Cry32Ma1 | KC156690 | ARP256 |
| Cry32Mb1 | KC156704 | ARP242 |
| Cry32Na1 | KC156691 | ARP179 |
| Cry32Oa1 | KC156703 | ARP218 |
| Cry32Pa1 | KC156705 | ARP277 |
| Cry32Qa1 | KC156706 | ARP174 |
| Cry32Ra1 | KC156707 | ARP229 |
| Cry32Sa1 | KC156709 | ARP185 |
| Cry32Ta1 | KC156710 | ARP220 |
| Cry32Ua1 | KC156655 | ARP050 |
| Cry32Va1 | | LM1212 |
| Cry32Wa1 | | LM1212 |
| Cry32Wa2 | AHN52957 | Bt B3 |
| Cry32Xa1 | KX094974 | |
| Cry32Ya1 | KX094973 | |
| Cry33Aa1 | AA126871 | Bt dakota |
| Cry34Aa1 | AAG50341 | Bt PS80JJ1 |
| Cry34Aa2 | AAK64560 | Bt EG5899 |
| Cry34Aa3 | AAT29032 | Bt PS69Q |
| Cry34Aa4 | AAT29030 | Bt PS185GG |
| Cry34Ab1 | AAG41671 | Bt PS149B1 |
| Cry34Ac1 | AAG50118 | Bt PS167H2 |
| Cry34Ac2 | AAK64562 | Bt EG9444 |
| Cry34Ac3 | AAT29029 | Bt KR1369 |
| Cry34Ba1 | AAK64565 | Bt EG4851 |
| Cry34Ba2 | AAT29033 | Bt PS201L3 |
| Cry34Ba3 | AAT29031 | Bt PS201HH2 |
| Cry35Aa1 | AAG50342 | Bt PS80JJ1 |
| Cry35Aa2 | AAK64561 | Bt EG5899 |
| Cry35Aa3 | AAT29028 | Bt PS69Q |
| Cry35Aa4 | AAT29025 | Bt PS185GG |
| Cry35Ab1 | AAG41672 | Bt PS149B1 |
| Cry35Ab2 | AAK64563 | Bt EG9444 |
| Cry35Ab3 | AY536891 | Bt KR1369 |
| Cry35Ac1 | AAG50117 | Bt PS167H2 |
| Cry35Ba1 | AAK64566 | Bt EG4851 |
| Cry35Ba2 | AAT29027 | Bt PS201L3 |
| Cry35Ba3 | AAT29026 | Bt PS201HH2 |
| Cry36Aa1 | AAK64558 | Bt |
| Cry37Aa1 | AAF76376 | Bt |
| Cry38Aa1 | AAK64559 | Bt |
| Cry39Aa1 | BAB72016 | Bt aizawai |
| Cry40Aa1 | BAB72018 | Bt aizawai |
| Cry40Ba1 | BAC77648 | Bun1-14 |
| Cry40Ca1 | EU381045 | Bt Y41 |
| Cry40Da1 | ACF15199 | Bt S2096-2 |
| Cry41Aa1 | BAD35157 | Bt A1462 |
| Cry41Abl | BAD35163 | Bt A1462 |
| Cry41Ba1 | HM461871 | Sbt021 |
| Cry41Ba2 | ZP_04099652 | BGSC 4AW1 |
| Cry41Ca1 | | LM1212 |
| Cry42Aa1 | BAD35166 | Bt A1462 |
| Cry43Aa1 | BAD15301 | P. lentimorbus semadara |
| Cry43Aa2 | BAD95474 | P. popilliae popilliae |
| Cry43Ba1 | BAD15303 | P. lentimorbus semadara |
| Cry43Ca1 | KC156676 | ARP132 |
| Cry43Cb1 | KC156695 | ARP252 |
| Cry43Cc1 | KC156696 | ARP191 |
| Cry43-like | BAD15305 | P. lentimorbus semadara |
| Cry44Aa1 | BAD08532 | Bt entomocidus INA288 |
| Cry45Aa1 | BAD22577 | Bt 89-T-34-22 |
| Cry45Ba1 | | LM1212 |
| Cry46Aa1 | BAC79010 | Bt dakota |
| Cry46Aa2 | BAG68906 | Bt A1470 |
| Cry46Ab1 | BAD35170 | Bt |
| Cry47Aa1 | AAY24695 | Bt CAA890 |
| Cry48Aa1 | CAJ18351 | Bs IAB59 |
| Cry48Aa2 | CAJ86545 | Bs 47-6B |
| Cry48Aa3 | CAJ86546 | Bs NHA15b |
| Cry48Ab1 | CAJ86548 | Bs LP1G |
| Cry48Ab2 | CAJ86549 | Bs 2173 |
| Cry49Aa1 | CAH56541 | Bs IAB59 |
| Cry49Aa2 | CAJ86541 | Bs 47-6B |
| Cry49Aa3 | CAJ86543 | BsNHA15b |
| Cry49Aa4 | CAJ86544 | Bs 2173 |
| Cry49Ab1 | CAJ86542 | Bs LP1G |
| Cry50Aa1 | BAE86999 | Bt sotto |
| Cry50Ba1 | GU446675 | Bt S2160-1 |
| Cry50Ba2 | GU446676 | Bt S3161-3 |
| Cry51Aa1 | ABI14444 | Bt F14-1 |
| Cry51Aa2 | GU570697 | EG2934 |
| Cry52Aa1 | EF613489 | Bt Y41 |
| Cry52Ba1 | FJ361760 | Bt BM59-2 |
| Cry52Ca1 | KM053253 | Bt SK700 |
| Cry53Aa1 | EF633476 | Bt Y41 |
| Cry53Ab1 | FJ361759 | Bt MC28 |
| Cry54Aa1 | ACA52194 | Bt MC28 |
| Cry54Aa2 | GQ140349 | Bt FBG25 |
| Cry54Ab1 | JQ916908 | Bt MC28 |
| Cry54Ba1 | GU446677 | Bt S2160-1 |
| Cry54Ba2 | KJ740650 | BN15-6 |
| Cry55Aa1 | ABW88932 | YBT 1518 |
| Cry55Aa2 | AAE33526 | Bt Y41 |
| Cry55Aa3 | HG764207 | Bt T44 |
| Cry56Aa1 | ACU57499 | Bt Ywc2-8 |
| Cry56Aa2 | GQ483512 | Bt G7-1 |
| Cry56Aa3 | JX025567 | Bt HS18-1 |
| Cry56Aa4 | KJ740651 | BN7-5 |
| Cry57Aa1 | ACN87261 | Bt kim |
| Cry57Ab1 | KF638650 | Bt LTS290 |
| Cry58Aa1 | ACN87260 | Bt entomocidus |
| Cry59Ba1 | JN790647 | Bt Bm59-2 |
| Cry59Aa1 | ACR43758 | Bt kim LBIT-980 |
| Cry60Aa1 | ACU24782 | Bt jegathesan |
| Cry60Aa2 | EAO57254 | Bt israelensis |
| Cry60Aa3 | EEM99278 | Bt IBL 4222 |
| Cry60Ba1 | GU810818 | Bt malayensis |
| Cry60Ba2 | EAO57253 | Bt israelensis |
| Cry60Ba3 | EEM99279 | Bt IBL 4222 |
| Cry61Aa1 | HM035087 | Sbt009 |
| Cry61Aa2 | HM132125 | HD868 (E5) |
| Cry61Aa3 | EEM19308 | BGSC 4Y1 |
| Cry62Aa1 | HM054509 | ST7 |
| Cry63Aa1 | BAI44028 | MO19 |
| Cry64Aa1 | BAJ05397 | Bt tohokuensis |
| Cry64Ba1 | AGT29559 | BT 210-8-45 |
| Cry64Ca1 | AGT29560 | BT 210-8-45 |
| Cry65Aa1 | HM461868 | SBt 003 |
| Cry65Aa2 | ZP_04123838 | T13001 |
| Cry66Aa1 | AEB52311 | SBt 021 |
| Cry66Aa2 | ZP_04099945 | BGSC 4AW1 |
| Cry67Aa1 | HM485582 | SBt 009 |
| Cry67Aa2 | ZP_04148882 | BGSC 4Y1 |
| Cry68Aa1 | HQ113114 | Bt MC28 |
| Cry69Aa1 | HQ401006 | Bt MC28 |
| Cry69Aa2 | JQ821388 | Bt MC28 |
| Cry69Ab1 | JN209957 | Bt hs18-1 |
| Cry70Aa1 | JN646781 | Bt hs18-1 |
| Cry70Ba1 | ADO51070 | Bt MC28 |
| Cry70Bb1 | EEL67276 | Bc AH603 |
| Cry71Aa1 | JX025568 | Bt Hs18-1 |
| Cry72Aa1 | JX025569 | Bt Hs18-1 |
| Cry72Aa2 | KX094975 | |
| Cry73Aa1 | AEH76822 | Sbt Sbt029 |
| Cry74Aa | | LM1212 |
| Cry75Aa1 | ASY04853 | Bl EG5553 |
| Cry75Aa2 | ASY04852 | Bl EG5551 |
| Cry75Aa3 | ASY04851 | Bl EG5552 |
| Cry76Aa1 | MH810248 | |
| Cry77Aa1 | MH810249 | |
| Cry78Aa1 | KY780623 | Bt C9F1 |

Any of the Cry proteins described herein are suitable for use in a combination of the present disclosure.

### Cyt Proteins

In some embodiments, a combination of the present disclosure comprises an AMP and a Bt toxin, wherein the Bt toxin can be one or more of the following Cyt proteins: Cyt1Aa1, Cyt1Aa2, Cyt1Aa3, Cyt1Aa4, Cyt1Aa5, Cyt1Aa6, Cyt1Aa7, Cyt1Aa8, Cyt1Aa-like, Cyt1Ab1, Cyt1Ba1, Cyt1Ca1, Cyt1Da1, Cyt1Da2, Cyt2Aa1, Cyt2Aa2, Cyt2Aa3, Cyt2Aa4, Cyt2Ba1, Cyt2Ba2, Cyt2Ba3, Cyt2Ba4, Cyt2Ba5, Cyt2Ba6, Cyt2Ba7, Cyt2Ba8, Cyt2Ba9, Cyt2Ba10, Cyt2Ba11, Cyt2Ba12, Cyt2Ba13, Cyt2Ba14, Cyt2Ba15, Cyt2Ba16, Cyt2Ba-like, Cyt2Bb1, Cyt2Bc1, Cyt2B-like, Cyt2Ca1, and/or Cyt3Aa1.

In some embodiments, a combination of the present disclosure comprises an AMP and a Bt toxin, wherein the Bt toxin can be any Cyt toxin as described herein, or presented in Table 2.

TABLE 2
Non-limiting examples of Cyt toxins, their accession numbers on
NCBI, and strain. Here, if a cell is left blank, then the accession
number and/or strain is not applicable.

| Name | NCBI Accession No. | Strain / Other ID |
|---|---|---|
| Cyt1Aa1 | X03182 | Bt israelensis |
| Cyt1Aa2 | X04338 | Bt israelensis |
| Cyt1Aa3 | Y00135 | Bt morrisoni PG14 |
| Cyt1Aa4 | M35968 | Bt morrisoni PG14 |
| Cyt1Aa5 | AL731825 | Bt israelensis |
| Cyt1Aa6 | ABC17640 | Bt LLP29 |
| Cyt1Aa7 | KF152888 | Bt BRC-HQY1 |
| Cyt1Aa8 | MF893205 | |
| Cyt1Aa-like | ABB01172 | Bt LDC-9 |
| Cyt1Ab1 | X98793 | Bt medellin |
| Cyt1Ba1 | U37196 | Bt neoleoensis |
| Cyt1Ca1 | AL731825 | Bt israelensis |
| Cyt1Da1 | HQ113115 | Bt MC28 |
| Cyt1Da2 | JN226105 | hs18-1 |
| Cyt2Aa1 | Z14147 | Bt kyushuensis |
| Cyt2Aa2 | AF472606 | Bt darmstadiensis73E10 |
| Cyt2Aa3 | EU835185 | Bt MC28 |
| Cyt2Aa4 | AEG19547 | Bt WFS-97 |
| Cyt2Bal | U52043 | Bt israelensis 4Q2 |
| Cyt2Ba2 | AF020789 | Bt israelensis PG14 |
| Cyt2Ba3 | AF022884 | Bt fuokukaensis |
| Cyt2Ba4 | AF022885 | Bt morrisoni HD12 |
| Cyt2Ba5 | AF022886 | Bt morrisoni HD518 |
| Cyt2Ba6 | AF034926 | Bt tenebrionis |
| Cyt2Ba7 | AF215645 | Bt T301 |
| Cyt2Ba8 | AF215646 | Bt T36 |
| Cyt2Ba9 | AL731825 | Bt israelensis |
| Cyt2Ba10 | ACX54358 | Bti HD 567 |
| Cyt2Ba11 | ACX54359 | Bti HD 522 |
| Cyt2Ba12 | ACX54360 | Bti INTA H41-1 |
| Cyt2Ba13 | FJ205865 | INTA 160-2 |
| Cyt2Ba14 | FJ205866 | Bti IPS82 |
| Cyt2Ba15 | JF283552 | Bt LLP29 |
| Cyt2Ba16 | MG181950 | QL32-1 |
| Cyt2Ba-like | ABE99695 | Bt LDC-9 |
| Cyt2Bb1 | U82519 | Bt jegathesan |
| Cyt2Bc1 | CAC80987 | Bt medellin |
| Cyt2B-like | DQ341380 | |
| Cyt2Ca1 | AAK50455 | Bt |
| Cyt3Aa1 | HM596591 | Bt TD516 |

Any of the Cyt proteins described herein are suitable for use in a combination of the present disclosure.

### Vip Toxins

In some embodiments, a combination of the present disclosure comprises an AMP and a Bt toxin, wherein the Bt toxin can be a protein belonging to the Vip1, Vip2, Vip3, or Vip4 family. For example, in some embodiments, the Bt toxin can be one or more of the following Vip proteins: Vip1Aa1, Vip1Aa2, Vip1Aa3, Vip1Ab1, Vip1Ac1, Vip1Ad1, Vip1Ba1, Vip1Ba2, Vip1Bb1, Vip1Bb2, Vip1Bb3, Vip1Bc1, Vip1Ca1, Vip1Ca2, Vip1Da1, Vip2Aa1, Vip2Aa2, Vip2Aa3, Vip2Ab1, Vip2Ac1, Vip2Ac2, Vip2Ad1, Vip2Ae1, Vip2Ae2, Vip2Ae3, Vip2Af1, Vip2Af2, Vip2Ag1, Vip2Ag2, Vip2Ba1, Vip2Ba2, Vip2Bb1, Vip2Bb2, Vip2Bb3, Vip2Bb4, Vip3Aa1, Vip3Aa2, Vip3Aa3, Vip3Aa4, Vip3Aa5, Vip3Aa6, Vip3Aa7, Vip3Aa8, Vip3Aa9, Vip3Aa10, Vip3Aa11, Vip3Aa12, Vip3Aa13, Vip3Aa14, Vip3Aa15, Vip3Aa16, Vip3Aa17, Vip3Aa18, Vip3Aa19.0, Vip3Aa19, Vip3Aa20, Vip3Aa21, Vip3Aa22, Vip3Aa23, Vip3Aa24, Vip3Aa25, Vip3Aa26, Vip3Aa27, Vip3Aa28, Vip3Aa29, Vip3Aa30, Vip3Aa31, Vip3Aa32, Vip3Aa33, Vip3Aa34, Vip3Aa35, Vip3Aa36, Vip3Aa37, Vip3Aa38, Vip3Aa39, Vip3Aa40, Vip3Aa41, Vip3Aa42, Vip3Aa43, Vip3Aa44, Vip3Aa45, Vip3Aa46, Vip3Aa47, Vip3Aa48, Vip3Aa49, Vip3Aa50, Vip3Aa51, Vip3Aa52, Vip3Aa53, Vip3Aa54, Vip3Aa55, Vip3Aa56, Vip3Aa57, Vip3Aa58, Vip3Aa59, Vip3Aa60, Vip3Aa61, Vip3Aa62, Vip3Aa63, Vip3Aa64, Vip3Aa65, Vip3Aa66, Vip3Ab1, Vip3Ab2, Vip3Ac1, Vip3Ad1, Vip3Ad2, Vip3Ad3, Vip3Ad4, Vip3Ad5, Vip3Ad6, Vip3Ae1, Vip3Af1, Vip3Af2, Vip3Af3, Vip3Af4, Vip3Ag1, Vip3Ag2, Vip3Ag3, Vip3Ag4, Vip3Ag5, Vip3Ag6, Vip3Ag7, Vip3Ag8, Vip3Ag9, Vip3Ag10, Vip3Ag11, Vip3Ag12, Vip3Ag13, Vip3Ag14, Vip3Ag15, Vip3Ah1, Vip3Ah2, Vip3Ai1, Vip3Aj1, Vip3Aj2, Vip3Ba1, Vip3Ba2, Vip3Bb1, Vip3Bb2, Vip3Bb3, Vip3Bc, Vip3Ca1, Vip3Ca2, Vip3Ca3, Vip3Ca4, and/or Vip4Aa1.

In some embodiments, the Bt toxin can be any Vip protein as described herein, or presented in Table 3.

TABLE 3
Non-limiting examples of Vip proteins and their accession
numbers on NCBI. Here, if a cell is left blank, then the
accession number is not applicable.

| Name | NCBI Accession No. |
|---|---|
| Vip1Aa1 | |
| Vip1Aa2 | AAR81088 |
| Vip1Aa3 | GU992203 |
| Vip1Ab1 | |
| Vip1Ac1 | HM439098 |
| ViplAd1 | AGC08395 |
| Vip1Ba1 | AAR40886 |
| Vip1Ba2 | CAI43278 |
| Vip1Bb1 | AAR40282 |
| Vip1Bb2 | HM485584 |
| Vip1Bb3 | KR065727 |
| Vip1Bc1 | HM485583 |
| Vip1Ca1 | AAO86514 |
| Vip1Ca2 | KR065725 |
| Vip1Da1 | CAI40767 |
| Vip2Aa1 | 1QS1A |
| Vip2Aa2 | AAR81096 |
| Vip2Aa3 | HM439097 |
| Vip2Ab1 | |
| Vip2Ac1 | AAO86513 |
| Vip2Ac2 | KR065726 |
| Vip2Ad1 | CAI40768 |
| Vip2Ae1 | EF442245 |
| Vip2Ae2 | ACH42758 |
| Vip2Ae3 | HM439099 |
| Vip2Af1 | ACH42759 |
| Vip2Af2 | EU909204 |
| Vip2Ag1 | AGC08396 |
| Vip2Ag2 | KC951878 |
| Vip2Ba1 | AAR40887 |
| Vip2Ba2 | CAI43279 |
| Vip2Bb1 | |
| Vip2Bb2 | HM485585 |
| Vip2Bb3 | KJ868170 |
| Vip2Bb4 | KR065728 |
| Vip3Aa1 | AAC37036 |
| Vip3Aa2 | AAC37037 |
| Vip3Aa3 | |
| Vip3Aa4 | AAR81079 |
| Vip3Aa5 | AAR81080 |
| Vip3Aa6 | AAR81081 |
| Vip3Aa7 | AAK95326 |
| Vip3Aa8 | AAK97481 |
| Vip3Aa9 | CAA76665 |
| Vip3Aa10 | AAN60738 |
| Vip3Aa11 | AAR36859 |
| Vip3Aa12 | AAM22456 |
| Vip3Aa13 | AAL69542 |
| Vip3Aa14 | AAQ12340 |
| Vip3Aa15 | AAP51131 |
| Vip3Aa16 | AAW65132 |
| Vip3Aa17 | |
| Vip3Aa18 | AAX49395 |
| Vip3Aa19.0 | DQ241674 |
| Vip3Aa19 | DQ539887 |
| Vip3Aa20 | DQ539888 |
| Vip3Aa21 | ABD84410 |
| Vip3Aa22 | AAY41427 |
| Vip3Aa23 | AAY41428 |
| Vip3Aa24 | BI 880913 |
| Vip3Aa25 | EF608501 |
| Vip3Aa26 | EU294496 |
| Vip3Aa27 | EU332167 |
| Vip3Aa28 | FJ494817 |
| Vip3Aa29 | FJ626674 |
| Vip3Aa30 | FJ626675 |
| Vip3Aa31 | FJ626676 |
| Vip3Aa32 | FJ626677 |
| Vip3Aa33 | GU073128 |
| Vip3Aa34 | GU073129 |
| Vip3Aa35 | GU733921 |
| Vip3Aa36 | GU951510 |
| Vip3Aa37 | HM132041 |
| Vip3Aa38 | HM117632 |
| Vip3Aa39 | HM117631 |
| Vip3Aa40 | HM132042 |
| Vip3Aa41 | HM132043 |
| Vip3Aa42 | HQ587048 |
| Vip3Aa43 | HQ594534 |
| Vip3Aa44 | HQ650163 |
| Vip3Aa45 | JF710269 |
| Vip3Aa46 | JQ228436 |
| Vip3Aa47 | JQ228435 |
| Vip3Aa48 | JQ731616 |
| Vip3Aa49 | JQ731617 |
| Vip3Aa50 | JQ946639 |
| Vip3Aa51 | KC156649 |
| Vip3Aa52 | KF826718 |
| Vip3Aa53 | KF826723 |
| Vip3Aa54 | AHK23264 |
| Vip3Aa55 | KJ868172 |
| Vip3Aa56 | LN624748 |
| Vip3Aa57 | AJD18609 |
| Vip3Aa58 | KR259139 |
| Vip3Aa59 | KR259140 |
| Vip3Aa60 | KR340473 |
| Vip3Aa61 | KU522245 |
| Vip3Aa62 | KT792883 |
| Vip3Aa63 | KY780302 |
| Vip3Aa64 | KY883694 |
| Vip3Aa65 | MH290720 |
| Vip3Aa66 | MK252100 |
| Vip3Ab1 | AAR40284 |
| Vip3Ab2 | AAY88247 |
| Vip3Ac1 | |
| Vip3Ad1 | |
| Vip3Ad2 | CAI43276 |
| Vip3Ad3 | KF826720 |
| Vip3Ad4 | KF826727 |
| Vip3Ad5 | KR263164 |
| Vip3Ad6 | KU761577 |
| Vip3Ae1 | CAI43277 |
| Vip3Af1 | CAI43275 |
| Vip3Af2 | |
| Vip3Af3 | HM117634 |
| Vip3Af4 | KM276664 |
| Vip3Ag1 | |
| Vip3Ag2 | FJ556803 |
| Vip3Ag3 | HM117633 |
| Vip3Ag4 | HQ414237 |
| Vip3Ag5 | HQ542193 |
| Vip3Ag6 | JQ397328 |
| Vip3Ag7 | KF826713 |
| Vip3Ag8 | KF826714 |
| Vip3Ag9 | KF826715 |
| Vip3Ag10 | KF826716 |
| Vip3Ag11 | KF826719 |
| Vip3Ag12 | KF826721 |
| Vip3Ag13 | KF826722 |
| Vip3Ag14 | KF826725 |
| Vip3Ag15 | KF826726 |
| Vip3Ah1 | DQ832323 |
| Vip3Ah2 | AQY42675 |
| Vip3Ai1 | KC156693 |
| Vip3Aj1 | KF826717 |
| Vip3Aj2 | KF826724 |
| Vip3Ba1 | AAV70653 |
| Vip3Ba2 | HM117635 |
| Vip3Bb1 | |
| Vip3Bb2 | ABO30520 |
| Vip3Bb3 | ADI48120 |
| Vip3Bc | MF543028 |
| Vip3Ca1 | ADZ46178 |
| Vip3Ca2 | AEE98106 |
| Vip3Ca3 | HQ876489 |
| Vip3Ca4 | JN836992 |
| Vip4Aa1 | HM044666 |

Any of the Vip proteins described herein are suitable for use in a combination of the present disclosure.

Any of the aforementioned Bt toxins can be used to create a combination and/or composition of the present disclosure, wherein said combination and/or composition comprises at least one AMP, and at least one Bt toxin.

### Agriculturally Acceptable Salts

As used herein, the term “pharmaceutically acceptable salt” and “agriculturally acceptable salt” are synonymous.

In some embodiments, agriculturally acceptable salts, hydrates, solvates, crystal forms and individual isomers, enantiomers, tautomers, diastereomers and prodrugs of the AMP described herein can be utilized.

In some embodiments, an agriculturally acceptable salt of the present disclosure possesses the desired pharmacological activity of the parent compound. Such salts include: acid addition salts, formed with inorganic acids; acid addition salts formed with organic acids; or salts formed when an acidic proton present in the parent compound is replaced by a metal ion, e.g., an alkali metal ion, aluminum ion; or coordinates with an organic base such as ethanolamine, and the like.

In some embodiments, agriculturally acceptable salts include conventional toxic or non-toxic salts. For example, in some embodiments, convention non-toxic salts include those such as fumarate, phosphate, citrate, chlorydrate, and the like. In some embodiments, the agriculturally acceptable salts of the present disclosure can be synthesized from a parent compound by conventional chemical methods. In some embodiments, such salts can be prepared by reacting the free acid or base forms of these compounds with a stoichiometric amount of the appropriate base or acid in water or in an organic solvent, or in a mixture of the two. In some embodiments, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is incorporated herein by reference in its entirety.

In some embodiments, an agriculturally acceptable salt can be one of the following: hydrochloride; sodium; sulfate; acetate; phosphate or diphosphate; chloride; potassium; maleate; calcium; citrate; mesylate; nitrate; tartrate; aluminum; or gluconate.

In some embodiments, a list of agriculturally acceptable acids that can be used to form salts can be: glycolic acid; hippuric acid; hydrobromic acid; hydrochloric acid; isobutyric acid; lactic acid (DL); lactobionic acid; lauric acid; maleic acid; malic acid (− L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (− L); salicylic acid; sebacic acid; stearic acid; succinic acid; sulfuric acid; tartaric acid (+ L); thiocyanic acid; toluenesulfonic acid (p); undecylenic acid; a 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; or glycerophosphoric acid.

In some embodiments, agriculturally acceptable salt can be any organic or inorganic addition salt.

In some embodiments, the salt may use an inorganic acid and an organic acid as a free acid. The inorganic acid may be hydrochloric acid, bromic acid, nitric acid, sulfuric acid, perchloric acid, phosphoric acid, etc. The organic acid may be citric acid, acetic acid, lactic acid, maleic acid, fumaric acid, gluconic acid, methane sulfonic acid, gluconic acid, succinic acid, tartaric acid, galacturonic acid, embonic acid, glutamic acid, aspartic acid, oxalic acid, (D) or (L) malic acid, maleic acid, methane sulfonic acid, ethane sulfonic acid, 4-toluene sulfonic acid, salicylic acid, citric acid, benzoic acid, malonic acid, etc.

In some embodiments, the salts include alkali metal salts (sodium salts, potassium salts, etc.) and alkaline earth metal salts (calcium salts, magnesium salts, etc.). For example, the acid addition salt may include acetate, aspartate, benzoate, besylate, bicarbonate/carbonate, bisulfate/sulfate, borate, camsylate, citrate, edisilate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthalate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salt, etc., and among them, hydrochloride or trifluoroacetate may be used.

In yet other embodiments, the agriculturally acceptable salt can be a salt with an acid such as acetic acid, propionic acid, butyric acid, formic acid, trifluoroacetic acid, maleic acid, tartaric acid, citric acid, stearic acid, succinic acid, ethylsuccinic acid, lactobionic acid, gluconic acid, glucoheptonic acid, benzoic acid, methanesulfonic acid, ethanesulfonic acid, 2-hydroxyethanesulfonic acid, benzenesulfonic acid, p-toluenesulfonic acid, laurylsulfuric acid, malic acid, aspartic acid, glutaminic acid, adipic acid, cysteine, N-acetylcysteine, hydrochloric acid, hydrobromic acid, phosphoric acid, sulfuric acid, hydroiodic acid, nicotinic acid, oxalic acid, picric acid, thiocyanic acid, undecanoic acid, polyacrylate or carboxyvinyl polymer.

In some embodiments, the agriculturally acceptable salt can be prepared from either inorganic or organic bases. Salts derived from inorganic bases include, but are not limited to, the sodium, potassium, lithium, ammonium, calcium, magnesium, ferrous, zinc, copper, manganous, aluminum, ferric, manganic salts, and the like. Preferred inorganic salts are the ammonium, sodium, potassium, calcium, and magnesium salts. Salts derived from organic bases include, but are not limited to, salts of primary, secondary, and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Preferred organic bases are isopropylamine, diethylamine, ethanolamine, piperidine, tromethamine, and choline.

In some embodiments, agriculturally acceptable salt refers to those salts which are, within the scope of sound medical judgment, suitable for use in contact with the tissues of humans and lower animals without undue toxicity, irritation, allergic response and the like, and are commensurate with a reasonable benefit/risk ratio. Agriculturally acceptable salts are well known in the art. For example, S. M. Berge, et al. describe agriculturally acceptable salts in detail in J. Pharmaceutical Sciences, 66: 1-19 (1977), the disclosure of which is incorporated herein by reference in its entirety.

In some embodiments, the salts of the present disclosure can be prepared in situ during the final isolation and purification of the compounds of the present disclosure, or separately by reacting the free base function with a suitable organic acid. Examples of agriculturally acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other agriculturally acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further agriculturally acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Exemplary descriptions of pharmaceutically acceptable salts is provided in P. H. Stahl and C. G. Wermuth, (editors), Handbook of Pharmaceutical Salts: Properties, Selection and Use, John Wiley & Sons, August 23, (2002), the disclosure of which is incorporated herein by reference in its entirety.

### Methods of Making Peptides of the Present Disclosure

Any of the peptides, polypeptides, or proteins described herein, e.g., an AMP, AMP-insecticidal protein, and/or a Bt toxin of the present disclosure can be made using any of the well-known methods known to those having ordinary skill in the art.

In some embodiments, a peptide of the present disclosure can be made from the expression and translation of chemically synthesized polynucleotides. Exemplary method for generating DNA and or custom chemically synthesized polynucleotides are well known in the art, and are illustratively provided in U.S. Pat. No. 5,736,135, Ser. No. 08/389,615, filed on Feb. 13, 1995, the disclosure of which is incorporated herein by reference in its entirety. See also Agarwal, et al., Chemical synthesis of polynucleotides. Angew Chem Int Ed Engl. 1972 June; 11(6):451-9; Ohtsuka et al., Recent developments in the chemical synthesis of polynucleotides. Nucleic Acids Res. 1982 Nov. 11; 10(21): 6553-6570; Sondek & Shortle. A general strategy for random insertion and substitution mutagenesis: substoichiometric coupling of trinucleotide phosphoramidites. Proc Natl Acad Sci USA. 1992 Apr. 15; 89(8): 3581-3585; Beaucage S. L., et al., Advances in the Synthesis of Oligonucleotides by the Phosphoramidite Approach. Tetrahedron, Elsevier Science Publishers, Amsterdam, NL, vol. 48, No. 12, 1992, pp. 2223-2311; Agrawal (1993) Protocols for Oligonucleotides and Analogs: Synthesis and Properties; Methods in Molecular Biology Vol. 20, the disclosures of which are incorporated herein by reference in their entireties.

In some embodiments, a mutation in a wild-type Av3 polynucleotide sequence and/or an Av3b polynucleotide sequence can be made by various means that are well known to those having ordinary skill in the art. Methods of mutagenesis include Kunkel's method; cassette mutagenesis; PCR site-directed mutagenesis; the “perfect murder” technique (delitto perfetto); direct gene deletion and site-specific mutagenesis with PCR and one recyclable marker; direct gene deletion and site-specific mutagenesis with PCR and one recyclable marker using long homologous regions; transplacement “pop-in pop-out” method; and CRISPR-Cas 9.

Exemplary methods of site-directed mutagenesis can be found in Ruvkun & Ausubel, A general method for site-directed mutagenesis in prokaryotes. Nature. 1981 Jan. 1; 289(5793):85-8; Wallace et al., Oligonucleotide directed mutagenesis of the human beta-globin gene: a general method for producing specific point mutations in cloned DNA. Nucleic Acids Res. 1981 Aug. 11; 9(15):3647-56; Dalbadie-McFarland et al., Oligonucleotide-directed mutagenesis as a general and powerful method for studies of protein function. Proc Natl Acad Sci USA. 1982 November; 79(21):6409-13; Bachman. Site-directed mutagenesis. Methods Enzymol. 2013; 529:241-8; Carey et al., PCR-mediated site-directed mutagenesis. Cold Spring Harb Protoc. 2013 Aug. 1; 2013(8):738-42; and Cong et al., Multiplex genome engineering using CRISPR/Cas systems. Science. 2013 Feb. 15; 339(6121):819-23, the disclosures of all of the aforementioned references are incorporated herein by reference in their entireties.

In some embodiments, peptides of the present disclosure can be chemically synthesized. Exemplary methods of peptide synthesis can be found in Anderson G. W. and McGregor A. C. (1957) T-butyloxycarbonylamino acids and their use in peptide synthesis. Journal of the American Chemical Society. 79, 6180-3; Carpino L. A. (1957) Oxidative reactions of hydrazines. Iv. Elimination of nitrogen from 1,1-disubstituted-2-arenesulfonhydrazides1-4. Journal of the American Chemical Society. 79, 4427-31; McKay F. C. and Albertson N. F. (1957) New amine-masking groups for peptide synthesis. Journal of the American Chemical Society. 79, 4686-90; Merrifield R. B. (1963) Solid phase peptide synthesis. I. The synthesis of a tetrapeptide. Journal of the American Chemical Society. 85, 2149-54; Carpino L. A. and Han G. Y. (1972) 9-fluorenylmethoxycarbonyl amino-protecting group. The Journal of Organic Chemistry. 37, 3404-9; and A Lloyd-Williams P. et al. (1997) Chemical approaches to the synthesis of peptides and proteins. Boca Raton: CRC Press. 278; U.S. Pat. No. 3,714,140 (filed Mar. 16, 1971); U.S. Pat. No. 4,411,994 (filed Jun. 8, 1978); U.S. Pat. No. 7,785,832 (filed Jan. 20, 2006); U.S. Pat. No. 8,314,208 (filed Feb. 10, 2006); and 10,442,834 (filed Oct. 2, 2015); and United States Patent Application 2005/0165215 (filed Dec. 23, 2004), the disclosures of which are incorporated herein by reference in their entirety.

In some embodiments, a polynucleotide encoding a peptide of the present disclosure can be transformed into cell culture expression system. Exemplary methods regarding transfection and/or transformation techniques can be found in Makrides (2003), Gene Transfer and Expression in Mammalian Cells, Elvesier; Wong, TK & Neumann, E. Electric field mediated gene transfer. Biochem. Biophys. Res. Commun. 107, 584-587 (1982); Potter & Heller, Transfection by Electroporation. Curr Protoc Mol Biol. 2003 May; CHAPTER: Unit-9.3; Kim & Eberwine, Mammalian cell transfection: the present and the future. Anal Bioanal Chem. 2010 August; 397(8): 3173-3178, each of these references are incorporated herein by reference in their entireties. In some embodiments, the cell culture expression system can be a yeast cell culture expression system. Exemplary methods of yeast cell culture can be found in Evans, Yeast Protocols. Springer (1996); Bill, Recombinant Protein Production in Yeast. Springer (2012); Hagan et al., Fission Yeast: A Laboratory Manual, CSH Press (2016); Konishi et al., Improvement of the transformation efficiency of Saccharomyces cerevisiae by altering carbon sources in pre-culture. Biosci Biotechnol Biochem. 2014; 78(6):1090-3; Dymond, Saccharomyces cerevisiae growth media. Methods Enzymol. 2013; 533:191-204; Looke et al., Extraction of genomic DNA from yeasts for PCR-based applications. Biotechniques. 2011 May; 50(5):325-8; and Romanos et al., Culture of yeast for the production of heterologous proteins. Curr Protoc Cell Biol. 2014 Sep. 2; 64:20.9.1-16, the disclosure of which is incorporated herein by reference in its entirety. Exemplary culture methods are provided in U.S. Pat. Nos. 3,933,590; 3,946,780; 4,988,623; 5,153,131; 5,153,133; 5,155,034; 5,316,905; 5,330,908; 6,159,724; 7,419,801; 9,320,816; 9,714,408; and 10,563,169; the disclosures of which are incorporated herein by reference in their entireties.

In some embodiments, peptides of the present disclosure can be purified using any method known in the art. Exemplary methods of protein purification are provided in: U.S. Pat. Nos. 6,339,142; 7,585,955; 8,946,395; 9,067,990; 10,246,484; and Marshak et al., “Strategies for Protein Purification and Characterization—A Laboratory Course Manual” CSHL Press (1996); the disclosures of which are incorporated herein by reference in their entireties. Likewise, quantification of peptides can also be achieved using any method known in the art. Exemplary methods of protein quantification are provided in Stoscheck, C. 1990 “Quantification of Protein” Methods in Enzymology, 182:50-68; Lowry, O. Rosebrough, A., Farr, A. and Randall, R. 1951 J. Biol. Chem. 193:265; Smith, P. et al., (1985) Anal. Biochem. 150:76-85; Bradford, M. 1976 “A Rapid and Sensitive Method for the Quantitation of Microgram Quantities of Protein Utilizing the Principle of Protein-Dye Binding” Anal. Biochem. 72:248-254; Cabib, E. and Polacheck, I. 1984 “Protein assay for dilute solutions.” Methods in Enzymology, 104:318-328; Turcanu, Victor; Williams, Neil A. (2001). “Cell identification and isolation on the basis of cytokine secretion: A novel tool for investigating immune responses.” Nature Medicine. 7 (3): 373-376; U.S. Pat. No. 6,391,649; the disclosures of which are incorporated herein by reference in their entireties.

### Compositions, and Formulations

As used herein, “v/v” or “% v/v” or “volume per volume” refers to the volume concentration of a solution (“v/v” stands for volume per volume). Here, v/v can be used when both components of a solution are liquids. For example, when 50 mL of ingredient X is diluted with 50 mL of water, there will be 50 mL of ingredient X in a total volume of 100 mL; therefore, this can be expressed as “ingredient X 50% v/v.” Percent volume per volume (% v/v) is calculated as follows: (volume of solute (mL)/volume of solution (100 mL)); e.g., % v/v=mL of solute/100 mL of solution.

As used herein, “w/w” or “% w/w” or “weight per weight” refers to the weight concentration of a solution, i.e., percent weight in weight (“w/w” stands for weight per weight). Here, w/w expresses the number of grams (g) of a constituent in 100 g of solution or mixture. For example, a mixture consisting of 30 g of ingredient X, and 70 g of water would be expressed as “ingredient X 30% w/w.” Percent weight per weight (% w/w) is calculated as follows: (weight of solute (g)/weight of solution (g))×100; or (mass of solute (g)/mass of solution (g))×100.

As used herein, “w/v” or “% w/v” or “weight per volume” refers to the mass concentration of a solution, i.e., percent weight in volume (“w/v” stands for weight per volume). Here, w/v expresses the number of grams (g) of a constituent in 100 mL of solution. For example, if 1 g of ingredient X is used to make up a total volume of 100 mL, then a “1% w/v solution of ingredient X” has been made. Percent weight per volume (% w/v) is calculated as follows: (Mass of solute (g)/Volume of solution (mL))×100.

Any of the AMP, AMP-insecticidal proteins, or an agriculturally acceptable salt thereof described herein, and/or any of the Bt toxins described herein, can be used to create a combination and/or a composition, wherein said combination and/or composition comprises, consists essentially of, or consists of at least one AMP and at least one Bt toxin.

In some embodiments, the present disclosure comprises, consists essentially of, or consists of, a combination, a mixture, or a composition comprising, consisting essentially of, or consisting of, an AMP, one or more AMP-insecticidal proteins, and/or combinations thereof, and one or more Bt toxins.

In some embodiments, the present disclosure contemplates a mixture of an AMP, one or more AMP-insecticidal proteins, and/or one or more Bt toxins. For example, in some embodiments an AMP, one or more AMP-insecticidal proteins, and/or one or more Bt toxins, can be blended together in in varying proportions.

In some embodiments, the present disclosure contemplates a combination of an AMP, one or more AMP-insecticidal proteins, and/or one or more Bt toxins. For example, in some embodiments, an AMP and/or one or more AMP-insecticidal proteins, or an agriculturally acceptable salt thereof, and one or more Bt toxins, can be provided as a combination, e.g., in the same container, or in different containers.

In some embodiments, the present disclosure contemplates a composition of an AMP and/or one or more AMP-insecticidal proteins, or an agriculturally acceptable salt thereof, and one or more Bt toxins. For example, in some embodiments, an AMP, one or more AMP-insecticidal proteins, and/or combinations thereof, can be provided as a composition further comprising an excipient.

In some embodiments, the combination, mixture, or composition comprises, consists essentially of, or consists of, an Av3 mutant polypeptide (AMP) having insecticidal activity against one or more insect species, and a Bt toxin, said AMP comprising an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 910% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof.

In some embodiments, a combination, mixture, or composition of the present disclosure can comprise, consist essentially of, or consist of, an AMP, wherein said AMP is a homopolymer of two or more AMPs, wherein the amino acid sequence of each AMP is the same.

In some embodiments, a combination, mixture, or composition of the present disclosure can comprise, consist essentially of, or consist of, an AMP that is a fused protein comprising two or more AMPs separated by a cleavable or non-cleavable linker, and wherein the amino acid sequence of each AMP is the same.

In some embodiments, a combination, mixture, or composition of the present disclosure can comprise, consist essentially of, or consist of, an AMP having a linker, wherein the linker is a cleavable linker.

In some embodiments, a combination, mixture, or composition of the present disclosure can comprise, consist essentially of, or consist of, an AMP having a linker, wherein the linker has an amino acid sequence as set forth in any one of SEQ ID NOs: 4-16.

In some embodiments, a combination, mixture, or composition of the present disclosure can comprise, consist essentially of, or consist of, an AMP having a linker, wherein the linker is cleavable inside at least one of (i) the gut or hemolymph of an insect, and (ii) cleavable inside the gut of a mammal.

Any of the combinations and/or compositions comprising an AMP and a Bt toxin, and/or plants transformed with polynucleotides operable to express an AMP and/or a Bt toxin, and described herein, can be used to control pests, their growth, and/or the damage caused by their actions, especially their damage to plants.

Compositions comprising a combination of: (1) an AMP, an AMP-insecticidal protein, or an agriculturally acceptable salt thereof; and (2) a Bt toxin, for example, agrochemical compositions, can include, but are not limited to, aerosols and/or aerosolized products, e.g., sprays, fumigants, powders, dusts, and/or gases; seed dressings; oral preparations (e.g., insect food, etc.); transgenic organisms expressing and/or producing an AMP, an AMP-insecticidal protein, an AMP ORF and/or a Bt toxin (either transiently and/or stably), e.g., a plant or an animal.

The composition may be formulated as a powder, dust, pellet, granule, spray, emulsion, colloid, solution, or such like, and may be prepared by such conventional means as desiccation, lyophilization, homogenization, extraction, filtration, centrifugation, sedimentation, or concentration of a culture of cells comprising the polypeptide. In all such compositions that contain at least one such AMP, AMP-insecticidal protein or combinations thereof, may be present in a concentration of from about 10% to about 99% by weight.

In some embodiments, the pesticide compositions described herein may be made by formulating either the combination of: (1) an AMP, an AMP-insecticidal protein, or an agriculturally acceptable salt thereof; and (2) a Bt toxin, with the desired agriculturally-acceptable carrier. The compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, the disclosure of which is incorporated by reference herein in its entirety.

In some embodiments a composition of the present disclosure can comprise a combination of: (1) an AMP, an AMP-insecticidal protein, or an agriculturally acceptable salt thereof, (2) a Bt toxin; and (3) at least one excipient.

In some embodiments, a composition can comprise, consist essentially of, or consist of, an AMP, a Bt toxin, and an excipient.

In some embodiments, a composition can comprise, consist essentially of, or consist of, an AMP-insecticidal protein, a Bt toxin, and an excipient.

In some embodiments, a composition can comprise, consist essentially of, or consist of: (1) an AMP, an AMP-insecticidal protein, or an agriculturally acceptable salt thereof and/or a combination thereof; (2) one or more Bt toxins; and (3) at least one excipient.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the AMP, AMP-insecticidal protein, or agriculturally acceptable salt thereof is in an amount ranging from about 0.0000010% w/w to about 99.99999% w/w of the total composition, or from about 0.010% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.910%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 410% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 510% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 910% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a composition of the present disclosure can comprise. (1) an AMP or an agriculturally acceptable salt thereof, and/or an AMP-insecticidal protein, or an agriculturally acceptable salt thereof; (2) a Bt toxin; and (3) an excipient; wherein the concentration of the AMP, AMP-insecticidal protein, or agriculturally acceptable salt thereof ranges from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 910%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 710%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 410%; from about 60% to about 40%; from about 610% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 910% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the concentration of the AMP, AMP-insecticidal protein, or agriculturally acceptable salt thereof is about 0.000001%, 0.00001%, 0.0001%, 0.001%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, 99.9999%, or 99.99999% by weight of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the Bt toxin is in an amount ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 210%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the concentration of the Bt toxin ranges from about 0.010% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 210%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the concentration of the Bt toxin is about 0.000001%, 0.00001%, 0.0001%, 0.001%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, 99.9999%, or 99.99999% by weight of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the excipient is in an amount ranging from about 0.0000010% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the concentration of the excipient ranges from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 910%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 810%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 510%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a composition of the present disclosure can comprise: (1) an AMP or an agriculturally acceptable salt thereof, and/or an AMP-insecticidal protein, or an agriculturally acceptable salt thereof; (2) a Bt toxin; and (3) an excipient; wherein the concentration of the excipient is about 0.000001%, 0.00001%, 0.00010%, 0.0010%, 0.010%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%1, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, 99.9999%, or 99.99999% by weight of the total composition.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio ranging from 0.0001:10,000 to 10,000:0.0001.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio ranging from 0.0001:10,000 to 1:1; or 0.001:10,000 to 1:1; or 0.01:10,000 to 1:1; or 0.1:10,000 to 1:1; or 1:10,000 to 1:1; or 0.0001:1000 to 1:1; or 0.0001:100 to 1:1; or 0.0001:10 to 1:1; or 0.0001:1 to 1:1.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio ranging from 0.0001:10,000 to 1:1; or 0.001:10,000 to 1:1; or 0.01:10,000 to 1:1; or 0.1:10,000 to 1:1; or 1:10,000 to 1:1; or 0.0001:1000 to 1:1; or 0.0001:100 to 1:1; or 0.0001:10 to 1:1; or 0.0001:1 to 1:1.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio ranging from 1:1 to 10,000:1; or 1:1 to 1000:1; or 1:1 to 100:1; or 1:1 to 90:1; or 1:1 to 80:1; or 1:1 to 70:1; or 1:1 to 60:1; or 1:1 to 50:1; or 1:1 to 40:1; or 1:1 to 30:1; or 1:1 to 20:1; or 1:1 to 10:1; or 1:1 to 9:1; or 1:1 to 8:1; or 1:1 to 7:1; or 1:1 to 6:1; or 1:1 to 5:1; or 1:1 to 4:1; or 1:1 to 3:1; or 1:1 to 2:1; or 2:1 to 1:1; or 3:1 to 1:1; or 4:1 to 1:1; or 5:1 to 1:1; or 6:1 to 1:1; or 7:1 to 1:1; or 8:1 to 1:1; or 9:1 to 1:1; or 10:1 to 1:1; or 20:1 to 1:1; or 30:1 to 1:1; or 40:1 to 1:1; or 50:1 to 1:1; or 60:1 to 1:1; or 70:1 to 1:1 or 80:1 to 1:1; or 90:1 to 1:1; or 100:1 to 1:1; or 1000:1 to 1:1 or 10,000:1 to 1:1.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio ranging from 0.0001:10,000 to 10,000:0.0001, or 0.0001:10,000 to 1,000:0.0001, or 0.0001:10,000 to 100:0.0001, or 0.0001:10,000 to 100:0.0001; or 0.0001:10,000 to 95:0.0001; or 0.0001:10,000 to 90:0.0001; or 0.0001:10,000 to 85:0.0001; or 0.0001:10,000 to 80:0.0001; or 0.0001:10,000 to 75:0.0001; or 0.0001:10,000 to 70:0.0001; or 0.0001:10,000 to 65:0.0001; or 0.0001:10,000 to 60:0.0001; or 0.0001:10,000 to 55:0.0001; or 0.0001:10,000 to 50:0.0001; or 0.0001:10,000 to 45:0.0001; or 0.0001:10,000 to 40:0.0001; or 0.0001:10,000 to 35:0.0001; or 0.0001:10,000 to 30:0.0001; or 0.0001:10,000 to 25:0.0001; or 0.0001:10,000 to 20:0.0001; or 0.0001:10,000 to 15:0.0001; or 0.0001:10,000 to 10:0.0001.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio of about 10:20, 10:25, 10:30, 10:35, 10:40, 10:45, 10:50, 10:55, 10:60, 10:65, 10:70, 10:75, 10:80, 10:85, 10:90, 10:95, 10:100, 20:25, 20:30, 20:35, 20:40, 20:45, 20:50, 20:55, 20:60, 20:65, 20:70, 20:75, 20:80, 20:85, 20:90, 20:95, 20:100, 30:35, 30:40, 30:45, 30:50, 30:55, 30:60, 30:65, 30:70, 30:75, 30:80, 30:85, 30:90, 30:95, 30:100, 40:45, 40:50, 40:55, 40:60, 40:65, 40:70, 40:75, 40:80, 40:85, 40:90, 40:95, 40:100, 50:55, 50:60, 50:65, 50:70, 50:75, 50:80, 50:85, 50:90, 50:95, 50:100, 60:65, 60:70, 60:75, 60:80, 60:85, 60:90, 60:95, 60:100, 70:75, 70:80, 70:85, 70:90, 70:95, 70:100, 80:85, 80:90, 80:95, 80:100, 90:95, 90:100, or 95:100.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (1):(2) is a ratio of about 100:95, 100:90, 95:90, 100:80, 95:80, 90:80, 85:80, 100:70, 95:70, 90:70, 85:70, 80:70, 75:70, 100:60, 95:60, 90:60, 85:60, 80:60, 75:60, 70:60, 65:60, 100:50, 95:50, 90:50, 85:50, 80:50, 75:50, 70:50, 65:50, 60:50, 55:50, 100:40, 95:40, 90:40, 85:40, 80:40, 75:40, 70:40, 65:40, 60:40, 55:40, 50:40, 45:40, 100:30, 95:30, 90:30, 85:30, 80:30, 75:30, 70:30, 65:30, 60:30, 55:30, 50:30, 45:30, 40:30, 35:30, 100:20, 95:20, 90:20, 85:20, 80:20, 75:20, 70:20, 65:20, 60:20, 55:20, 50:20, 45:20, 40:20, 35:20, 30:20, 25:20, 100:10, 95:10, 90:10, 85:10, 80:10, 75:10, 70:10, 65:10, 60:10, 55:10, 50:10, 45:10, 40:10, 35:10, 30:10, 25:10, 20:10, or 15:10.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio ranging from 0.0001:10,000 to 10,000:0.0001.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio ranging from 0.0001:10,000 to 1:1; or 0.001:10,000 to 1:1; or 0.01:10,000 to 1:1; or 0.1:10,000 to 1:1; or 1:10,000 to 1:1; or 0.0001:1000 to 1:1; or 0.0001:100 to 1:1; or 0.0001:10 to 1:1; or 0.0001:1 to 1:1.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio ranging from 0.0001:10,000 to 1:1; or 0.001:10,000 to 1:1; or 0.01:10,000 to 1:1; or 0.1:10,000 to 1:1; or 1:10,000 to 1:1; or 0.0001:1000 to 1:1; or 0.0001:100 to 1:1; or 0.0001:10 to 1:1; or 0.0001:1 to 1:1.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio ranging from 1:1 to 10,000:1; or 1:1 to 1000:1; or 1:1 to 100:1; or 1:1 to 90:1; or 1:1 to 80:1; or 1:1 to 70:1; or 1:1 to 60:1; or 1:1 to 50:1; or 1:1 to 40:1; or 1:1 to 30:1; or 1:1 to 20:1; or 1:1 to 10:1; or 1:1 to 9:1; or 1:1 to 8:1; or 1:1 to 7:1; or 1:1 to 6:1; or 1:1 to 5:1; or 1:1 to 4:1; or 1:1 to 3:1; or 1:1 to 2:1; or 2:1 to 1:1; or 3:1 to 1:1; or 4:1 to 1:1; or 5:1 to 1:1; or 6:1 to 1:1; or 7:1 to 1:1; or 8:1 to 1:1; or 9:1 to 1:1; or 10:1 to 1:1; or 20:1 to 1:1; or 30:1 to 1:1; or 40:1 to 1:1; or 50:1 to 1:1; or 60:1 to 1:1; or 70:1 to 1:1 or 80:1 to 1:1; or 90:1 to 1:1; or 100:1 to 1:1; or 1000:1 to 1:1 or 10,000:1 to 1:1.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio ranging from 0.0001:10,000 to 10,000:0.0001, or 0.0001:10,000 to 1,000:0.0001, or 0.0001:10,000 to 100:0.0001, or 0.0001:10,000 to 100:0.0001; or 0.0001:10,000 to 95:0.0001; or 0.0001:10,000 to 90:0.0001; or 0.0001:10,000 to 85:0.0001; or 0.0001:10,000 to 80:0.0001; or 0.0001:10,000 to 75:0.0001; or 0.0001:10,000 to 70:0.0001; or 0.0001:10,000 to 65:0.0001; or 0.0001:10,000 to 60:0.0001; or 0.0001:10,000 to 55:0.0001; or 0.0001:10,000 to 50:0.0001; or 0.0001:10,000 to 45:0.0001; or 0.0001:10,000 to 40:0.0001; or 0.0001:10,000 to 35:0.0001; or 0.0001:10,000 to 30:0.0001; or 0.0001:10,000 to 25:0.0001; or 0.0001:10,000 to 20:0.0001; or 0.0001:10,000 to 15:0.0001; or 0.0001:10,000 to 10:0.0001.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio of about 10:20, 10:25, 10:30, 10:35, 10:40, 10:45, 10:50, 10:55, 10:60, 10:65, 10:70, 10:75, 10:80, 10:85, 10:90, 10:95, 10:100, 20:25, 20:30, 20:35, 20:40, 20:45, 20:50, 20:55, 20:60, 20:65, 20:70, 20:75, 20:80, 20:85, 20:90, 20:95, 20:100, 30:35, 30:40, 30:45, 30:50, 30:55, 30:60, 30:65, 30:70, 30:75, 30:80, 30:85, 30:90, 30:95, 30:100, 40:45, 40:50, 40:55, 40:60, 40:65, 40:70, 40:75, 40:80, 40:85, 40:90, 40:95, 40:100, 50:55, 50:60, 50:65, 50:70, 50:75, 50:80, 50:85, 50:90, 50:95, 50:100, 60:65, 60:70, 60:75, 60:80, 60:85, 60:90, 60:95, 60:100, 70:75, 70:80, 70:85, 70:90, 70:95, 70:100, 80:85, 80:90, 80:95, 80:100, 90:95, 90:100, or 95:100.

In some embodiments, a composition of the present disclosure can comprise: (1) an AMP or an agriculturally acceptable salt thereof, and/or an AMP-insecticidal protein, or an agriculturally acceptable salt thereof; and (2) a Bt toxin; wherein the ratio of (2):(1) is a ratio of about 100:95, 100:90, 95:90, 100:80, 95:80, 90:80, 85:80, 100:70, 95:70, 90:70, 85:70, 80:70, 75:70, 100:60, 95:60, 90:60, 85:60, 80:60, 75:60, 70:60, 65:60, 100:50, 95:50, 90:50, 85:50, 80:50, 75:50, 70:50, 65:50, 60:50, 55:50, 100:40, 95:40, 90:40, 85:40, 80:40, 75:40, 70:40, 65:40, 60:40, 55:40, 50:40, 45:40, 100:30, 95:30, 90:30, 85:30, 80:30, 75:30, 70:30, 65:30, 60:30, 55:30, 50:30, 45:30, 40:30, 35:30, 100:20, 95:20, 90:20, 85:20, 80:20. 75:20, 70:20, 65:20, 60:20, 55:20, 50:20, 45:20, 40:20, 35:20, 30:20, 25:20, 100:10, 95:10, 90:10, 85:10, 80:10, 75:10, 70:10, 65:10, 60:10, 55:10, 50:10, 45:10, 40:10, 35:10, 30:10, 25:10, 20:10, or 15:10.

### Sprayable Compositions

Examples of spray products of the present disclosure can include field sprayable formulations for agricultural usage and indoor sprays for use in interior spaces in a residential or commercial space. In some embodiments, residual sprays or space sprays comprising a combination of: (1) an AMP, an AMP-insecticidal protein, or an agriculturally acceptable salt thereof; and (2) a Bt toxin; can be used to reduce or eliminate insect pests in an interior space.

Surface spraying indoors (SSI) is the technique of applying a variable volume sprayable volume of an insecticide onto indoor surfaces where vectors rest, such as on walls, windows, floors and ceilings. The primary goal of variable volume sprayable volume is to reduce the lifespan of the insect pest, (for example, a fly, a flea, a tick, or a mosquito vector) and thereby reduce or interrupt disease transmission. The secondary impact is to reduce the density of insect pests within the treatment area. SSI can be used as a method for the control of insect pest vector diseases, such as Lyme disease, Salmonella, Chikungunva virus, Zika virus, and malaria, and can also be used in the management of parasites carried by insect vectors, such as Leishmaniasis and Chagas disease. Many mosquito vectors that harbor Zika virus, Chikungunya virus, and malaria include endophilic mosquito vectors, resting inside houses after taking a blood meal. These mosquitoes are particularly susceptible to control through surface spraying indoors (SSI) with a sprayable composition comprising an AMP, an AMP-insecticidal protein, or an agriculturally acceptable salt thereof; a Bt toxin; and an excipient. As its name implies, SSI involves applying the composition onto the walls and other surfaces of a house with a residual insecticide.

In one embodiment, the composition comprising a combination of: (1) an AMP, an AMP-insecticidal protein, or an agriculturally acceptable salt thereof; and (2) a Bt toxin; and an excipient will knock down insect pests that come in contact with these surfaces. SSI does not directly prevent people from being bitten by mosquitoes. Rather, it usually controls insect pests after they have blood fed, if they come to rest on the sprayed surface. SSI thus prevents transmission of infection to other persons. To be effective, SSI must be applied to a very high proportion of households in an area (usually greater than 40-80 percent). Therefore, sprays in accordance with the present disclosure having good residual efficacy and acceptable odor are particularly suited as a component of integrated insect pest vector management or control solutions.

In contrast to SSI, which requires that the active AMP or AMP-insecticidal protein and/or Bt toxin be bound to surfaces of dwellings, such as walls or ceilings, as with a paint, for example, space spray products of the present disclosure rely on the production of a large number of small insecticidal droplets intended to be distributed through a volume of air over a given period of time. When these droplets impact on a target insect pest, they deliver a knockdown effective dose of the combination of an AMP or AMP-insecticidal protein and Bt toxin effective to control the insect pest. The traditional methods for generating a space-spray include thermal fogging (whereby a dense cloud of a composition comprising the combination of the present disclosure is produced giving the appearance of a thick fog) and Ultra Low Volume (ULV), whereby droplets are produced by a cold, mechanical aerosol-generating machine. Ready-to-use aerosols such as aerosol cans may also be used.

Because large areas can be treated at any one time, the foregoing method is a very effective way to rapidly reduce the population of flying insect pests in a specific area. And, because there is very limited residual activity from the application, it must be repeated at intervals of 5-7 days in order to be fully effective. This method can be particularly effective in epidemic situations where rapid reduction in insect pest numbers is required. As such, it can be used in urban dengue control campaigns.

Effective space-spraying is generally dependent upon the following specific principles. Target insects are usually flying through the spray cloud (or are sometimes impacted whilst resting on exposed surfaces). The efficiency of contact between the spray droplets and target insects is therefore crucial. This is achieved by ensuring that spray droplets remain airborne for the optimum period of time and that they contain the right dose of insecticide. These two issues are largely addressed through optimizing the droplet size. If droplets are too big they drop to the ground too quickly and don't penetrate vegetation or other obstacles encountered during application (limiting the effective area of application). If one of these big droplets impacts an individual insect then it is also “overkill,” because a high dose will be delivered per individual insect. If droplets are too small then they may either not deposit on a target insect (no impaction) due to aerodynamics or they can be carried upwards into the atmosphere by convection currents. The optimum size of droplets for space-spray application are droplets with a Volume Median Diameter (VMD) of 10-25 microns.

In some embodiments, a sprayable composition may contain an amount of an AMP, or an agriculturally acceptable salt thereof, ranging from about 0.005 wt % to about 99 wt %.

In some embodiments, a sprayable composition may contain an amount of an AMP-insecticidal protein, or an agriculturally acceptable salt thereof, ranging from about 0.005 wt % to about 99 wt %.

In some embodiments, a sprayable composition may contain an amount of a Bt toxin, ranging from about 0.005 wt % to about 99 wt %.

### Foams

The active compositions of the present disclosure comprising (1) an AMP, an AMP-insecticidal protein, or an agriculturally acceptable salt thereof; (2) a Bt toxin; and (3) an excipient, may be made available in a spray product as an aerosol-based application, including aerosolized foam applications. Pressurized cans are the typical vehicle for the formation of aerosols. In some embodiments, an aerosol propellant that is compatible with the AMP, AMP-insecticidal protein, and/or Bt toxin is used. Preferably, a liquefied-gas type propellant is used.

Suitable propellants include compressed air, carbon dioxide, butane and nitrogen. The concentration of the propellant in the active compound composition is from about 5 percent to about 40 percent by weight of the pyridine composition, preferably from about 15 percent to about 30 percent by weight of the comprising (1) an AMP, an AMP-insecticidal protein, or an agriculturally acceptable salt thereof; (2) a Bt toxin; and (3) an excipient.

In one embodiment, formulations comprising a combination of: (1) an AMP, an AMP-insecticidal protein, or an agriculturally acceptable salt thereof; and (2) a Bt toxin; and (3) at least one excipient, can also include one or more foaming agents. Foaming agents that can be used include sodium laureth sulfate, cocamide DEA, and cocamidopropyl betaine. Preferably, the sodium laureth sulfate, cocamide DEA and cocamidopropyl are used in combination. The concentration of the foaming agent(s) in the active compound composition is from about 10 percent to about 25 percent by weight, more preferably 15 percent to 20 percent by weight of the composition.

When such formulations are used in an aerosol application not containing foaming agents, the active compositions of the present disclosure can be used without the need for mixing directly prior to use. However, aerosol formulations containing the foaming agents do require mixing (i.e., shaking) immediately prior to use. In addition, if the formulations containing foaming agents are used for an extended time, they may require additional mixing at periodic intervals during use.

In some embodiments, an aerosolized foam may contain an amount of an AMP, or an agriculturally acceptable salt thereof, ranging from about 0.005 wt % to about 99 wt %.

In some embodiments, an aerosolized foam may contain an amount of an AMP-insecticidal protein, or an agriculturally acceptable salt thereof, ranging from about 0.005 wt % to about 99 wt %.

In some embodiments, an aerosolized foam may contain an amount of a Bt toxin ranging from about 0.005 wt % to about 99 wt %.

### Burning Formulations

In some embodiments, a dwelling area may also be treated with an active combination of: (1) an AMP, an AMP-insecticidal protein, or an agriculturally acceptable salt thereof; and (2) a Bt toxin, by using a burning formulation, such as a candle, a smoke coil or a piece of incense containing the composition. For example, the composition may be formulated into household products such as “heated” air fresheners in which insecticidal compositions are released upon heating, e.g., electrically, or by burning. The active compound compositions of the present disclosure comprising (1) an AMP, an AMP-insecticidal protein, or an agriculturally acceptable salt thereof, and (2) a Bt toxin, may be made available in a spray product as an aerosol, a mosquito coil, and/or a vaporizer or fogger.

In some embodiments, a burning formulation may contain an amount of an AMP, or an agriculturally acceptable salt thereof, ranging from about 0.005 wt % to about 99 wt %.

In some embodiments, a burning formulation may contain an amount of an AMP-insecticidal protein, or an agriculturally acceptable salt thereof, ranging from about 0.005 wt % to about 99 wt %.

In some embodiments, a burning formulation may contain an amount of a Bt toxin ranging from about 0.005 wt % to about 99 wt %.

### Fabric Treatments

In some embodiments, fabrics and garments may be made containing a pesticidal effective composition comprising (1) an AMP, an AMP-insecticidal protein, or an agriculturally acceptable salt thereof, (2) a Bt toxin; and (3) an excipient. In some embodiments, the concentration of the AMP or AMP-insecticidal protein in the polymeric material, fiber, yarn, weave, net, or substrate described herein, can be varied within a relatively wide concentration range from, for example, 0.05 to 15 percent by weight, preferably 0.2 to 10 percent by weight, more preferably 0.4 to 8 percent by weight, especially 0.5 to 5, such as 1 to 3, percent by weight.

Similarly, the concentration of the composition comprising (1) an AMP, an AMP-insecticidal protein, or an agriculturally acceptable salt thereof; (2) a Bt toxin; and (3) an excipient (whether for treating surfaces or for coating a fiber, yarn, net, weave) can be varied within a relatively wide concentration range from, for example 0.1 to 70 percent by weight, such as 0.5 to 50 percent by weight, preferably 1 to 40 percent by weight, more preferably 5 to 30 percent by weight, especially 10 to 20 percent by weight.

The concentration of the AMP or AMP-insecticidal protein may be chosen according to the field of application such that the requirements concerning knockdown efficacy, durability and toxicity are met. Adapting the properties of the material can also be accomplished and so custom-tailored textile fabrics are obtainable in this way.

Accordingly, an effective amount of (1) an AMP, an AMP-insecticidal protein, or an agriculturally acceptable salt thereof, and (2) a Bt toxin can depend on the specific use pattern, the insect pest against which control is most desired and the environment in which the AMP or AMP-insecticidal protein will be used. Therefore, an effective amount of (1) an AMP, an AMP-insecticidal protein, or an agriculturally acceptable salt thereof, and (2) a Bt toxin is sufficient that control of an insect pest is achieved.

In some embodiments, a fabric treatment may contain an amount of an AMP, or an agriculturally acceptable salt thereof, ranging from about 0.005 wt % to about 99 wt %.

In some embodiments, a fabric treatment may contain an amount of an AMP-insecticidal protein, or an agriculturally acceptable salt thereof, ranging from about 0.005 wt % to about 99 wt %.

In some embodiments, a fabric treatment may contain an amount of a Bt toxin ranging from about 0.005 wt % to about 99 wt %.

### Surface-Treatment Compositions

In some embodiments, the present disclosure provides compositions or formulations comprising a combination of: (1) an AMP, an AMP-insecticidal protein, or an agriculturally acceptable salt thereof; and (2) a Bt toxin, for coating walls, floors and ceilings inside of buildings, and for coating a substrate or non-living material. In some embodiments, compositions comprising (1) an AMP, an AMP-insecticidal protein, or an agriculturally acceptable salt thereof; (2) a Bt toxin; and (3) an excipient, can be prepared using known techniques for the purpose in mind. Preparations of compositions comprising a combination of the present disclosure could be so formulated to also contain a binder to facilitate the binding of the compound to the surface or other substrate. Agents useful for binding are known in the art and tend to be polymeric in form. The type of binder suitable for a compositions to be applied to a wall surface having particular porosities and/or binding characteristics would be different compared to a fiber, yarn, weave or net thus, a skilled person, based on known teachings, would select a suitable binder based on the desired surface and/or substrate.

Typical binders are poly vinyl alcohol, modified starch, poly vinyl acrylate, polyacrylic, polyvinyl acetate co polymer, polyurethane, and modified vegetable oils. Suitable binders can include latex dispersions derived from a wide variety of polymers and co-polymers and combinations thereof. Suitable latexes for use as binders in the inventive compositions comprise polymers and copolymers of styrene, alkyl styrenes, isoprene, butadiene, acrylonitrile lower alkyl acrylates, vinyl chloride, vinylidene chloride, vinyl esters of lower carboxylic acids and alpha, beta-ethylenically unsaturated carboxylic acids, including polymers containing three or more different monomer species copolymerized therein, as well as post-dispersed suspensions of silicones or polyurethanes. Also suitable may be a polytetrafluoroethylene (PTFE) polymer for binding the active ingredient to other surfaces.

In some embodiments, a surface-treatment composition may contain an amount of an AMP, or an agriculturally acceptable salt thereof, ranging from about 0.005 wt % to about 99 wt %.

In some embodiments, a surface-treatment composition may contain an amount of an AMP-insecticidal protein, or an agriculturally acceptable salt thereof ranging from about 0.005 wt % to about 99 wt %.

In some embodiments, a surface-treatment composition may contain an amount of a Bt toxin ranging from about 0.005 wt % to about 99 wt %.

### Dispersants

In some exemplary embodiments, an insecticidal formulation according to the present disclosure may comprise a combination of: (1) an AMP, an AMP-insecticidal protein, or an agriculturally acceptable salt thereof; (2) a Bt toxin; and (3) an excipient; and further comprise a diluent or carrier (e.g., such as water), a polymeric binder, and/or additional components such as a dispersing agent, a polymerizing agent, an emulsifying agent, a thickener, an alcohol, a fragrance, or any other inert excipients used in the preparation of sprayable insecticides known in the art.

In some embodiments, a composition comprising (1) an AMP, an AMP-insecticidal protein, or an agriculturally acceptable salt thereof; (2) a Bt toxin; and (3) an excipient, can be prepared in a number of different forms or formulation types, such as suspensions or capsules suspensions. And a person skilled in the art can prepare the relevant composition based on the properties of the particular peptide (e.g., AMP, AMP-insecticidal protein, and/or Bt toxin), its uses, and also its application type. For example, the AMP, AMP-insecticidal protein, and/or Bt toxin used in the methods, embodiments, and other aspects of the present disclosure, may be encapsulated in a suspension or capsule suspension formulation. An encapsulated AMP, AMP-insecticidal protein, and/or Bt toxin can provide improved wash-fastness, and also a longer period of activity. The formulation can be organic based or aqueous based, preferably aqueous based.

In some embodiments, a dispersant may contain an amount of an AMP, or an agriculturally acceptable salt thereof, ranging from about 0.005 wt % to about 99 wt %.

In some embodiments, a dispersant may contain an amount of an AMP-insecticidal protein, or an agriculturally acceptable salt thereof, ranging from about 0.005 wt % to about 99 wt %.

In some embodiments, a dispersant may contain an amount of a Bt toxin ranging from about 0.005 wt % to about 99 wt %.

### Microencapsulation

Microencapsulated AMP or AMP-insecticidal protein and/or Bt toxin suitable for use in the compositions and methods according to the present disclosure may be prepared with any suitable technique known in the art. For example, various processes for microencapsulating material have been previously developed. These processes can be divided into three categories: physical methods, phase separation, and interfacial reaction. In the physical methods category, microcapsule wall material and core particles are physically brought together and the wall material flows around the core particle to form the microcapsule. In the phase separation category, microcapsules are formed by emulsifying or dispersing the core material in an immiscible continuous phase in which the wall material is dissolved and caused to physically separate from the continuous phase, such as by coacervation, and deposit around the core particles. In the interfacial reaction category. microcapsules are formed by emulsifying or dispersing the core material in an immiscible continuous phase and then an interfacial polymerization reaction is caused to take place at the surface of the core particles. The concentration of the AMP or AMP-insecticidal protein or Bt toxin present in the microcapsules can vary from 0.1 to 60% by weight of the microcapsule.

In some embodiments, a microencapsulation may contain an amount of an AMP, or an agriculturally acceptable salt thereof, ranging from about 0.005 wt % to about 99 wt %.

In some embodiments, a microencapsulation may contain an amount of an AMP-insecticidal protein, or an agriculturally acceptable salt thereof, ranging from about 0.005 wt % to about 99 wt %.

In some embodiments, a microencapsulation may contain an amount of a Bt toxin ranging from about 0.005 wt % to about 99 wt %.

### Formulations, Dispersants, Kits, and the Ingredients Thereof

The formulation used in the compositions comprising a combination of (1) an AMP, AMP-insecticidal proteins, or agricultural salts thereof, (2) one or more Bt toxins, and (3) one or more excipients, according to the present disclosure, may be formed by mixing all ingredients together with water, and optionally using suitable mixing and/or dispersing aggregates. In general, such a formulation is formed at a temperature of from 10 to 70° C., preferably 15 to 50° C., more preferably 20 to 40° C. Generally, a formulation comprising one or more of (A), (B), (C), and/or (D) is possible, wherein it is possible to use: an AMP, AMP-insecticidal protein, agricultural salt thereof and a Bt toxin (as pesticidal combination) (A); solid polymer (B); optional additional additives (D); and to disperse them in the aqueous component (C). If a binder is present in a composition of the present disclosure (comprising a combination of (1) an AMP, an AMP-insecticidal protein, or an agriculturally acceptable salt thereof; (2) a Bt toxin; and (3) an excipient), it is preferred to use dispersions of the polymeric binder (B) in water as well as aqueous formulations of the AMP, an AMP-insecticidal protein, or an agriculturally acceptable salt thereof, and/or Bt toxin (A) in water which have been separately prepared before. Such separate formulations may contain additional additives for stabilizing (A) and/or (B) in the respective formulations and are commercially available. In a second process step, such raw formulations and optionally additional water (component (C)) are added. Also, combinations of the abovementioned ingredients based on the foregoing scheme are likewise possible, e.g., using a pre-formed dispersion of (A) and/or (B) and mixing it with solid (A) and/or (B). A dispersion of the polymeric binder (B) may be a pre-manufactured dispersion already made by a chemicals manufacturer.

Moreover, it is also within the scope of the present disclosure to use “hand-made” dispersions, i.e., dispersions made in small-scale by an end-user. Such dispersions may be made by providing a mixture of about 20 percent of the binder (B) in water, heating the mixture to temperature of 90° C. to 100° C. and intensively stirring the mixture for several hours. It is possible to manufacture the formulation as a final product so that it can be readily used by the end-user for the process according to the present disclosure. And, it is of course similarly possible to manufacture a concentrate, which may be diluted by the end-user with additional water (C) to the desired concentration for use.

In an embodiment, a composition comprising a combination of (1) an AMP, an AMP-insecticidal protein, or an agriculturally acceptable salt thereof; (2) a Bt toxin; and (3) an excipient; suitable for SSI application or a coating formulation (comprising a combination of (1) an AMP, an AMP-insecticidal protein, or an agriculturally acceptable salt thereof, (2) a Bt toxin; and (3) an excipient), contains the active ingredient and a carrier, such as water, and may also one or more co-formulants selected from a dispersant, a wetter, an anti-freeze, a thickener, a preservative, an emulsifier and a binder or sticker.

In some embodiments, an exemplary solid formulation of a composition comprising an AMP and a Bt toxin, is generally milled to a desired particle size, such as the particle size distribution d(0.5) is generally from 3 to 20, preferably 5 to 15, especially 7 to 12, μm.

Furthermore, it may be possible to ship the formulation to the end-user as a kit comprising at least a first component comprising an AMP, AMP-insecticidal proteins, or an agricultural salt thereof (A1); a second component comprising a Bt toxin (A2); and a third component comprising at least one polymeric binder (B). In other embodiments, it may be possible to ship the formulation to the end-user as a kit comprising at least a first component comprising a combination of (1) an AMP, AMP-insecticidal proteins, or an agricultural salt thereof, (2) one or more Bt toxins (A); and a second component comprising at least one polymeric binder (B). Further additives (D) may be a third separate component of the kit, or may be already mixed with components (A) and/or (B). The end-user may prepare the formulation for use by just adding water (C) to the components of the kit and mixing. The components of the kit may also be formulations in water. Of course it is possible to combine an aqueous formulation of one of the components with a dry formulation of the other component(s). As an example, the kit can consist of at least one formulation comprising a combination of (1) an AMP, an AMP-insecticidal proteins, or an agriculturally acceptable salt thereof, and (2) one or more Bt toxins (A); and optionally water (C); and a second, separate formulation of at least one polymeric binder (B), water as component (C) and optionally components (D).

The concentrations of the components (A1/A2) or (A), (B), (C) and optionally (D) will be selected by the skilled artisan depending of the technique to be used for coating/treating. In general, the amount of a combination of (1) an AMP, an AMP-insecticidal proteins, or an agriculturally acceptable salt thereof, and (2) one or more Bt toxins, (A) may be up to 50, preferably 1 to 50, such as 10 to 40, especially 15 to 30, percent by weight, based on weight of the composition. The amount of polymeric binder (B) may be in the range of 0.01 to 30, preferably 0.5 to 15, more preferably 1 to 10, especially 1 to 5, percent by weight, based on weight of the composition. If present, in general the amount of additional components (D) is from 0.1 to 20, preferably 0.5 to 15, percent by weight, based on weight of the composition. If present, suitable amounts of pigments and/or dyestuffs and/or fragrances are in general 0.01 to 5, preferably 0.1 to 3, more preferably 0.2 to 2, percent by weight, based on weight of the composition. A typical formulation ready for use comprises 0.1 to 40, preferably 1 to 30, percent of components (A), (B), and optionally (D), the residual amount being water (C). A typical concentration of a concentrate to be diluted by the end-user may comprise 5 to 70, preferably 10 to 60, percent of components (A), (B), and optionally (D), the residual amount being water (C).

Any of the AMPs or AMP-insecticidal proteins, and/or Bt toxins as described herein; and/or any of the methods regarding the same, can be used to create any of the foregoing sprayable compositions, formulations, and/or kits as described herein.

### Illustrative Mixtures, Compositions, Products, and Transgenic Organisms

The present disclosure contemplates combinations, mixtures, compositions, products, and transgenic organisms that contain—or, in the case of transgenic organisms, express or otherwise produce—an AMP, one or more AMP-insecticidal proteins, and/or one or more Bt toxins.

In some embodiments, a composition of the present disclosure comprises: (1) an AMP, an AMP-insecticidal proteins, or an agriculturally acceptable salt thereof; (2) a Bt toxin; and (3) an excipient (e.g., any of the excipients described herein).

In some embodiments, a composition of the present disclosure comprises, consists essentially of, or consists of: (1) an AMP, an AMP-insecticidal proteins, or an agriculturally acceptable salt thereof, (2) a Bt toxin; and (3) an excipient (e.g., any of the excipients described herein); wherein either of the foregoing (1), (2), or (3) can be used concomitantly, or sequentially.

Any of the combinations, mixtures, products, polypeptides and/or plants utilizing an AMP, an AMP-insecticidal protein, or Bt toxin (as described herein), can be used to control pests, their growth, and/or the damage caused by their actions, especially their damage to plants.

Compositions comprising a combination of: (1) an AMP, an AMP-insecticidal protein, or an agriculturally acceptable salt thereof; and (2) a Bt toxin; and (3) at least one excipient, can include agrochemical compositions. For example, in some embodiments, agrochemical compositions can include, but is not limited to, aerosols and/or aerosolized products (e.g., sprays, fumigants, powders, dusts, and/or gases); seed dressings; oral preparations (e.g., insect food, etc.); or a transgenic organisms (e.g., a cell, a plant, or an animal) expressing and/or producing an AMP, an AMP-insecticidal protein, or a Bt toxin, either transiently and/or stably.

In some embodiments, the active ingredients of the present disclosure can be applied in the form of compositions and can be applied to the crop area or plant to be treated, simultaneously or in succession, with other non-active compounds. These compounds can be fertilizers, weed killers, cryoprotectants, surfactants, detergents, soaps, dormant oils, polymers, and/or time-release or biodegradable carrier formulations that permit long-term dosing of a target area following a single application of the formulation. One or more of these non-active compounds can be prepared, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

Methods of applying an active ingredient of the present disclosure or an agrochemical composition of the present disclosure that comprises a combination of: (1) an AMP, an AMP-insecticidal protein, or an agriculturally acceptable salt thereof; and (2) a Bt toxin; and (3) at least one excipient, as produced by the methods described herein of the present disclosure, include stem, flower, or leaf application, seed coating and soil application. In some embodiments, the number of applications and the rate of application depend on the intensity of infestation by the corresponding pest.

The composition comprising an AMP, an AMP-insecticidal protein, or an agriculturally acceptable salt thereof; a Bt toxin; and an excipient may be formulated as a powder, dust, pellet, granule, spray, emulsion, colloid, solution, or such like, and may be prepared by such conventional means as desiccation, lyophilization, homogenization, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

In some embodiments, compositions containing a combination of: (1) an AMP, an AMP-insecticidal protein, or an agriculturally acceptable salt thereof; and (2) a Bt toxin; and (3) at least one excipient, may be prophylactically applied to an environmental area to prevent infestation by a susceptible pest, for example, a lepidopteran and/or coleopteran pest, which may be killed or reduced in numbers in a given area by the methods of the disclosure. In some embodiments, the pest ingests, or comes into contact with, a pesticidally-effective amount of the polypeptide.

In some embodiments, the pesticide compositions described herein may be made by formulating either the AMP or AMP-insecticidal-protein or an agriculturally acceptable salt thereof, or Bt toxin, a transformed bacterial, yeast, or other cell; or a crystal and/or spore suspension, or isolated protein component, with the desired agriculturally-acceptable carrier. The compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, the disclosure of which is incorporated herein by reference in its entirety.

### Methods of Using the Present Disclosure

Any of the methods of using the present disclosure, e.g., methods of protecting plants, plant parts, and seeds; or methods of making and/or using the combinations and compositions of the present disclosure; can be implemented using the AMP, AMP-insecticidal proteins, or Bt toxins as described herein. For example, any of the methods of using the present disclosure as described herein can be implemented using, e.g., an AMP having an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence “KSCCPCYWGGCPWGQNCYPEGCGGPG” (SEQ ID NO: 1), in combination with any of the Bt toxins which are likewise described herein.

### Methods for Protecting Plants, Plant Parts, and Seeds

In some embodiments, the present disclosure provides a method for controlling an invertebrate pest in agronomic and/or nonagronomic applications, comprising contacting the invertebrate pest or its environment, a solid surface, including a plant surface, or part thereof, with a pesticidally effective amount of a combination comprising: (1) an AMP, AMP-insecticidal proteins, or agricultural salts thereof; and (2) one or more Bt toxins.

In some embodiments, the present disclosure provides a method for controlling an invertebrate pest in agronomic and/or nonagronomic applications, comprising contacting the invertebrate pest or its environment, a solid surface, including a plant surface or part thereof, with a pesticidally effective amount of a composition comprising at least one AMP of the present disclosure; at least one Bt toxin; and at least one excipient.

Examples of suitable compositions comprising: (1) at least one AMP of the present disclosure; an AMP-insecticidal protein; an agriculturally acceptable salt thereof; or a combination thereof; (2) at least one Bt toxin of the present disclosure; two or more Bt toxins of the present disclosure; and (3) one or more excipients; include compositions formulated with inactive ingredients to be delivered in the form of: a liquid solution, an emulsion, a powder, a granule, a nanoparticle, a microparticle, or a combination thereof.

In some embodiments, to achieve contact with a compound, mixture, or composition of the present disclosure to protect a field crop from invertebrate pests, the combination or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a combination of (1) an AMP or agricultural salt thereof and/or an AMP-insecticidal protein or agricultural salt thereof; (2) a Bt toxin; and (3) an excipient, can be applied to the plant foliage or the soil. Compounds of this disclosure can also be effectively delivered through plant uptake by contacting the plant with a composition comprising a combination of the present disclosure applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of a combination of (1) an AMP or agricultural salt thereof and/or an AMP-insecticidal protein or agricultural salt thereof; and (2) a Bt toxin. Of further note, the illustrative method contemplates a soil environment, wherein the composition is applied to the soil as a soil drench formulation. Of further note is that a combination of (1) an AMP or agricultural salt thereof and/or an AMP-insecticidal protein or agricultural salt thereof; and (2) a Bt toxin, is also effective by localized application to the locus of infestation. Other methods of contact include application of a combination or a composition of the disclosure by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the present disclosure. The compounds of this disclosure can also be impregnated into materials for fabricating invertebrate control devices (e.g., insect netting, application onto clothing, application into candle formulations and the like).

In some embodiments, a combination of (1) an AMP or agricultural salt thereof and/or an AMP-insecticidal protein or agricultural salt thereof; and (2) a Bt toxin, is also useful in seed treatments for protecting seeds from invertebrate pests. In the context of the present disclosure and claims, treating a seed means contacting the seed with a biologically effective amount of a combination of (1) an AMP or agricultural salt thereof and/or an AMP-insecticidal protein or agricultural salt thereof; and (2) a Bt toxin, which is typically formulated as a composition of the present disclosure. This seed treatment protects the seed from invertebrate soil pests and generally can also protect roots and other plant parts in contact with the soil of the seedling developing from the germinating seed. The seed treatment may also provide protection of foliage by translocation of the AMP or AMP-insecticidal protein or Bt toxin within the developing plant. Seed treatments can be applied to all types of seeds, including those from which plants genetically transformed to express specialized traits will germinate. In addition, an AMP or an AMP-insecticidal protein can be transformed into a plant or part thereof, for example a plant cell, or plant seed, that is already transformed, e.g., those expressing herbicide resistance such as glyphosate acetyltransferase, which provides resistance to glyphosate.

One method of seed treatment is by spraying or dusting the seed with a combination of (1) an AMP or agricultural salt thereof and/or an AMP-insecticidal protein or agricultural salt thereof; and (2) a Bt toxin, before sowing the seeds. Compositions formulated for seed treatment generally comprise a combination of (1) an AMP or agricultural salt thereof and/or an AMP-insecticidal protein or agricultural salt thereof; and (2) a Bt toxin, and a film former or adhesive agent. Therefore, typically, a seed coating composition of the present disclosure consists of a biologically effective amount of a combination of (1) an AMP or agricultural salt thereof and/or an AMP-insecticidal protein or agricultural salt thereof; and (2) a Bt toxin, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Monograph No. 57, and references listed therein, the disclosures of which are incorporated herein by reference in their entireties.

The treated seed typically comprises a combination of (1) an AMP or agricultural salt thereof and/or an AMP-insecticidal protein or agricultural salt thereof; and (2) a Bt toxin, in an amount ranging from about 0.01 g to 1 kg per 100 kg of seed (i.e. from about 0.00001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

In some embodiments, the present disclosure provides a method for controlling insects and/or for protecting against a pest, wherein the pest is selected from the group consisting of: group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola); Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex (Various Leps.); Saddleback Caterpillar (Sibine stimulea); Saddle Prominent Caterpillar Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and Xanthogaleruca luteola.

### Methods of Using Combinations and Compositions

In some embodiments, the present disclosure provides a method of combating, controlling, or inhibiting a pest comprising, applying a pesticidally effective amount of the combination or composition comprising, consisting essentially of, or consisting of applying a combination of: (1) an AMP or agricultural salt thereof and/or an AMP-insecticidal protein or agricultural salt thereof, and (2) a Bt toxin; to the following: (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination of any one of (i)-(iii).

In some embodiments, the present disclosure provides a method of using a combination, or agricultural composition thereof, comprising: (1) an AMP or agricultural salt thereof and/or an AMP-insecticidal protein or agricultural salt thereof, (2) a Bt toxin; and optionally (3) an excipient; to control insects, wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein said method comprises, preparing the combination and then applying said combination, either simultaneously or sequentially, to (i) the insect, a locus of the insect, a food supply of the insect, a habitat of the insect, or a breeding ground of the insect; (ii) a plant, a seed, a plant part, a locus of a plant, or an environment of a plant that is susceptible to an attack by the insect; (iii) an animal, a locus of an animal, or an environment of an animal susceptible to an attack by the insect; or (iv) a combination of any one of (i)-(iii).

In some embodiments, the present disclosure provides a method to control insects comprising the use of a combination, or agricultural composition thereof comprising: (1) an AMP or agricultural salt thereof and/or an AMP-insecticidal protein or agricultural salt thereof; (2) a Bt toxin; and optionally (3) an excipient; wherein the insects are selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia calfornica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and/or Xanthogaleruca luteola.

In some embodiments, the present disclosure provides a method of protecting a plant from insects comprising, providing a plant which expresses an AMP, one or more AMP-insecticidal proteins, and/or one or more Bt toxins, or polynucleotides encoding the same.

In some embodiments, the present disclosure provides a method of protecting a plant from insects comprising, providing a plant that expresses an AMP and/or a Bt toxin, or polynucleotide encoding the same, wherein said AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1); or an agriculturally acceptable salt thereof.

In some embodiments, the present disclosure provides a method of protecting a plant from insects comprising, providing a plant that expresses an AMP, or polynucleotide encoding the same, wherein said AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1); and applying one or more Bt toxins of the present disclosure.

In some embodiments, the present disclosure provides a method of protecting a plant from insects comprising, providing a plant that expresses a Bt toxin of the present disclosure, and applying an AMP comprising an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof.

In some embodiments, the present disclosure provides a method of protecting a plant from insects comprising, providing a plant that expresses an AMP, or polynucleotide encoding the same, wherein the polynucleotide encodes an AMP having an amino acid sequence as set forth in SEQ ID NO: 1, or a complementary nucleotide sequence thereof.

In some embodiments, the present disclosure provides a method of protecting a plant from insects comprising, providing a plant that expresses an AMP, or polynucleotide encoding the same, wherein the AMP further comprises a homopolymer of two or more AMPs, wherein the amino acid sequence of each AMP is the same.

In some embodiments, the present disclosure provides a method of protecting a plant from insects comprising, providing a plant that expresses an AMP, or polynucleotide encoding the same, wherein the AMP is a fused protein comprising two or more AMPs separated by a cleavable or non-cleavable linker, and wherein the amino acid sequence of each AMP is the same.

In some embodiments, the present disclosure provides a method of protecting a plant from insects comprising, providing a plant that expresses an AMP, or polynucleotide encoding the same, wherein the AMP is a fused protein comprising two or more AMPs separated by a cleavable linker. In some embodiments, the linker has an amino acid sequence as set forth in any one of SEQ ID NOs: 4-16.

In some embodiments, the present disclosure provides a method of protecting a plant from insects comprising, providing a plant that expresses an AMP, or polynucleotide encoding the same, wherein the AMP is a fused protein comprising two or more AMPs separated by a linker, wherein the linker is cleavable inside at least one of (i) the gut or hemolymph of an insect, and (ii) cleavable inside the gut of a mammal.

In some embodiments, the present disclosure provides a method for controlling insects comprising, providing to said insect a transgenic plant that comprises in its genome a stably incorporated expression cassette, wherein said stably incorporated expression cassette comprises polynucleotide operable to encode an AMP and a Bt toxin.

In some embodiments, the present disclosure provides a method for controlling insects comprising, providing to said insect a transgenic plant that comprises in its genome a stably incorporated expression cassette, wherein said stably incorporated expression cassette comprises polynucleotide operable to encode an AMP, and the method further comprising the application of one or more Bt toxins.

In some embodiments, the present disclosure provides a method for controlling insects comprising, providing to said insect a transgenic plant that comprises in its genome a stably incorporated expression cassette, wherein said stably incorporated expression cassette comprises polynucleotide operable to encode a Bt toxin, and the method further comprising the application of an AMP, AMP-insecticidal protein, or agricultural salt thereof.

In some embodiments, the present disclosure provides a method of combating, controlling, or inhibiting a pest comprising, applying a pesticidally effective amount of a composition comprising: (1) an AMP or agricultural salt thereof and/or an AMP-insecticidal protein or agricultural salt thereof; (2) a Bt toxin; and (3) an excipient; wherein the AMP has an amino acid sequence as set forth in SEQ ID NO: 1, or an agriculturally acceptable salt thereof, wherein the combination is applied to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination of any one of (i)-(iii).

In some embodiments, the present disclosure provides a method of combating, controlling, or inhibiting a pest comprising, applying a pesticidally effective amount of a composition comprising: (1) an AMP or agricultural salt thereof and/or an AMP-insecticidal protein or agricultural salt thereof; (2) a Bt toxin; and (3) an excipient; to (i) the pest, a locus of the pest, a food supply of the pest, a habitat of the pest, or a breeding ground of the pest; (ii) a plant, a seed, a plant part, a locus of a plant, or an environment of a plant that is susceptible to an attack by the pest; (iii) an animal, a locus of an animal, or an environment of an animal susceptible to an attack by the pest; or (iv) a combination of any one of (i)-(iii), wherein the pest is selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and/or Xanthogaleruca luteola.

### Crops and Pests

Specific crop pests and insects that may be controlled by these methods include the following: Dictyoptera (cockroaches); Isoptera (termites); Orthoptera (locusts, grasshoppers and crickets); Diptera (house flies, mosquito, tsetse fly, crane-flies and fruit flies); Hymenoptera (ants, wasps, bees, saw-flies, ichneumon flies and gall-wasps); Anoplura (biting and sucking lice); Siphonaptera (fleas); and Hemiptera (bugs and aphids), as well as arachnids such as Acari (ticks and mites), and the parasites that each of these organisms harbor.

“Pest” includes, but is not limited to: insects, fungi, bacteria, nematodes, mites, ticks, and the like.

Insect pests include, but are not limited to, insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. More particularly, insect pests include Coleoptera, Lepidoptera, and Diptera.

Insects of suitable agricultural, household and/or medical/veterinary importance for treatment with the insecticidal peptides described herein include, but are not limited to, members of the following classes and orders:

The order Coleoptera includes the suborders Adephaga and Polyphaga. Suborder Adephaga includes the superfamilies Caraboidea and Gyrinoidea. Suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

Examples of Coleoptera include, but are not limited to: the American bean weevil Acanthoscelides obtectus, the leaf beetle Agelastica alni, click beetles (Agriotes lineatus, Agriotes obscurus, Agriotes bicolor), the grain beetle Ahasverus advena, the summer schafer Amphimallon solstitialis, the furniture beetle Anobium punctatum, Anthonomus spp. (weevils), the Pygmy mangold beetle Atomaria linearis, carpet beetles (Anthrenus spp., Attagenus spp.), the cowpea weevil Callosobruchus maculates, the fried fruit beetle Carpophilus hemipterus, the cabbage seedpod weevil Ceutorhynchus assimilis, the rape winter stem weevil Ceutorhynchus picitarsis, the wireworms Conoderus vespertinus and Conoderus falli, the banana weevil Cosmopolites sordidus, the New Zealand grass grub Costelytra zealandica, the June beetle Cotinis nitida, the sunflower stem weevil Cylindrocopturus adspersus, the larder beetle Dermestes lardarius, the corn rootworms Diabrotica virgifera, Diabrotica virgifera virgifera, and Diabrotica barberi, the Mexican bean beetle Epilachna varivestis, the old house borer Hylotropes bajulus, the luceme weevil Hypera postica, the shiny spider beetle Gibbium psylloides, the cigarette beetle Lasioderma serricorne, the Colorado potato beetle Leptinotarsa decemlineata, Lyctus beetles (Lyctus spp.), the pollen beetle Meligethes aeneus, the common cockshafer Melolontha melolontha, the American spider beetle Mezium americanum, the golden spider beetle Niptus hololeucus, the grain beetles Oryzaephilus surinamensis and Oryzaephilus mercator, the black vine weevil Otiorhynchus sulcatus, the mustard beetle Phaedon cochleariae, the crucifer flea beetle Phyllotreta cruciferae, the striped flea beetle Phyllotreta striolata, the cabbage steam flea beetle Psylliodes chrysocephala. Ptinus spp. (spider beetles), the lesser grain borer Rhizopertha dominica, the pea and been weevil Sitona lineatus, the rice and granary beetles Sitophilus oryzae and Sitophilus granaries, the red sunflower seed weevil Smicronyx fulvus, the drugstore beetle Stegobium paniceum, the yellow mealworm beetle Tenebrio molitor, the flour beetles Tribolium castaneum and Tribolium confusum, warehouse and cabinet beetles (Trogoderma spp.), and the sunflower beetle Zygogramma exclamationis.

Examples of Dermaptera (earwigs) include, but are not limited to: the European earwig, Forficula auricularia, and the striped earwig, Labidura riparia.

Examples of Dictvontera include, but are not limited to: the oriental cockroach, Blatta orientalis, the German cockroach, Blatella germanica, the Madeira cockroach, Leucophaea maderae, the American cockroach, Periplaneta americana, and the smokybrown cockroach Periplaneta fuliginosa.

Examples of Diplonoda include, but are not limited to: the spotted snake millipede Blaniulus guttulatus, the flat-back millipede Brachydesmus superus, and the greenhouse millipede Oxidus gracilis.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simu/idae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidac, and Drosophilidae. Section Calyptratae includes the families Hippoboscidae, Oestridae, Tachinidae, Anthomyiidae, Muscidae, Calliphoridae, and Sarcophagidae.

Examples of Diptera include, but are not limited to: the house fly (Musca domestica), the African tumbu fly (Cordylobia anthropophaga), biting midges (Culicoides spp.), bee louse (Braula spp.), the beet fly Pegomyza betae, blackflies (Cnephia spp., Eusimulium spp., Simulium spp.), bot flies (Cuterebra spp., Gastrophilus spp., Oestrus spp.), craneflies (Tipula spp.), eye gnats (Hippelates spp.), filth-breeding flies (Calliphora spp., Fannia spp., Hermetia spp., Lucilia spp., Musca spp., Muscina spp., Phaenicia spp., Phormia spp.), flesh flies (Sarcophaga spp., Wohlfahrtia spp.); the flit fly Oscinella frit, fruitflies (Dacus spp., Drosophila spp.), head and canon flies (Hydrotea spp.), the hessian fly Mayetiola destructor, horn and buffalo flies (Haematobia spp.), horse and deer flies (Chrysops spp., Haematopota spp., Tabanus spp.), louse flies (Lipoptena spp., Lynchia spp., and Pseudolynchia spp.), medflies (Ceratitus spp.), mosquitoes (Aedes spp., Anopheles spp., Culex spp., Psorophora spp.), sandflies (Phlebotomus spp., Lutzomyia spp.), screw-worm flies (Chtysomya bezziana and Cochhomyia hominivorax), sheep keds (Melophagus spp.); stable flies (Stomoxys spp.), tsetse flies (Glossina spp.), and warble flies (Hypoderma spp.).

Examples of Isontera (termites) include, but are not limited to: species from the familes Hodotennitidae, Kalotermitidae, Mastotermitidae, Rhinotennitidae, Serritermitidae, Termitidae, and Termopsidae.

Examples of Heteroptera include, but are not limited to: the bed bug Cimex lectularius, the cotton stainer Dysdercus intermedius, the Sunn pest Eurygaster integriceps, the tarnished plant bug Lygus lineolaris, the green stink bug Nezara antennata, the southern green stink bug Nezara viridula, and the triatomid bugs Panstrogylus megistus, Rhodnius ecuadoriensis, Rhodnius pallescans, Rhodnius prolixus, Rhodnius robustus, Triatoma dimidiata, Triatoma infestans, and Triatoma sordida.

Examples of Homoptera include, but are not limited to: the California red scale Aonidiella aurantii, the black bean aphid Aphis fabae, the cotton or melon aphid Aphis gossypii, the green apple aphid Aphis pomi, the citrus spiny whitefly Aleurocanthus spiniferus, the oleander scale Aspidiotus hederae, the sweet potato whitefly Bemesia tabaci, the cabbage aphid Brevicoryne brassicae, the pear psylla Cacopsylla pyricola, the currant aphid Cryptomyzus ribis, the grape phylloxera Daktulosphaira vitifoliae, the citrus psylla Diaphorina citri, the potato leafhopper Empoasca fabae, the bean leafhopper Empoasca solana, the vine leafhopper Empoasca vitis, the woolly aphid Eriosoma lanigerum, the European fruit scale Eulecanium corni, the mealy plum aphid Hyalopterus arundinis, the small brown planthopper Laodelphax striatellus, the potato aphid Macrosiphum euphorbiae, the green peach aphid Myzus persicae, the green rice leafhopper Nephotettix cinticeps, the brown planthopper Nilaparvata lugens, gall-forming aphids (Pemphigus spp.), the hop aphid Phorodon humuli, the bird-cherry aphid Rhopalosiphum padi, the black scale Saissetia oleae, the greenbug Schizaphis graminum, the grain aphid Sitobion avenae, and the greenhouse whitefly Trialeurodes vaporariorum.

Examples of Isopoda include, but are not limited to: the common pillbug Armadillidium vulgare and the common woodlouse Oniscus asellus.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Examples of Lepidoptera include, but are not limited to: Adoxophyes orana (summer fruit tortrix moth), Agrotis ipsolon (black cutworm), Archips podana (fruit tree tortrix moth), Bucculatrix pyrivorella (pear leafminer), Bucculatrix thurberiella (cotton leaf perforator), Bupalus piniarius (pine looper), Carpocapsa pomonella (codling moth), Chilo suppressalis (striped rice borer), Choristoneura fumiferana (eastern spruce budworm), Cochylis hospes (banded sunflower moth), Diatraea grandiosella (southwestern corn borer), Earls insulana (Egyptian bollworm), Euphestia kuehniella (Mediterranean flour moth), Eupoecilia ambiguella (European grape berry moth), Euproctis chrysorrhoea (brown-tail moth), Euproctis subflava (oriental tussock moth), Galleria mellonella (greater wax moth), Helicoverpa armigera (cotton bollworm), Helicoverpa zea (cotton bollworm), Heliothis virescens (tobacco budworm), Hofmannophila pseudopretella (brown house moth), Homeosoma electellum (sunflower moth), Homona magnanima (oriental tea tree tortrix moth), Lithocolletis blancardella (spotted tentiform leafminer), Lymantria dispar (gypsy moth), Malacosoma neustria (tent caterpillar), Mamestra brassicae (cabbage armyworm), Mamestra configurata (Bertha armyworm), the hornworms Manduca sexta and Manuduca quinquemaculata, Operophtera brumata (winter moth), Ostrinia nubilalis (European corn borer), Panolis, lammea (pine beauty moth), Pectinophora gossypiella (pink bollworm), Phyllocnistis citrella (citrus leafminer), Pieris brassicae (cabbage white butterfly), Plutella xylostella (diamondback moth), Rachiplusia ni (soybean looper), Spilosoma virginica (yellow bear moth), Spodoptera exigua (beet armyworm), Spodoptera frugiperda (fall armyworm), Spodoptera littoralis (cotton leafworin), Spodoptera litura (common cutworm), Spodoptera praefica (yellowstriped armyworm), Sylepta derogata (cotton leaf roller), Tineola bisselliella (webbing clothes moth), Tineola pellionella (case-making clothes moth), Tortrix viridana (European oak leafroller), Trichoplusia ni (cabbage looper), and Yponomeuta padella (small ermine moth).

Examples of Orthoptera include, but are not limited to: the common cricket Acheta domesticus, tree locusts (Anacridium spp.), the migratory locust Locusta migratoria, the twostriped grasshopper Melanoplus bivittatus, the differential grasshopper Melanoplus dfferentialis, the redlegged grasshopper Melanoplus femurrubrum, the migratory grasshopper Melanoplus sanguinipes, the northern mole cricket Neocurtilla hexadectyla, the red locust Nomadacris septemfasciata, the shortwinged mole cricket Scapteriscus abbreviatus, the southern mole cricket Scapteriscus borellii, the tawny mole cricket Scapteriscus vicinus, and the desert locust Schistocerca gregaria.

Examples of Phthiraptera include, but are not limited to: the cattle biting louse Bovicola bovis, biting lice (Damalinia spp.), the cat louse Felicola subrostrata, the shortnosed cattle louse Haematopinus eloysternus, the tail-switch louse Haematopinus quadriperiussus, the hog louse Haematopinus suis, the face louse Linognathus ovillus, the foot louse Linognathus pedalis, the dog sucking louse Linognathus setosus, the long-nosed cattle louse Linognathus vituli, the chicken body louse Menacanthus stramineus, the poultry shaft louse Menopon gallinae, the human body louse Pediculus humanus, the pubic louse Phthirus pubis, the little blue cattle louse Solenopotes capillatus, and the dog biting louse Trichodectes canis.

Examples of Psocoptera include, but are not limited to: the booklice Liposcelis bostrychophila, Liposcelis decolor, Liposcelis entomophila, and Trogium pulsatorium. Examples of Siphonaptera include, but are not limited to: the bird flea Ceratophyllus gallinae, the dog flea Ctenocephalides canis, the cat flea Ctenocephalides fells, the human flea Pulex irritans, and the oriental rat flea Xenopsylla cheopis.

Examples of Symphyla include, but are not limited to: the garden symphylan Scutigerella immaculate.

Examples of Thysanura include, but are not limited to: the gray silverfish Ctenolepisma longicaudata, the four-lined silverfish Ctenolepisma quadriseriata, the common silverfish Lepisma saccharina, and the firebrat Thennobia domestica;

Examples of Thysanoptera include, but are not limited to: the tobacco thrips Frankliniella fusca, the flower thrips Frankliniella intonsa, the western flower thrips Frankliniella occidentalis, the cotton bud thrips Frankliniella schultzei, the banded greenhouse thrips Hercinothrips femoralis, the soybean thrips Neohydatothrips variabilis, Kelly's citrus thrips Pezothrips kellyanus, the avocado thrips Scirtothrips perseae, the melon thrips, Thrips palmi, and the onion thrips, Thrips tabaci.

Examples of Nematodes include, but are not limited to: parasitic nematodes such as root-knot, cyst, and lesion nematodes, including Heterodera spp., Meloidogyne spp., and Globodera spp.; particularly members of the cyst nematodes, including, but not limited to: Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include, but are not limited to: Pratylenchus spp.

Other insect species susceptible to the present disclosure include: athropod pests that cause public and animal health concerns, for example, mosquitos for example, mosquitoes from the genera Aedes, Anopheles and Culex, from ticks, flea, and flies etc.

In one embodiment, an AMP, an AMP-insecticidal protein, or an agriculturally acceptable salt thereof can be employed to treat ectoparasites. Ectoparasites include, but are not limited to: fleas, ticks, mange, mites, mosquitoes, nuisance and biting flies, lice, and combinations comprising one or more of the foregoing ectoparasites. The term “fleas” includes the usual or accidental species of parasitic flea of the order Siphonaptera, and in particular the species Ctenocephalides, in particular C. fells and C. cams, rat fleas (Xenopsylla cheopis) and human fleas (Pulex irritans).

The present disclosure may be used to control, inhibit, and/or kill insect pests of major crops, e.g., in some embodiments, the major crops and corresponding insect pest include, but are not limited to: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis, corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper: Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, banded winged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Soybean: Pseudoplusia includens, soybean looper; Anticarsia gemmatalis, velvet bean caterpillar; Plathypena scabra, green clover worm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In some embodiments, an AMP, an AMP-insecticidal protein, or an agriculturally acceptable salt thereof can be employed to treat any one or more of the foregoing insects, or any of the insects described herein.

The insects that are susceptible to present disclosure include but are not limited to the following: familes such as: Blattaria, Coleoptera, Collembola, Diptera, Echinostomida, Hemiptera, Hymenoptera, Isoptera, Lepidoptera, Neuroptera, Orthoptera, Rhabditida, Siphonoptera, and Thysanoptera. Genus Species are indicated as follows: Actebia fennica, Agrotis ipsilon, A. segetum, Anticarsia gemmatalis, Argyrotaenia citrana, Artogeia rapae, Bombyx mori, Busseola fusca, Cacyreus marshall, Chilo suppressalis, Christoneura fumiferana, C. occidentalis, C. pinus pinus, C. rosacena, Cnaphalocrocis medinalis, Conopomorpha cramerella, Ctenopsuestis obliquana, Cydia pomonella, Danaus plexippus, Diatraea saccharallis, D. grandiosella, Earias vittella, Elasmolpalpus lignoselius, Eldana saccharina, Ephestia kuehniella, Epinotia aporema, Epiphyas postvittana, Galleria mellonella, Genus-Species, Helicoverpa zea, H. punctigera, H. armigera, Heliothis virescens, Hyphantria cunea, Lambdina fiscellaria, Leguminivora glycinivorella, Lobesia botrana, Lymantria dispar, Malacosoma disstria, Mamestra brassicae, M. configurata, Manduca sexta, Marasmia patnalis, Maruca vitrata, Orgyia leucostigma, Ostrinia nubilalis, O. furnacalis, Pandemis pyrusana, Pectinophora gossypiella, Perileucoptera coffeella, Phthorimaea opercullela, Pianotortrix octo, Piatynota stultana, Pieris brassicae, Plodia interpunctala, Plutella xylostella, Pseudoplusia includens, Rachiplusia nu, Sciropophaga incertulas, Sesamia calamistis, Spilosoma virginica, Spodoptera exigua, Spodoptera frugiperda, Spodoptera littoralis, Spodoptera exempta, Spodoptera litura, Tecia solanivora, Thaumetopoea pityocampa, Trichoplusia ni, Wiseana cervinata, Wiseana copularis, Wiseana jocosa, Blattaria blattella, Collembola xenylla, Collembola folsomia, Folsomia candida, Echinostomida fasciola, Hemiptera oncopeltrus, Hemiptera bemisia, Hemiptera macrosiphum, Hemiptera rhopalosiphum, Hemiptera myzus, Hymenoptera diprion, Hymenoptera apis, Hymenoptera Macrocentrus, Hymenoptera Meteorus, Hymenoptera Nasonia, Hymenoptera Solenopsis, Isopoda porcellio, Isoptera reticulitermes, Orthoptera Achta, Prostigmata tetranychus, Rhabitida acrobeloides, Rhabitida caenorhabditis, Rhabitida distolabrellus, Rhabitida panagrellus, Rhabitida pristionchus, Rhabitida pratylenchus, Rhabitida ancylostoma, Rhabitida nippostrongylus, Rhabitida panagrellus, Rhabitida haemonchus, Rhabitida meloidogyne, and Siphonaptera ctenocephalides.

The present disclosure provides methods for plant transformation, which may be used for transformation of any plant species, including, but not limited to, monocots and dicots. Crops for which a transgenic approach would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

The present disclosure provides methods for plant transformation, which may be used for transformation of any plant species, including, but not limited to, monocots and dicots. Crops for which a transgenic approach or plaint incorporated protectants (PIP) would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

In some embodiments, the compositions, mixtures, and/or methods of the present disclosure can be applied to the locus of an insect and/or pest selected from the group consisting of: Loopers; Omnivorous Leafroller; Hornworms; Imported Cabbageworm; Diamondback Moth; Green Cloverworm; Webworm; Saltmarsh Caterpillar; Armyworms; Cutworms; Cross-Striped Cabbageworm; Podworms; Velvetbean Caterpillar; Soybean Looper; Tomato Fruitworm; Variegated Cutworm; Melonworms; Rindworm complex; Fruittree Leafroller; Citrus Cutworm; Heliothis; Orangedog; Citrus Cutworm; Redhumped Caterpillar; Tent Caterpillars; Fall Webworm; Walnut Caterpillar; Cankerworms; Gypsy Moth; Variegated Leafroller; Redbanded Leafroller; Tufted Apple Budmoth; Oriental Fruit Moth); Filbert Leafroller; Obliquebanded Leafroller; Codling Moth; Twig Borer; Grapeleaf Skeletonizer; Grape Leafroller; Achema Sphinx Moth (Hornworm); Orange Tortrix; Tobacco Budworm); Grape Berry Moth; Spanworm; Alfalfa Caterpillar; Cotton Bollworm; Head Moth; Amorbia Moth; Omnivorous Looper; Ello Moth (Hornworm); Io Moth; Oleander Moth; Azalea Caterpillar; Hornworm; Leafrollers; Banana Skipper; Batrachedra comosae (Hodges); Thecla Moth; Artichoke Plume Moth; Thistle Butterfly; Bagworm; Spring & Fall Cankerworm; Elm Spanworm; California Oakworm; Pine Butterfly; Spruce Budworms; Saddle Prominent Caterpillar; Douglas Fir Tussock Moth; Western Tussock Moth; Blackheaded Budworm; Mimosa Webworm; Jack Pine Budworm; Saddleback Caterpillar; Greenstriped Mapleworm; or Hemlock Looper.

In some embodiments, the peptides, proteins, compositions, mixtures, and/or methods of the present disclosure can be applied to the locus of an insect and/or pest selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola) (Essex Skipper); Fall Webworm (Melissopus latiferreanus); Filbert Leafroller (Archips rosanus); Fruittree Leafroller (Archips argyrospilia); Grape Berry Moth (Paralobesia viteana); Grape Leafroller (Platynota stultana); Grapeleaf Skeletonizer (Harrisina americana) (ground only); Green Cloverworm (Plathypena scabra); Greenstriped Mapleworm (Dryocampa rubicunda); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Redbanded Leafroller (Argyrotaenia velutinana); Redhumped Caterpillar (Schizura concinna); Rindworm Complex (Various Leps.); Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and/or Xanthogaleruca luteola.

In some embodiments, the peptides, proteins, compositions, mixtures, and/or methods of the present disclosure can be applied to the locus of an adult beetle selected from the group consisting of: Asiatic garden beetle (Maladera castanea); Gold spotted oak borer (Agrilus coxalis auroguttatus); Green June beetle (Cotinis nitida); Japanese beetle (Popillia japonica); May or June beetle (Phyllophaga sp.); Oriental beetle (Anomala orientalis); and/or Soap berry-borer (Agrilus prionurus).

In some embodiments, the compositions, mixtures, and/or methods of the present disclosure can be applied to the locus of an insect and/or pest that is a larvae (annual white grub) selected from the group consisting of: Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); and Billbug (Curculionoidea).

### Illustrative Combination Embodiments

In some embodiments, a combination of the present disclosure comprises, consists essentially of, or consists of: an Av3 mutant polypeptide (AMP), and a Bacillus thuringiensis var. kurstaki (Btk) toxin; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical. at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP comprises an amino acid sequence that is at least 95%, 96%, 97%, 97%, 98%, 99% or 100% identical to the amino acid sequence set forth in SEQ ID NO: 1, or a agriculturally acceptable salt thereof.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP consists essentially of an amino acid sequence that is at least 95%, 96%, 97%, 97%, 98%, 99% or 100% identical to the amino acid sequence set forth in SEQ ID NO: 1, or a agriculturally acceptable salt thereof.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP consists of an amino acid sequence that is at least 95%, 96%, 97%, 97%, 98%, 99% or 100% identical to the amino acid sequence set forth in SEQ ID NO: 1, or a agriculturally acceptable salt thereof.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP consists of an amino acid sequence as set forth in SEQ ID NO: 1, or a agriculturally acceptable salt thereof.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP is a homopolymer of two or more AMPs, wherein the amino acid sequence of each AMP is the same.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP is a fused protein comprising two or more AMPs separated by a linker, and wherein the amino acid sequence of each AMP is the same.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP is a fused protein comprising two or more AMPs separated by a linker, and wherein the amino acid sequence of each AMP is the same; and wherein the linker is a cleavable or non-cleavable linker.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP is a fused protein comprising two or more AMPs separated by a linker, wherein the amino acid sequence of each AMP is the same; and wherein the linker has an amino acid sequence as set forth in any one of SEQ ID NOs: 4-16.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP is a fused protein comprising two or more AMPs separated by a linker, and wherein the amino acid sequence of each AMP is the same; and wherein the linker is cleavable inside at least one of (i) the gut or hemolymph of an insect, and (ii) cleavable inside the gut of a mammal.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk).

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk) strain EVB-113-19, or ABTS-351.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk) strain EVB-113-19.

In some embodiments, a combination of the present disclosure comprises, consists essentially of, or consists of: a combination comprising an Av3 mutant polypeptide (AMP), and a Bacillus thuringiensis var. kurstaki (Btk) toxin; wherein the AMP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk) strain ABTS-351.

In some embodiments, a combination of the present disclosure comprises, consists essentially of, or consists of: one or more fermentation solids, spores, or toxins isolated from a Bacillus thuringiensis ssp. kurstaki strain EVB-113-19, and an Av3 mutant polypeptide (AMP) having an amino acid sequence set forth in SEQ ID NO: 1.

In some embodiments, a combination of the present disclosure comprises, consists essentially of, or consists of: one or more fermentation solids, spores, or toxins isolated from a Bacillus thuringiensis ssp. kurstaki strain ABTS-351, and an Av3 mutant polypeptide (AMP) having an amino acid sequence set forth in any one of SEQ ID NO: 1.

### Illustrative Composition Embodiments

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP comprises an amino acid sequence that is at least 95%, 96%, 97%, 97%, 98%, 99% or 100% identical to the amino acid sequence set forth in SEQ ID NO: 1, or a agriculturally acceptable salt thereof.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP consists essentially of an amino acid sequence that is at least 95%, 96%, 97%, 97%, 98%, 99% or 100% identical to the amino acid sequence set forth in SEQ ID NO: 1, or a agriculturally acceptable salt thereof.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP consists of an amino acid sequence that is at least 95%, 96%, 97%, 97%, 98%, 99% or 100% identical to the amino acid sequence set forth in SEQ ID NO: 1, or a agriculturally acceptable salt thereof.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP consists of an amino acid sequence as set forth in SEQ ID NO: 1, or a agriculturally acceptable salt thereof.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP is a homopolymer of two or more AMPs, wherein the amino acid sequence of each AMP is the same.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP is a fused protein comprising two or more AMPs separated by a linker, and wherein the amino acid sequence of each AMP is the same.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP is a fused protein comprising two or more AMPs separated by a linker, and wherein the amino acid sequence of each AMP is the same; and wherein the linker is a cleavable or non-cleavable linker.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP is a fused protein comprising two or more AMPs separated by a linker, wherein the amino acid sequence of each AMP is the same; and wherein the linker has an amino acid sequence as set forth in any one of SEQ ID NOs: 4-16.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP is a fused protein comprising two or more AMPs separated by a linker, and wherein the amino acid sequence of each AMP is the same; and wherein the linker is cleavable inside at least one of (i) the gut or hemolymph of an insect, and (ii) cleavable inside the gut of a mammal.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk).

In some embodiments, a composition of the present disclosure comprises, consists essentially of, or consists of an Av3 mutant polypeptide (AMP), a Bacillus thuringiensis var. kurstaki (Btk) toxin; and at least one excipient; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk) strain EVB-113-19, or ABTS-351.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk) strain EVB-113-19.

In some embodiments, a composition of the present disclosure comprises, consists essentially of, or consists of: an Av3 mutant polypeptide (AMP), a Bacillus thuringiensis var. kurstaki (Btk) toxin; and at least one excipient; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; and wherein the Btk toxin is one or more fermentation solids, spores, or toxins isolated from a Bacillus thuringiensis var. kurstaki (Btk) strain ABTS-351.

In some embodiments, a composition of the present disclosure comprises, consists essentially of, or consists of one or more fermentation solids, spores, or toxins isolated from a Bacillus thuringiensis ssp. kurstaki strain EVB-113-19; an Av3 mutant polypeptide (AMP) having an amino acid sequence set forth in SEQ ID NO: 1; and an excipient.

In some embodiments, a composition of the present disclosure comprises, consists essentially of, or consists of one or more fermentation solids, spores, or toxins isolated from a Bacillus thuringiensis ssp. kurstaki strain ABTS-351; an Av3 mutant polypeptide (AMP) having an amino acid sequence set forth in any one of SEQ ID NO: 1; and an excipient.

### Illustrative Method Embodiments

In some embodiments, the present disclosure provides a method of combating, controlling, or inhibiting a pest comprising, applying (1) a pesticidally effective amount of a combination comprising, consisting essentially of, or consisting of an Av3 mutant polypeptide (AMP), and a Bacillus thuringiensis var. kurstaki (Btk) toxin; wherein the AMP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; or (2) a pesticidally effective amount of a composition comprising, consisting essentially of, or consisting of: an Av3 mutant polypeptide (AMP), a Bacillus thuringiensis var. kurstaki (Btk) toxin and an excipient; wherein the AMP comprises an amino acid sequence that is at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “KSCCPCYWGGCPWGQNCYPEGCGGPG” (SEQ ID NO: 1), or an agriculturally acceptable salt thereof; to the following: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof.

In some embodiments, the present disclosure provides a method of combating, controlling, or inhibiting a pest comprising, applying a pesticidally effective amount of a combination of an AMP and a Btk toxin, or an agricultural composition thereof; to the following: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the pest is selected from the group consisting of group consisting of: Amyelois transitella; Eumorpha achemon; Colias eurytheme; Caudra cautella; Amorbia humerosana; Pseudaletia unipuncta; Platyptilia carduidactyla; Datana major; Thyridopteryx ephemeraeformis; Hypercompe scribonia; Erionota thrax; Acleris gloverana; Phryganidia californica; Paleacrita merriccata; Grapholita packardi; Nymphula stagnata; Xylomyges curialis; Cydia pomonella; Acrobasis vaccinii; Evergestis rimosalis; Noctuid species; Agrotis ipsilon; Orgyia pseudotsugata; Erinnyis ello; Ennomos subsignaria; Lobesia botrana; Thymelicus lineola; Melissopus latiferreanus; Archips rosanus; Archips argyrospilia; Paralobesia viteana); Platynota stultana; Harrisina americana; Plathypena scabra; Dryocampa rubicunda; Batrachedra comosae; Lymantria dispar; Lambdina fiscellaria; Manduca quinquemaculata; Manduca sexta; Pieris rapae; Automeris io; Choristoneura pinus; Epiphyas postvittana; Diaphania hyalinata; Homadaula anisocentra; Choristoneura rosaceana; Syntomeida epilais; Playnota stultana; Sabulodes aegrotata; Papilio cresphontes; Argyrotaenia citrana; Grapholita molesta; Anarsia lineatella; Neophasia menapia; Argyrotaenia velutinana; Schizura concinna; Sibine stimulea; Heterocampa guttivitta; Estigmene acrea; Crambus sp.; Ennomos subsignaria; Alsophila pometaria; Choristoneura fumiferana; Lasiocampidae sp.; Thecla basilides; Ephestia elutella; Platynota idaeusalis; Anarsia lineatella; Peridroma saucia; Platynota flavedana; Anticarsia gemmatalis; Datana integerrima; Hyphantria cunea; Orgyia vetusta; Southern Diatraea crambidoides; Cylas formicarius; Anthonomus eugenii; Diaprepes abbreviatus; Otiorhynchus ovatus; Curculio caryae; Curculio occidentis; Lissorhoptrus oryzophilus; Hypera postica; Hypera zoilus; Euwallacea fornicatus; Euetheola humilis; Hypothenemus hampei; Listronotus maculicollis; Maladera castanea; Rhizotroqus majalis; Cotinis nitida; Popillia japonica; Phyllophaga sp.; Cyclocephala borealis; Anomala orientalis; Cyclocephala lurida; Sphenophorus parvulus; Sphenophorus apicalis; Sphenophorus cariosus; Sphenophorus inaequalis; Sphenophorus minimus; Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and Xanthogaleruca luteola.

In some embodiments, the present disclosure provides a method of combating, controlling, or inhibiting a pest comprising, applying a pesticidally effective amount of a combination of an AMP and a Btk toxin, or an agricultural composition thereof; to the following: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the pest is selected from the group consisting of: Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; Amyelois transitella; and Xanthogaleruca luteola.

In some embodiments, the present disclosure provides a method of combating, controlling, or inhibiting a pest comprising, applying a pesticidally effective amount of a combination of an AMP and a Btk toxin, or an agricultural composition thereof; to the following: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the pest is selected from the group consisting of: Plutella xylostella, Spodoptera exigua, and Amyelois transitella.

In some embodiments, the present disclosure provides a method of combating, controlling, or inhibiting a pest comprising, applying a pesticidally effective amount of a combination of an AMP and a Btk toxin, or an agricultural composition thereof; to the following: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the plant is a plant belonging to the genera: Brassica, Solanum, or Prunus.

In some embodiments, the present disclosure provides a method of combating, controlling, or inhibiting a pest comprising, applying a pesticidally effective amount of a combination of an AMP and a Btk toxin, or an agricultural composition thereof; to the following: the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof; wherein the plant is Brassica oleraceae, Solanum lycopersicum, or Prunus amygdalus.

### EXAMPLES

The Examples in this specification are not intended to, and should not be used to, limit the invention; they are provided only to illustrate the invention.

### Example 1. Av3b+Btk Toxins: Diamondback Moths on Cabbage

A randomized complete block design (RCBD) field experiment was performed to evaluate the effect of a combination of Av3b with Bacillus thuringiensis ssp. kurstaki (Btk) toxins on diamondback moth (Plutella xylostella) larvae number, in a field of cabbage (Brassica oleraceae var. capitata f. alba).

### Materials

The Av3b used here has an amino acid sequence of: “KSCCPCYWGGCPWGQNCYPEGCSGPK” (SEQ ID NO: 3). An exemplary method of obtaining Av3b is disclosed in PCT Application No. PCT/US2019/051093, the disclosure of which is incorporated herein by reference in its entirety.

To evaluate Btk toxins, the commercial product Leprotec® was used. Leprotec® consists of 14.49% Bacillus thuringiensis ssp. kurstaki (Btk) strain EVB-113-19 fermentation solids, spores, and insecticidal toxins, with a potency of 17,500 Cabbage Looper Units (CLU) per mg of product (equivalent to 76 billion CLU per gallon of product); and 85.51% other/inactive ingredients. Leprotec® is available from Vestaron Corporation (4717 Campus Dr. Suite 1200, Kalamazoo, MI 49008; website: https://www.vestaron.com/leprotec/; CAS number: 68038-71-1; lot number: 23J19M.).

### Doses

Doses of Av3b (active ingredient (AI) used in this example) were evaluated as follows:
Low: 9 grams of Active ingredient per acre (g AI/acre)
Med: 18 g AI/acre
High: 36 g AI/acre

Each dose of Av3b (i.e., low, med, and high) was combined with Btk toxins at an amount of 1 pint (pt) per acre (pt/acre). Spray solutions of either the low, med, or high dose of Av3b, were combined in a tank with 1 pt/acre of Btk toxins, and a non-ionic surfactant (NIS) at 0.125% v:v, for a total spray volume to be applied of 30 gallons/acre. NIS with water was used as the untreated control (UTC). NIS does not have insecticidal activity on its own and allowed the spray liquid to spread across the leaves (data not shown).

The RCBD consisted field plots that were 12×30 feet. Plants (cabbage) were sprayed in the field at various field rates of Av3b and observed for the presence of Plutella xylostella larvae. Spray treatments were applied 4 times (treatments: A, B, C, and D), with each spray treatment separated by 7 days. Larvae count per plant was taken 7 days after treatment D (i.e., 7 DA-D). The treatment scheme was replicated 4 times (n=4). Significance was determined by the ANOVA post-hoc test Fisher's Least Significant Difference (LSD) with α=0.05.

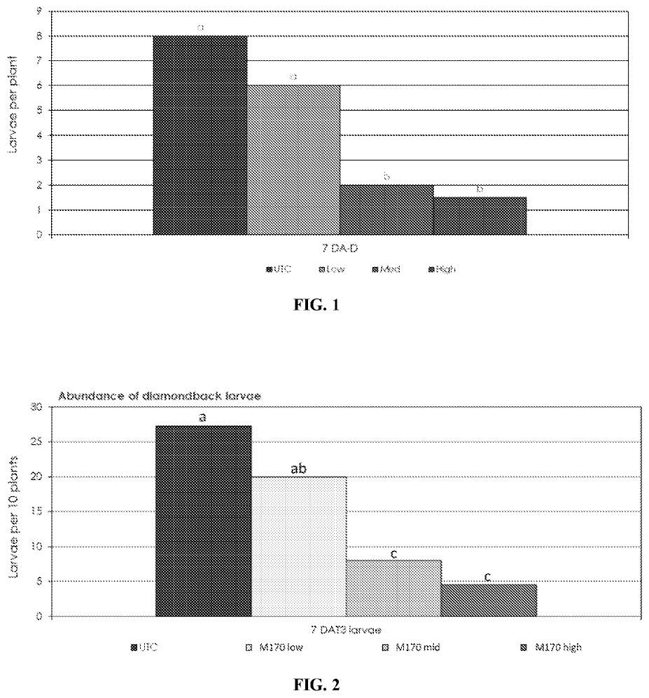

As shown in FIG. 1, plants treated with the med (18 g AI/acre) and high (36 g AI/acre) dose of Av3b had significantly fewer Plutella xylostella larvae as compared to the amount of larvae on plants treated with the low (9 g AI/acre) and control (UTC).

### Example 2. Av3bM170+Btk Toxins: Diamondback Moths on Cabbage

A randomized complete block design (RCBD) field experiment was performed to evaluate the effect of a combination of the Av3 Mutant Polypeptide (AMP), “Av3bM170,” with Bacillus thuringiensis ssp. kurstaki (Btk) toxins, on diamondback moth (Plutella xylostella) larvae number, in a field of cabbage (Brassica oleraceae var. capitata f. alba).

### Materials

The Av3bM170 has an amino acid sequence of:

(SEQ ID NO: 1)
“KSCCPCYWGGCPWGQNCYPEGCGGPG”.

To evaluate Btk toxins, the commercial product Leprotec® was used. Leprotec® consists of 14.49% Bacillus thuringiensis ssp. kurstaki (Btk) strain EVB-113-19 fermentation solids, spores, and insecticidal toxins, with a potency of 17,500 Cabbage Looper Units (CLU) per mg of product (equivalent to 76 billion CLU per gallon of product); and 85.51% other/inactive ingredients. Leprotec® is available from Vestaron Corporation (4717 Campus Dr. Suite 1200, Kalamazoo, MI 49008; website: https://www.vestaron.com/leprotec/; CAS number: 68038-71-1; lot number: 23J19M).

### Doses

Doses of Av3bM170 (AI used in this example) were evaluated as follows:
Low: 1 g AI/acre
Mid: 2 g AI/acre
High: 5 g AI/acre

Each dose of Av3bM170 (i.e., low, mid, and high) was combined with Btk toxins at an amount of 1 pint (pt) per acre (pt/acre). Spray solutions of either the low, med, or high dose of Av3bM170, were combined in a tank with 1 pt/acre of Btk toxins, and a non-ionic surfactant (NIS) at 0.25% v:v, for a total spray volume to be applied of 40 gallons/acre. NIS with water was used as the untreated control (UTC). NIS does not have insecticidal activity on its own and allowed the spray liquid to spread across the leaves (data not shown).

The RCBD consisted field plots that were 12×30 feet. Plants (cabbage) were sprayed in the field at various field rates of Av3bM170 and observed for the presence of Plutella xylostella larvae. Spray treatments were applied 3 times (treatments: 1, 2, and 3), with each spray treatment separated by 7 days. Larvae count per 10 plants was taken 7 days after treatment 3 (i.e., 7 DAT3). The treatment scheme was replicated 4 times (n=4). Significance was determined by the ANOVA post-hoc test Fisher's Least Significant Difference (LSD) with α=0.05.

As shown in FIG. 2, plants treated with the mid (2 g AI/acre) and high (5 g AI/acre) dose of Av3bM170had significantly fewer Plutella xylostella larvae as compared to the amount of larvae on plants treated with the low (1 g AI/acre) and control (UTC).

### Example 3. Comparison of Av3bM170+Btk Toxins

The results of Examples 1 and 2 were compared to determine the effect of (1) Av3b+Btk toxins vs. (2) Av3bM170+Btk toxins, on larval abundance in a randomized complete block design (RCBD) field experiment.

To make the comparison, the control relative to the UTC for all treatment rates was calculated according to Formula (II):

$$\% control relative to UTC = 100 - \frac{No . larvae per plant in treatment plot}{No . larvae per plant in UTC plot} * 100 \qquad Formula ( II )$$

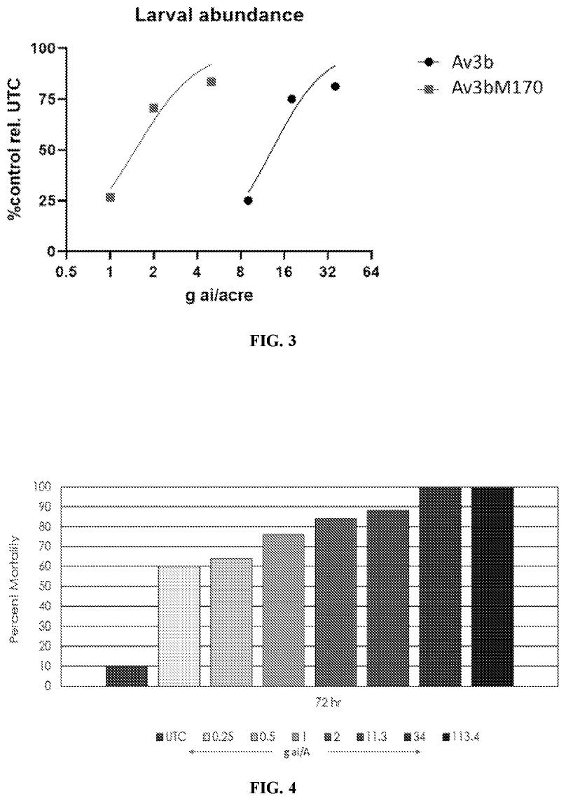

Next, the data was analyzed by logistic regression to calculate the dose resulting 50% larval control per plant relative to the UTC (EC50). FIG. 3.

When using Av3b+Btk toxins, the rate that caused 50% larval control per plant relative to the UTC was 13.17 g AI/acre. However, when using Av3bM170+Btk toxins, the rate that caused 50% larval control per plant relative to the UTC was 1.50 g AI/acre.

### Example 4. Av3b+Btk Toxins: Beet Armyworm on Tomato

A field-to-lab experiment was performed to evaluate the effect of a combination of Av3b and Btk toxins, against Beet armyworm (Spodoptera exigua) on tomato (Solanum lycopersicum).

The term “field-to-lab” refers to the following experimental design: spray solutions were applied to an agricultural field at various field rates (grams of active ingredient per acre or “g AI/acre”). The recently sprayed plant material was then brought into the lab (after less than 1 day), and insects (beet armyworm) were placed onto the leaves. Insect mortality was observed 72-hours after placement onto the sprayed leaves.

### Materials

The Av3b used here has an amino acid sequence of: “KSCCPCYWGGCPWGQNCYPEGCSGPK” (SEQ ID NO:3). An exemplary method of obtaining Av3b is disclosed in PCT Application No. PCT/US2019/051093, the disclosure of which is incorporated herein by reference in its entirety.

To evaluate Btk toxins, the commercial product DiPel® was used. DiPel consists of 54% Bacillus thuringiensis ssp. kurstaki (Btk) strain ABTS-351 fermentation solids, spores, and insecticidal toxins, with a potency of 32,000 Cabbage Looper Units (CLU) per mg of product (equivalent to 14.5 billion CLU per pound); and 46% other/inactive ingredients. DiPel® is available from Valent BioSciences® (1910 Innovation Way, Suite 100, Libertyville, IL 60049; https://www.valentbiosciences.com/).

### Doses

Doses of Av3b were evaluated as follows: 0.25 g AI/acre, 0.5 g AI/acre, 1 g AI/acre, 2 g AI/acre, 11.3 g AI/acre, 34 g AI/acre, and 113.4 g AI/acre.

Each dose of Av3b was combined with Btk toxins at an amount of 0.5 lbs per acre. Spray solutions of a given dose of Av3b, were combined in a tank with Btk toxins, and a non-ionic surfactant (NIS) at 0.125% v:v, for a total spray volume to be applied of 30 gallons/acre. NIS with water was used as the untreated control (UTC). NIS does not have insecticidal activity on its own and allowed the spray liquid to spread across the leaves (data not shown).

Spray treatments were applied to crops a single time. Then, within 24-hours, the sprayed leaves were harvested. Harvested leaves were placed in a petri dish with five 1^{st} instar Spodoptera exigua larvae. After 72-hours, percent mortality of the Spodoptera exigua larvae was assessed.

As shown in FIG. 4, was a positive rate-response to increasing doses of Av3b (i.e., the mortality was higher on leaves treated with higher concentrations of Av3b).

### Example 5. Av3b+Btk Toxins: Navel Orangeworm on Almonds

A randomized complete block design (RCBD) field experiment was performed to evaluate the effect of a combination of Av3bM170 with Bacillus thuringiensis ssp. kurstaki (Btk) toxins on percent almond (Prunus amygdalus) nut damage, when used against Navel Orangeworm (Amyelois transitella).

### Materials

The Av3bM170 has an amino acid sequence of:

(SEQ ID NO: 1)
“KSCCPCYWGGCPWGQNCYPEGCGGPG”.

To evaluate Btk toxins, the commercial product Leprotec® was used. Leprotec® consists of 14.49% Bacillus thuringiensis ssp. kurstaki (Btk) strain EVB-113-19 fermentation solids, spores, and insecticidal toxins, with a potency of 17,500 Cabbage Looper Units (CLU) per mg of product (equivalent to 76 billion CLU per gallon of product); and 85.51% other/inactive ingredients. Leprotec® is available from Vestaron Corporation (4717 Campus Dr. Suite 1200, Kalamazoo, MI 49008; website: https://www.vestaron.com/leprotec/; CAS number: 68038-71-1; lot number: 23J19M).

### Doses

Doses of Av3bM170 (AI used in this example) were evaluated as follows:
Low: 1 g AI/acre
Mid: 2 g AI/acre
High: 5 g AI/acre

Each dose of Av3bM170 (i.e., low, mid, and high) was combined with Btk toxins at an amount of 1 pint (pt) per acre (pt/acre). Spray solutions of either the low, med, or high dose of Av3bM170, were combined in a tank with 1 pt/acre of Btk toxins, and a non-ionic surfactant (NIS) at 0.125% v:v, for a total spray volume to be applied of 100 gallons/acre. NIS with water was used as the untreated control (UTC). NIS does not have insecticidal activity on its own and allowed the spray liquid to spread across the leaves (data not shown).

The RCBD consisted of 4 replicate single-tree plots. Percent nut damage was assessed in 100 randomly selected nuts, per treatment, per replicate (400 nuts in total per treatment).

Spray treatments were applied twice (A and B), each after hull-split occurrence (i.e., Jul. 9, 2021, and Jul. 22, 2021). “Hull-split” refers to the time of the growing season when the almond hulls split open to reveal the nut; this happens as the fruit ripens. After hull-split occurs, it is possible for pests to damage the nut and is often the beginning of pesticide treatment in almonds. Percent nut damage was evaluated after the second spray treatment application. Significance between treatment means was determined by Tukey's HSD test (p<0.05).

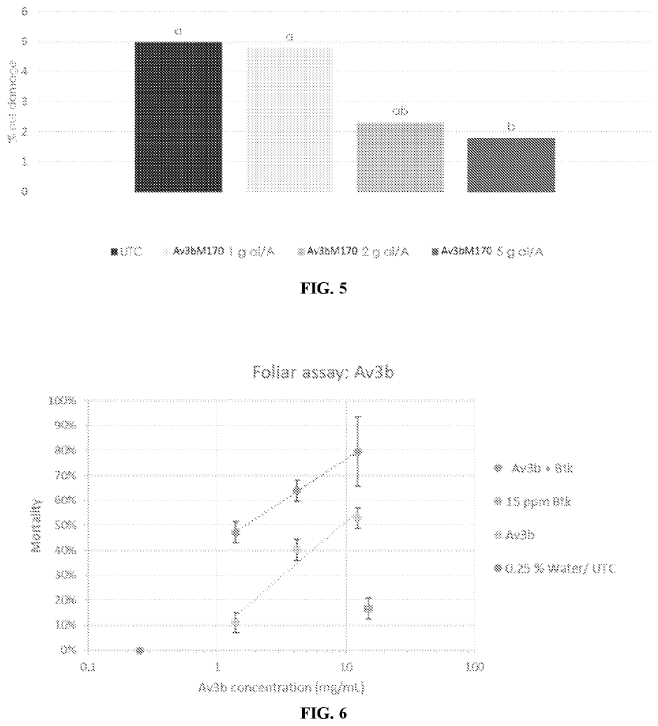

As shown in FIG. 5, plants treated with the highest dose of Av3bM170 (5 g AI/acre) had significantly less nut damage as compared to the untreated control (UTC), and the low (1 g AI/acre), and medium (2 g AI/acre) dose of Av3bM170.

### Example 6. Foliar Spray Assay: Av3b+Btk Toxins Against Fall Armyworm

A foliar spray bioassay was performed to evaluate the effect of a combination of Av3b with Bacillus thuringiensis ssp. kurstaki (Btk) toxins, on fall armyworm (Spodoptera frugiperda) mortality.

Briefly, Av3b (SEQ ID NO: 3) alone or in combination with Btk toxins, was sprayed on lettuce leaf disks and fed to neonate Spodoptera frugiperda. Three experimental replicates were performed. Percent mortality and LC₅₀ were assessed after 4 days. The treatments were as follows:
(1) Av3b+Btk
(2) Btk alone
(3) Av3b alone
(4) Control (0.25% NIS in water)

Av3b was evaluated at concentrations of 1.4 mg/mL, 4.2 mg/mL, and 12.4 mg/mL. To evaluate Btk toxins, the commercial product Leprotec® was used. Leprotec® consists of 14.49% Bacillus thuringiensis ssp. kurstaki (Btk) strain EVB-113-19 fermentation solids, spores, and insecticidal toxins, with a potency of 17,500 Cabbage Looper Units (CLU) per mg of product (equivalent to 76 billion CLU per gallon of product); and 85.51% other/inactive ingredients. Leprotec® is available from Vestaron Corporation (4717 Campus Dr. Suite 1200, Kalamazoo, MI 49008; website: https://www.vestaron.com/leprotec/; CAS number: 68038-71-1; lot number: 23J19M).

The sublethal dose (i.e., a dose resulting in approximately 20% of the population being killed, or ˜LD₂₀) of Leprotec® in fall armyworm was determined to be 15 ppm. Accordingly, this was the dose of Leprotec® used in combination with Av3b. A 0.25% solution of NIS with water was used as the untreated control (UTC).

FIG. 6 shows the results of the foliar spray bioassay. As shown in FIG. 6, the LC₅₀ for Av3b when combined with Bacillus thuringiensis ssp. kurstaki (Btk) toxins is 1.68 mg/mL.

### Example 7. Foliar Spray Assay: Av3bM170+Btk Toxins Against Fall Armyworm

A foliar spray bioassay was performed to evaluate the effect of a combination of Av3bM170 with Bacillus thuringiensis ssp. kurstaki (Btk) toxins, on fall armyworm (Spodoptera frugiperda) mortality.

Briefly, Av3bM170 (SEQ ID NO: 1) alone or in combination with Btk toxins, was sprayed on lettuce leaf disks and fed to neonate Spodoptera frugiperda. Three experimental replicates were performed. Percent mortality and LC₅₀ were assessed after 4 days. The treatments were as follows:
(1) Av3bM170+Btk
(2) Btk alone
(3) Av3bM170 alone
(4) Control (0.25% NIS in water)

Av3bM170 was evaluated at concentrations of 0.3 mg/mL, 1 mg/mL, and 3 mg/mL. To evaluate Btk toxins, the commercial product Leprotec® was used. Leprotec® consists of 14.49% Bacillus thuringiensis ssp. kurstaki (Btk) strain EVB-113-19 fermentation solids, spores, and insecticidal toxins, with a potency of 17,500 Cabbage Looper Units (CLU) per mg of product (equivalent to 76 billion CLU per gallon of product); and 85.51% other/inactive ingredients. Leprotec® is available from Vestaron Corporation (4717 Campus Dr. Suite 1200, Kalamazoo, MI 49008; website: https://www.vestaron.com/leprotec/; CAS number: 68038-71-1; lot number: 23J19M).

The sublethal dose (i.e., a dose resulting in approximately 20% of the population being killed, or ˜LD₂₀) of Leprotec® in fall armyworm was determined to be 15 ppm. Accordingly, this was the dose of Leprotec® used in combination with Av3b. A 0.25% solution of NIS with water was used as the untreated control (UTC).

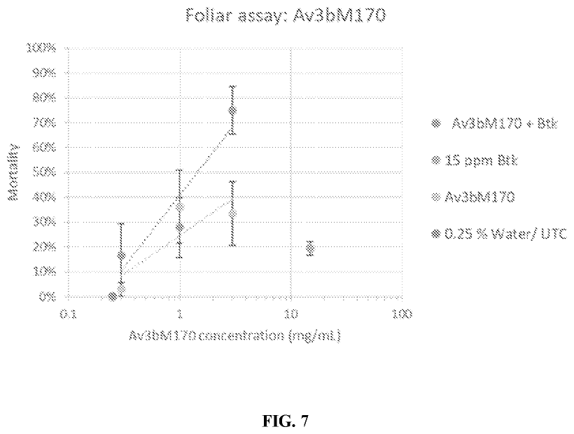

FIG. 7 shows the results of the foliar spray bioassay. As shown in FIG. 7, the LC₅₀ for Av3bM170 when combined with Bacillus thuringiensis ssp. kurstaki (Btk) toxins is 1.51 mg/mL.

1. A combination comprising an Av3 mutant polypeptide (AMP), and a Bacillus thuringiensis ssp. kurstaki (Btk) toxin; wherein the AMP comprises an amino acid sequence that is at least 95%, 96%, 97%, 97%, 98%, 99% or 100% identical to the amino acid sequence set forth in SEQ ID NO: 1, or a agriculturally acceptable salt thereof.

2. The combination of claim 1, wherein the AMP is a homopolymer of two or more AMPs, wherein the amino acid sequence of each AMP is the same, or different.

3. The combination of claim 1, wherein the AMP is a fused protein comprising two or more AMPs separated by a linker, and wherein the amino acid sequence of each AMP is the same, or different.

4. The combination of claim 3, wherein the linker is a cleavable or non-cleavable linker.

5. The combination of claim 4, wherein the linker has an amino acid sequence as set forth in any one of SEQ ID NOs: 4-16.

6. The combination or claim 5, wherein the linker is cleavable inside at least one of (i) the gut or hemolymph of an insect, and (ii) cleavable inside the gut of a mammal.

7. The combination of claim 1, wherein the Btk toxin is one or more fermentation solids, spores, or toxins isolated from a Bacillus thuringiensis ssp. kurstaki (Btk).

8. The combination of claim 7, wherein the Btk toxin is one or more fermentation solids, spores, or toxins isolated from a Bacillus thuringiensis ssp. kurstaki strain EVB-113-19, or ABTS-351.

9. A combination comprising one or more fermentation solids, spores, or toxins isolated from a Bacillus thuringiensis ssp. kurstaki strain EVB-113-19, and an Av3 mutant polypeptide (AMP) having an amino acid sequence set forth in SEQ ID NO: 1.

10. A combination comprising one or more fermentation solids, spores, or toxins isolated from a Bacillus thuringiensis ssp. kurstaki strain ABTS-351, and an Av3 mutant polypeptide (AMP) having an amino acid sequence set forth in any one of SEQ ID NO: 1.

11. A composition comprising the combination of claim 1, and at least one excipient.

12. A method of combating, controlling, or inhibiting a pest comprising, applying a pesticidally effective amount of the combination of claim 1, to the pest, a locus of the pest, a food supply of the pest, a habitat of the pest, or a breeding ground of the pest; a plant, a seed, a plant part, a locus of a plant, or an environment of a plant that is susceptible to an attack by the pest; an animal, a locus of an animal, or an environment of an animal susceptible to an attack by the pest; or a combination thereof.

13. The method of claim 12, wherein the pest is selected from the group consisting of: group consisting of: Amyelois transitella; Eumorpha achemon; Colias eurytheme; Caudra cautella; Amorbia humerosana; Pseudaletia unipuncta; Platyptilia carduidactyla; Datana major; Thyridopteryx ephemeraeformis; Hypercompe scribonia; Erionota thrax; Acleris gloverana; Phryganidia californica; Paleacrita merriccata; Grapholita packardi; Nymphula stagnata; Xylomyges curialis; Cydia pomonella; Acrobasis vaccinii; Evergestis rimosalis; Noctuid species; Agrotis ipsilon; Orgyia pseudotsugata; Erinnyis ello; Ennomos subsignaria; Lobesia botrana; Thymelicus lineola; Melissopus latiferreanus; Archips rosanus; Archips argyrospilia; Paralobesia viteana); Platynota stultana; Harrisina americana; Plathypena scabra; Dryocampa rubicunda; Batrachedra comosae; Lymantria dispar; Lambdina fiscellaria; Manduca quinquemaculata; Manduca sexta; Pieris rapae; Automeris io; Choristoneura pinus; Epiphyas postvittana; Diaphania hyalinata; Homadaula anisocentra; Choristoneura rosaceana; Syntomeida epilais; Playnota stultana; Sabulodes aegrotata; Papilio cresphontes; Argyrotaenia citrana; Grapholita molesta; Anarsia lineatella; Neophasia menapia; Argyrotaenia velutinana; Schizura concinna; Sibine stimulea; Heterocampa guttivitta; Estigmene acrea; Crambus sp.; Ennomos subsignaria; Alsophila pometaria; Choristoneura fumiferana; Lasiocampidae sp.; Thecla basilides; Ephestia elutella; Platynota idaeusalis; Anarsia lineatella; Peridroma saucia; Platynota flavedana; Anticarsia gemmatalis; Datana integerrima; Hyphantria cunea; Orgyia vetusta; Southern Diatraea crambidoides; Cylas formicarius; Anthonomus eugenii; Diaprepes abbreviatus; Otiorhynchus ovatus; Curculio caryae; Curculio occidentis; Lissorhoptrus oryzophilus; Hypera postica; Hypera zoilus; Euwallacea fornicatus; Euetheola humilis; Hypothenemus hampei; Listronotus maculicollis; Maladera castanea; Rhizotroqus majalis; Cotinis nitida; Popillia japonica; Phyllophaga sp.; Cyclocephala borealis; Anomala orientalis; Cyclocephala lurida; Sphenophorus parvulus; Sphenophorus apicalis; Sphenophorus cariosus; Sphenophorus inaequalis; Sphenophorus minimus; Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and Xanthogaleruca luteola.

14. The method of claim 13, wherein the pest is selected from the group consisting of: Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; Amyelois transitella; and Xanthogaleruca luteola.

15. The method of claim 14, wherein the pest is selected from the group consisting of: Plutella xylostella, Spodoptera exigua, and Amyelois transitella.

16. The method of claim 12, wherein the plant is a plant belonging to the genera: Brassica, Solanum, or Prunus.

17. The method of claim 16, wherein the plant is Brassica oleraceae, Solanum lycopersicum, or Prunus amygdalus.
